(19) United States
(12) Patent Application Publication
(10) Pub. No.: US 2023/0337532 A1
(43) Pub. Date: Oct. 19, 2023

(54) TRIAZINE COMPOUND, MATERIAL FOR ORGANIC LIGHT EMITTING DIODE, ELECTRON TRANSPORT MATERIAL FOR ORGANIC LIGHT EMITTING DIODE, AND ORGANIC LIGHT EMITTING DIODE

(71) Applicant: TOSOH CORPORATION, Yamaguchi (JP)

(72) Inventors: Youhei ONO, Kanagawa (JP); Kana OIKE, Kanagawa (JP); Fuminari UEHARA, Kanagawa (JP); Kazuki HATTORI, Kanagawa (JP); Yasuhiro TAKAHASHI, Kanagawa (JP); Tomohiro SHONO, Yamaguchi (JP); Naoki UCHIDA, Kanagawa (JP)

(73) Assignee: TOSOH CORPORATION, Yamaguchi (JP)

(21) Appl. No.: 18/027,785

(22) PCT Filed: Oct. 1, 2021
(86) PCT No.: PCT/JP2021/036442

§ 371 Date: Mar. 22, 2023

(30) Foreign Application Priority Data
Oct. 5, 2020 (JP) 2020-168389
Oct. 5, 2020 (JP) 2020-168719
Oct. 29, 2020 (JP) 2020-181703

(51) Int. Cl. H10K 85/60; C07D 251/24; C09K 11/06

(52) U.S. Cl. CPC H10K 85/654; C07D 251/24; C09K 11/06; H10K 85/626; H10K 85/615; H10K 50/16

(57) ABSTRACT

A triazine compound represented by formula (1) is provided. In the formula (1),

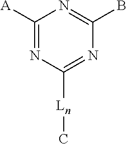

A and B each independently represent an aryl group having 6 to 20 carbon atoms;
L represents a phenyl group or a naphthyl group;
n is 0 or 1; and
C represents any one of groups X, Y, and Z below,

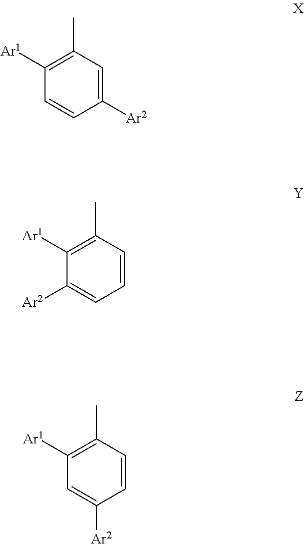

wherein Ar¹ and Ar² each independently represent an aromatic hydrocarbon group or a pyridyl group.

### TECHNICAL FIELD

The present invention relates to a triazine compound, a material for an organic light emitting diode, an electron transport material for an organic light emitting diode, and an organic light emitting diode.

### BACKGROUND ART

Organic light emitting diodes have been used in applications including not only small-sized displays but also large-sized televisions and lighting fixtures, and have been intensively developed.
Patent Document 1: Japanese Unexamined Patent Application (Translation of PCT application), Publication No. 2019-512499
Patent Document 2: Japanese Unexamined Patent Application, Publication No. 2018-95562
Patent Document 3: PCT International Publication No. WO2015/111848

### DISCLOSURE OF THE INVENTION

### Problems to be Solved by the Invention

In recent years, market demands for organic light emitting diodes have become increasingly stringent, and there is a need to develop a material exhibiting excellent characteristics in terms of current efficiency, driving voltage, and long life. In this regard, Patent Document 1 discloses a triazine compound substituted with different substituents at 2-, 4-, and 6-positions, Patent Document 2 discloses a triazine compound having a 1,2-phenylene moiety, and Patent Document 3 discloses an azine compound substituted with a phenyl group at 2-position.

However, organic light emitting diodes which are provided with an electron transport layer containing the compounds disclosed in Patent Documents 1 to 3 are insufficient in terms of driving voltage characteristics and driving lifetime characteristics, and there is a desire for further improvements.

An aspect of the present invention is directed to providing a triazine compound, a material for an organic light emitting diode, and an electron transport material for an organic light emitting diode, which contribute to the manufacture of an organic light emitting diode that has a low driving voltage and excellent durability.

In addition, another aspect of the present invention is directed to providing an organic light emitting diode that has a low driving voltage and excellent durability.

### Means for Solving the Problems

According to an aspect of the present invention, a triazine compound represented by formula (1) is provided: Triazine compound represented by formula (1).

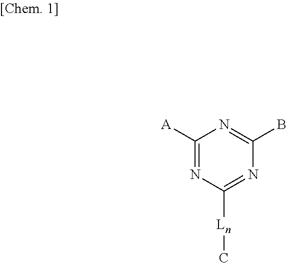

wherein in the formula (1),
A and B each independently represent an aryl group having 6 to 20 carbon atoms;
L represents a phenyl group or a naphthyl group;
n is 0 or 1; and
C represents any one of groups X, Y, and Z below.

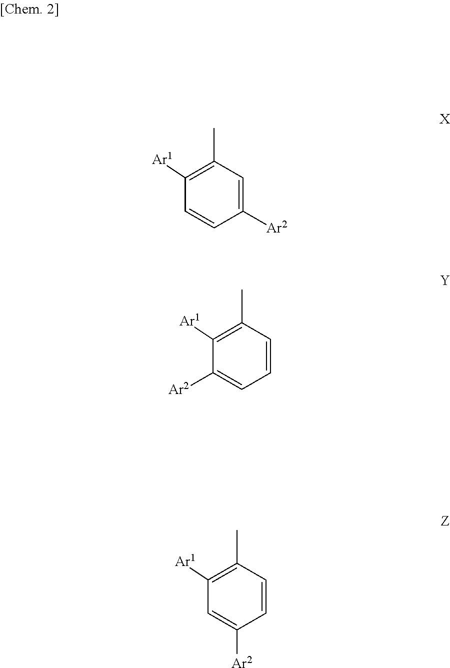

Ar¹ and Ar² each independently represent an aromatic hydrocarbon group or a pyridyl group.

According to an aspect of the present invention, the triazine compound represented by the formula (1) includes triazine compounds represented by the following formulas X(1), Y(1), and Z(1). A, B, L, n, Ar¹, and Ar² are each defined in the triazine compounds represented by the formulas X(1), Y(1), and Z(1).

According to an aspect of the present invention, a triazine compound represented by the formula X(1) is provided:

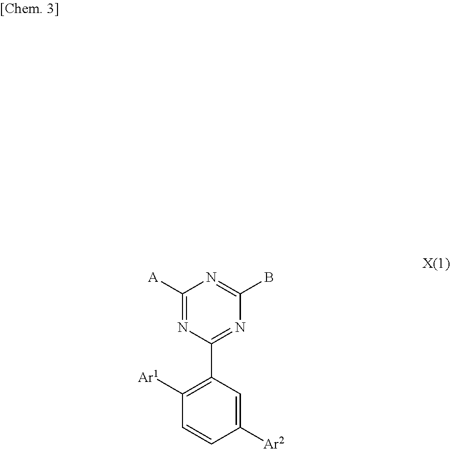

wherein in the case of the formula X(1),
A represents any one group selected from formulas X(A-1) to X(A-9):

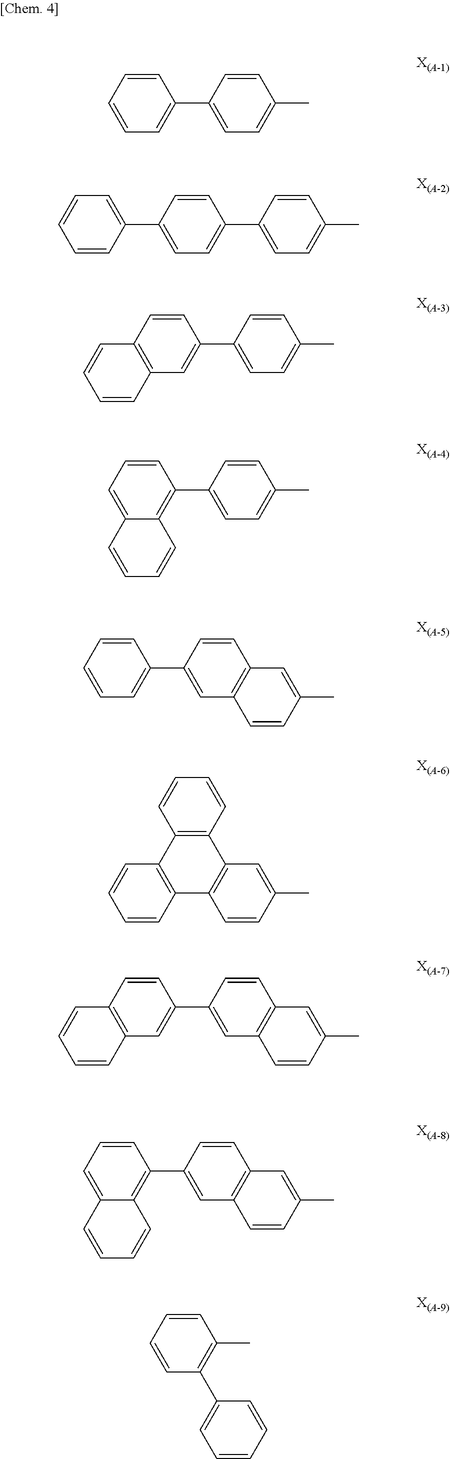

B represents any one group selected from formulas X(B-1) to X(B-15):

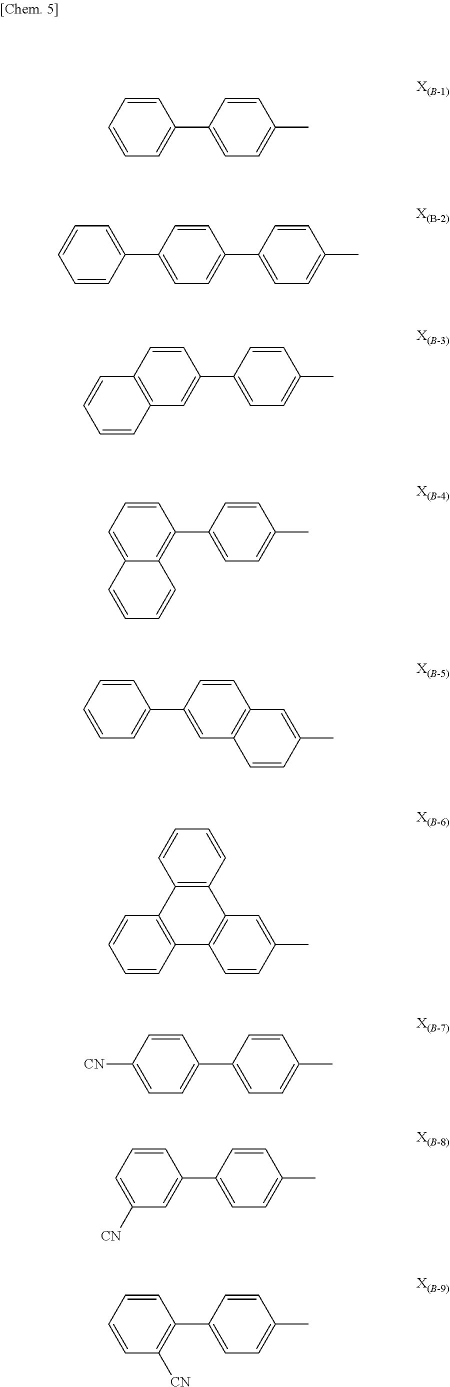

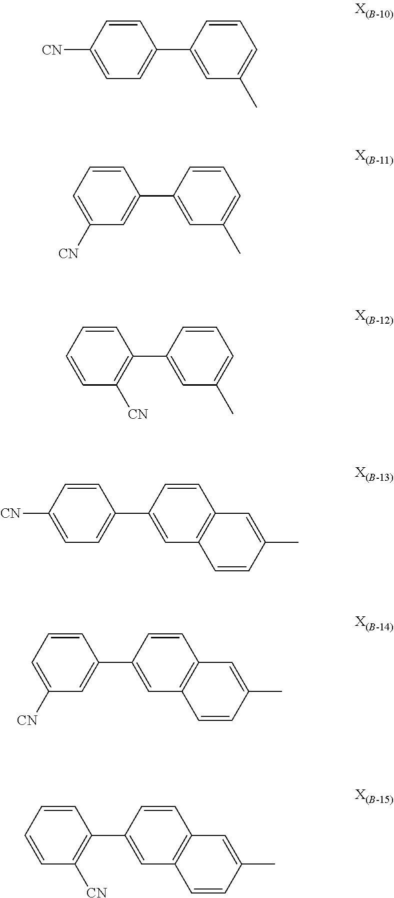

Ar¹ represents:
an aryl group having 6 to 30 carbon atoms, optionally substituted with one or more selected from the group consisting of an alkyl group having 1 to 12 carbon atoms, a cycloalkyl group having 3 to 20 carbon atoms, a cyano group, a diarylboryl group, and a phosphine oxide group, or
a pyridyl group optionally substituted with a methyl group or
a phenyl group, and Are represents: an aryl group having 6 to 30 carbon atoms, optionally substituted with one or more selected from the group consisting of an alkyl group having 1 to 12 carbon atoms, a cycloalkyl group having 3 to 20 carbon atoms, a cyano group, a diarylboryl group, and a phosphine oxide group, or a pyridyl group optionally substituted with a methyl group or a phenyl group.

According to another aspect of the present invention, a triazine compound represented by formula Y(1) is provided:

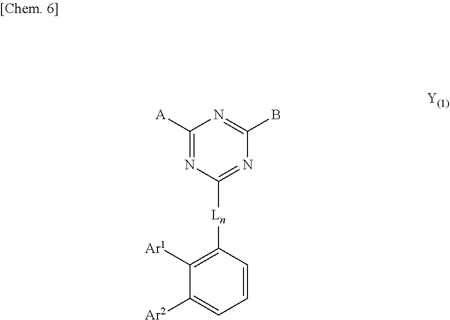

wherein in the case of the formula Y(1),
A represents an aryl group having 6 to 20 carbon atoms;
B represents:
   an aryl group having 6 to 20 carbon atoms, optionally substituted with one or more selected from the group consisting of an alkyl group having 1 to 12 carbon atoms, a cycloalkyl group having 3 to 20 carbon atoms, a cyano group, a diarylboryl group, and a phosphine oxide group, or
   a heteroaryl group having 4 to 30 carbon atoms and an oxygen or sulfur atom;
Ar¹ and Ar² each independently represent:
   an aryl group having 6 to 26 carbon atoms, optionally substituted with one or more selected from the group consisting of an alkyl group having 1 to 12 carbon atoms, a cycloalkyl group having 3 to 20 carbon atoms, a cyano group, a diarylboryl group, and a phosphine oxide group, or
   a pyridinyl group optionally substituted with a methyl group or a phenyl group;
   n represents an integer of 0 to 1; and L represents any one group selected from formulas Y(2-1) to Y(2-5).

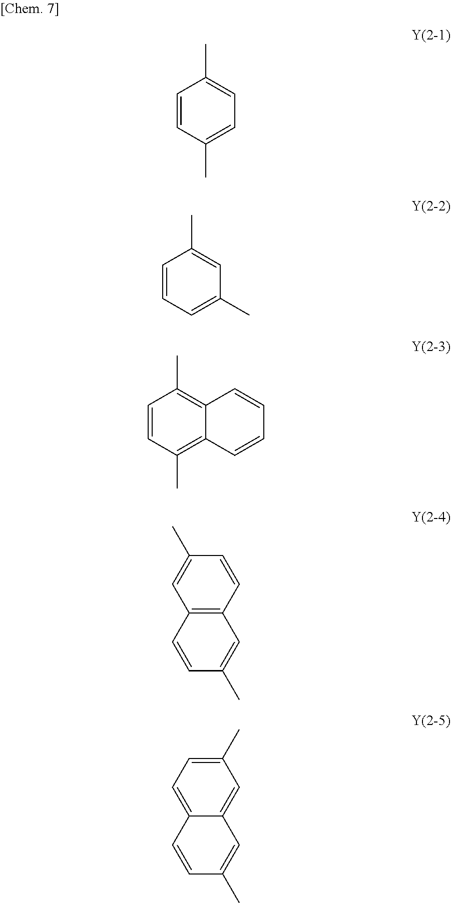

According to still another aspect of the present invention, a triazine compound represented by formula Z(1) is provided:

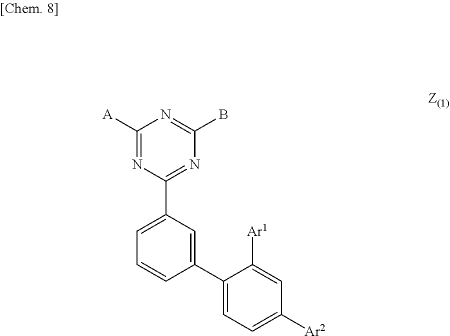

wherein in the case of the formula Z(1),
A represents a phenyl group, a biphenylyl group, or a naphthyl group, each optionally substituted with one or more substituent(s) selected from the group consisting of a fluorine atom, a methyl group, and a cyano group;
B represents a phenyl group, a biphenylyl group, or a naphthyl group, each optionally substituted with one or more substituent(s) selected from the group consisting of a fluorine atom, a methyl group, and a cyano group;
Ar¹ represents a phenyl group or a naphthyl group, each optionally substituted with one or more substituent(s) selected from the group consisting of a fluorine atom, a methyl group, and a cyano group; and Are represents a phenyl group or a naphthyl group, each optionally substituted with one or more substituent(s) selected from the group consisting of a fluorine atom, a methyl group, and a cyano group.

According to still another aspect of the present invention, a material for an organic light emitting diode which contains the triazine compound is provided. According to still another aspect of the present invention, an electron transport material for an organic light emitting diode which contains the triazine compound is provided. According to still another aspect of the present invention, an organic light emitting diode containing the triazine compound is provided.

### Effects of the Invention

According to an aspect of the present invention, a triazine compound, a material for an organic light emitting diode, and an electron transport material for an organic light emitting diode, which contribute to the manufacture of an organic light emitting diode that has a low driving voltage and excellent durability, can be provided. According to another aspect of the present invention, an organic light emitting diode that has a low driving voltage and excellent durability can be provided.

### BRIEF DESCRIPTION OF THE DRAWINGS

FIG. 1 is a schematic cross-sectional view showing an example of a layered structure of an organic light emitting diode containing a triazine compound according to an aspect of the present invention; and

FIG. 2 is a schematic cross-sectional view showing an example of a layered structure of an organic light emitting diode containing a triazine compound according to an aspect of the present invention (Element Example—1, etc.).

### PREFERRED MODE FOR CARRYING OUT THE INVENTION

Hereinafter, a triazine compound according to an aspect of the present invention will be described in more detail.

### <Triazine Compound>

According to an aspect of the present invention, a triazine compound represented by formula (1) is provided: Triazine compound represented by formula (1).

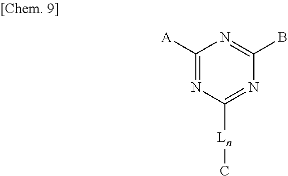

wherein in the formula (1),
A and B each independently represent an aryl group having 6 to 20 carbon atoms;
L represents a phenyl group or a naphthyl group;
n is 0 or 1; and
C represents any one of groups X, Y, and Z below.

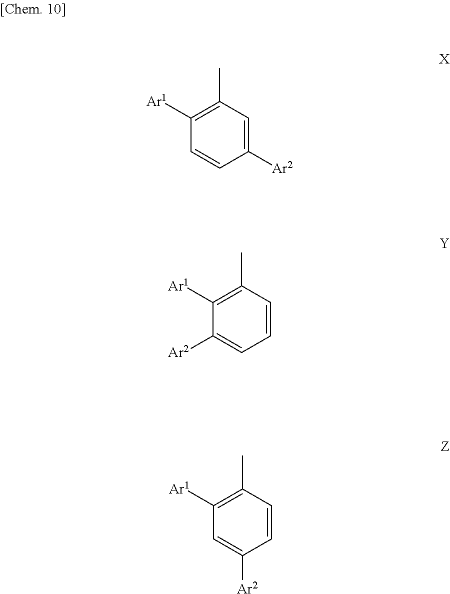

Ar¹ and Ar² each independently represent an aromatic hydrocarbon group or a pyridyl group.

The triazine compound represented by the formula (1) includes embodiments represented by the following formulas X(1), Y(1), and Z(1). Hereinafter, the embodiments represented by the formulas X(1), Y(1), and Z(1) are described.

### <Triazine Compound X(1)>

A triazine compound according to an aspect of the present invention is represented by formula X(1):

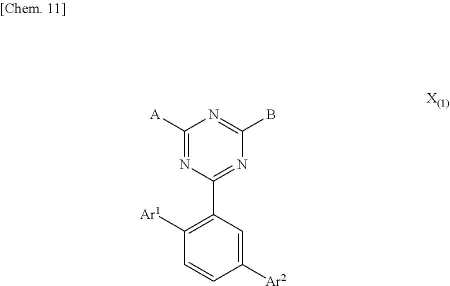

wherein in the case of the formula X(1),
A represents any one group selected from formulas X(A-1) to X(A-9):

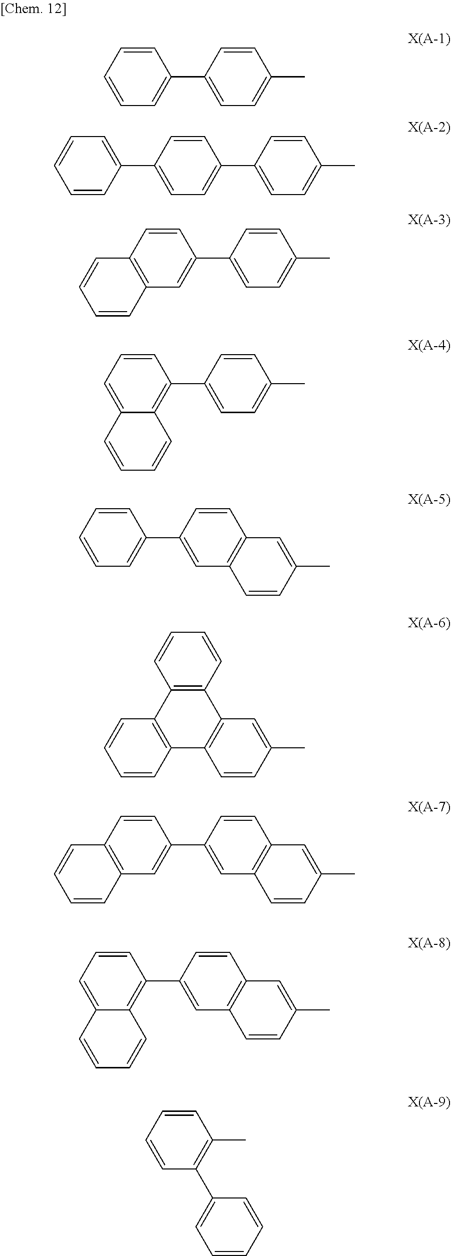

B represents any one group selected from formulas X(B-1) to X(B-15):

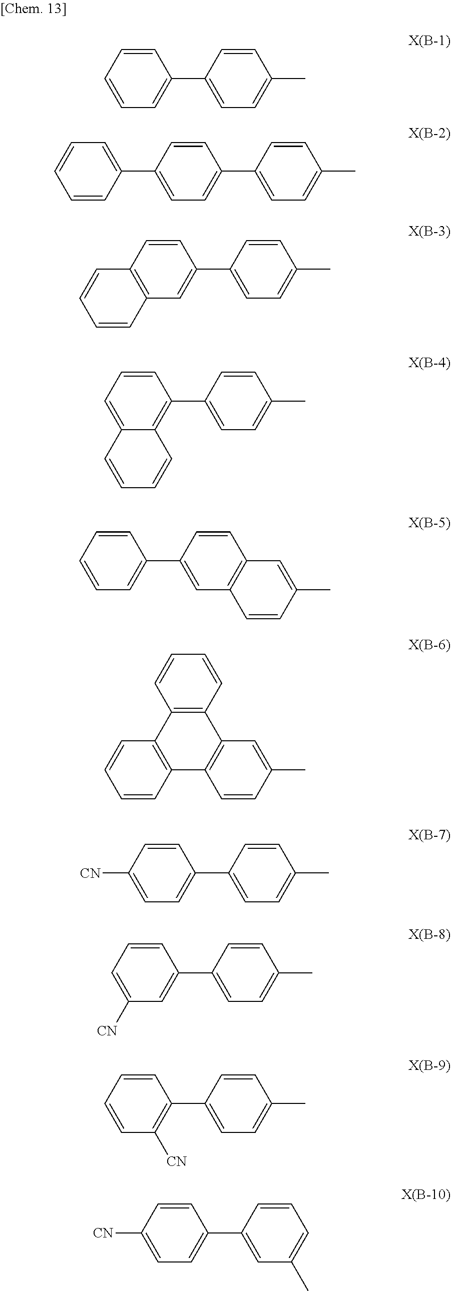

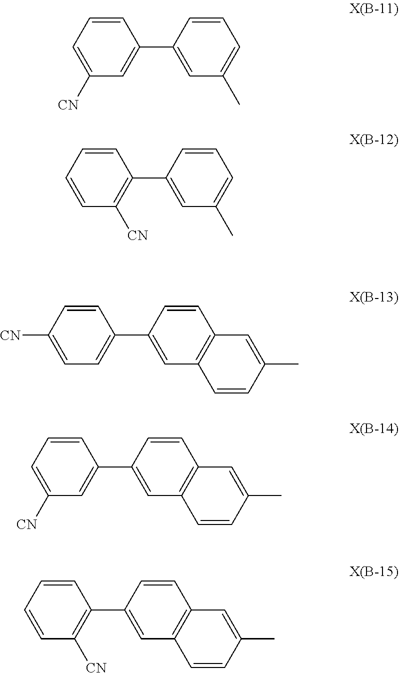

Ar¹ represents: an aryl group having 6 to 30 carbon atoms, optionally substituted with one or more selected from the group consisting of an alkyl group having 1 to 12 carbon atoms, a cycloalkyl group having 3 to 20 carbon atoms, a cyano group, a diarylboryl group, and a phosphine oxide group, or a pyridyl group optionally substituted with a methyl group or a phenyl group; and Ar² represents: an aryl group having 6 to 30 carbon atoms, optionally substituted with one or more selected from the group consisting of an alkyl group having 1 to 12 carbon atoms, a cycloalkyl group having 3 to 20 carbon atoms, a cyano group, a diarylboryl group, and a phosphine oxide group, or
a pyridyl group optionally substituted with a methyl group or
a phenyl group.
   [Preferable Combinations of A, B, Ar¹, and Ar²]

In the triazine compound represented by the formula X(1), first to eighth aspects, which are preferable combinations of A, B, Ar¹, and Ar² are as follows.

### First Aspect

A represents any one group selected from the formulas X(A-1) to X(A-9);
Ar¹ represents an aryl group having 6 to 30 carbon atoms, optionally substituted with one or more selected from the group consisting of an alkyl group having 1 to 12 carbon atoms, a cycloalkyl group having 3 to 20 carbon atoms, a cyano group, a diarylboryl group, and a phosphine oxide group; and
Ar² represents an aryl group having 6 to 30 carbon atoms, optionally substituted with one or more selected from the group consisting of an alkyl group having 1 to 12 carbon atoms, a cycloalkyl group having 3 to 20 carbon atoms, a cyano group, a diarylboryl group, and a phosphine oxide group.

### Second Aspect

B represents any one group selected from the formulas X(B-1) to X(B-4), (B-7) to X(B-8), and (B-10) to X(B-11).

### Third Aspect

A represents any one group selected from the formulas X(A-1) to X(A-9);
Ar¹ represents an aryl group having 6 to 30 carbon atoms, optionally substituted with one or more selected from the group consisting of an alkyl group having 1 to 12 carbon atoms, a cycloalkyl group having 3 to 20 carbon atoms, a cyano group, a diarylboryl group, and a phosphine oxide group; and
Ar² represents an aryl group having 6 to 30 carbon atoms, optionally substituted with one or more selected from the group consisting of an alkyl group having 1 to 12 carbon atoms, a cycloalkyl group having 3 to 20 carbon atoms, a cyano group, a diarylboryl group, and a phosphine oxide group.

### Fourth Aspect

A represents any one group selected from the formulas X(A-1) to X(A-4);
B represents a group selected from the formulas X(B-1) to X(B-4), X(B-7) to X(B-8), and X(B-10) to X(B-11);
Ar¹ represents an aryl group having 6 to 30 carbon atoms, optionally substituted with one or more selected from the group consisting of an alkyl group having 1 to 12 carbon atoms, a cycloalkyl group having 3 to 20 carbon atoms, a cyano group, a diarylboryl group, and a phosphine oxide group; and
Ar² represents an aryl group having 6 to 30 carbon atoms, optionally substituted with one or more selected from the group consisting of an alkyl group having 1 to 12 carbon atoms, a cycloalkyl group having 3 to 20 carbon atoms, a cyano group, a diarylboryl group, and a phosphine oxide group.

### Fifth Aspect

Ar¹ represents a phenyl group, a 1-naphthalenyl group, a 2-naphthalenyl group, a 2-biphenylyl group, a 3-biphenylyl group, a 4-biphenylyl group, a 2-(1-naphthalenyl)phenyl group, a 3-(1-naphthalenyl)phenyl group, a 4-(1-naphthalenyl)phenyl group, a 2-(2-naphthalenyl)phenyl group, a 3-(2-naphthalenyl)phenyl group, a 4-(2-naphthalenyl)phenyl group, a 4-phenylnaphthalen-1-yl group, a 5-phenylnaphthalen-1-yl group, a 6-phenylnaphthalen-2-yl group, or a 7-phenylnaphthalen-2-yl group, each optionally substituted with one or more selected from the group consisting of an alkyl group having 1 to 12 carbon atoms, a cycloalkyl group having 3 to 20 carbon atoms, a cyano group, a diarylboryl group, and a phosphine oxide group; and
Ar² represents a phenyl group, a 1-naphthalenyl group, a 2-naphthalenyl group, a 2-biphenylyl group, a 3-biphenylyl group, a 4-biphenylyl group, a 2-(1-naphthalenyl)phenyl group, a 3-(1-naphthalenyl)phenyl group, a 4-(1-naphthalenyl)phenyl group, a 2-(2-naphthalenyl)phenyl group, a 3-(2-naphthalenyl)phenyl group, a 4-(2-naphthalenyl)phenyl group, a 4-phenylnaphthalen-1-yl group, a 5-phenylnaphthalen-1-yl group, a 6-phenylnaphthalen-2-yl group, or a 7-phenylnaphthalen-2-yl group, each optionally substituted with one or more selected from the group consisting of an alkyl group having 1 to 12 carbon atoms, a cycloalkyl group having 3 to 20 carbon atoms, a cyano group, a diarylboryl group, and a phosphine oxide group.

### Sixth Aspect

Ar¹ represents a phenyl group, a 1-naphthalenyl group, a 2-naphthalenyl group, a 2-biphenylyl group, a 3-biphenylyl group, or a 4-biphenylyl group, each optionally substituted with one or more selected from the group consisting of an alkyl group having 1 to 12 carbon atoms, a cycloalkyl group having 3 to 20 carbon atoms, a cyano group, a diarylboryl group, and a phosphine oxide group; and
Ar² represents a phenyl group, a 1-naphthalenyl group, a 2-naphthalenyl group, a 2-biphenylyl group, a 3-biphenylyl group, or a 4-biphenylyl group, each optionally substituted with one or more selected from the group consisting of an alkyl group having 1 to 12 carbon atoms, a cycloalkyl group having 3 to 20 carbon atoms, a cyano group, a diarylboryl group, and a phosphine oxide group.

### Seventh Aspect

A represents any one group selected from the formulas X(A-1) to X(A-4);
B represents any one group selected from the formulas X(B-1) to X(B-4);
Ar¹ represents a phenyl group, a 1-naphthalenyl group, a 2-naphthalenyl group, a 2-biphenylyl group, a 3-biphenylyl group, or a 4-biphenylyl group; and
Ar² represents a phenyl group, a 1-naphthalenyl group, a 2-naphthalenyl group, a 2-biphenylyl group, a 3-biphenylyl group, or a 4-biphenylyl group.

### Eighth Aspect

A represents a group represented by the formula X(A-1);
B represents a group represented by the formula X(B-1);
Ar¹ represents a phenyl group, a 1-naphthalenyl group, a 2-naphthalenyl group, a 2-biphenylyl group, a 3-biphenylyl group, or a 4-biphenylyl group; and
Ar² represents a phenyl group, a 1-naphthalenyl group, a 2-naphthalenyl group, a 2-biphenylyl group, a 3-biphenylyl group, or a 4-biphenylyl group.

Hereinafter, the triazine compound represented by the formula X(1) may be referred to as a triazine compound X(1). Definitions of the substituents in the triazine compound X(1), and preferable specific examples thereof are as follows.

### [A and B]

In the case of the formula X(1),
A represents any one group selected from the formulas X(A-1) to X(A-9),
B represents any one group selected from the formulas X(B-1) to X(B-15).

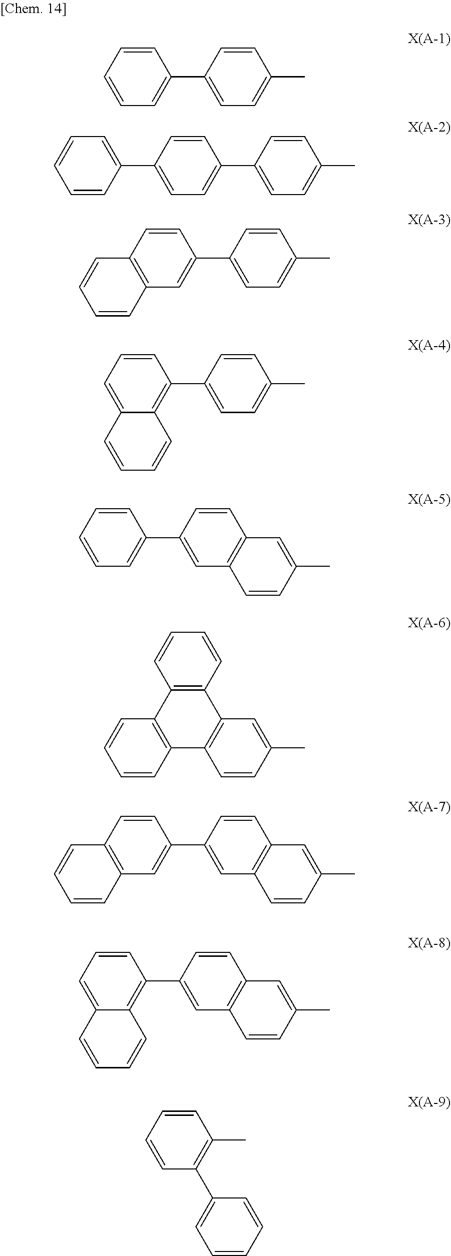

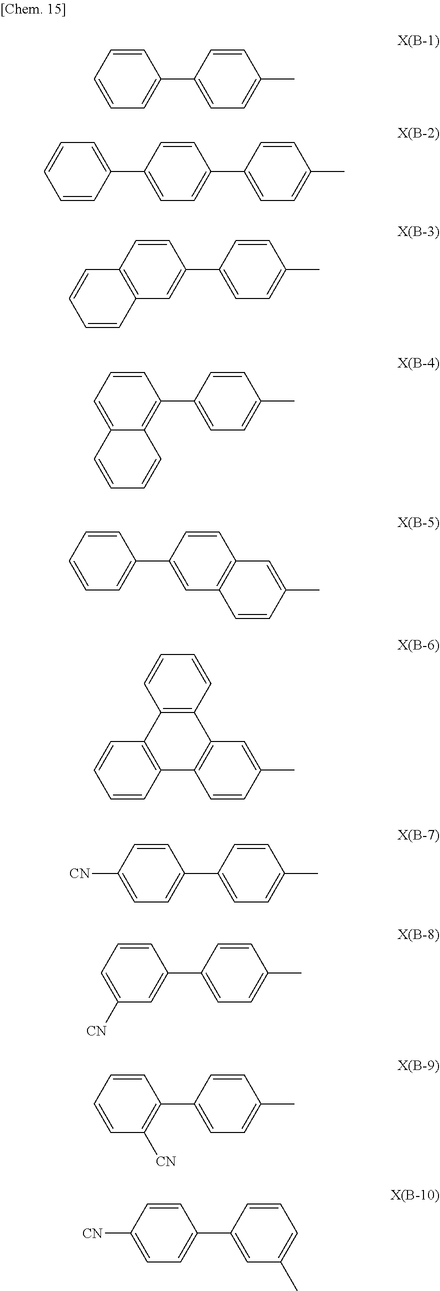

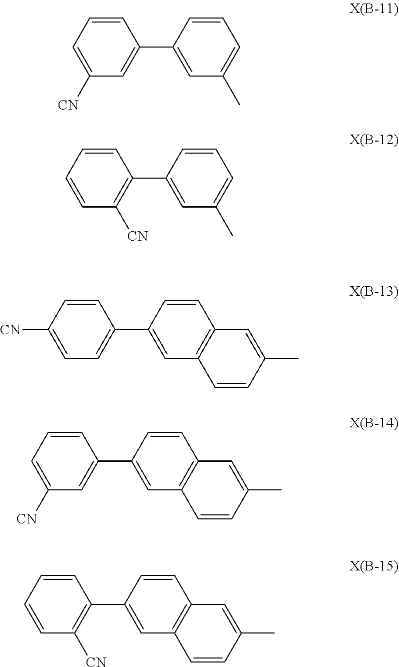

A represents any one group selected from the formulas X(A-1) to X(A-9), and preferably represents any one group selected from the formulas X(A-1) to X(A-4).

B represents any one group selected from the formulas X(B-1) to X(B-15), preferably from the formulas X(B-1) to X(B-4), X(B-7), X(B-8), X(B-10), and X(B-11), and particularly preferably from formulas X(B-1) to X(B-4).

When a group selected as B includes no cyano group, the combination of A and B is preferably a combination of identical groups, like, for example, the combination of the group represented by the X(A-1) and the group represented by X(B-1).

[Ar¹ and Ar²]

Ar¹ represents:
an aryl group having 6 to 30 carbon atoms, optionally substituted with one or more selected from the group consisting of an alkyl group having 1 to 12 carbon atoms, a cycloalkyl group having 3 to 20 carbon atoms, a cyano group, a diarylboryl group, and a phosphine oxide group, or
a pyridyl group optionally substituted with a methyl group or
a phenyl group;
Ar² represents:
an aryl group having 6 to 30 carbon atoms, optionally substituted with one or more selected from the group consisting of an alkyl group having 1 to 12 carbon atoms, a cycloalkyl group having 3 to 20 carbon atoms, a cyano group, a diarylboryl group, and a phosphine oxide group, or
a pyridyl group optionally substituted with a methyl group or
a phenyl group.

Preferable examples of the aryl group having 6 to 30 carbon atoms in Ar¹ and Ar² include a phenyl group, a 1-naphthalenyl group, a 2-naphthalenyl group, a 2-biphenylyl group, a 3-biphenylyl group, a 4-biphenylyl group, a 2-(1-naphthalenyl)phenyl group, a 3-(1-naphthalenyl)phenyl group, a 4-(1-naphthalenyl)phenyl group, a 2-(2-naphthalenyl)phenyl group, a 3-(2-naphthalenyl)phenyl group, a 4-(2-naphthalenyl)phenyl group, a 4-phenylnaphthalen-1-yl group, a 5-phenylnaphthalen-1-yl group, a 6-phenylnaphthalen-2-yl group, a 7-phenylnaphthalen-2-yl group, a 2-phenanthrenyl group, a 3-phenanthrenyl group, a 9-phenanthrenyl group, a 9-anthracenyl group, a p-terphenyl group, and a 2-triphenylenyl group.

These groups are optionally substituted with an alkyl group having 1 to 12 carbon atoms, a cycloalkyl group having 3 to 20 carbon atoms, a cyano group, a diarylboryl group, or a phosphine oxide group, more preferably are an unsubstituted phenyl group, an unsubstituted 1-naphthalenyl group, an unsubstituted 2-naphthalenyl group, an unsubstituted 2-biphenylyl group, an unsubstituted 3-biphenylyl group, or an unsubstituted 4-biphenylyl group, and particularly preferably a phenyl group, a 2-naphthalenyl group, a 2-biphenylyl group, or 4-biphenylyl group.

### Specific Examples of Triazine Compound X(1)

Among the triazine compounds according to an aspect of the present invention represented by the formula X(1), specific examples of particularly preferable compounds include the following X(1-1) to X(1-80), but the triazine compound according to an aspect of the present invention is not limited thereto.

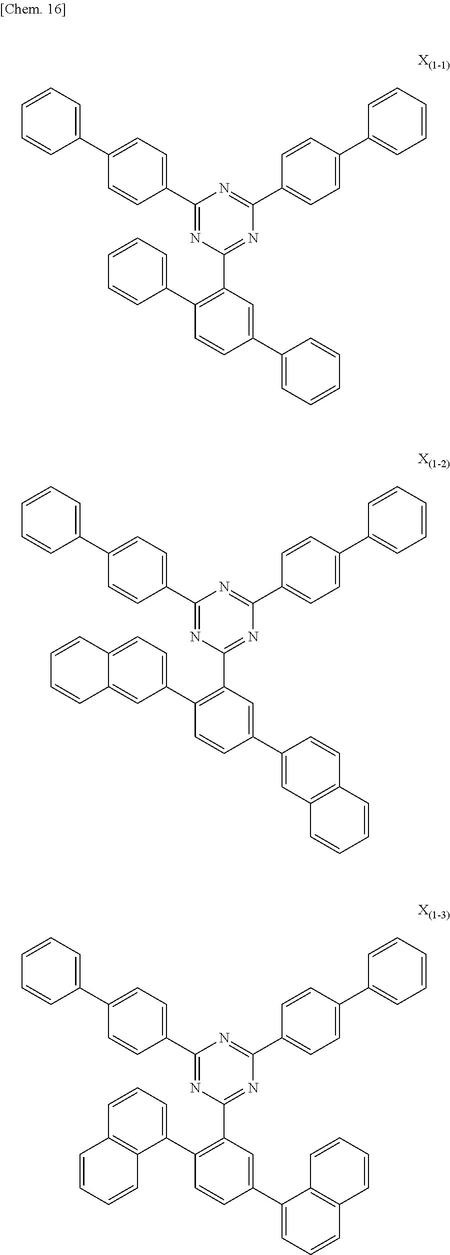 [SMILES: c1ccc(-c2ccc(-c3nc(-c4ccc(-c5ccccc5)cc4)nc(-c4cc(-c5ccccc5)ccc4-c4ccccc4)n3)cc2)cc1 ; c1ccc(-c2ccc(-c3nc(-c4ccc(-c5ccccc5)cc4)nc(-c4cc(-c5ccc6ccccc6c5)ccc4-c4ccc5ccccc5c4)n3)cc2)cc1 ; c1ccc(-c2ccc(-c3nc(-c4ccc(-c5ccccc5)cc4)nc(-c4cc(-c5cccc6ccccc56)ccc4-c4cccc5ccccc45)n3)cc2)cc1]

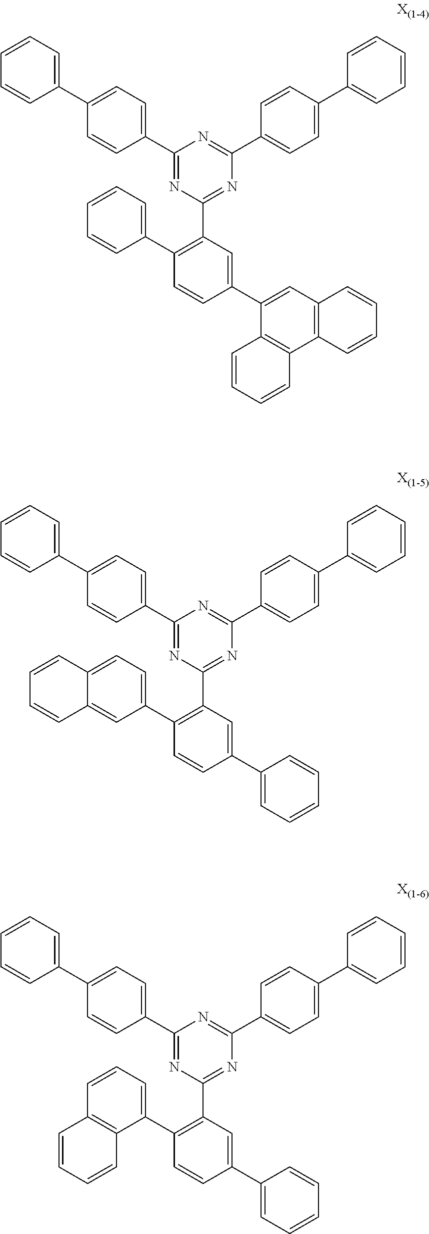 [SMILES: c1ccc(-c2ccc(-c3nc(-c4ccc(-c5ccccc5)cc4)nc(-c4cc(-c5cc6ccccc6c6ccccc56)ccc4-c4ccccc4)n3)cc2)cc1 ; c1ccc(-c2ccc(-c3nc(-c4ccc(-c5ccccc5)cc4)nc(-c4cc(-c5ccccc5)ccc4-c4ccc5ccccc5c4)n3)cc2)cc1 ; c1ccc(-c2ccc(-c3nc(-c4ccc(-c5ccccc5)cc4)nc(-c4cc(-c5ccccc5)ccc4-c4cccc5ccccc45)n3)cc2)cc1]

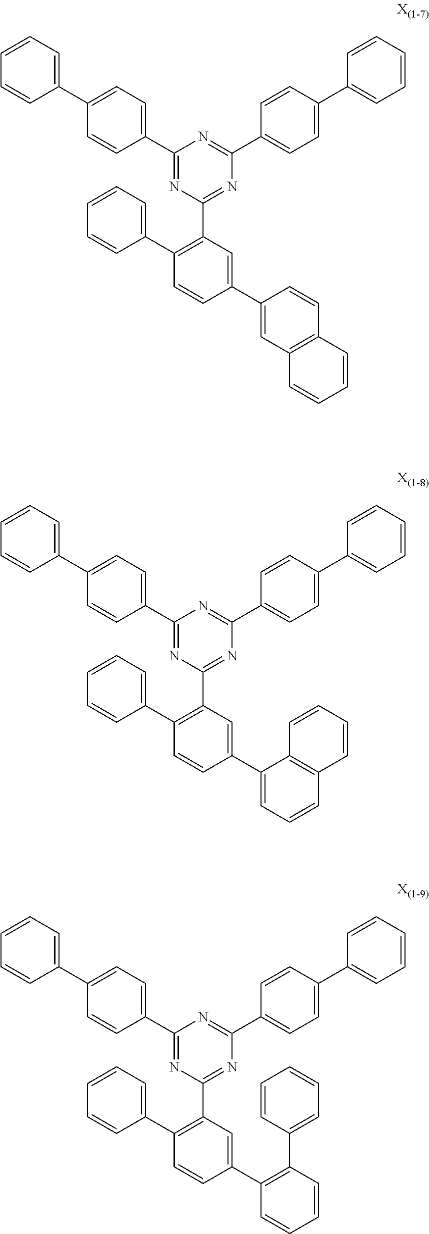 [SMILES: c1ccc(-c2ccc(-c3nc(-c4ccc(-c5ccccc5)cc4)nc(-c4cc(-c5ccc6ccccc6c5)ccc4-c4ccccc4)n3)cc2)cc1 ; c1ccc(-c2ccc(-c3nc(-c4ccc(-c5ccccc5)cc4)nc(-c4cc(-c5cccc6ccccc56)ccc4-c4ccccc4)n3)cc2)cc1 ; c1ccc(-c2ccc(-c3nc(-c4ccc(-c5ccccc5)cc4)nc(-c4cc(-c5ccccc5-c5ccccc5)ccc4-c4ccccc4)n3)cc2)cc1]

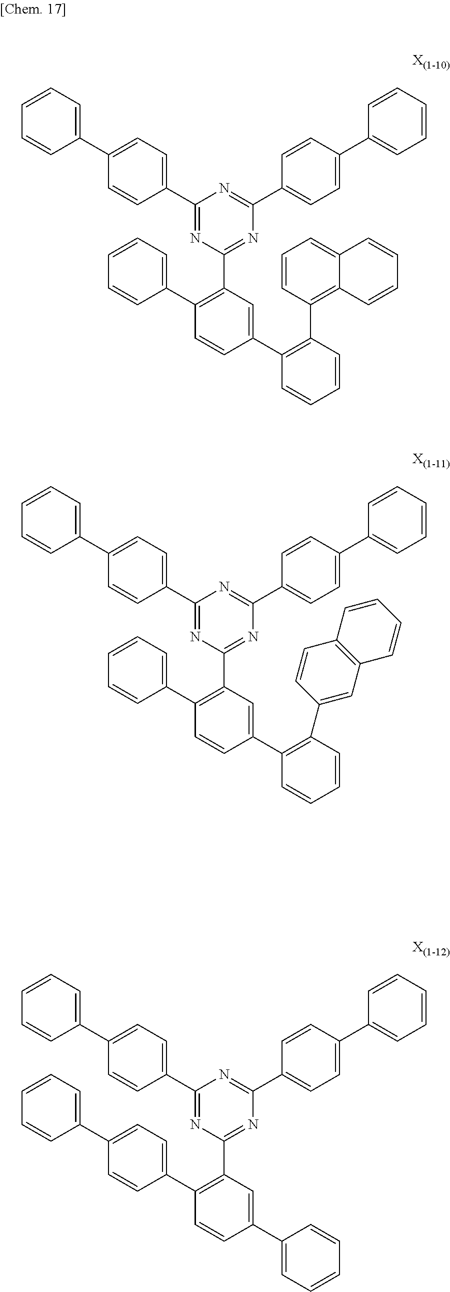 [SMILES: c1ccc(-c2ccc(-c3nc(-c4ccc(-c5ccccc5)cc4)nc(-c4cc(-c5ccccc5-c5cccc6ccccc56)ccc4-c4ccccc4)n3)cc2)cc1 ; c1ccc(-c2ccc(-c3nc(-c4ccc(-c5ccccc5)cc4)nc(-c4cc(-c5ccccc5-c5ccc6ccccc6c5)ccc4-c4ccccc4)n3)cc2)cc1 ; c1ccc(-c2ccc(-c3nc(-c4ccc(-c5ccccc5)cc4)nc(-c4cc(-c5ccccc5)ccc4-c4ccc(-c5ccccc5)cc4)n3)cc2)cc1]

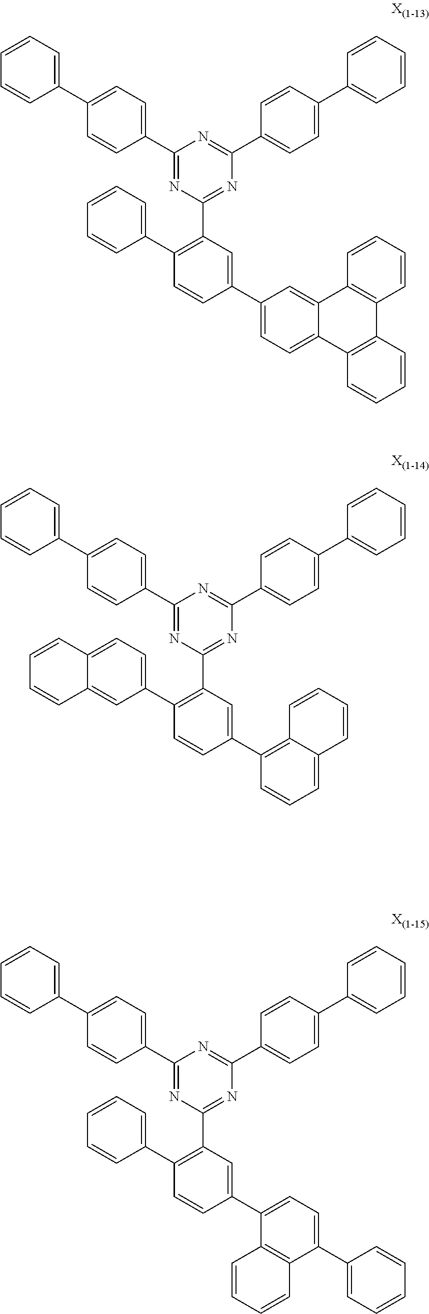 [SMILES: c1ccc(-c2ccc(-c3nc(-c4ccc(-c5ccccc5)cc4)nc(-c4cc(-c5ccc6c7ccccc7c7ccccc7c6c5)ccc4-c4ccccc4)n3)cc2)cc1 ; c1ccc(-c2ccc(-c3nc(-c4ccc(-c5ccccc5)cc4)nc(-c4cc(-c5cccc6ccccc56)ccc4-c4ccc5ccccc5c4)n3)cc2)cc1 ; c1ccc(-c2ccc(-c3nc(-c4ccc(-c5ccccc5)cc4)nc(-c4cc(-c5ccc(-c6ccccc6)c6ccccc56)ccc4-c4ccccc4)n3)cc2)cc1]

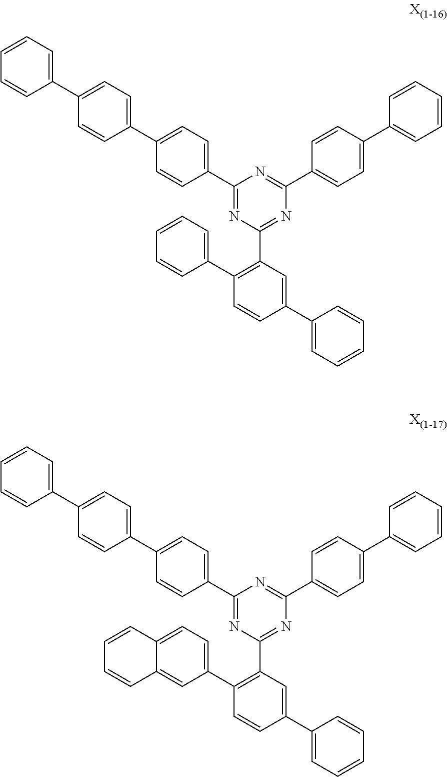 [SMILES: c1ccc(-c2ccc(-c3ccc(-c4nc(-c5ccc(-c6ccccc6)cc5)nc(-c5cc(-c6ccccc6)ccc5-c5ccccc5)n4)cc3)cc2)cc1 ; c1ccc(-c2ccc(-c3ccc(-c4nc(-c5ccc(-c6ccccc6)cc5)nc(-c5cc(-c6ccccc6)ccc5-c5ccc6ccccc6c5)n4)cc3)cc2)cc1]

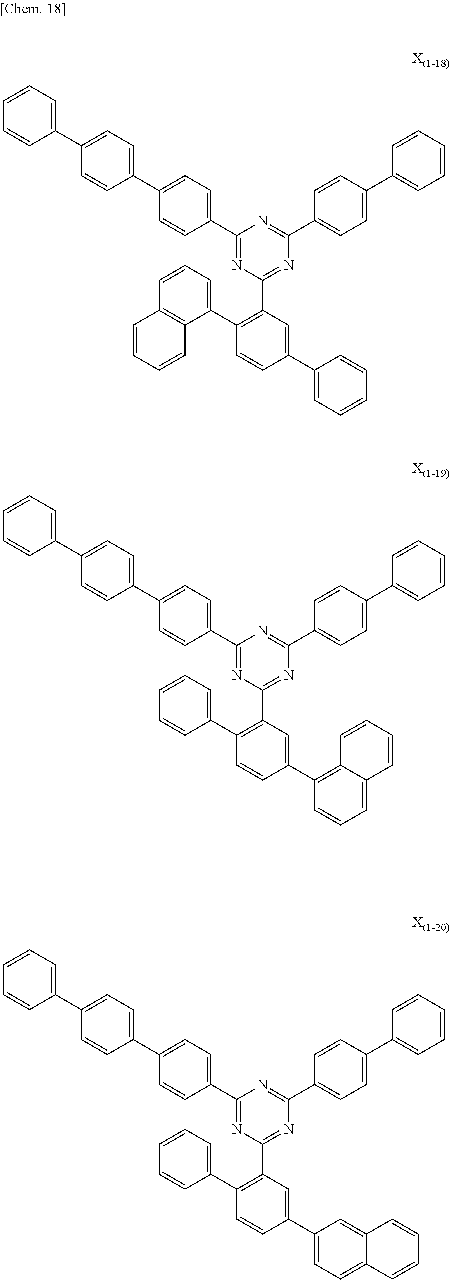 [SMILES: c1ccc(-c2ccc(-c3ccc(-c4nc(-c5ccc(-c6ccccc6)cc5)nc(-c5cc(-c6ccccc6)ccc5-c5cccc6ccccc56)n4)cc3)cc2)cc1 ; c1ccc(-c2ccc(-c3ccc(-c4nc(-c5ccc(-c6ccccc6)cc5)nc(-c5cc(-c6cccc7ccccc67)ccc5-c5ccccc5)n4)cc3)cc2)cc1 ; c1ccc(-c2ccc(-c3ccc(-c4nc(-c5ccc(-c6ccccc6)cc5)nc(-c5cc(-c6ccc7ccccc7c6)ccc5-c5ccccc5)n4)cc3)cc2)cc1]

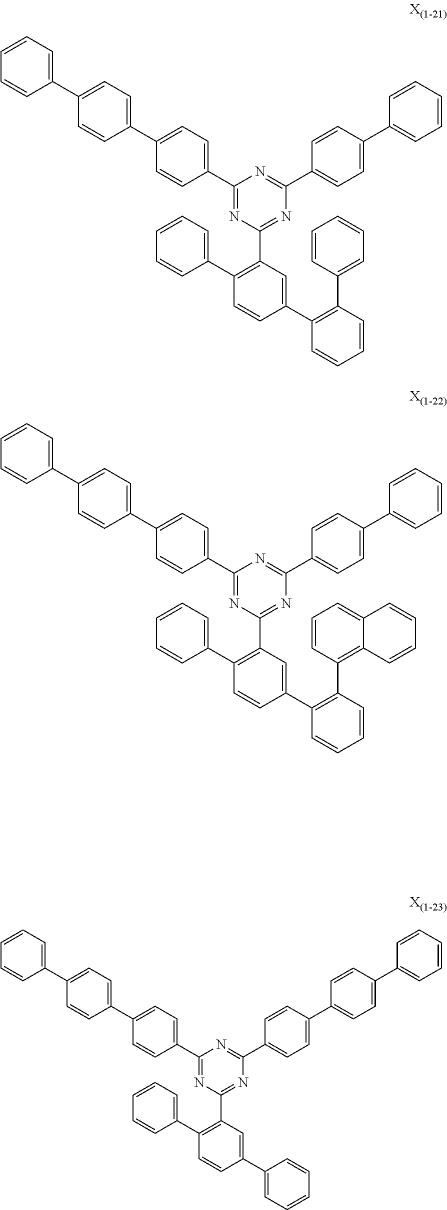 [SMILES: c1ccc(-c2ccc(-c3ccc(-c4nc(-c5ccc(-c6ccccc6)cc5)nc(-c5cc(-c6ccccc6-c6ccccc6)ccc5-c5ccccc5)n4)cc3)cc2)cc1 ; c1ccc(-c2ccc(-c3ccc(-c4nc(-c5ccc(-c6ccccc6)cc5)nc(-c5cc(-c6ccccc6-c6cccc7ccccc67)ccc5-c5ccccc5)n4)cc3)cc2)cc1 ; c1ccc(-c2ccc(-c3ccc(-c4nc(-c5ccc(-c6ccc(-c7ccccc7)cc6)cc5)nc(-c5cc(-c6ccccc6)ccc5-c5ccccc5)n4)cc3)cc2)cc1]

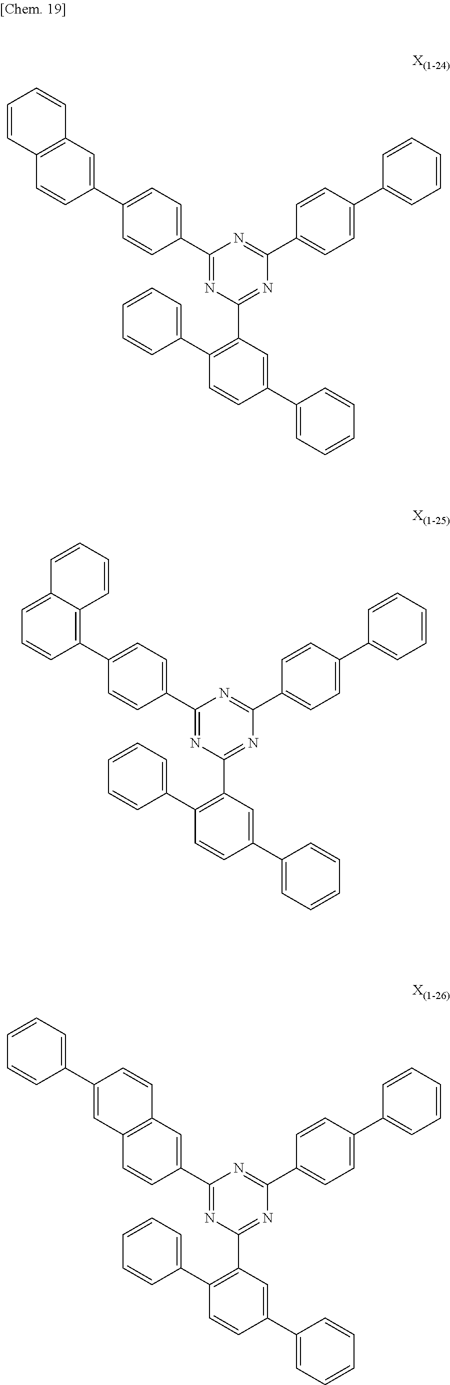 [SMILES: c1ccc(-c2ccc(-c3nc(-c4ccc(-c5ccc6ccccc6c5)cc4)nc(-c4cc(-c5ccccc5)ccc4-c4ccccc4)n3)cc2)cc1 ; c1ccc(-c2ccc(-c3nc(-c4ccc(-c5cccc6ccccc56)cc4)nc(-c4cc(-c5ccccc5)ccc4-c4ccccc4)n3)cc2)cc1 ; c1ccc(-c2ccc(-c3nc(-c4ccc5cc(-c6ccccc6)ccc5c4)nc(-c4cc(-c5ccccc5)ccc4-c4ccccc4)n3)cc2)cc1]

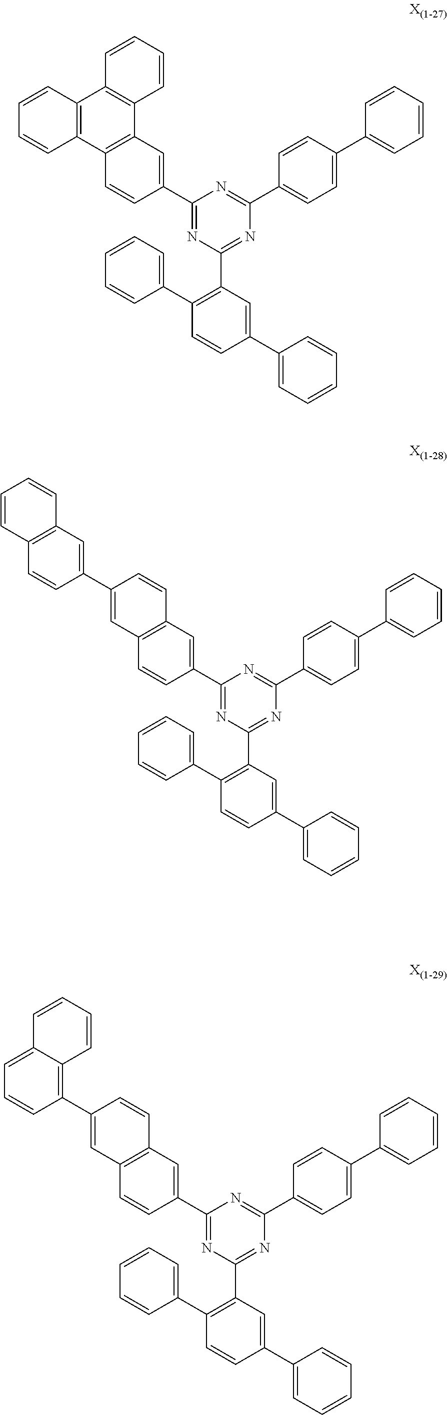 [SMILES: c1ccc(-c2ccc(-c3nc(-c4ccc5c6ccccc6c6ccccc6c5c4)nc(-c4cc(-c5ccccc5)ccc4-c4ccccc4)n3)cc2)cc1 ; c1ccc(-c2ccc(-c3nc(-c4ccc5cc(-c6ccc7ccccc7c6)ccc5c4)nc(-c4cc(-c5ccccc5)ccc4-c4ccccc4)n3)cc2)cc1 ; c1ccc(-c2ccc(-c3nc(-c4ccc5cc(-c6cccc7ccccc67)ccc5c4)nc(-c4cc(-c5ccccc5)ccc4-c4ccccc4)n3)cc2)cc1]

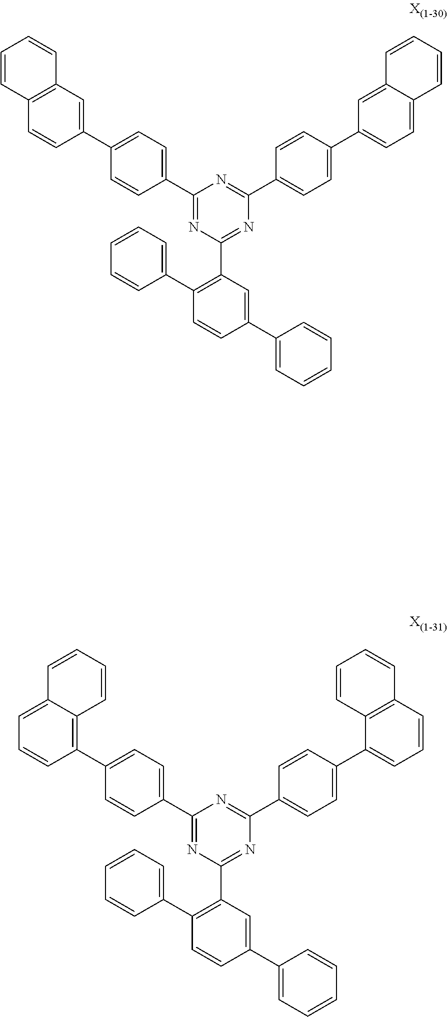 [SMILES: c1ccc(-c2ccc(-c3ccccc3)c(-c3nc(-c4ccc(-c5ccc6ccccc6c5)cc4)nc(-c4ccc(-c5ccc6ccccc6c5)cc4)n3)c2)cc1 ; c1ccc(-c2ccc(-c3ccccc3)c(-c3nc(-c4ccc(-c5cccc6ccccc56)cc4)nc(-c4ccc(-c5cccc6ccccc56)cc4)n3)c2)cc1]

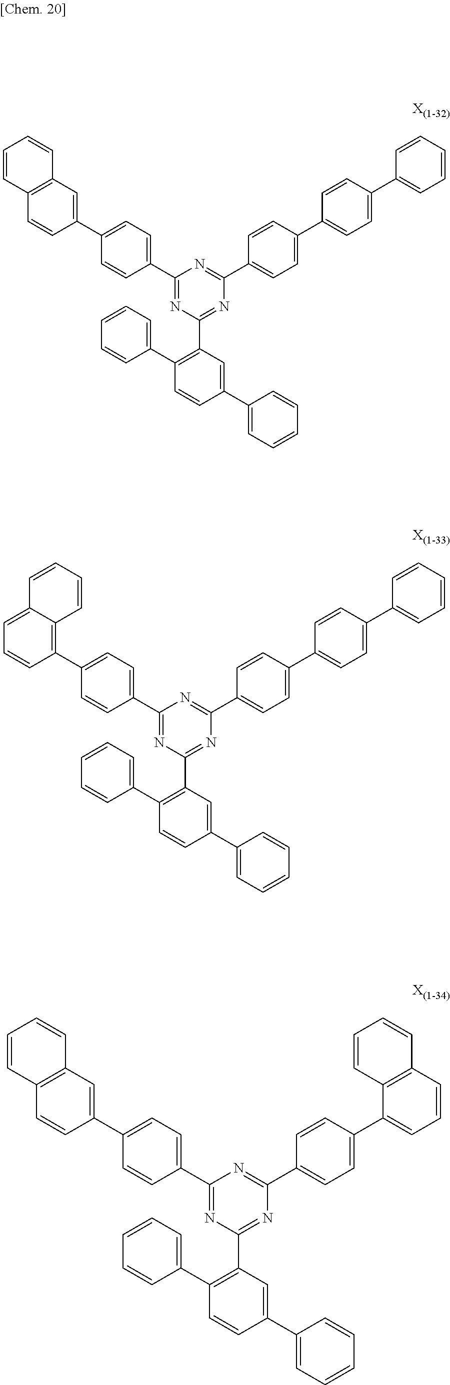 [SMILES: c1ccc(-c2ccc(-c3ccc(-c4nc(-c5ccc(-c6ccc7ccccc7c6)cc5)nc(-c5cc(-c6ccccc6)ccc5-c5ccccc5)n4)cc3)cc2)cc1 ; c1ccc(-c2ccc(-c3ccc(-c4nc(-c5ccc(-c6cccc7ccccc67)cc5)nc(-c5cc(-c6ccccc6)ccc5-c5ccccc5)n4)cc3)cc2)cc1 ; c1ccc(-c2ccc(-c3ccccc3)c(-c3nc(-c4ccc(-c5ccc6ccccc6c5)cc4)nc(-c4ccc(-c5cccc6ccccc56)cc4)n3)c2)cc1]

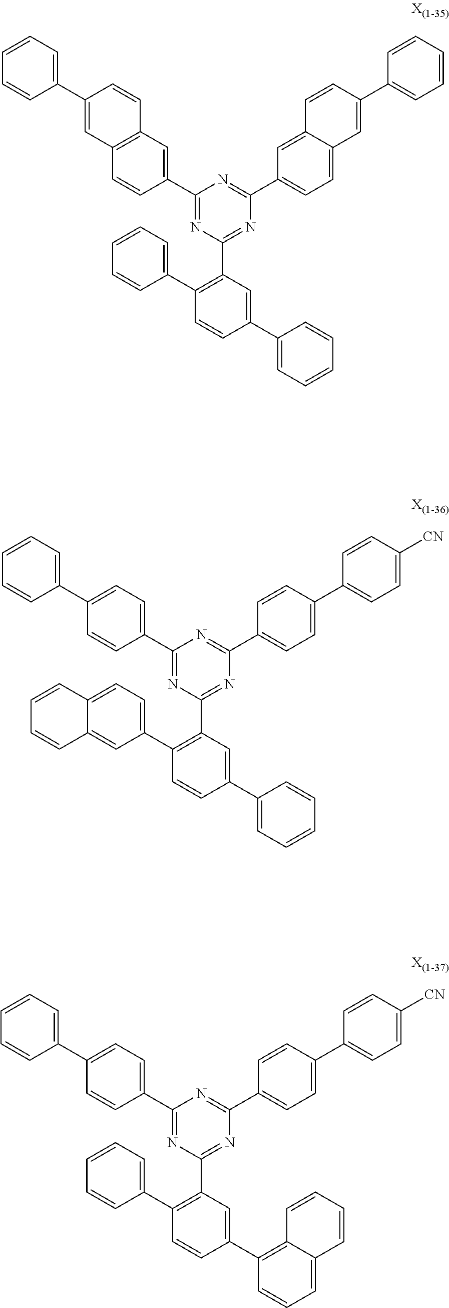 [SMILES: c1ccc(-c2ccc(-c3ccccc3)c(-c3nc(-c4ccc5cc(-c6ccccc6)ccc5c4)nc(-c4ccc5cc(-c6ccccc6)ccc5c4)n3)c2)cc1 ; N#Cc1ccc(-c2ccc(-c3nc(-c4ccc(-c5ccccc5)cc4)nc(-c4cc(-c5ccccc5)ccc4-c4ccc5ccccc5c4)n3)cc2)cc1 ; N#Cc1ccc(-c2ccc(-c3nc(-c4ccc(-c5ccccc5)cc4)nc(-c4cc(-c5cccc6ccccc56)ccc4-c4ccccc4)n3)cc2)cc1]

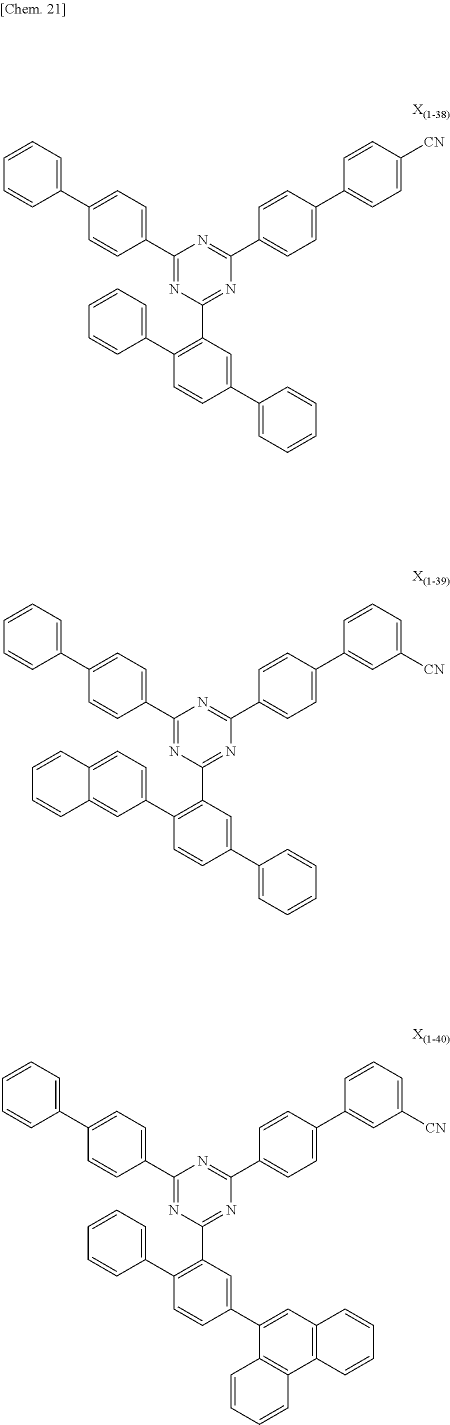 [SMILES: N#Cc1ccc(-c2ccc(-c3nc(-c4ccc(-c5ccccc5)cc4)nc(-c4cc(-c5ccccc5)ccc4-c4ccccc4)n3)cc2)cc1 ; N#Cc1cccc(-c2ccc(-c3nc(-c4ccc(-c5ccccc5)cc4)nc(-c4cc(-c5ccccc5)ccc4-c4ccc5ccccc5c4)n3)cc2)c1 ; N#Cc1cccc(-c2ccc(-c3nc(-c4ccc(-c5ccccc5)cc4)nc(-c4cc(-c5cc6ccccc6c6ccccc56)ccc4-c4ccccc4)n3)cc2)c1]

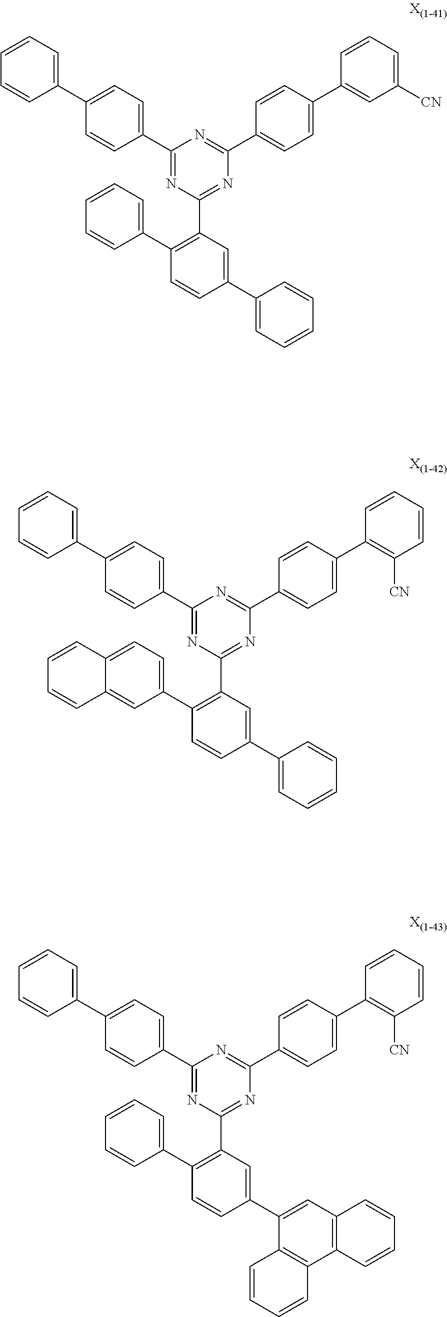 [SMILES: N#Cc1cccc(-c2ccc(-c3nc(-c4ccc(-c5ccccc5)cc4)nc(-c4cc(-c5ccccc5)ccc4-c4ccccc4)n3)cc2)c1 ; N#Cc1ccccc1-c1ccc(-c2nc(-c3ccc(-c4ccccc4)cc3)nc(-c3cc(-c4ccccc4)ccc3-c3ccc4ccccc4c3)n2)cc1 ; N#Cc1ccccc1-c1ccc(-c2nc(-c3ccc(-c4ccccc4)cc3)nc(-c3cc(-c4cc5ccccc5c5ccccc45)ccc3-c3ccccc3)n2)cc1]

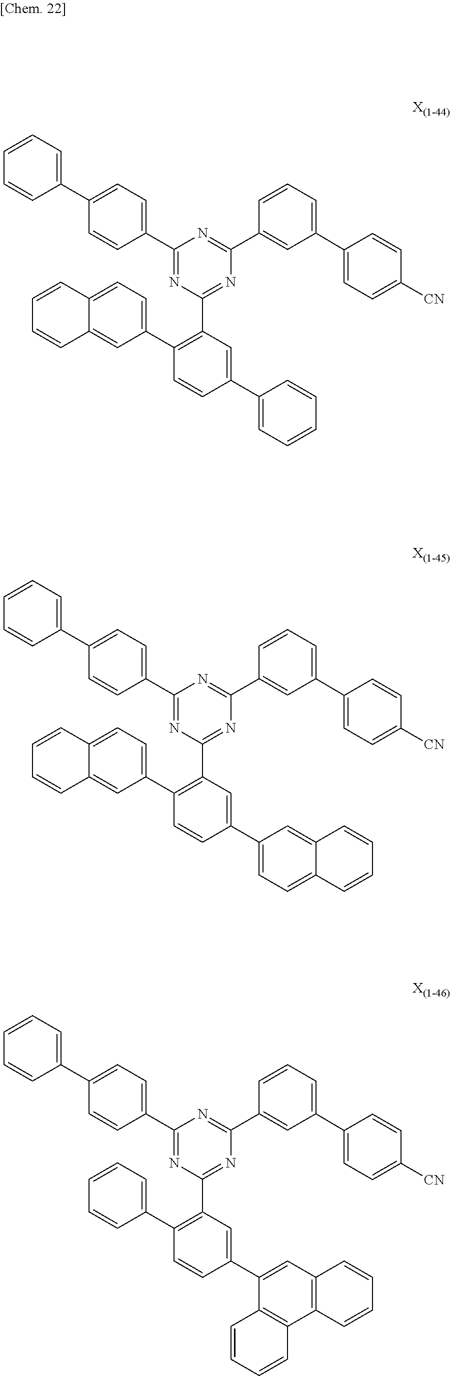 [SMILES: N#Cc1ccc(-c2cccc(-c3nc(-c4ccc(-c5ccccc5)cc4)nc(-c4cc(-c5ccccc5)ccc4-c4ccc5ccccc5c4)n3)c2)cc1 ; N#Cc1ccc(-c2cccc(-c3nc(-c4ccc(-c5ccccc5)cc4)nc(-c4cc(-c5ccc6ccccc6c5)ccc4-c4ccc5ccccc5c4)n3)c2)cc1 ; N#Cc1ccc(-c2cccc(-c3nc(-c4ccc(-c5ccccc5)cc4)nc(-c4cc(-c5cc6ccccc6c6ccccc56)ccc4-c4ccccc4)n3)c2)cc1]

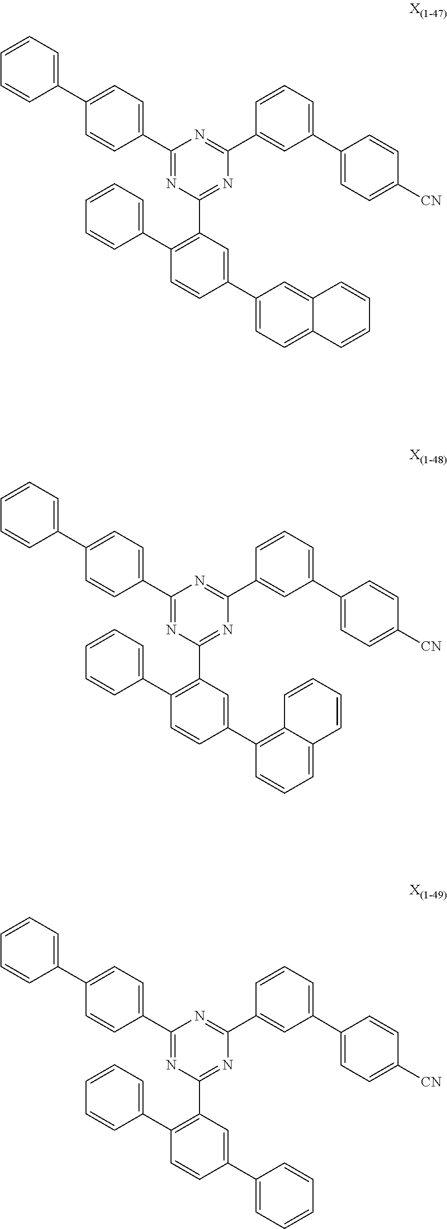 [SMILES: N#Cc1ccc(-c2cccc(-c3nc(-c4ccc(-c5ccccc5)cc4)nc(-c4cc(-c5ccc6ccccc6c5)ccc4-c4ccccc4)n3)c2)cc1 ; N#Cc1ccc(-c2cccc(-c3nc(-c4ccc(-c5ccccc5)cc4)nc(-c4cc(-c5cccc6ccccc56)ccc4-c4ccccc4)n3)c2)cc1 ; N#Cc1ccc(-c2cccc(-c3nc(-c4ccc(-c5ccccc5)cc4)nc(-c4cc(-c5ccccc5)ccc4-c4ccccc4)n3)c2)cc1]

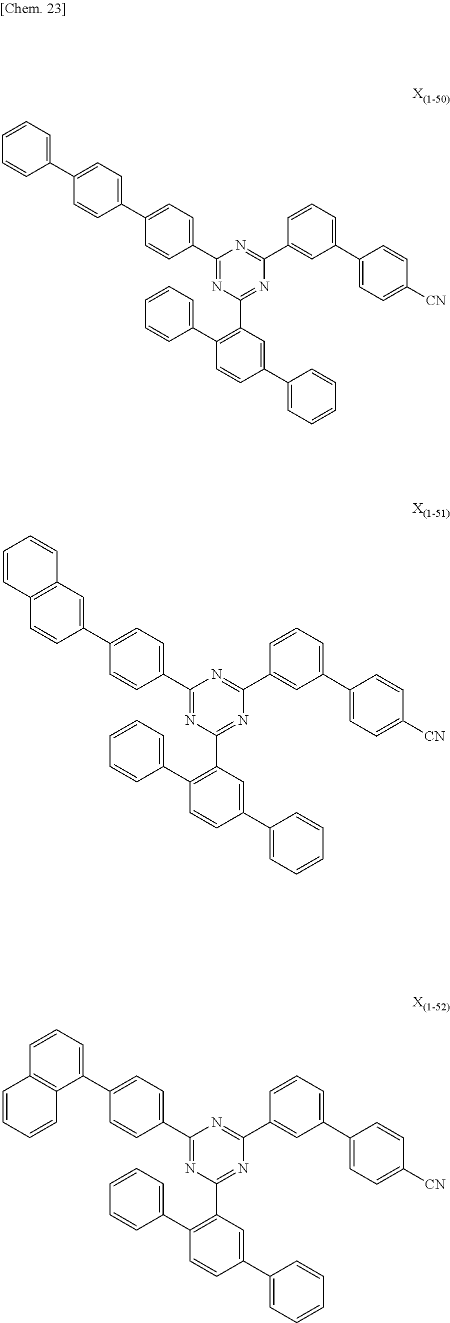 [SMILES: N#Cc1ccc(-c2cccc(-c3nc(-c4ccc(-c5ccc(-c6ccccc6)cc5)cc4)nc(-c4cc(-c5ccccc5)ccc4-c4ccccc4)n3)c2)cc1 ; N#Cc1ccc(-c2cccc(-c3nc(-c4ccc(-c5ccc6ccccc6c5)cc4)nc(-c4cc(-c5ccccc5)ccc4-c4ccccc4)n3)c2)cc1 ; N#Cc1ccc(-c2cccc(-c3nc(-c4ccc(-c5cccc6ccccc56)cc4)nc(-c4cc(-c5ccccc5)ccc4-c4ccccc4)n3)c2)cc1]

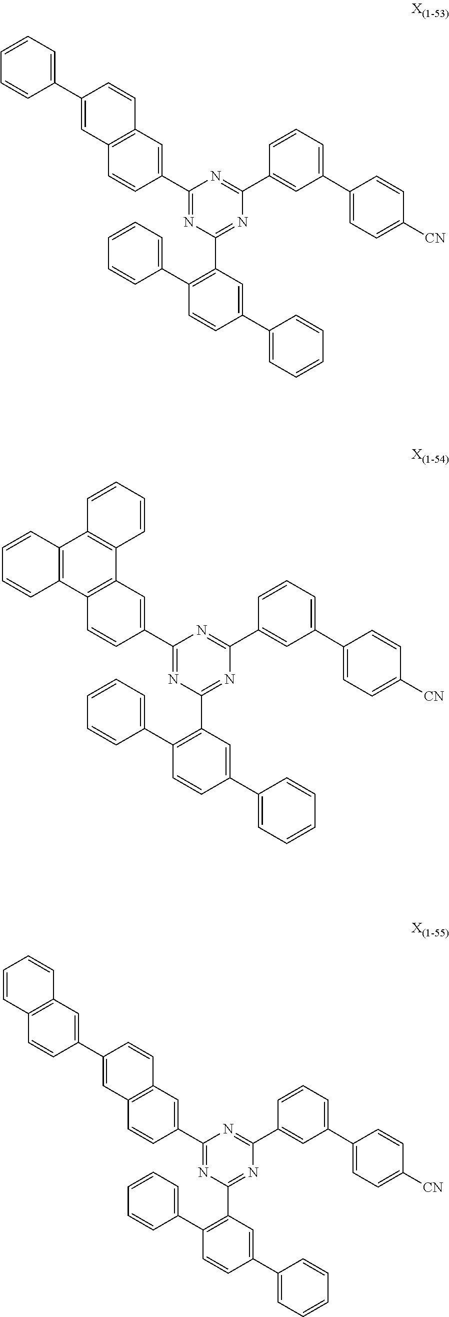 [SMILES: N#Cc1ccc(-c2cccc(-c3nc(-c4ccc5cc(-c6ccccc6)ccc5c4)nc(-c4cc(-c5ccccc5)ccc4-c4ccccc4)n3)c2)cc1 ; N#Cc1ccc(-c2cccc(-c3nc(-c4ccc5c6ccccc6c6ccccc6c5c4)nc(-c4cc(-c5ccccc5)ccc4-c4ccccc4)n3)c2)cc1 ; N#Cc1ccc(-c2cccc(-c3nc(-c4ccc5cc(-c6ccc7ccccc7c6)ccc5c4)nc(-c4cc(-c5ccccc5)ccc4-c4ccccc4)n3)c2)cc1]

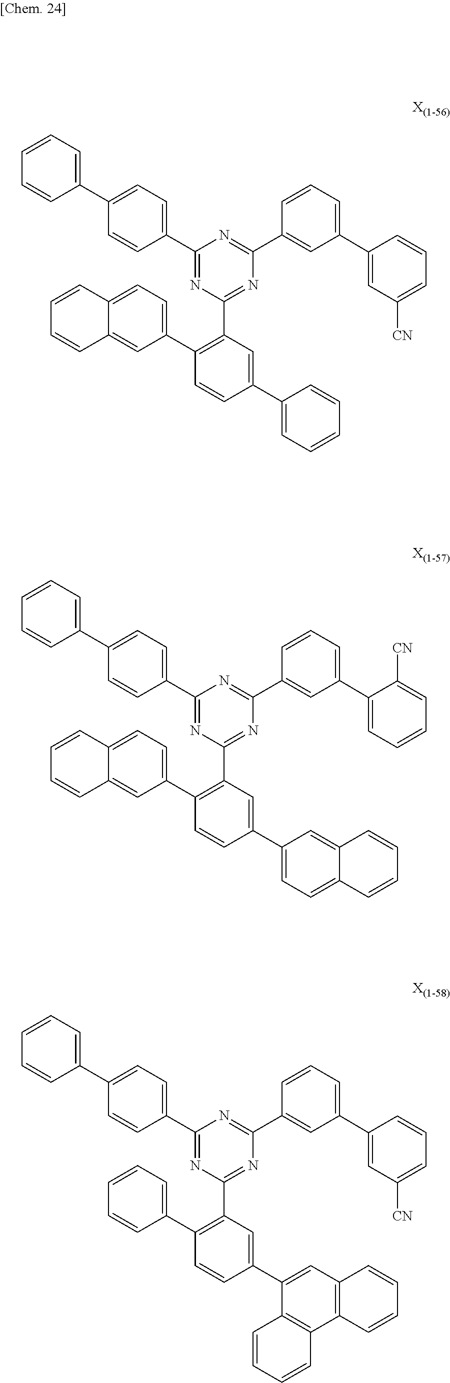 [SMILES: N#Cc1cccc(-c2cccc(-c3nc(-c4ccc(-c5ccccc5)cc4)nc(-c4cc(-c5ccccc5)ccc4-c4ccc5ccccc5c4)n3)c2)c1 ; N#Cc1ccccc1-c1cccc(-c2nc(-c3ccc(-c4ccccc4)cc3)nc(-c3cc(-c4ccc5ccccc5c4)ccc3-c3ccc4ccccc4c3)n2)c1 ; N#Cc1cccc(-c2cccc(-c3nc(-c4ccc(-c5ccccc5)cc4)nc(-c4cc(-c5cc6ccccc6c6ccccc56)ccc4-c4ccccc4)n3)c2)c1]

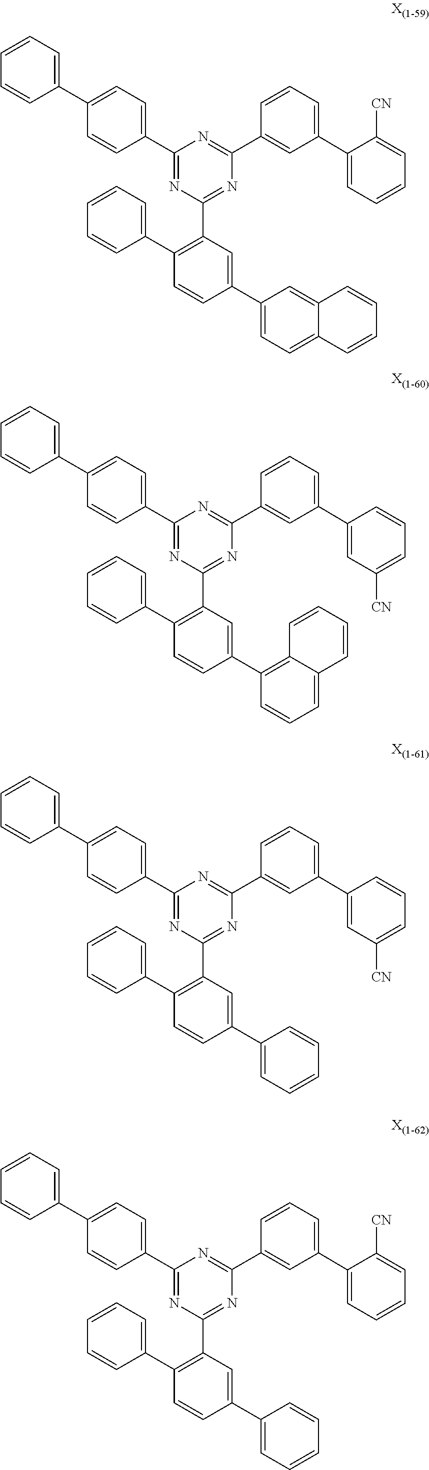 [SMILES: N#Cc1ccccc1-c1cccc(-c2nc(-c3ccc(-c4ccccc4)cc3)nc(-c3cc(-c4ccc5ccccc5c4)ccc3-c3ccccc3)n2)c1 ; N#Cc1cccc(-c2cccc(-c3nc(-c4ccc(-c5ccccc5)cc4)nc(-c4cc(-c5cccc6ccccc56)ccc4-c4ccccc4)n3)c2)c1 ; N#Cc1cccc(-c2cccc(-c3nc(-c4ccc(-c5ccccc5)cc4)nc(-c4cc(-c5ccccc5)ccc4-c4ccccc4)n3)c2)c1 ; N#Cc1ccccc1-c1cccc(-c2nc(-c3ccc(-c4ccccc4)cc3)nc(-c3cc(-c4ccccc4)ccc3-c3ccccc3)n2)c1]

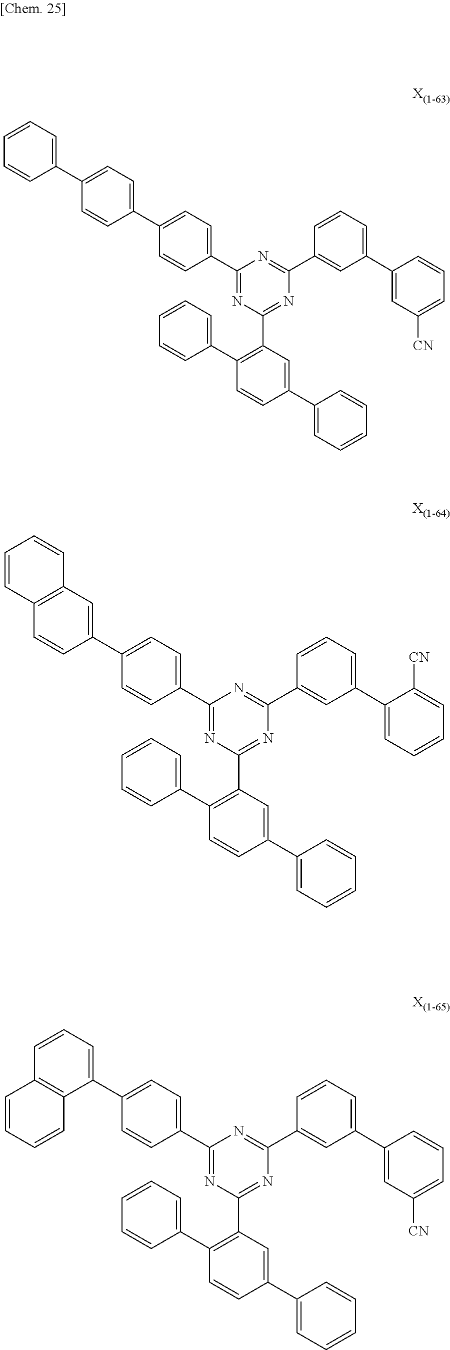 [SMILES: N#Cc1cccc(-c2cccc(-c3nc(-c4ccc(-c5ccc(-c6ccccc6)cc5)cc4)nc(-c4cc(-c5ccccc5)ccc4-c4ccccc4)n3)c2)c1 ; N#Cc1ccccc1-c1cccc(-c2nc(-c3ccc(-c4ccc5ccccc5c4)cc3)nc(-c3cc(-c4ccccc4)ccc3-c3ccccc3)n2)c1 ; N#Cc1cccc(-c2cccc(-c3nc(-c4ccc(-c5cccc6ccccc56)cc4)nc(-c4cc(-c5ccccc5)ccc4-c4ccccc4)n3)c2)c1]

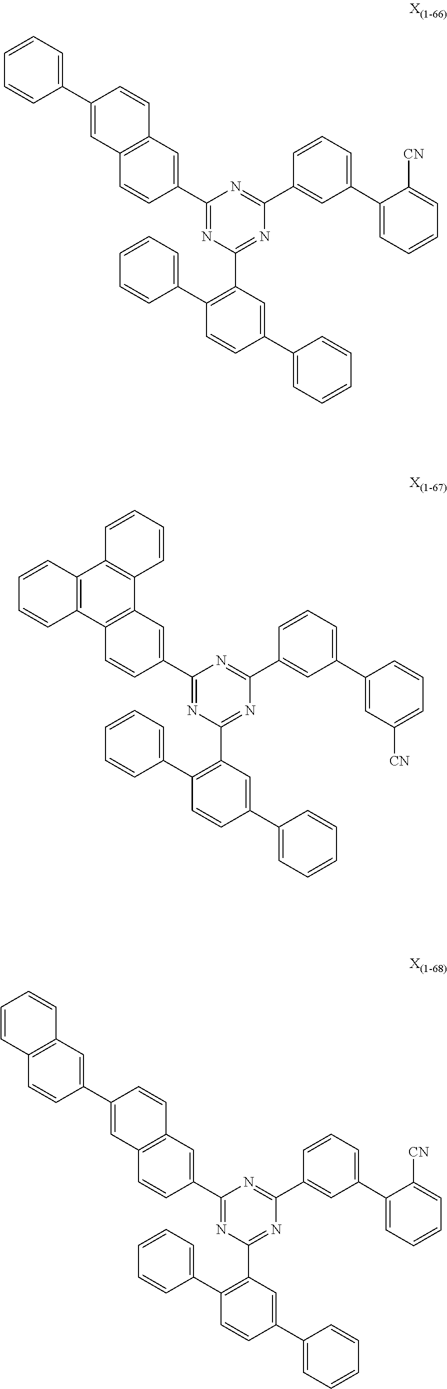 [SMILES: N#Cc1ccccc1-c1cccc(-c2nc(-c3ccc4cc(-c5ccccc5)ccc4c3)nc(-c3cc(-c4ccccc4)ccc3-c3ccccc3)n2)c1 ; N#Cc1cccc(-c2cccc(-c3nc(-c4ccc5c6ccccc6c6ccccc6c5c4)nc(-c4cc(-c5ccccc5)ccc4-c4ccccc4)n3)c2)c1 ; N#Cc1ccccc1-c1cccc(-c2nc(-c3ccc4cc(-c5ccc6ccccc6c5)ccc4c3)nc(-c3cc(-c4ccccc4)ccc3-c3ccccc3)n2)c1]

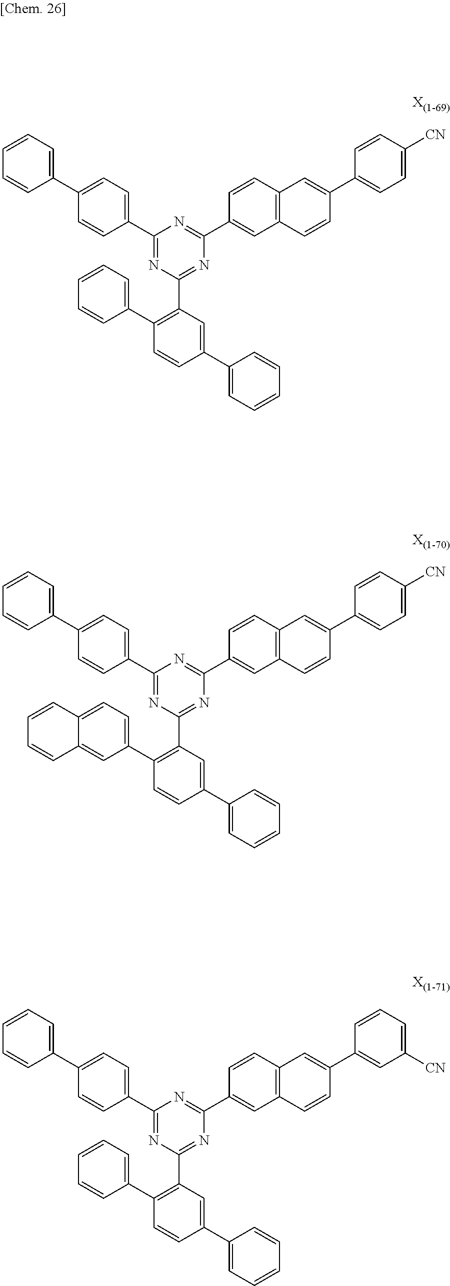 [SMILES: N#Cc1ccc(-c2ccc3cc(-c4nc(-c5ccc(-c6ccccc6)cc5)nc(-c5cc(-c6ccccc6)ccc5-c5ccccc5)n4)ccc3c2)cc1 ; N#Cc1ccc(-c2ccc3cc(-c4nc(-c5ccc(-c6ccccc6)cc5)nc(-c5cc(-c6ccccc6)ccc5-c5ccc6ccccc6c5)n4)ccc3c2)cc1 ; N#Cc1cccc(-c2ccc3cc(-c4nc(-c5ccc(-c6ccccc6)cc5)nc(-c5cc(-c6ccccc6)ccc5-c5ccccc5)n4)ccc3c2)c1]

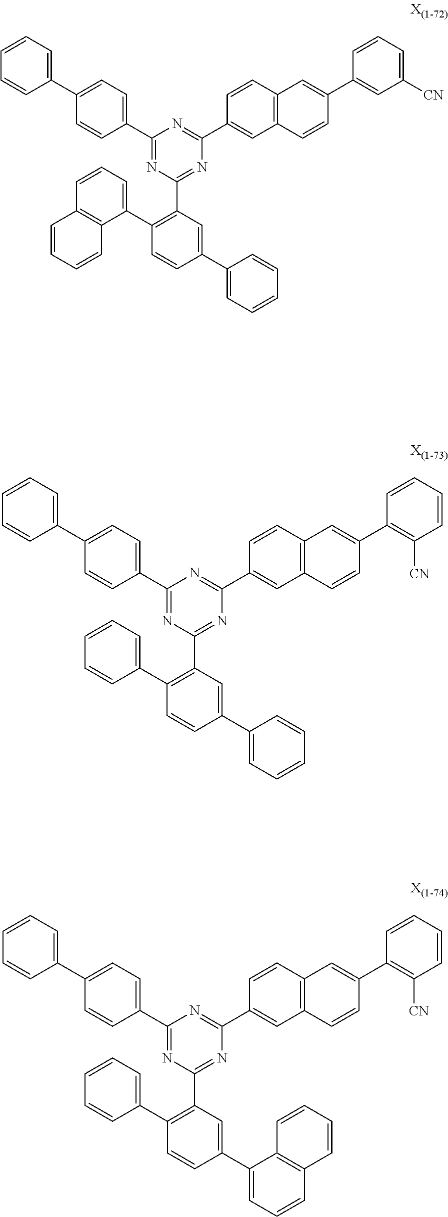 [SMILES: N#Cc1cccc(-c2ccc3cc(-c4nc(-c5ccc(-c6ccccc6)cc5)nc(-c5cc(-c6ccccc6)ccc5-c5cccc6ccccc56)n4)ccc3c2)c1 ; N#Cc1ccccc1-c1ccc2cc(-c3nc(-c4ccc(-c5ccccc5)cc4)nc(-c4cc(-c5ccccc5)ccc4-c4ccccc4)n3)ccc2c1 ; N#Cc1ccccc1-c1ccc2cc(-c3nc(-c4ccc(-c5ccccc5)cc4)nc(-c4cc(-c5cccc6ccccc56)ccc4-c4ccccc4)n3)ccc2c1]

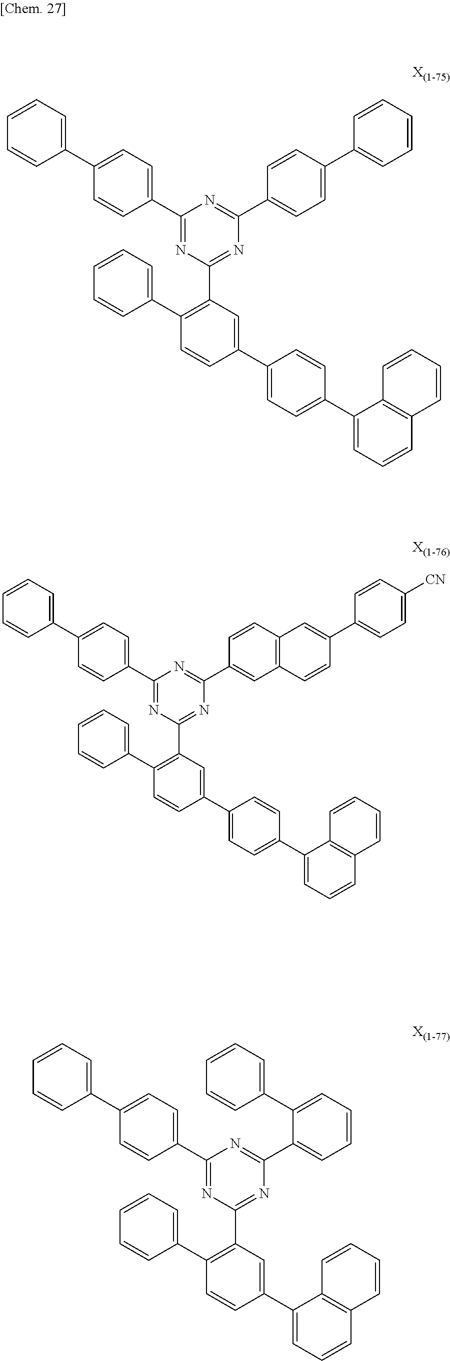 [SMILES: c1ccc(-c2ccc(-c3nc(-c4ccc(-c5ccccc5)cc4)nc(-c4cc(-c5ccc(-c6cccc7ccccc67)cc5)ccc4-c4ccccc4)n3)cc2)cc1 ; N#Cc1ccc(-c2ccc3cc(-c4nc(-c5ccc(-c6ccccc6)cc5)nc(-c5cc(-c6ccc(-c7cccc8ccccc78)cc6)ccc5-c5ccccc5)n4)ccc3c2)cc1 ; c1ccc(-c2ccc(-c3nc(-c4ccccc4-c4ccccc4)nc(-c4cc(-c5cccc6ccccc56)ccc4-c4ccccc4)n3)cc2)cc1]

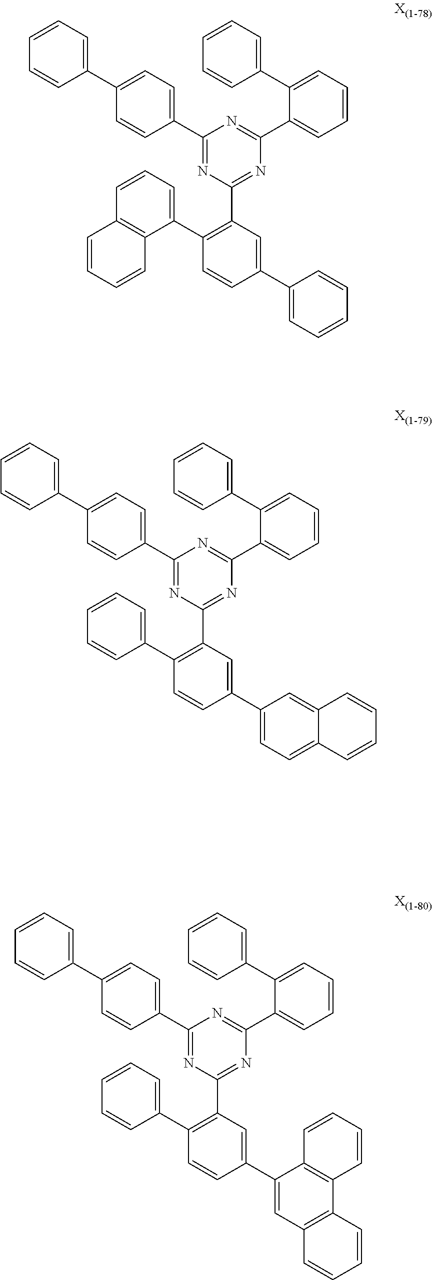 [SMILES: c1ccc(-c2ccc(-c3nc(-c4ccccc4-c4ccccc4)nc(-c4cc(-c5ccccc5)ccc4-c4cccc5ccccc45)n3)cc2)cc1 ; c1ccc(-c2ccc(-c3nc(-c4ccccc4-c4ccccc4)nc(-c4cc(-c5ccc6ccccc6c5)ccc4-c4ccccc4)n3)cc2)cc1 ; c1ccc(-c2ccc(-c3nc(-c4ccccc4-c4ccccc4)nc(-c4cc(-c5cc6ccccc6c6ccccc56)ccc4-c4ccccc4)n3)cc2)cc1]

### <Triazine Compound Y(1)>

A triazine compound Y(1) according to an aspect of the present invention is represented by formula Y(1):

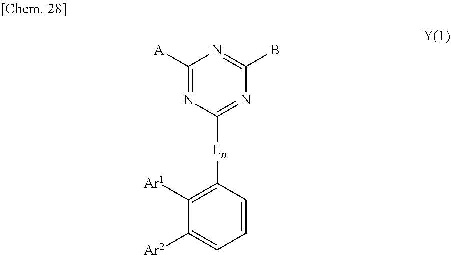

wherein in the case of the formula Y(1), L represents any one group selected from formulas Y(2-1) to Y(2-5).

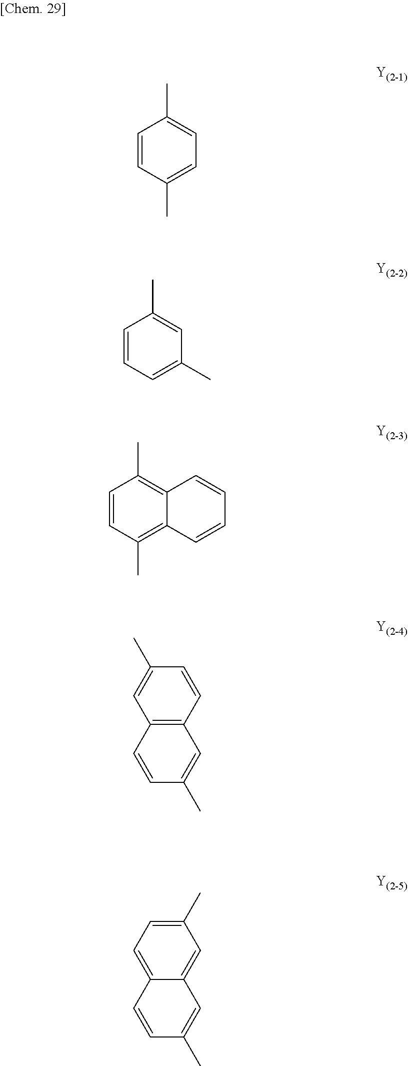

Most preferably, the group is any one group selected from formulas Y(A-1) to Y(A-10).

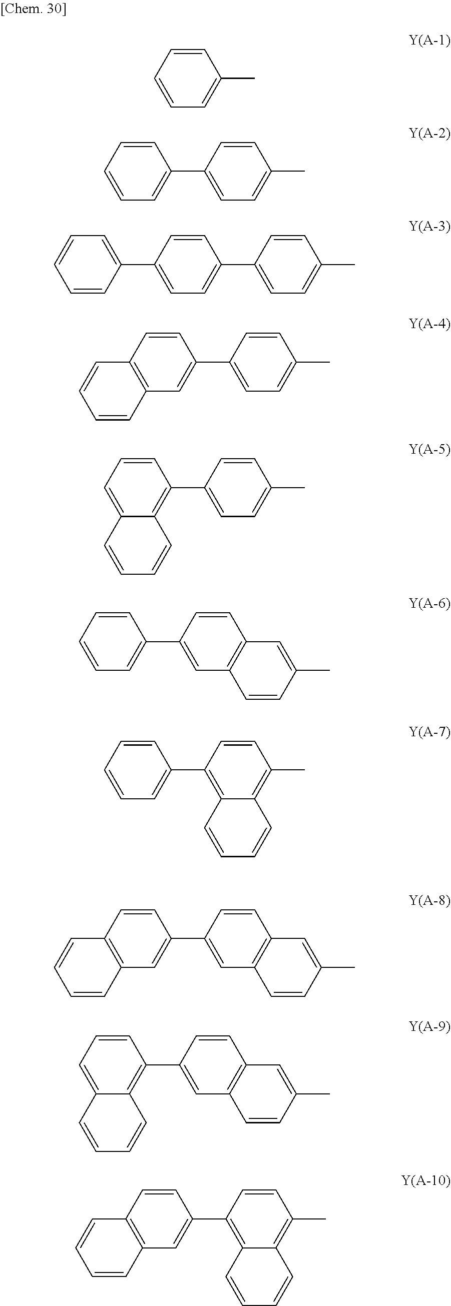

Most preferably, the group is any one group selected from formulas Y(B-1) to Y(B-28).

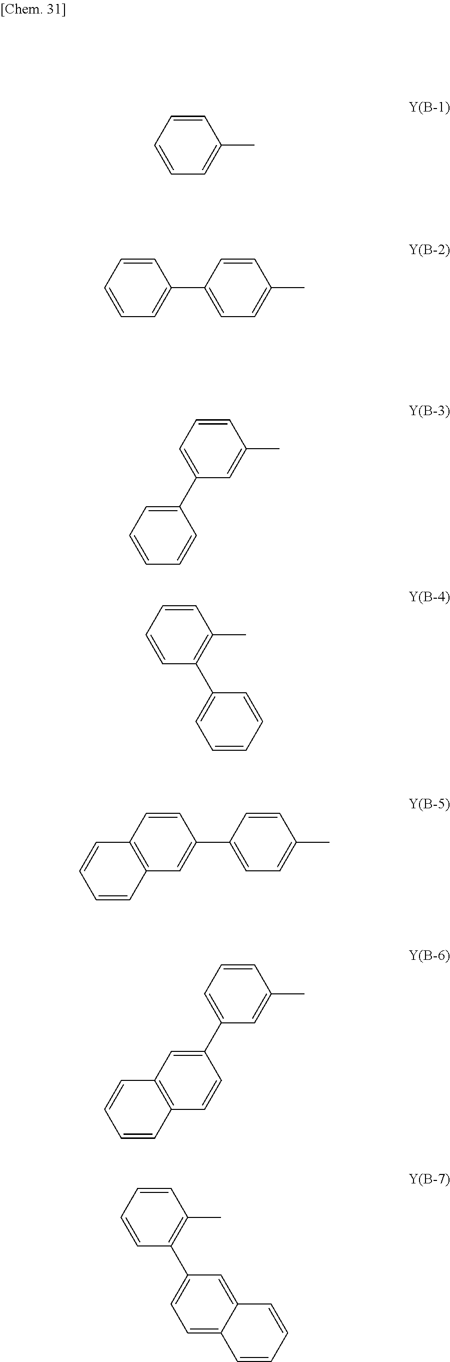

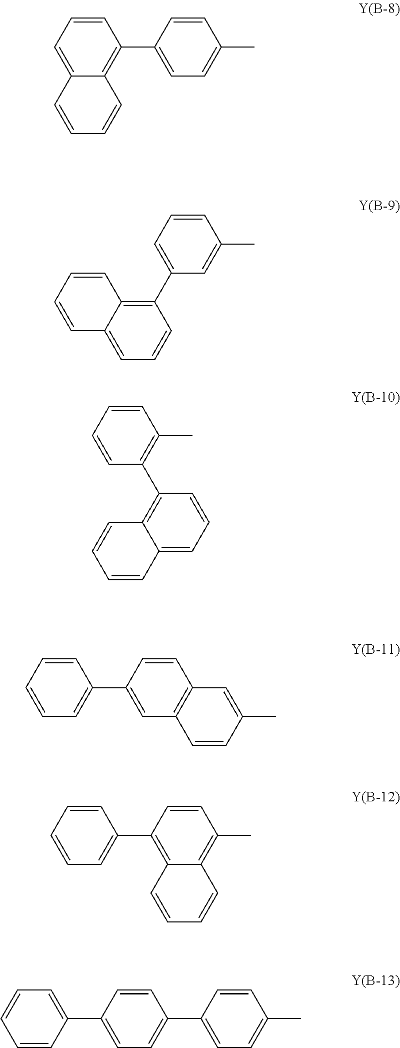

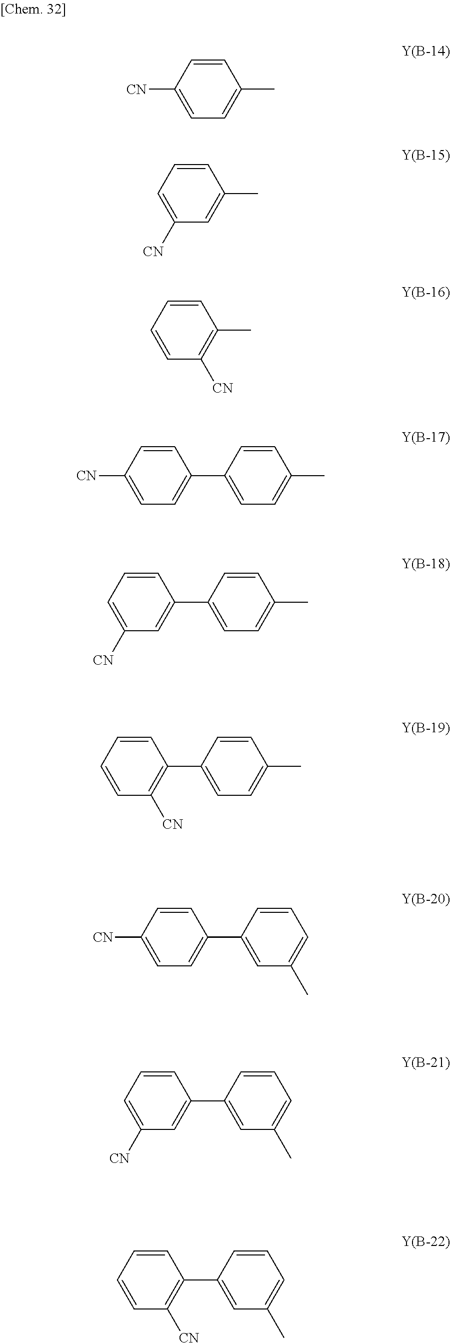

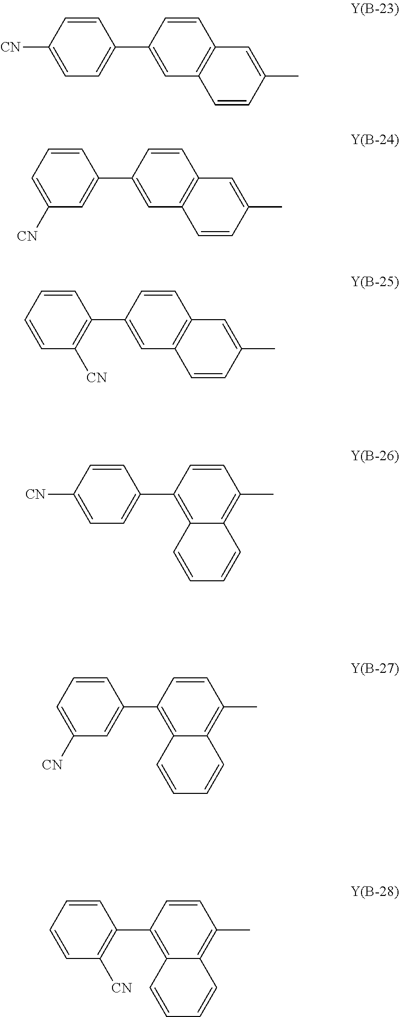

L represents any one group selected from the formulas Y(2-1) to Y(2-5).

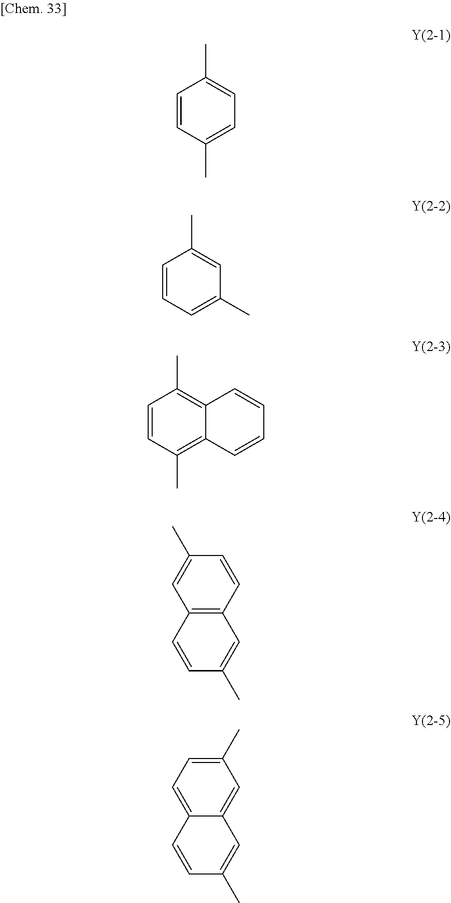

### Specific Examples of Triazine Compound Y(1)

Among the triazine compounds according to an aspect of the present invention represented by the formula Y(1), specific examples of particularly preferable compounds include the following Y(1-1) to Y(1-179), but the triazine compound according to an aspect of the present invention is not limited thereto.

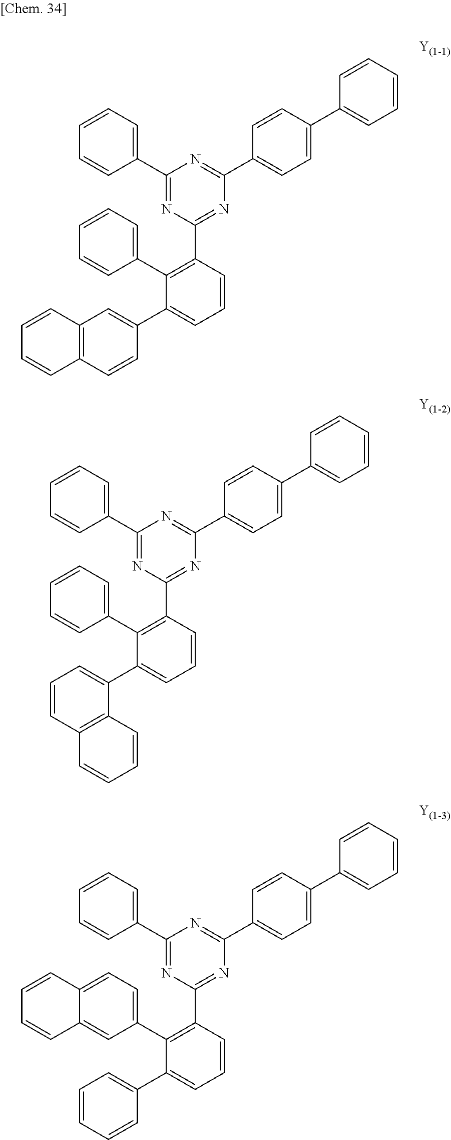 [SMILES: c1ccc(-c2ccc(-c3nc(-c4ccccc4)nc(-c4cccc(-c5ccc6ccccc6c5)c4-c4ccccc4)n3)cc2)cc1 ; c1ccc(-c2ccc(-c3nc(-c4ccccc4)nc(-c4cccc(-c5cccc6ccccc56)c4-c4ccccc4)n3)cc2)cc1 ; c1ccc(-c2ccc(-c3nc(-c4ccccc4)nc(-c4cccc(-c5ccccc5)c4-c4ccc5ccccc5c4)n3)cc2)cc1]

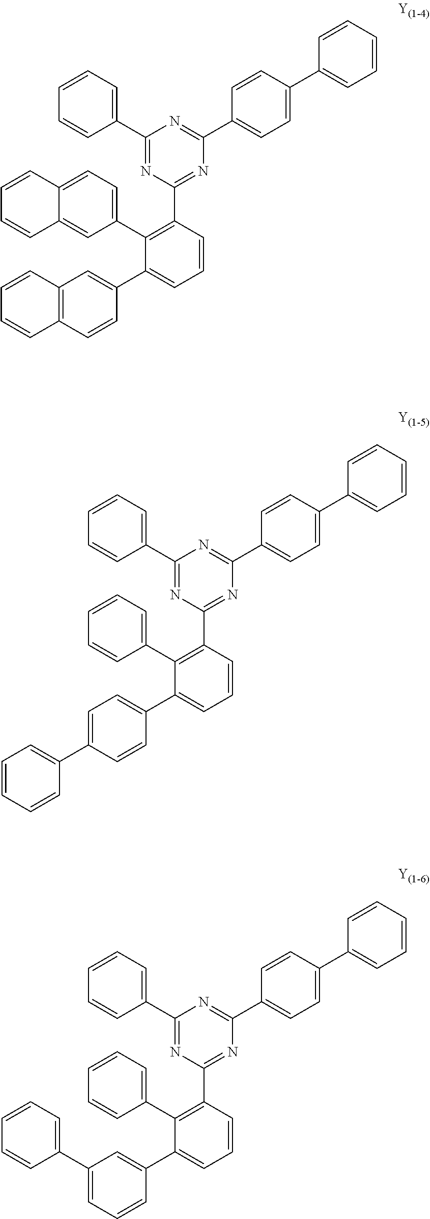 [SMILES: c1ccc(-c2ccc(-c3nc(-c4ccccc4)nc(-c4cccc(-c5ccc6ccccc6c5)c4-c4ccc5ccccc5c4)n3)cc2)cc1 ; c1ccc(-c2ccc(-c3nc(-c4ccccc4)nc(-c4cccc(-c5ccc(-c6ccccc6)cc5)c4-c4ccccc4)n3)cc2)cc1 ; c1ccc(-c2ccc(-c3nc(-c4ccccc4)nc(-c4cccc(-c5cccc(-c6ccccc6)c5)c4-c4ccccc4)n3)cc2)cc1]

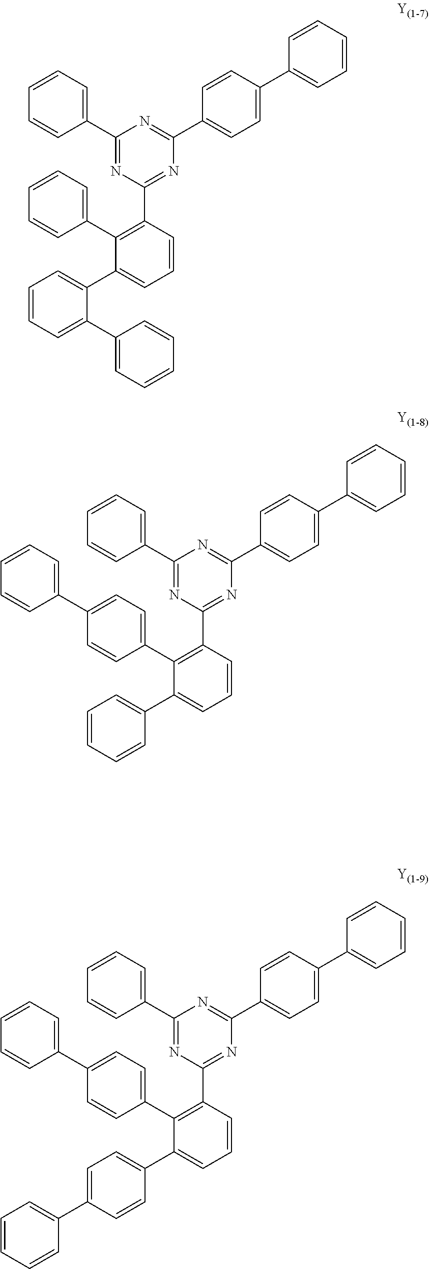 [SMILES: c1ccc(-c2ccc(-c3nc(-c4ccccc4)nc(-c4cccc(-c5ccccc5-c5ccccc5)c4-c4ccccc4)n3)cc2)cc1 ; c1ccc(-c2ccc(-c3nc(-c4ccccc4)nc(-c4cccc(-c5ccccc5)c4-c4ccc(-c5ccccc5)cc4)n3)cc2)cc1 ; c1ccc(-c2ccc(-c3nc(-c4ccccc4)nc(-c4cccc(-c5ccc(-c6ccccc6)cc5)c4-c4ccc(-c5ccccc5)cc4)n3)cc2)cc1]

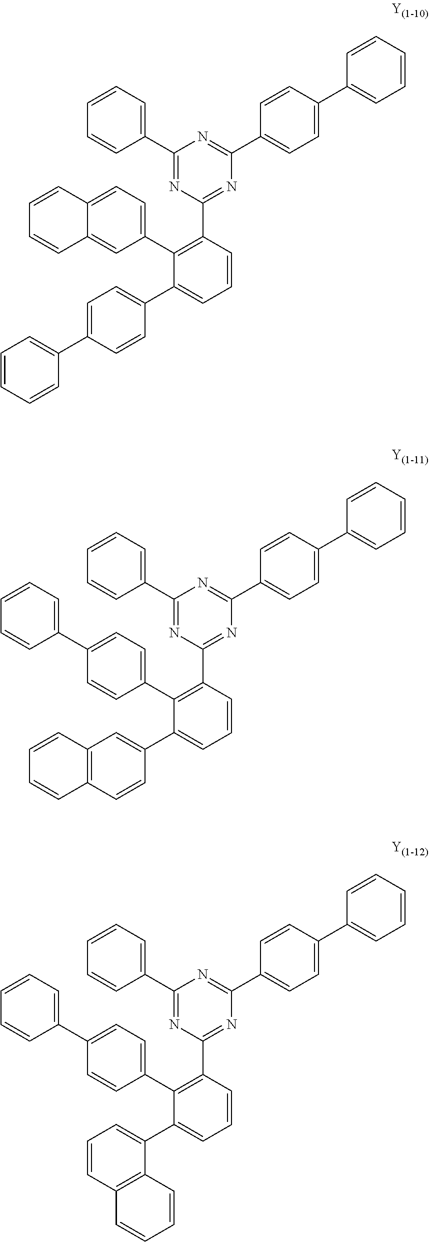 [SMILES: c1ccc(-c2ccc(-c3nc(-c4ccccc4)nc(-c4cccc(-c5ccc(-c6ccccc6)cc5)c4-c4ccc5ccccc5c4)n3)cc2)cc1 ; c1ccc(-c2ccc(-c3nc(-c4ccccc4)nc(-c4cccc(-c5ccc6ccccc6c5)c4-c4ccc(-c5ccccc5)cc4)n3)cc2)cc1 ; c1ccc(-c2ccc(-c3nc(-c4ccccc4)nc(-c4cccc(-c5cccc6ccccc56)c4-c4ccc(-c5ccccc5)cc4)n3)cc2)cc1]

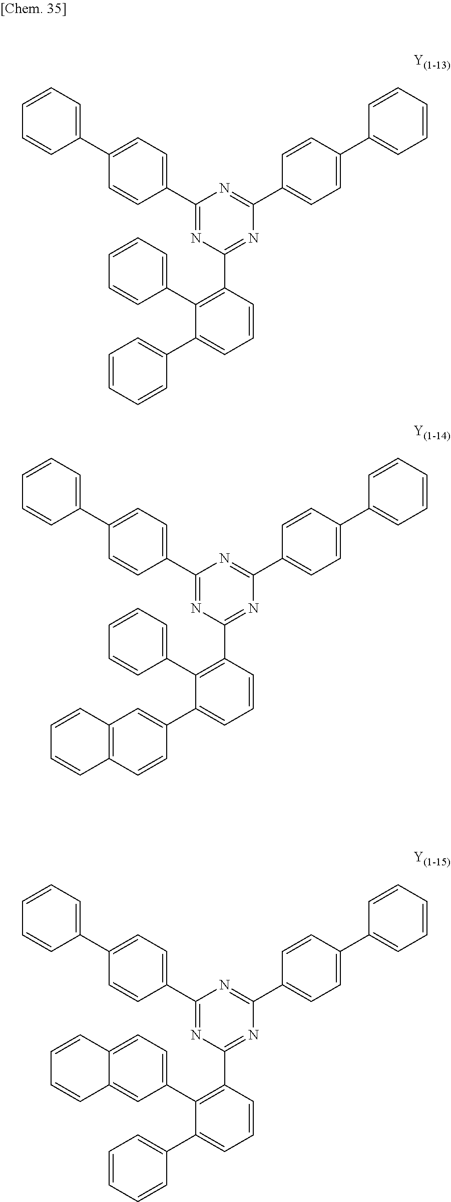 [SMILES: c1ccc(-c2ccc(-c3nc(-c4ccc(-c5ccccc5)cc4)nc(-c4cccc(-c5ccccc5)c4-c4ccccc4)n3)cc2)cc1 ; c1ccc(-c2ccc(-c3nc(-c4ccc(-c5ccccc5)cc4)nc(-c4cccc(-c5ccc6ccccc6c5)c4-c4ccccc4)n3)cc2)cc1 ; c1ccc(-c2ccc(-c3nc(-c4ccc(-c5ccccc5)cc4)nc(-c4cccc(-c5ccccc5)c4-c4ccc5ccccc5c4)n3)cc2)cc1]

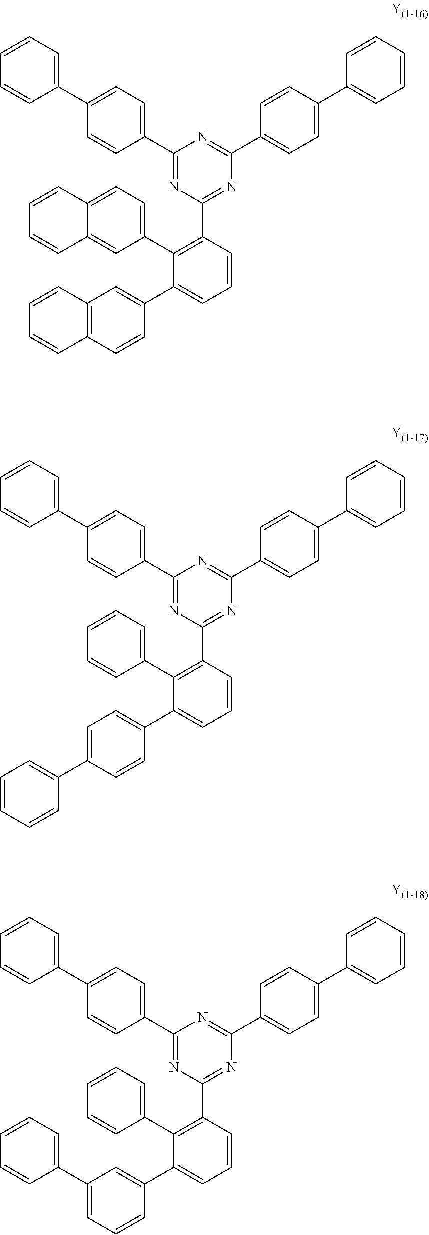 [SMILES: c1ccc(-c2ccc(-c3nc(-c4ccc(-c5ccccc5)cc4)nc(-c4cccc(-c5ccc6ccccc6c5)c4-c4ccc5ccccc5c4)n3)cc2)cc1 ; c1ccc(-c2ccc(-c3nc(-c4ccc(-c5ccccc5)cc4)nc(-c4cccc(-c5ccc(-c6ccccc6)cc5)c4-c4ccccc4)n3)cc2)cc1 ; c1ccc(-c2ccc(-c3nc(-c4ccc(-c5ccccc5)cc4)nc(-c4cccc(-c5cccc(-c6ccccc6)c5)c4-c4ccccc4)n3)cc2)cc1]

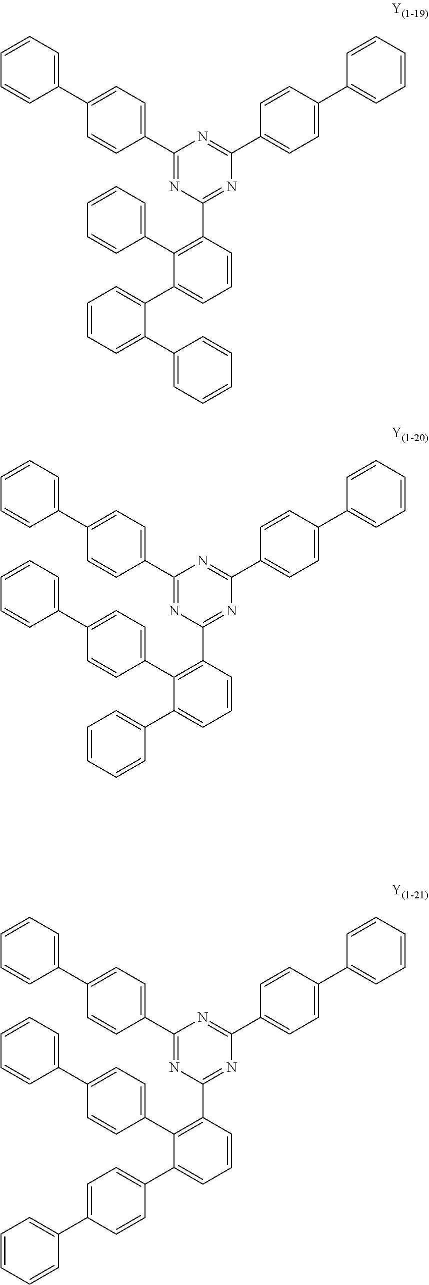 [SMILES: c1ccc(-c2ccc(-c3nc(-c4ccc(-c5ccccc5)cc4)nc(-c4cccc(-c5ccccc5-c5ccccc5)c4-c4ccccc4)n3)cc2)cc1 ; c1ccc(-c2ccc(-c3nc(-c4ccc(-c5ccccc5)cc4)nc(-c4cccc(-c5ccccc5)c4-c4ccc(-c5ccccc5)cc4)n3)cc2)cc1 ; c1ccc(-c2ccc(-c3nc(-c4ccc(-c5ccccc5)cc4)nc(-c4cccc(-c5ccc(-c6ccccc6)cc5)c4-c4ccc(-c5ccccc5)cc4)n3)cc2)cc1]

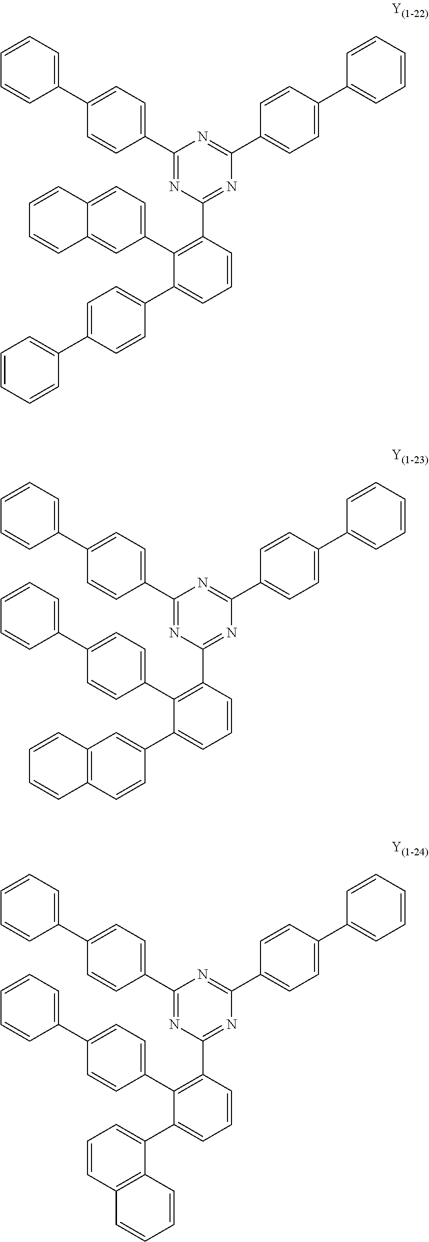 [SMILES: c1ccc(-c2ccc(-c3nc(-c4ccc(-c5ccccc5)cc4)nc(-c4cccc(-c5ccc(-c6ccccc6)cc5)c4-c4ccc5ccccc5c4)n3)cc2)cc1 ; c1ccc(-c2ccc(-c3nc(-c4ccc(-c5ccccc5)cc4)nc(-c4cccc(-c5ccc6ccccc6c5)c4-c4ccc(-c5ccccc5)cc4)n3)cc2)cc1 ; c1ccc(-c2ccc(-c3nc(-c4ccc(-c5ccccc5)cc4)nc(-c4cccc(-c5cccc6ccccc56)c4-c4ccc(-c5ccccc5)cc4)n3)cc2)cc1]

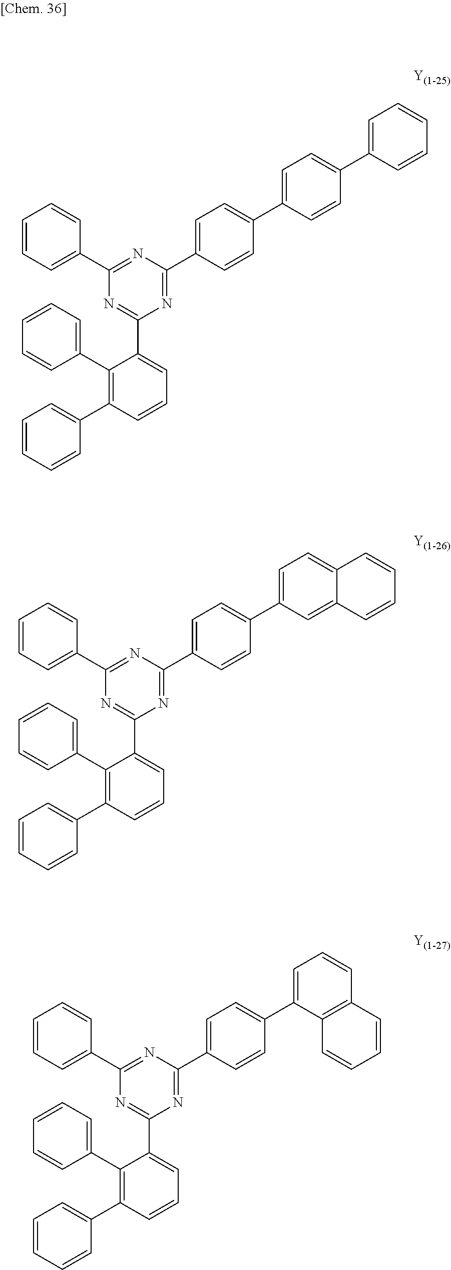 [SMILES: c1ccc(-c2ccc(-c3ccc(-c4nc(-c5ccccc5)nc(-c5cccc(-c6ccccc6)c5-c5ccccc5)n4)cc3)cc2)cc1 ; c1ccc(-c2nc(-c3ccc(-c4ccc5ccccc5c4)cc3)nc(-c3cccc(-c4ccccc4)c3-c3ccccc3)n2)cc1 ; c1ccc(-c2nc(-c3ccc(-c4cccc5ccccc45)cc3)nc(-c3cccc(-c4ccccc4)c3-c3ccccc3)n2)cc1]

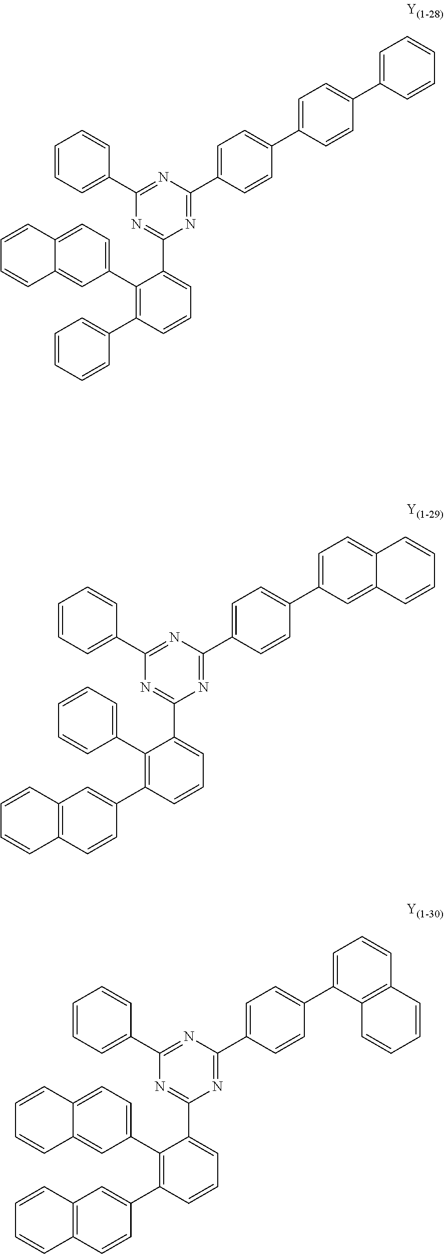 [SMILES: c1ccc(-c2ccc(-c3ccc(-c4nc(-c5ccccc5)nc(-c5cccc(-c6ccccc6)c5-c5ccc6ccccc6c5)n4)cc3)cc2)cc1 ; c1ccc(-c2nc(-c3ccc(-c4ccc5ccccc5c4)cc3)nc(-c3cccc(-c4ccc5ccccc5c4)c3-c3ccccc3)n2)cc1 ; c1ccc(-c2nc(-c3ccc(-c4cccc5ccccc45)cc3)nc(-c3cccc(-c4ccc5ccccc5c4)c3-c3ccc4ccccc4c3)n2)cc1]

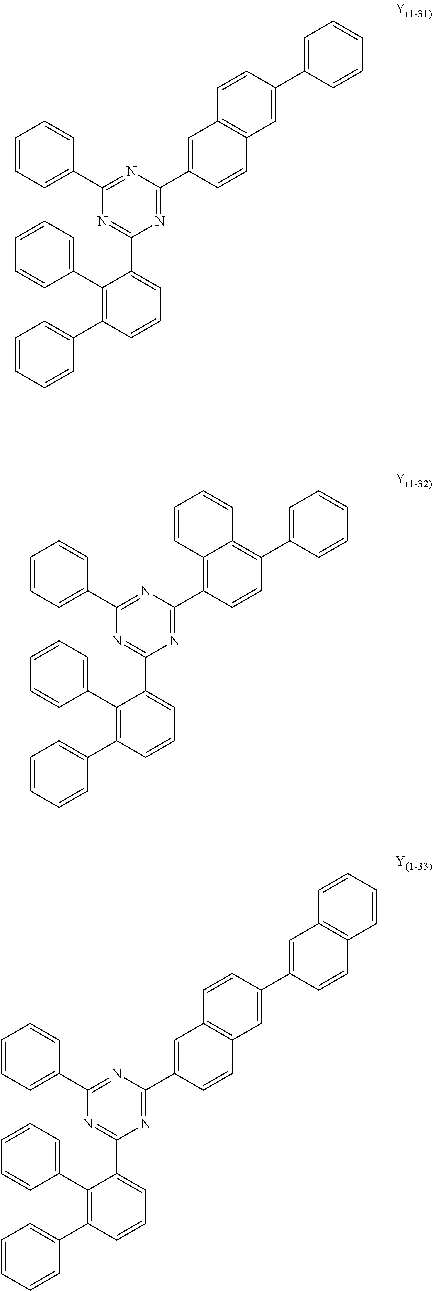 [SMILES: c1ccc(-c2ccc3cc(-c4nc(-c5ccccc5)nc(-c5cccc(-c6ccccc6)c5-c5ccccc5)n4)ccc3c2)cc1 ; c1ccc(-c2nc(-c3cccc(-c4ccccc4)c3-c3ccccc3)nc(-c3ccc(-c4ccccc4)c4ccccc34)n2)cc1 ; c1ccc(-c2nc(-c3ccc4cc(-c5ccc6ccccc6c5)ccc4c3)nc(-c3cccc(-c4ccccc4)c3-c3ccccc3)n2)cc1]

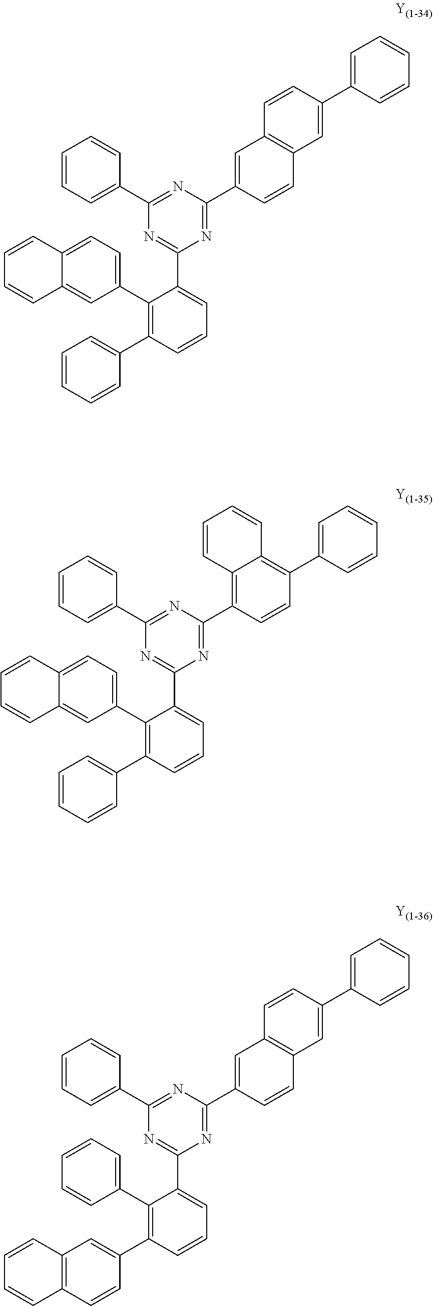 [SMILES: c1ccc(-c2ccc3cc(-c4nc(-c5ccccc5)nc(-c5cccc(-c6ccccc6)c5-c5ccc6ccccc6c5)n4)ccc3c2)cc1 ; c1ccc(-c2nc(-c3cccc(-c4ccccc4)c3-c3ccc4ccccc4c3)nc(-c3ccc(-c4ccccc4)c4ccccc34)n2)cc1 ; c1ccc(-c2ccc3cc(-c4nc(-c5ccccc5)nc(-c5cccc(-c6ccc7ccccc7c6)c5-c5ccccc5)n4)ccc3c2)cc1]

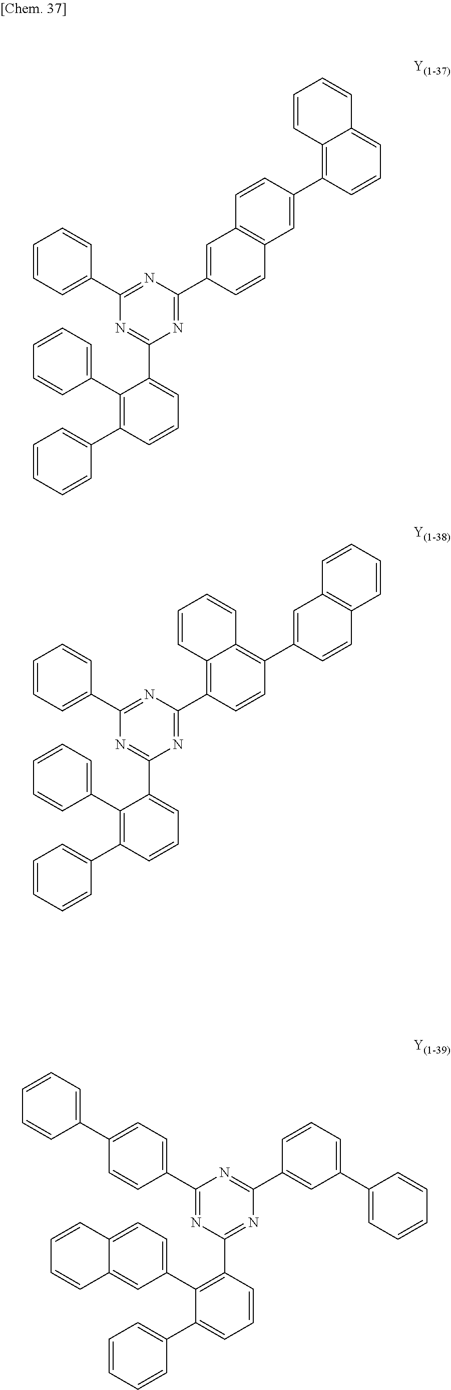 [SMILES: c1ccc(-c2nc(-c3ccc4cc(-c5cccc6ccccc56)ccc4c3)nc(-c3cccc(-c4ccccc4)c3-c3ccccc3)n2)cc1 ; c1ccc(-c2nc(-c3cccc(-c4ccccc4)c3-c3ccccc3)nc(-c3ccc(-c4ccc5ccccc5c4)c4ccccc34)n2)cc1 ; c1ccc(-c2ccc(-c3nc(-c4cccc(-c5ccccc5)c4)nc(-c4cccc(-c5ccccc5)c4-c4ccc5ccccc5c4)n3)cc2)cc1]

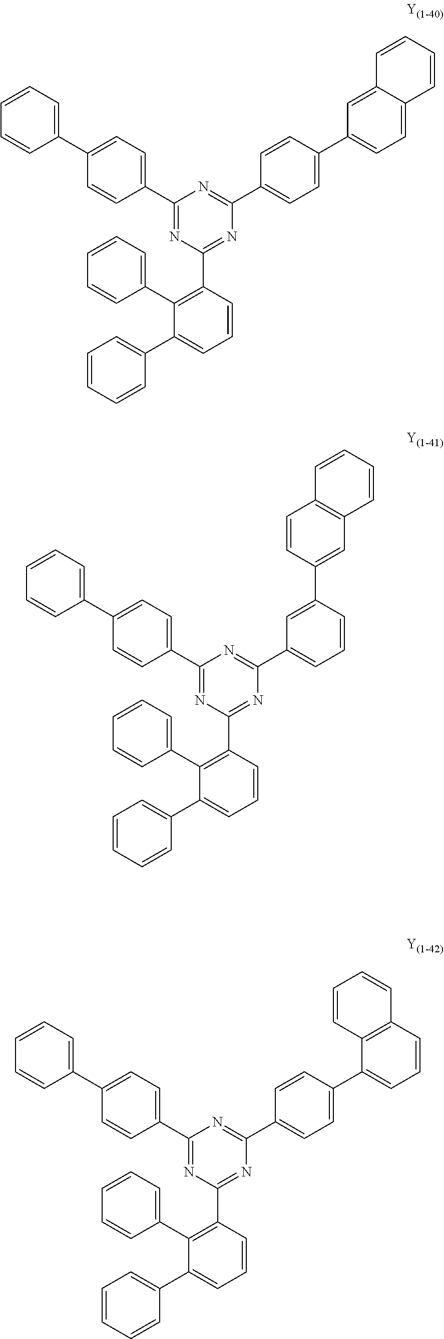 [SMILES: c1ccc(-c2ccc(-c3nc(-c4ccc(-c5ccc6ccccc6c5)cc4)nc(-c4cccc(-c5ccccc5)c4-c4ccccc4)n3)cc2)cc1 ; c1ccc(-c2ccc(-c3nc(-c4cccc(-c5ccc6ccccc6c5)c4)nc(-c4cccc(-c5ccccc5)c4-c4ccccc4)n3)cc2)cc1 ; c1ccc(-c2ccc(-c3nc(-c4ccc(-c5cccc6ccccc56)cc4)nc(-c4cccc(-c5ccccc5)c4-c4ccccc4)n3)cc2)cc1]

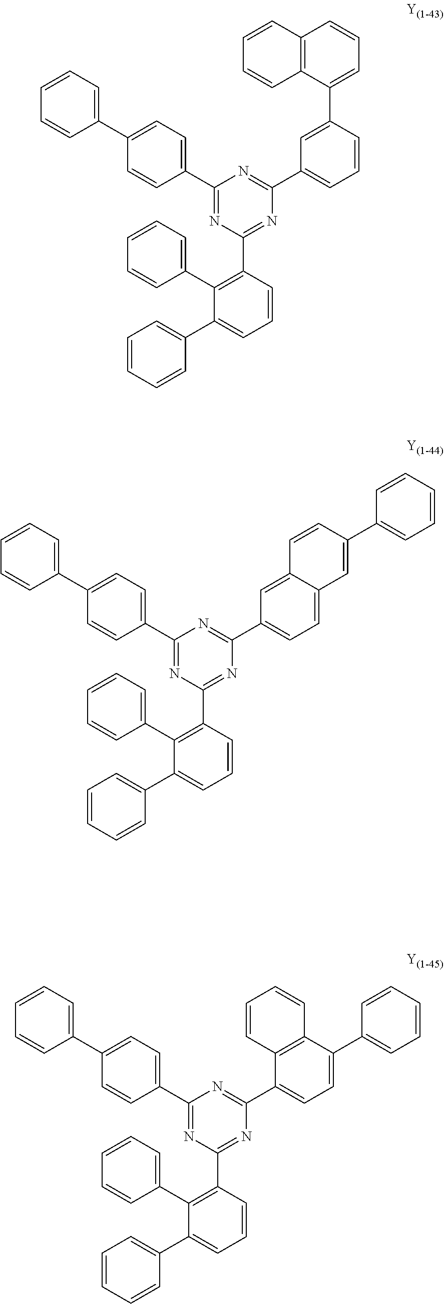 [SMILES: c1ccc(-c2ccc(-c3nc(-c4cccc(-c5cccc6ccccc56)c4)nc(-c4cccc(-c5ccccc5)c4-c4ccccc4)n3)cc2)cc1 ; c1ccc(-c2ccc(-c3nc(-c4ccc5cc(-c6ccccc6)ccc5c4)nc(-c4cccc(-c5ccccc5)c4-c4ccccc4)n3)cc2)cc1 ; c1ccc(-c2ccc(-c3nc(-c4cccc(-c5ccccc5)c4-c4ccccc4)nc(-c4ccc(-c5ccccc5)c5ccccc45)n3)cc2)cc1]

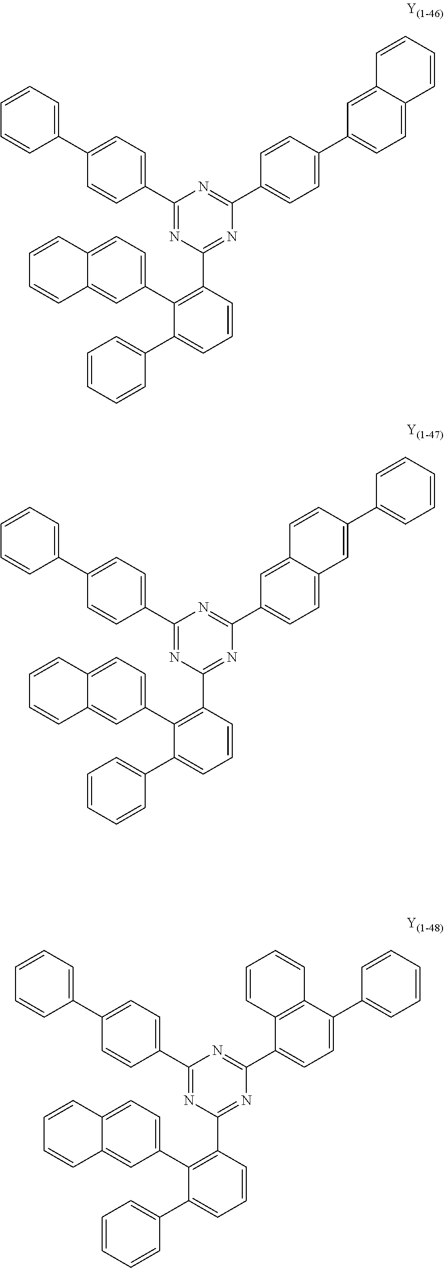 [SMILES: c1ccc(-c2ccc(-c3nc(-c4ccc(-c5ccc6ccccc6c5)cc4)nc(-c4cccc(-c5ccccc5)c4-c4ccc5ccccc5c4)n3)cc2)cc1 ; c1ccc(-c2ccc(-c3nc(-c4ccc5cc(-c6ccccc6)ccc5c4)nc(-c4cccc(-c5ccccc5)c4-c4ccc5ccccc5c4)n3)cc2)cc1 ; c1ccc(-c2ccc(-c3nc(-c4cccc(-c5ccccc5)c4-c4ccc5ccccc5c4)nc(-c4ccc(-c5ccccc5)c5ccccc45)n3)cc2)cc1]

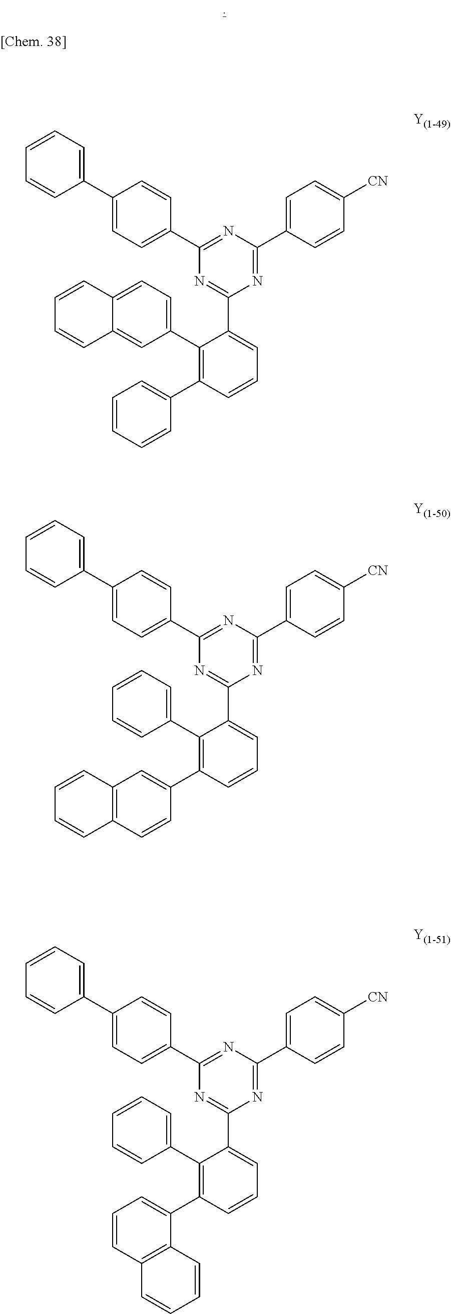 [SMILES: N#Cc1ccc(-c2nc(-c3ccc(-c4ccccc4)cc3)nc(-c3cccc(-c4ccccc4)c3-c3ccc4ccccc4c3)n2)cc1 ; N#Cc1ccc(-c2nc(-c3ccc(-c4ccccc4)cc3)nc(-c3cccc(-c4ccc5ccccc5c4)c3-c3ccccc3)n2)cc1 ; N#Cc1ccc(-c2nc(-c3ccc(-c4ccccc4)cc3)nc(-c3cccc(-c4cccc5ccccc45)c3-c3ccccc3)n2)cc1]

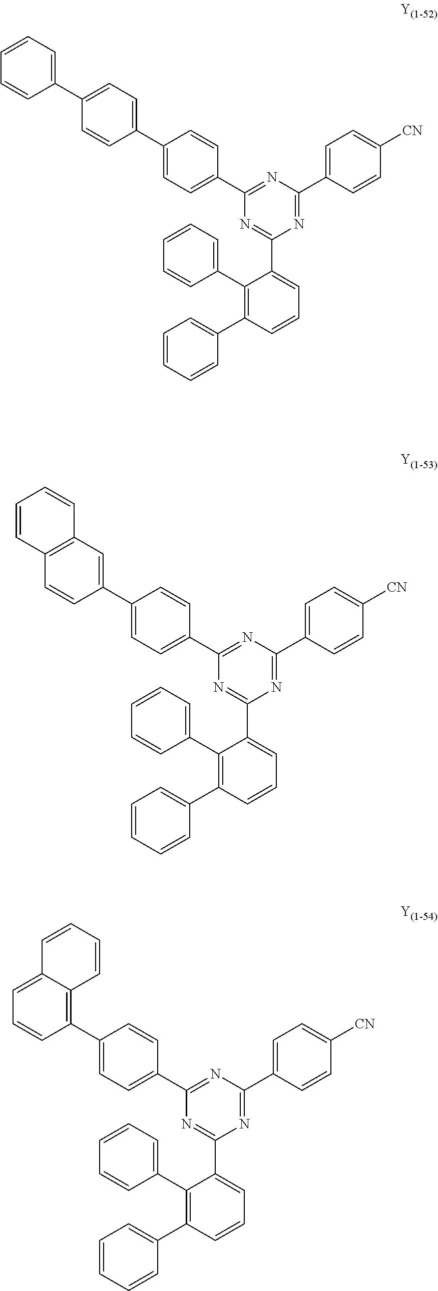 [SMILES: N#Cc1ccc(-c2nc(-c3ccc(-c4ccc(-c5ccccc5)cc4)cc3)nc(-c3cccc(-c4ccccc4)c3-c3ccccc3)n2)cc1 ; N#Cc1ccc(-c2nc(-c3ccc(-c4ccc5ccccc5c4)cc3)nc(-c3cccc(-c4ccccc4)c3-c3ccccc3)n2)cc1 ; N#Cc1ccc(-c2nc(-c3ccc(-c4cccc5ccccc45)cc3)nc(-c3cccc(-c4ccccc4)c3-c3ccccc3)n2)cc1]

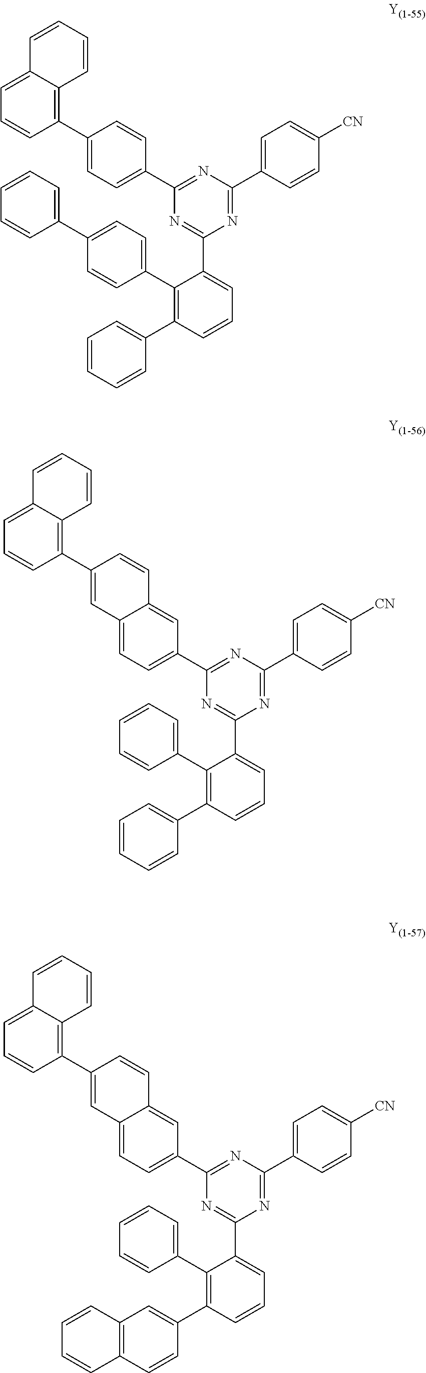 [SMILES: N#Cc1ccc(-c2nc(-c3ccc(-c4cccc5ccccc45)cc3)nc(-c3cccc(-c4ccccc4)c3-c3ccc(-c4ccccc4)cc3)n2)cc1 ; N#Cc1ccc(-c2nc(-c3ccc4cc(-c5cccc6ccccc56)ccc4c3)nc(-c3cccc(-c4ccccc4)c3-c3ccccc3)n2)cc1 ; N#Cc1ccc(-c2nc(-c3ccc4cc(-c5cccc6ccccc56)ccc4c3)nc(-c3cccc(-c4ccc5ccccc5c4)c3-c3ccccc3)n2)cc1]

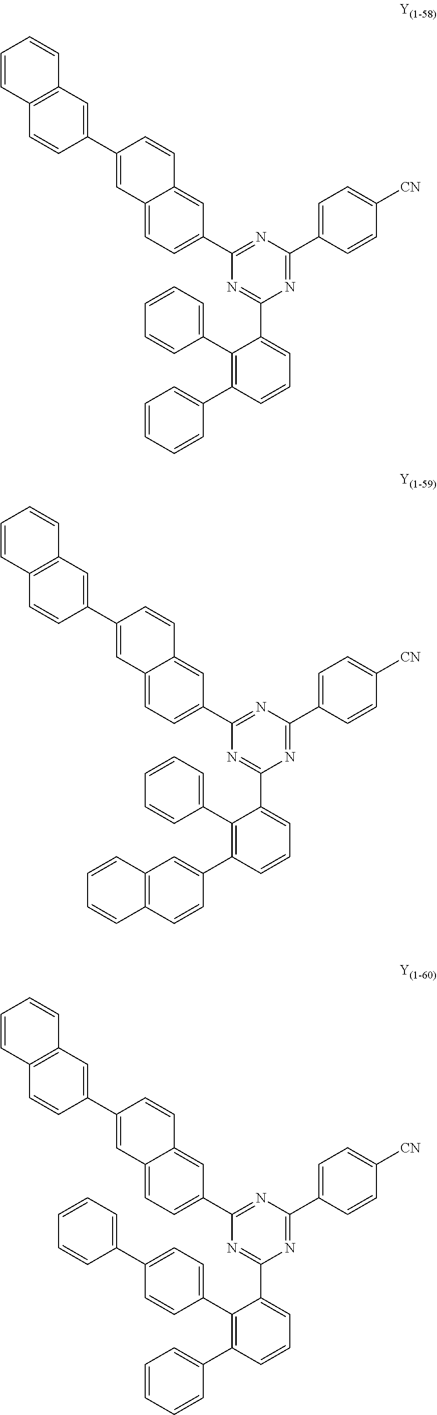 [SMILES: N#Cc1ccc(-c2nc(-c3ccc4cc(-c5ccc6ccccc6c5)ccc4c3)nc(-c3cccc(-c4ccccc4)c3-c3ccccc3)n2)cc1 ; N#Cc1ccc(-c2nc(-c3ccc4cc(-c5ccc6ccccc6c5)ccc4c3)nc(-c3cccc(-c4ccc5ccccc5c4)c3-c3ccccc3)n2)cc1 ; N#Cc1ccc(-c2nc(-c3ccc4cc(-c5ccc6ccccc6c5)ccc4c3)nc(-c3cccc(-c4ccccc4)c3-c3ccc(-c4ccccc4)cc3)n2)cc1]

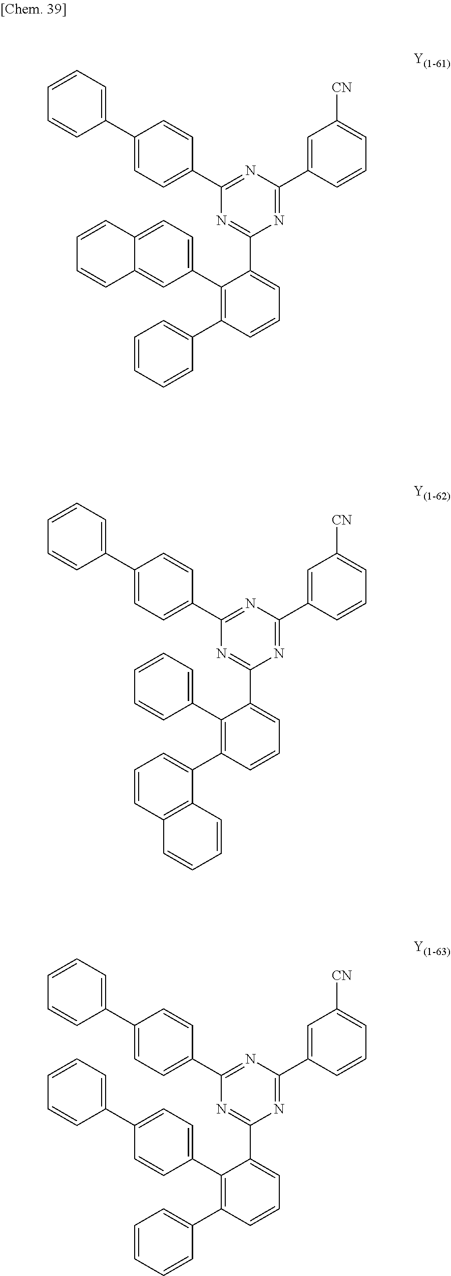 [SMILES: N#Cc1cccc(-c2nc(-c3ccc(-c4ccccc4)cc3)nc(-c3cccc(-c4ccccc4)c3-c3ccc4ccccc4c3)n2)c1 ; N#Cc1cccc(-c2nc(-c3ccc(-c4ccccc4)cc3)nc(-c3cccc(-c4cccc5ccccc45)c3-c3ccccc3)n2)c1 ; N#Cc1cccc(-c2nc(-c3ccc(-c4ccccc4)cc3)nc(-c3cccc(-c4ccccc4)c3-c3ccc(-c4ccccc4)cc3)n2)c1]

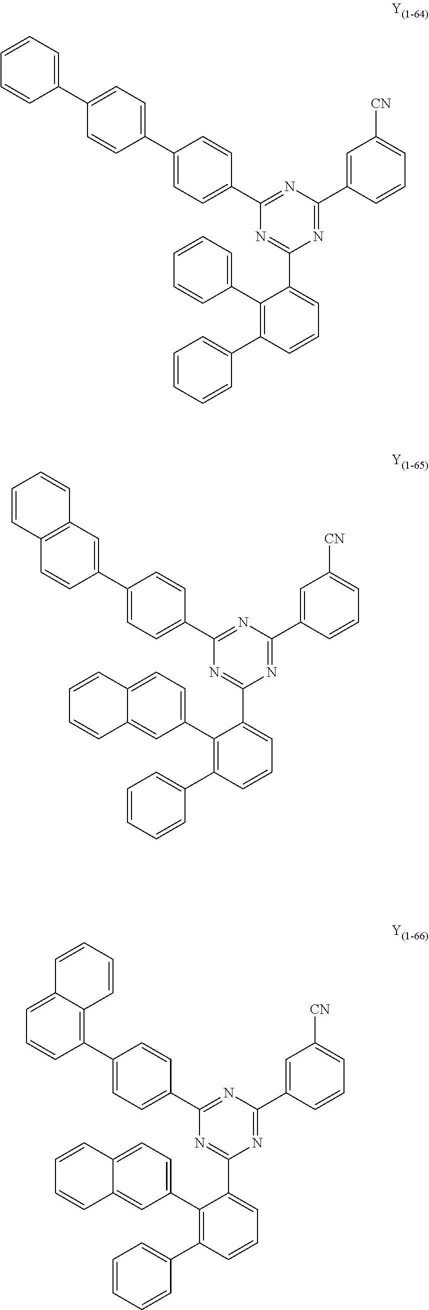 [SMILES: N#Cc1cccc(-c2nc(-c3ccc(-c4ccc(-c5ccccc5)cc4)cc3)nc(-c3cccc(-c4ccccc4)c3-c3ccccc3)n2)c1 ; N#Cc1cccc(-c2nc(-c3ccc(-c4ccc5ccccc5c4)cc3)nc(-c3cccc(-c4ccccc4)c3-c3ccc4ccccc4c3)n2)c1 ; N#Cc1cccc(-c2nc(-c3ccc(-c4cccc5ccccc45)cc3)nc(-c3cccc(-c4ccccc4)c3-c3ccc4ccccc4c3)n2)c1]

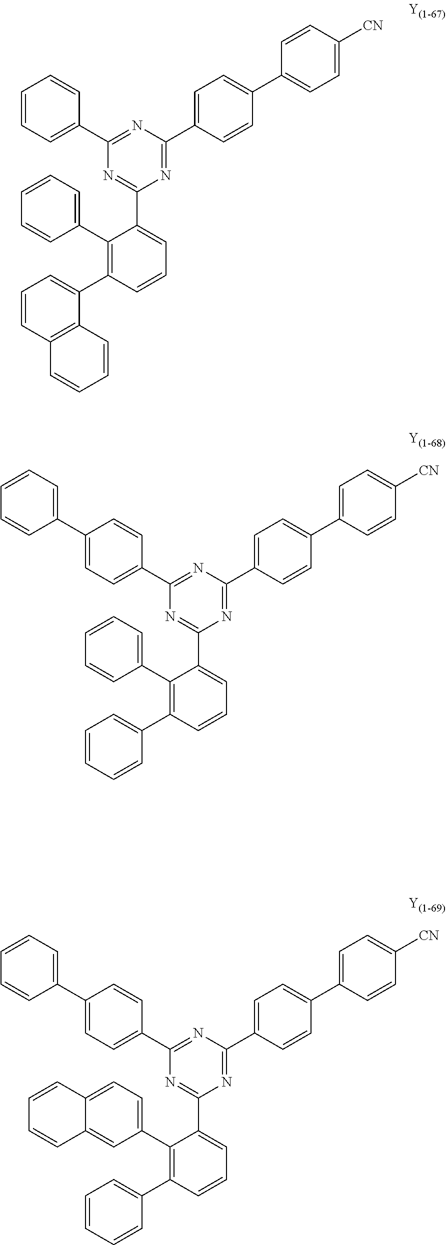 [SMILES: N#Cc1ccc(-c2ccc(-c3nc(-c4ccccc4)nc(-c4cccc(-c5cccc6ccccc56)c4-c4ccccc4)n3)cc2)cc1 ; N#Cc1ccc(-c2ccc(-c3nc(-c4ccc(-c5ccccc5)cc4)nc(-c4cccc(-c5ccccc5)c4-c4ccccc4)n3)cc2)cc1 ; N#Cc1ccc(-c2ccc(-c3nc(-c4ccc(-c5ccccc5)cc4)nc(-c4cccc(-c5ccccc5)c4-c4ccc5ccccc5c4)n3)cc2)cc1]

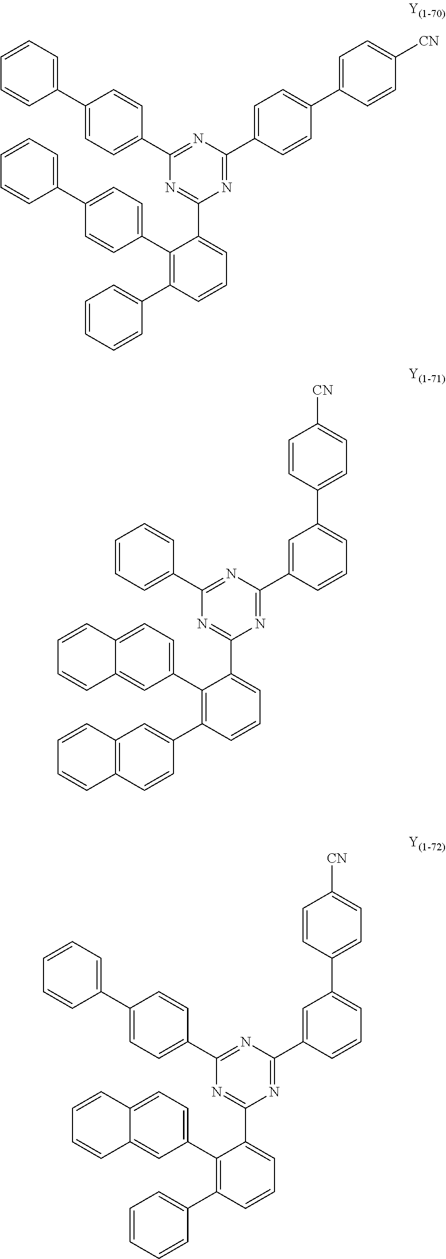 [SMILES: N#Cc1ccc(-c2ccc(-c3nc(-c4ccc(-c5ccccc5)cc4)nc(-c4cccc(-c5ccccc5)c4-c4ccc(-c5ccccc5)cc4)n3)cc2)cc1 ; N#Cc1ccc(-c2cccc(-c3nc(-c4ccccc4)nc(-c4cccc(-c5ccc6ccccc6c5)c4-c4ccc5ccccc5c4)n3)c2)cc1 ; N#Cc1ccc(-c2cccc(-c3nc(-c4ccc(-c5ccccc5)cc4)nc(-c4cccc(-c5ccccc5)c4-c4ccc5ccccc5c4)n3)c2)cc1]

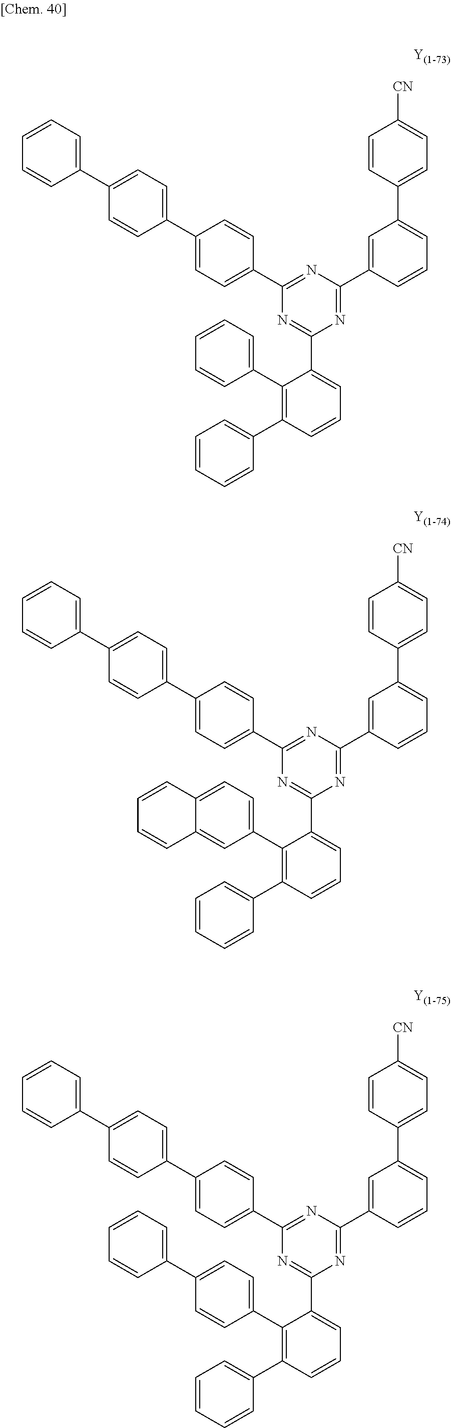 [SMILES: N#Cc1ccc(-c2cccc(-c3nc(-c4ccc(-c5ccc(-c6ccccc6)cc5)cc4)nc(-c4cccc(-c5ccccc5)c4-c4ccccc4)n3)c2)cc1 ; N#Cc1ccc(-c2cccc(-c3nc(-c4ccc(-c5ccc(-c6ccccc6)cc5)cc4)nc(-c4cccc(-c5ccccc5)c4-c4ccc5ccccc5c4)n3)c2)cc1 ; N#Cc1ccc(-c2cccc(-c3nc(-c4ccc(-c5ccc(-c6ccccc6)cc5)cc4)nc(-c4cccc(-c5ccccc5)c4-c4ccc(-c5ccccc5)cc4)n3)c2)cc1]

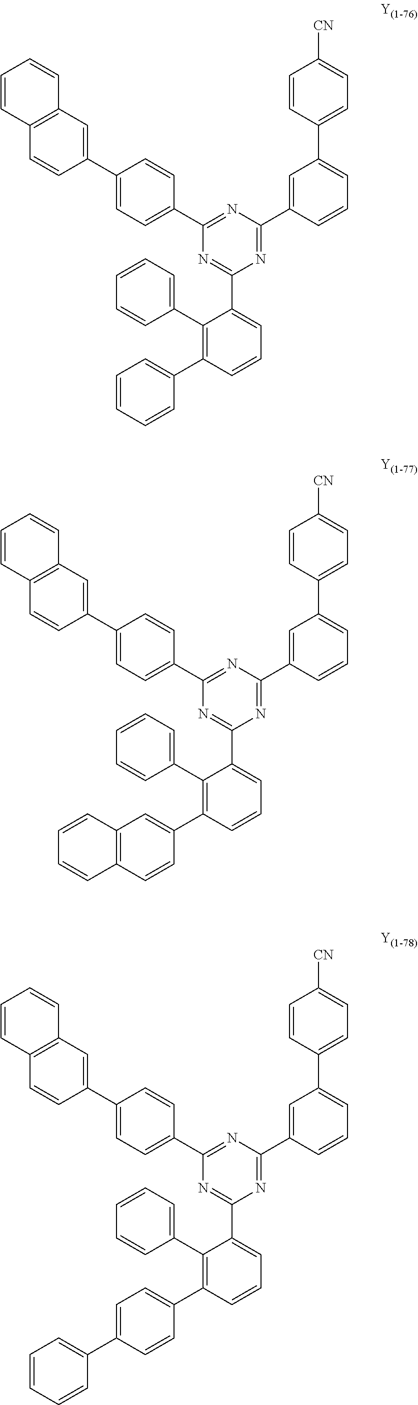 [SMILES: N#Cc1ccc(-c2cccc(-c3nc(-c4ccc(-c5ccc6ccccc6c5)cc4)nc(-c4cccc(-c5ccccc5)c4-c4ccccc4)n3)c2)cc1 ; N#Cc1ccc(-c2cccc(-c3nc(-c4ccc(-c5ccc6ccccc6c5)cc4)nc(-c4cccc(-c5ccc6ccccc6c5)c4-c4ccccc4)n3)c2)cc1 ; N#Cc1ccc(-c2cccc(-c3nc(-c4ccc(-c5ccc6ccccc6c5)cc4)nc(-c4cccc(-c5ccc(-c6ccccc6)cc5)c4-c4ccccc4)n3)c2)cc1]

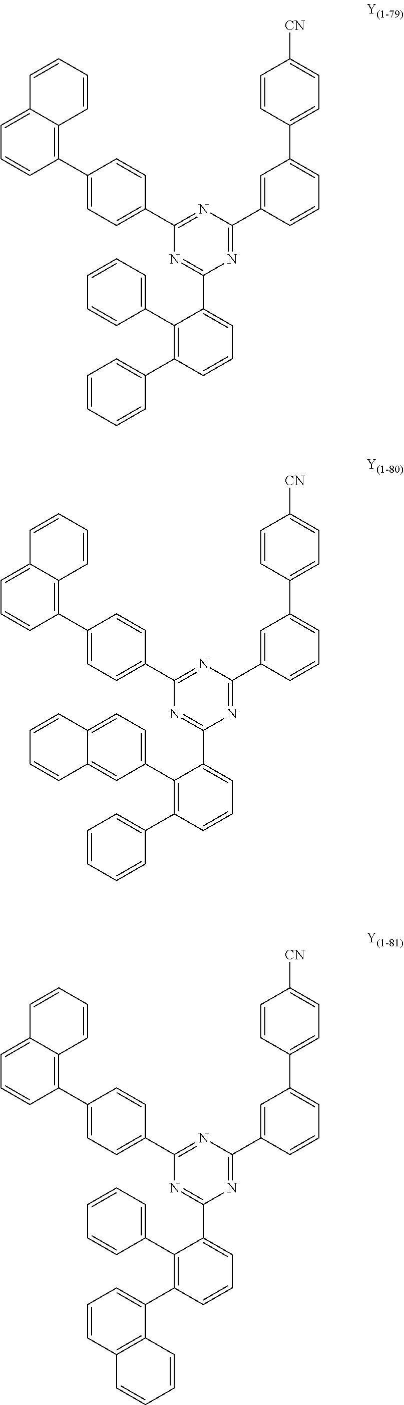 [SMILES: N#Cc1ccc(-c2cccc(-c3nc(-c4ccc(-c5cccc6ccccc56)cc4)nc(-c4cccc(-c5ccccc5)c4-c4ccccc4)n3)c2)cc1 ; N#Cc1ccc(-c2cccc(-c3nc(-c4ccc(-c5cccc6ccccc56)cc4)nc(-c4cccc(-c5ccccc5)c4-c4ccc5ccccc5c4)n3)c2)cc1 ; N#Cc1ccc(-c2cccc(-c3nc(-c4ccc(-c5cccc6ccccc56)cc4)nc(-c4cccc(-c5cccc6ccccc56)c4-c4ccccc4)n3)c2)cc1]

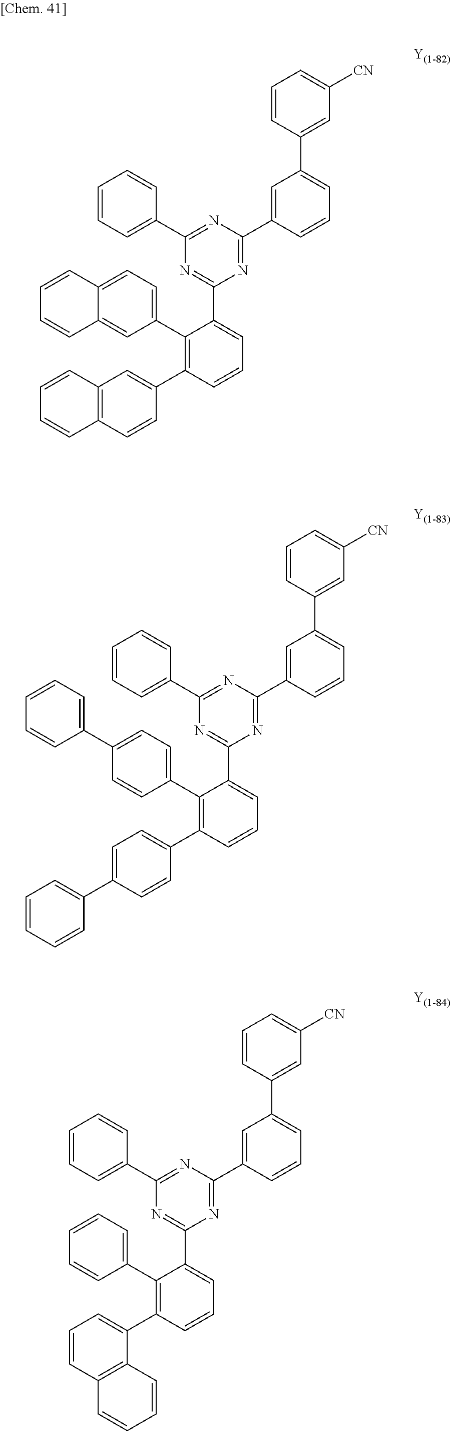 [SMILES: N#Cc1cccc(-c2cccc(-c3nc(-c4ccccc4)nc(-c4cccc(-c5ccc6ccccc6c5)c4-c4ccc5ccccc5c4)n3)c2)c1 ; N#Cc1cccc(-c2cccc(-c3nc(-c4ccccc4)nc(-c4cccc(-c5ccc(-c6ccccc6)cc5)c4-c4ccc(-c5ccccc5)cc4)n3)c2)c1 ; N#Cc1cccc(-c2cccc(-c3nc(-c4ccccc4)nc(-c4cccc(-c5cccc6ccccc56)c4-c4ccccc4)n3)c2)c1]

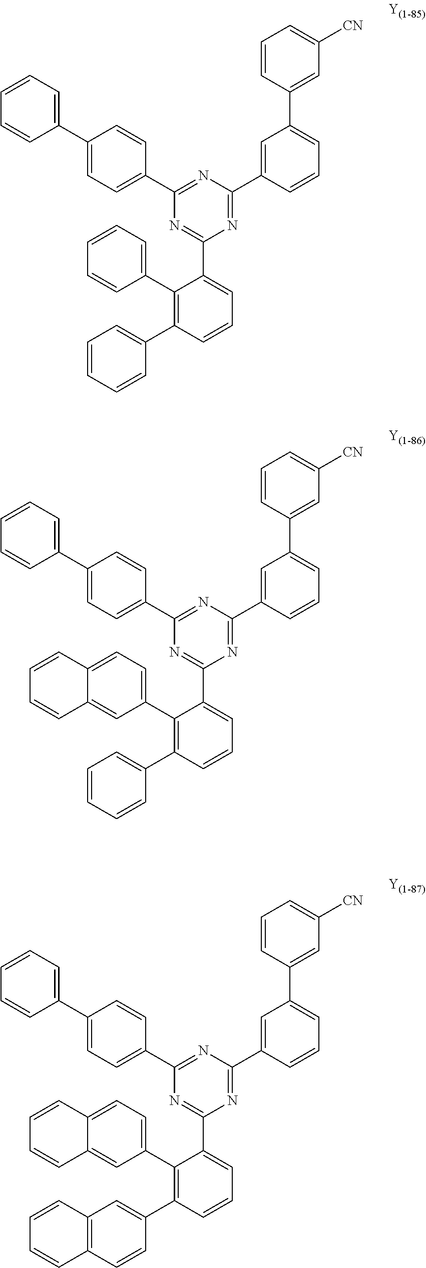 [SMILES: N#Cc1cccc(-c2cccc(-c3nc(-c4ccc(-c5ccccc5)cc4)nc(-c4cccc(-c5ccccc5)c4-c4ccccc4)n3)c2)c1 ; N#Cc1cccc(-c2cccc(-c3nc(-c4ccc(-c5ccccc5)cc4)nc(-c4cccc(-c5ccccc5)c4-c4ccc5ccccc5c4)n3)c2)c1 ; N#Cc1cccc(-c2cccc(-c3nc(-c4ccc(-c5ccccc5)cc4)nc(-c4cccc(-c5ccc6ccccc6c5)c4-c4ccc5ccccc5c4)n3)c2)c1]

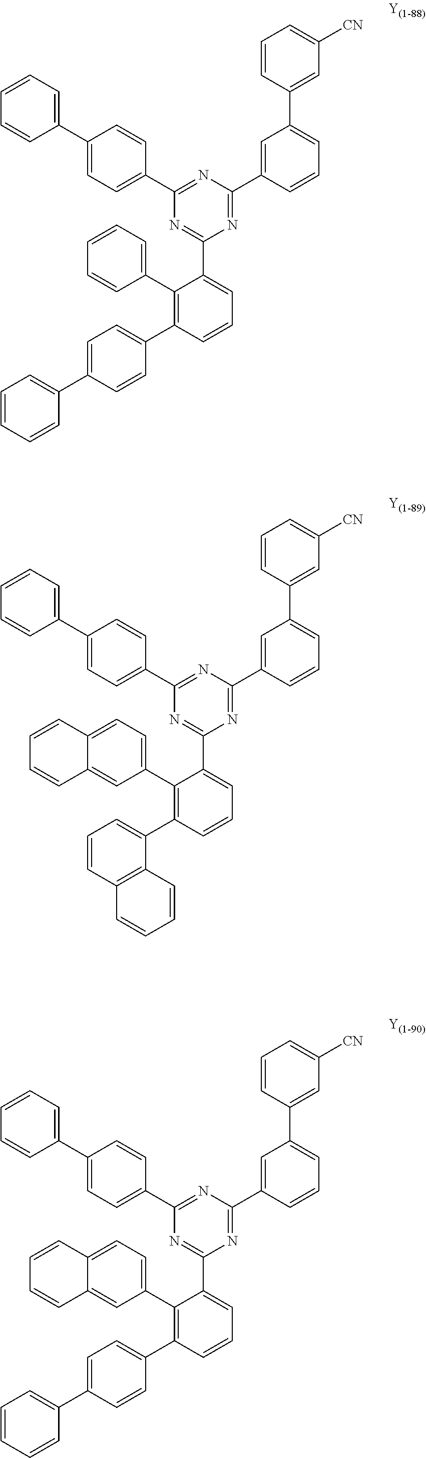 [SMILES: N#Cc1cccc(-c2cccc(-c3nc(-c4ccc(-c5ccccc5)cc4)nc(-c4cccc(-c5ccc(-c6ccccc6)cc5)c4-c4ccccc4)n3)c2)c1 ; N#Cc1cccc(-c2cccc(-c3nc(-c4ccc(-c5ccccc5)cc4)nc(-c4cccc(-c5cccc6ccccc56)c4-c4ccc5ccccc5c4)n3)c2)c1 ; N#Cc1cccc(-c2cccc(-c3nc(-c4ccc(-c5ccccc5)cc4)nc(-c4cccc(-c5ccc(-c6ccccc6)cc5)c4-c4ccc5ccccc5c4)n3)c2)c1]

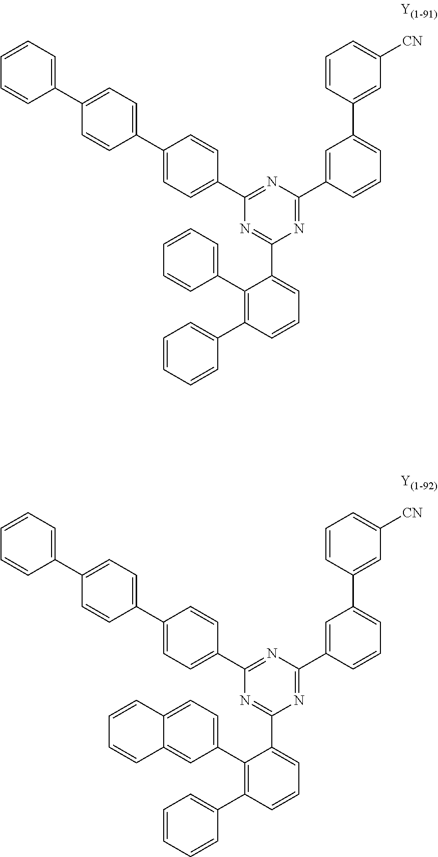 [SMILES: N#Cc1cccc(-c2cccc(-c3nc(-c4ccc(-c5ccc(-c6ccccc6)cc5)cc4)nc(-c4cccc(-c5ccccc5)c4-c4ccccc4)n3)c2)c1 ; N#Cc1cccc(-c2cccc(-c3nc(-c4ccc(-c5ccc(-c6ccccc6)cc5)cc4)nc(-c4cccc(-c5ccccc5)c4-c4ccc5ccccc5c4)n3)c2)c1]

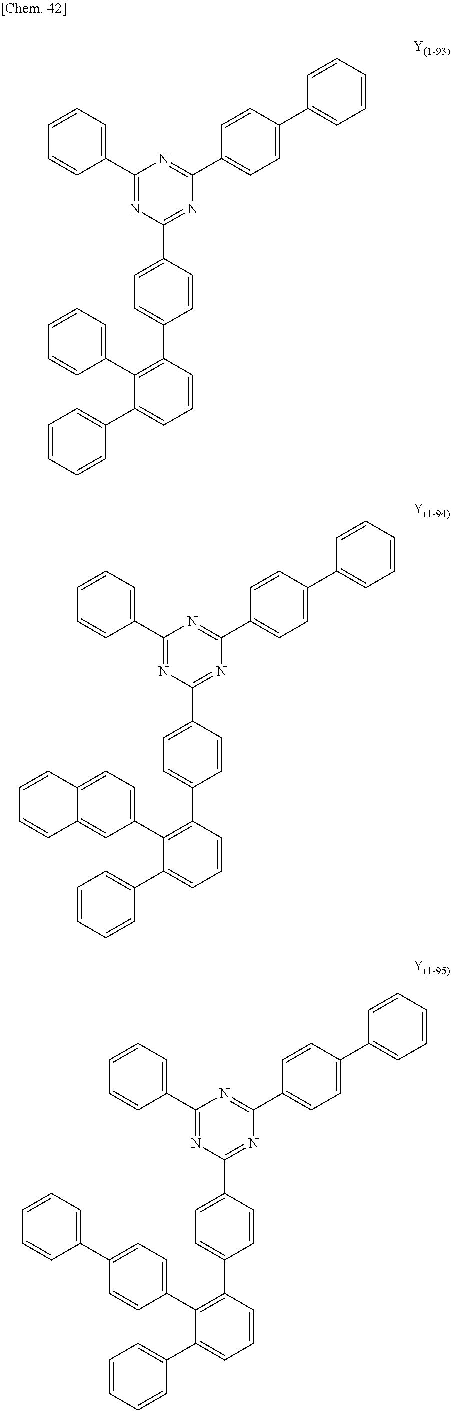 [SMILES: c1ccc(-c2ccc(-c3nc(-c4ccccc4)nc(-c4ccc(-c5cccc(-c6ccccc6)c5-c5ccccc5)cc4)n3)cc2)cc1 ; c1ccc(-c2ccc(-c3nc(-c4ccccc4)nc(-c4ccc(-c5cccc(-c6ccccc6)c5-c5ccc6ccccc6c5)cc4)n3)cc2)cc1 ; c1ccc(-c2ccc(-c3nc(-c4ccccc4)nc(-c4ccc(-c5cccc(-c6ccccc6)c5-c5ccc(-c6ccccc6)cc5)cc4)n3)cc2)cc1]

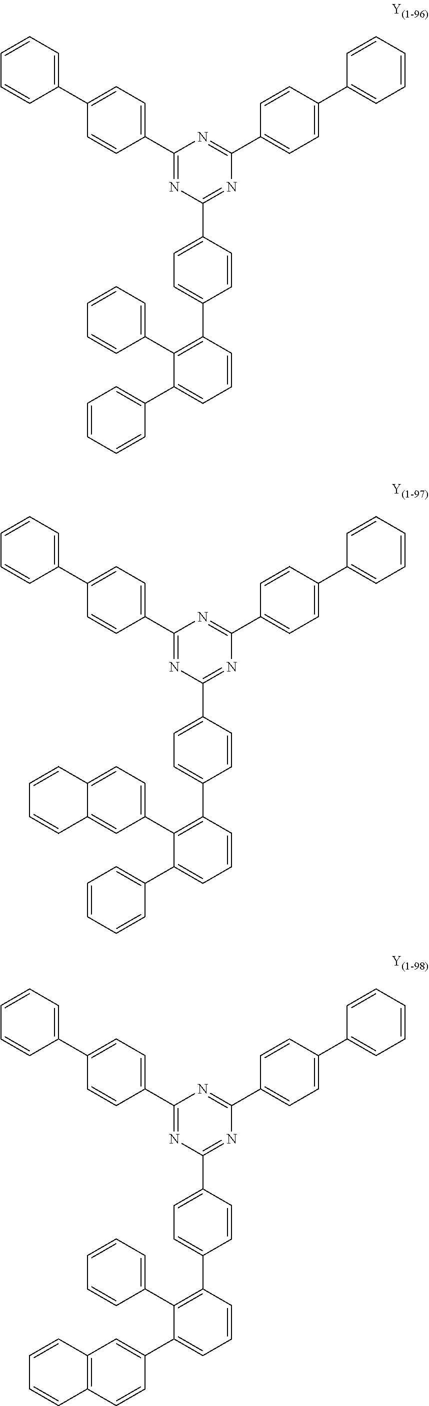 [SMILES: c1ccc(-c2ccc(-c3nc(-c4ccc(-c5ccccc5)cc4)nc(-c4ccc(-c5cccc(-c6ccccc6)c5-c5ccccc5)cc4)n3)cc2)cc1 ; c1ccc(-c2ccc(-c3nc(-c4ccc(-c5ccccc5)cc4)nc(-c4ccc(-c5cccc(-c6ccccc6)c5-c5ccc6ccccc6c5)cc4)n3)cc2)cc1 ; c1ccc(-c2ccc(-c3nc(-c4ccc(-c5ccccc5)cc4)nc(-c4ccc(-c5cccc(-c6ccc7ccccc7c6)c5-c5ccccc5)cc4)n3)cc2)cc1]

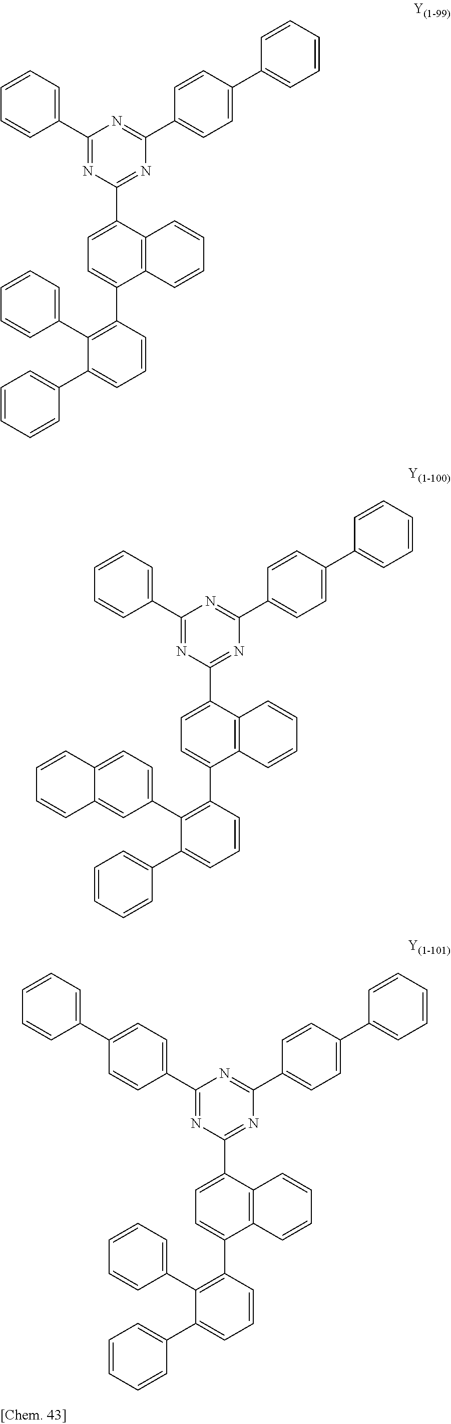 [SMILES: c1ccc(-c2ccc(-c3nc(-c4ccccc4)nc(-c4ccc(-c5cccc(-c6ccccc6)c5-c5ccccc5)c5ccccc45)n3)cc2)cc1 ; c1ccc(-c2ccc(-c3nc(-c4ccccc4)nc(-c4ccc(-c5cccc(-c6ccccc6)c5-c5ccc6ccccc6c5)c5ccccc45)n3)cc2)cc1 ; c1ccc(-c2ccc(-c3nc(-c4ccc(-c5ccccc5)cc4)nc(-c4ccc(-c5cccc(-c6ccccc6)c5-c5ccccc5)c5ccccc45)n3)cc2)cc1]

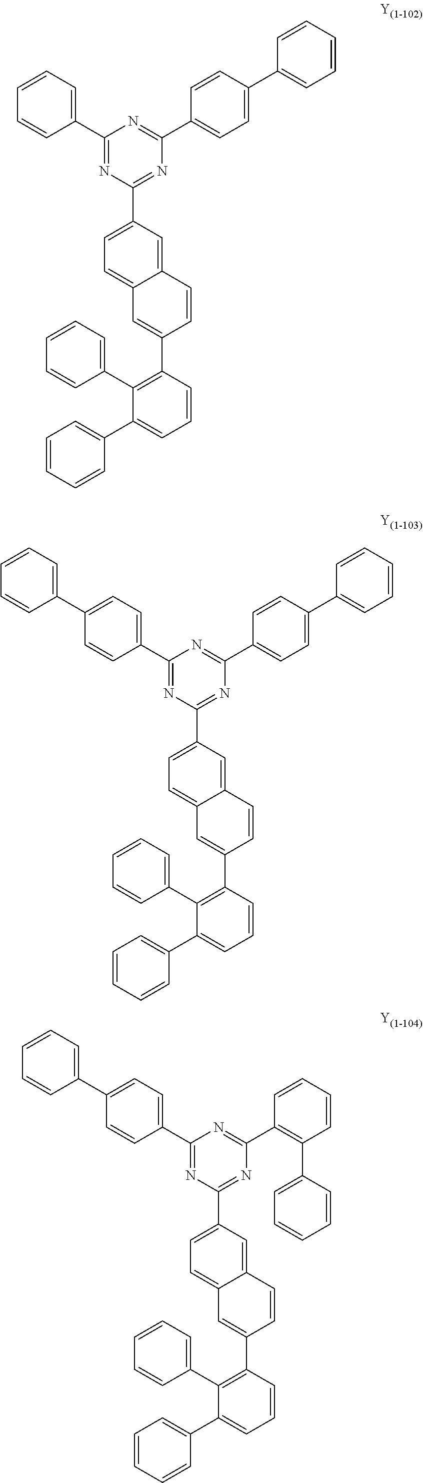 [SMILES: c1ccc(-c2ccc(-c3nc(-c4ccccc4)nc(-c4ccc5cc(-c6cccc(-c7ccccc7)c6-c6ccccc6)ccc5c4)n3)cc2)cc1 ; c1ccc(-c2ccc(-c3nc(-c4ccc(-c5ccccc5)cc4)nc(-c4ccc5cc(-c6cccc(-c7ccccc7)c6-c6ccccc6)ccc5c4)n3)cc2)cc1 ; c1ccc(-c2ccc(-c3nc(-c4ccc5cc(-c6cccc(-c7ccccc7)c6-c6ccccc6)ccc5c4)nc(-c4ccccc4-c4ccccc4)n3)cc2)cc1]

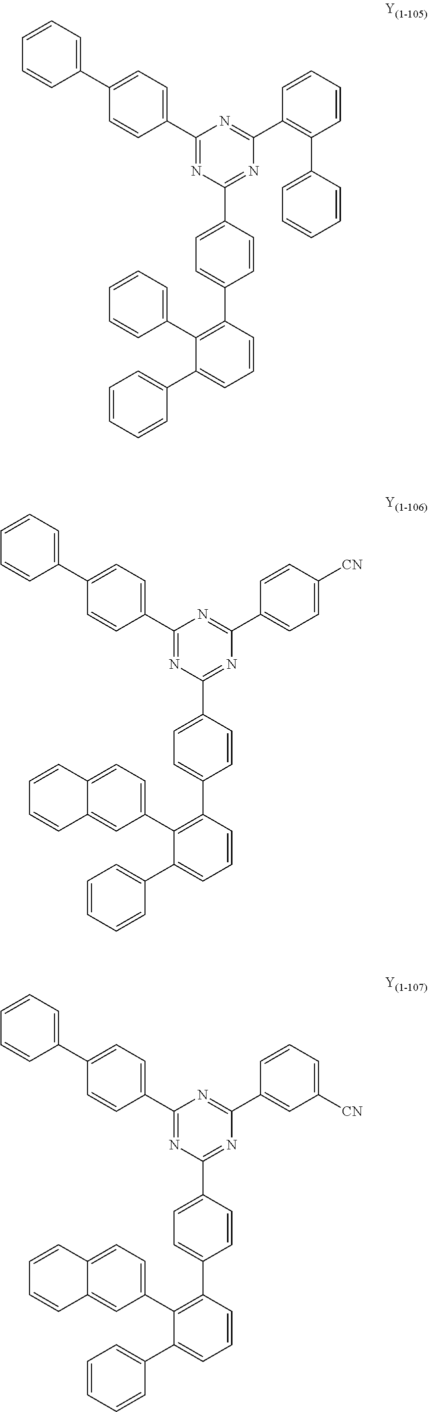 [SMILES: c1ccc(-c2ccc(-c3nc(-c4ccc(-c5cccc(-c6ccccc6)c5-c5ccccc5)cc4)nc(-c4ccccc4-c4ccccc4)n3)cc2)cc1 ; N#Cc1ccc(-c2nc(-c3ccc(-c4ccccc4)cc3)nc(-c3ccc(-c4cccc(-c5ccccc5)c4-c4ccc5ccccc5c4)cc3)n2)cc1 ; N#Cc1cccc(-c2nc(-c3ccc(-c4ccccc4)cc3)nc(-c3ccc(-c4cccc(-c5ccccc5)c4-c4ccc5ccccc5c4)cc3)n2)c1]

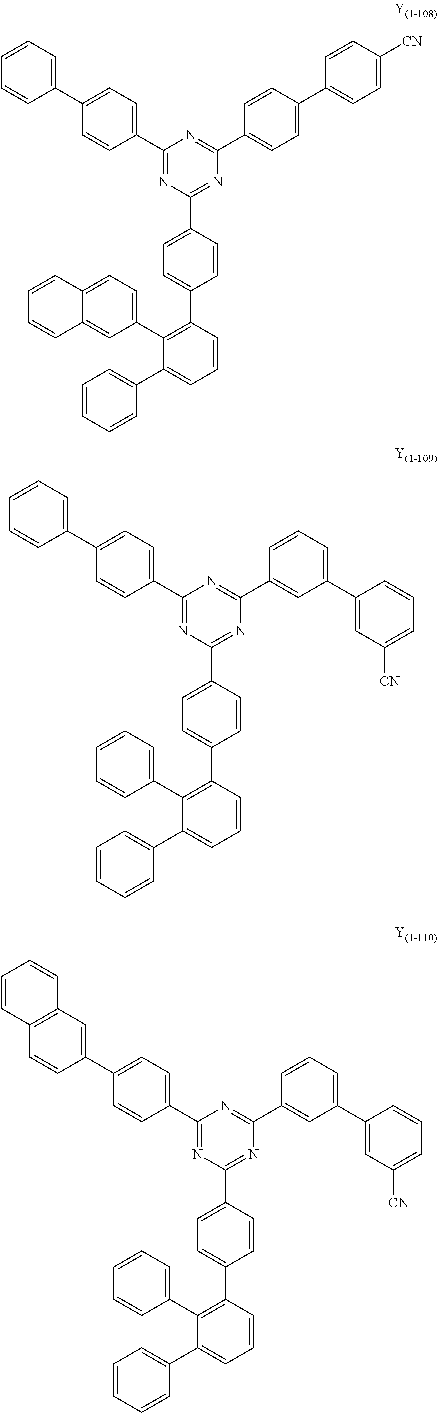 [SMILES: N#Cc1ccc(-c2ccc(-c3nc(-c4ccc(-c5ccccc5)cc4)nc(-c4ccc(-c5cccc(-c6ccccc6)c5-c5ccc6ccccc6c5)cc4)n3)cc2)cc1 ; N#Cc1cccc(-c2cccc(-c3nc(-c4ccc(-c5ccccc5)cc4)nc(-c4ccc(-c5cccc(-c6ccccc6)c5-c5ccccc5)cc4)n3)c2)c1 ; N#Cc1cccc(-c2cccc(-c3nc(-c4ccc(-c5ccc6ccccc6c5)cc4)nc(-c4ccc(-c5cccc(-c6ccccc6)c5-c5ccccc5)cc4)n3)c2)c1]

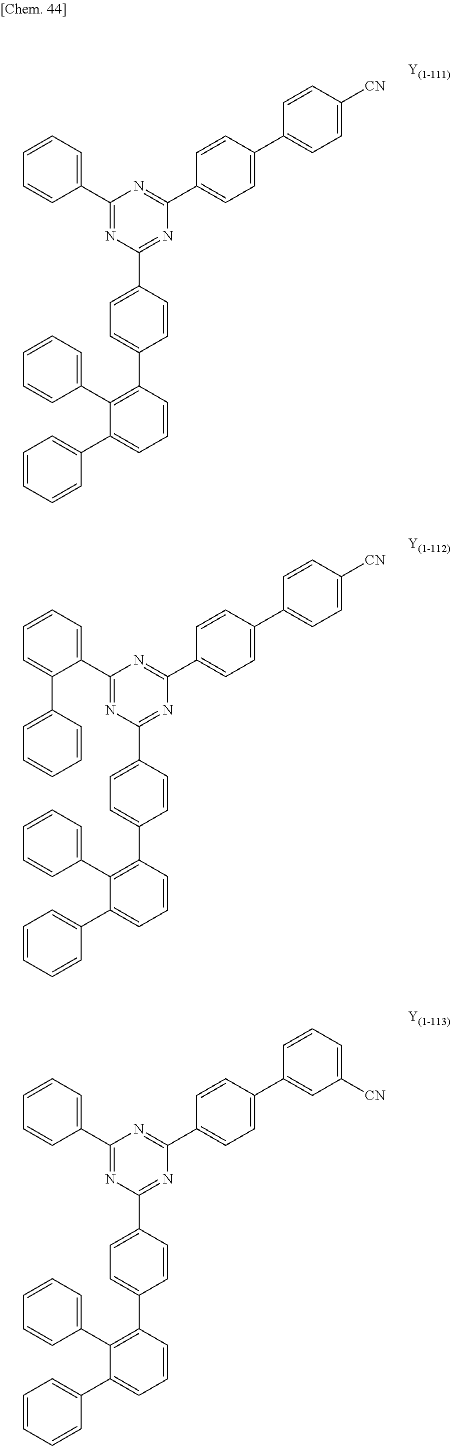 [SMILES: N#Cc1ccc(-c2ccc(-c3nc(-c4ccccc4)nc(-c4ccc(-c5cccc(-c6ccccc6)c5-c5ccccc5)cc4)n3)cc2)cc1 ; N#Cc1ccc(-c2ccc(-c3nc(-c4ccc(-c5cccc(-c6ccccc6)c5-c5ccccc5)cc4)nc(-c4ccccc4-c4ccccc4)n3)cc2)cc1 ; N#Cc1cccc(-c2ccc(-c3nc(-c4ccccc4)nc(-c4ccc(-c5cccc(-c6ccccc6)c5-c5ccccc5)cc4)n3)cc2)c1]

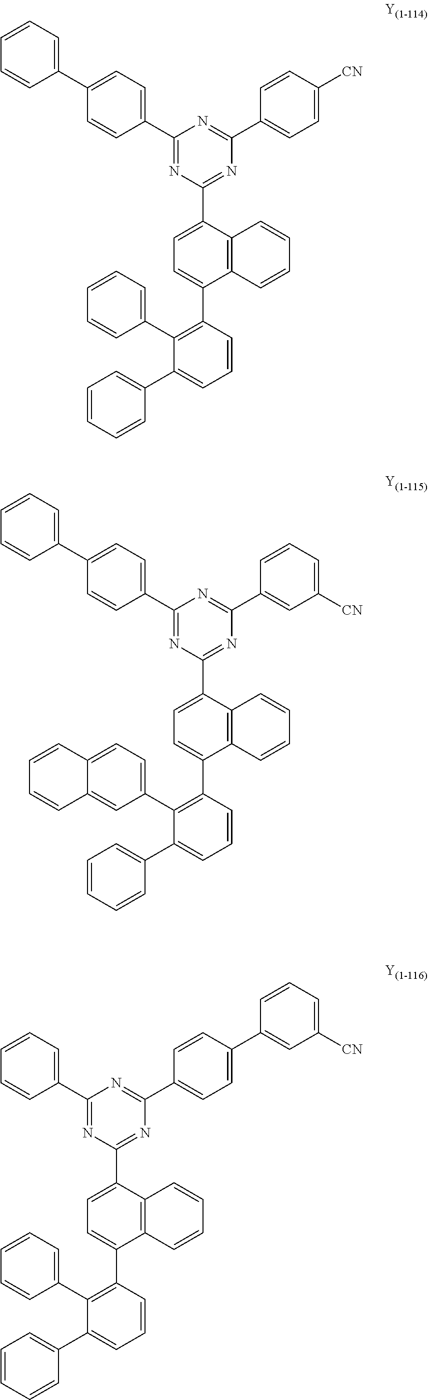 [SMILES: N#Cc1ccc(-c2nc(-c3ccc(-c4ccccc4)cc3)nc(-c3ccc(-c4cccc(-c5ccccc5)c4-c4ccccc4)c4ccccc34)n2)cc1 ; N#Cc1cccc(-c2nc(-c3ccc(-c4ccccc4)cc3)nc(-c3ccc(-c4cccc(-c5ccccc5)c4-c4ccc5ccccc5c4)c4ccccc34)n2)c1 ; N#Cc1cccc(-c2ccc(-c3nc(-c4ccccc4)nc(-c4ccc(-c5cccc(-c6ccccc6)c5-c5ccccc5)c5ccccc45)n3)cc2)c1]

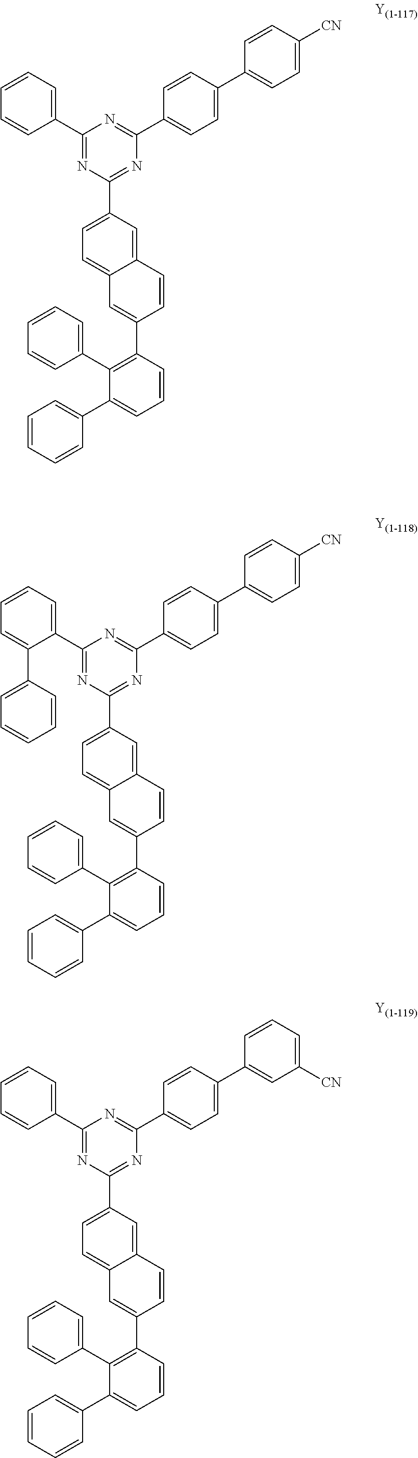 [SMILES: N#Cc1ccc(-c2ccc(-c3nc(-c4ccccc4)nc(-c4ccc5cc(-c6cccc(-c7ccccc7)c6-c6ccccc6)ccc5c4)n3)cc2)cc1 ; N#Cc1ccc(-c2ccc(-c3nc(-c4ccc5cc(-c6cccc(-c7ccccc7)c6-c6ccccc6)ccc5c4)nc(-c4ccccc4-c4ccccc4)n3)cc2)cc1 ; N#Cc1cccc(-c2ccc(-c3nc(-c4ccccc4)nc(-c4ccc5cc(-c6cccc(-c7ccccc7)c6-c6ccccc6)ccc5c4)n3)cc2)c1]

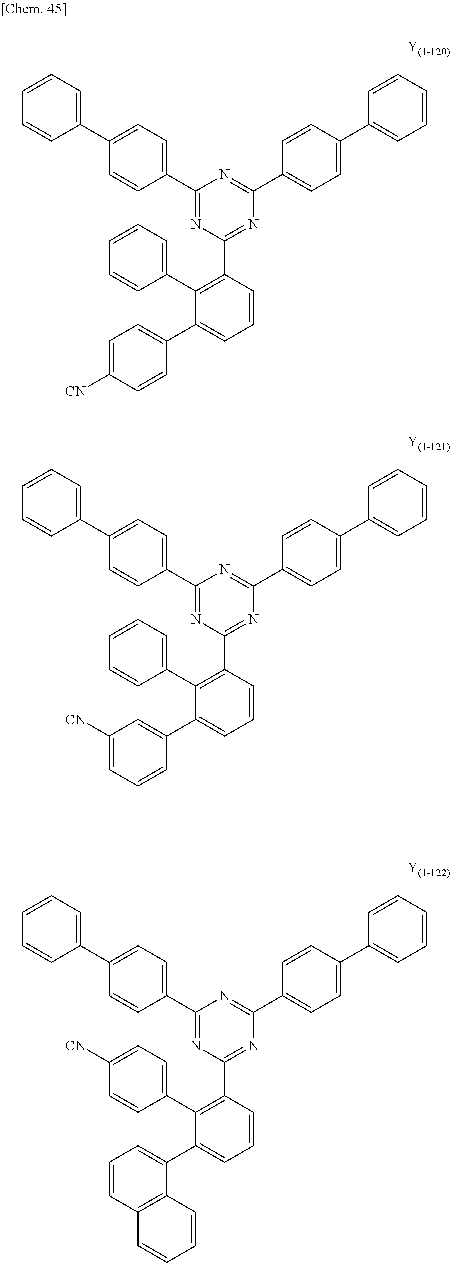

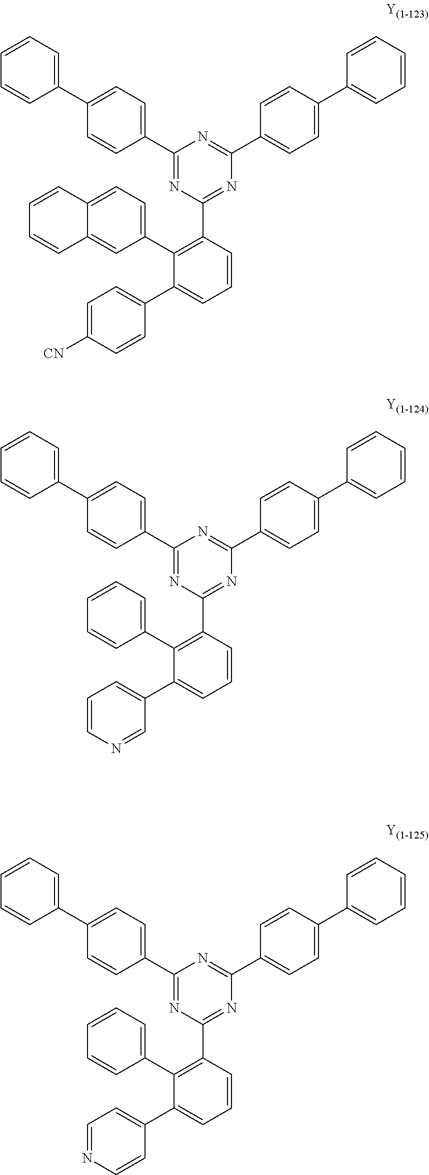

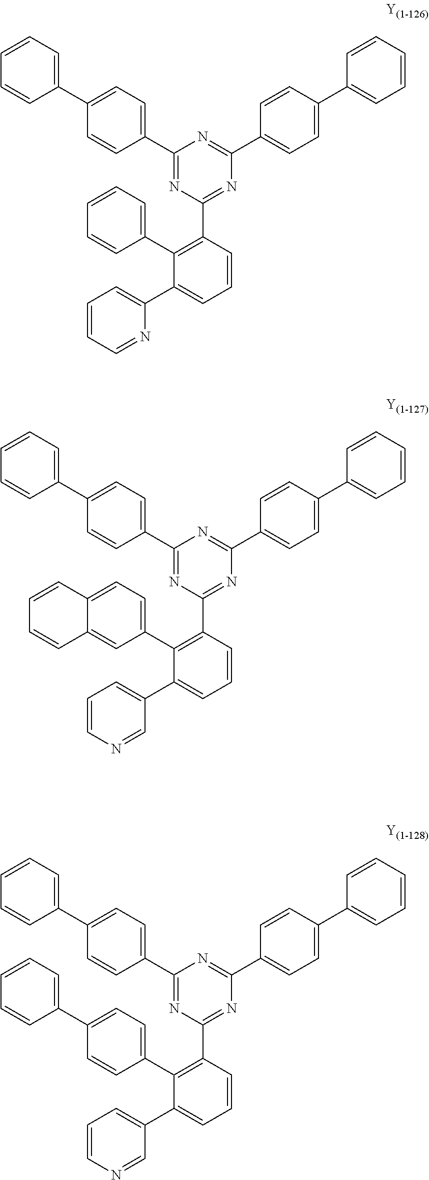 [SMILES: c1ccc(-c2ccc(-c3nc(-c4ccc(-c5ccccc5)cc4)nc(-c4cccc(-c5ccccn5)c4-c4ccccc4)n3)cc2)cc1 ; c1ccc(-c2ccc(-c3nc(-c4ccc(-c5ccccc5)cc4)nc(-c4cccc(-c5cccnc5)c4-c4ccc5ccccc5c4)n3)cc2)cc1 ; c1ccc(-c2ccc(-c3nc(-c4ccc(-c5ccccc5)cc4)nc(-c4cccc(-c5cccnc5)c4-c4ccc(-c5ccccc5)cc4)n3)cc2)cc1]

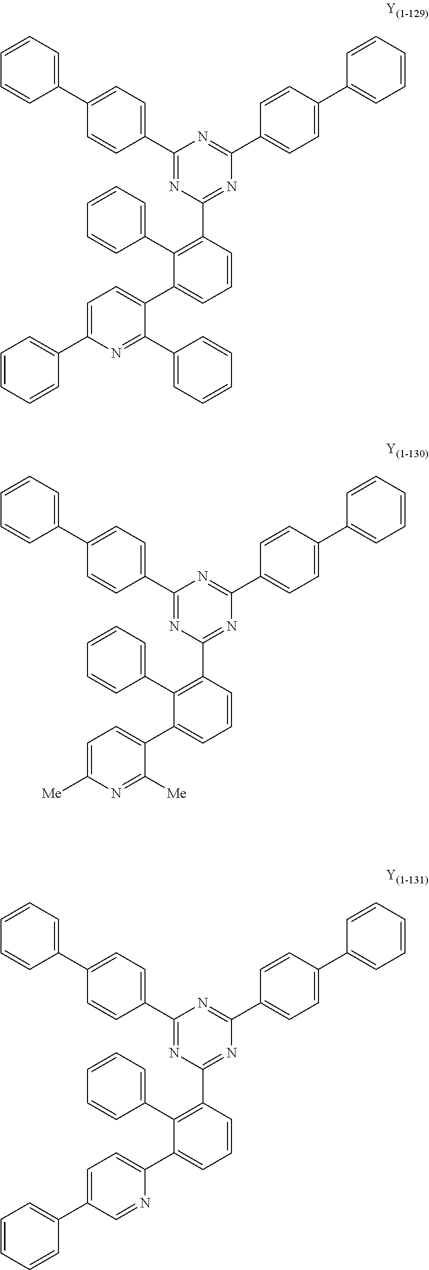 [SMILES: c1ccc(-c2ccc(-c3nc(-c4ccc(-c5ccccc5)cc4)nc(-c4cccc(-c5ccc(-c6ccccc6)nc5-c5ccccc5)c4-c4ccccc4)n3)cc2)cc1 ; Cc1ccc(-c2cccc(-c3nc(-c4ccc(-c5ccccc5)cc4)nc(-c4ccc(-c5ccccc5)cc4)n3)c2-c2ccccc2)c(C)n1 ; c1ccc(-c2ccc(-c3nc(-c4ccc(-c5ccccc5)cc4)nc(-c4cccc(-c5ccc(-c6ccccc6)cn5)c4-c4ccccc4)n3)cc2)cc1]

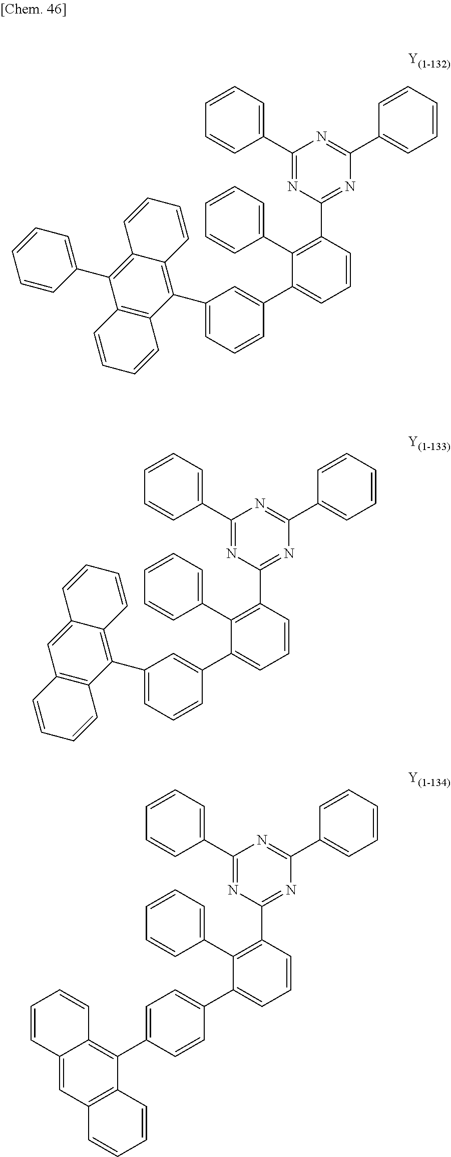 [SMILES: c1ccc(-c2nc(-c3ccccc3)nc(-c3cccc(-c4cccc(-c5c6ccccc6c(-c6ccccc6)c6ccccc56)c4)c3-c3ccccc3)n2)cc1 ; c1ccc(-c2nc(-c3ccccc3)nc(-c3cccc(-c4cccc(-c5c6ccccc6cc6ccccc56)c4)c3-c3ccccc3)n2)cc1 ; c1ccc(-c2nc(-c3ccccc3)nc(-c3cccc(-c4ccc(-c5c6ccccc6cc6ccccc56)cc4)c3-c3ccccc3)n2)cc1]

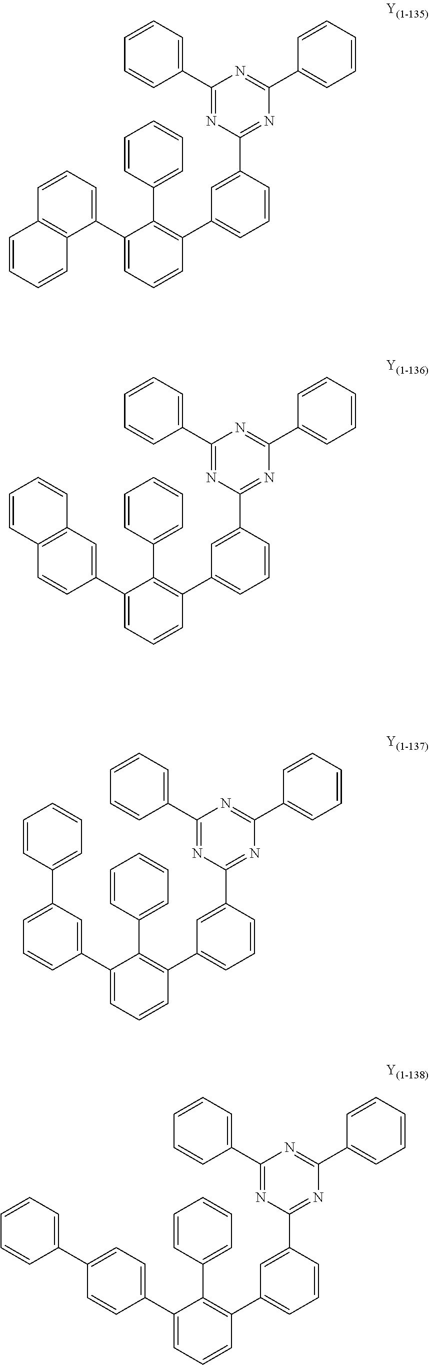 [SMILES: c1ccc(-c2nc(-c3ccccc3)nc(-c3cccc(-c4cccc(-c5cccc6ccccc56)c4-c4ccccc4)c3)n2)cc1 ; c1ccc(-c2nc(-c3ccccc3)nc(-c3cccc(-c4cccc(-c5ccc6ccccc6c5)c4-c4ccccc4)c3)n2)cc1 ; c1ccc(-c2cccc(-c3cccc(-c4cccc(-c5nc(-c6ccccc6)nc(-c6ccccc6)n5)c4)c3-c3ccccc3)c2)cc1 ; c1ccc(-c2ccc(-c3cccc(-c4cccc(-c5nc(-c6ccccc6)nc(-c6ccccc6)n5)c4)c3-c3ccccc3)cc2)cc1]

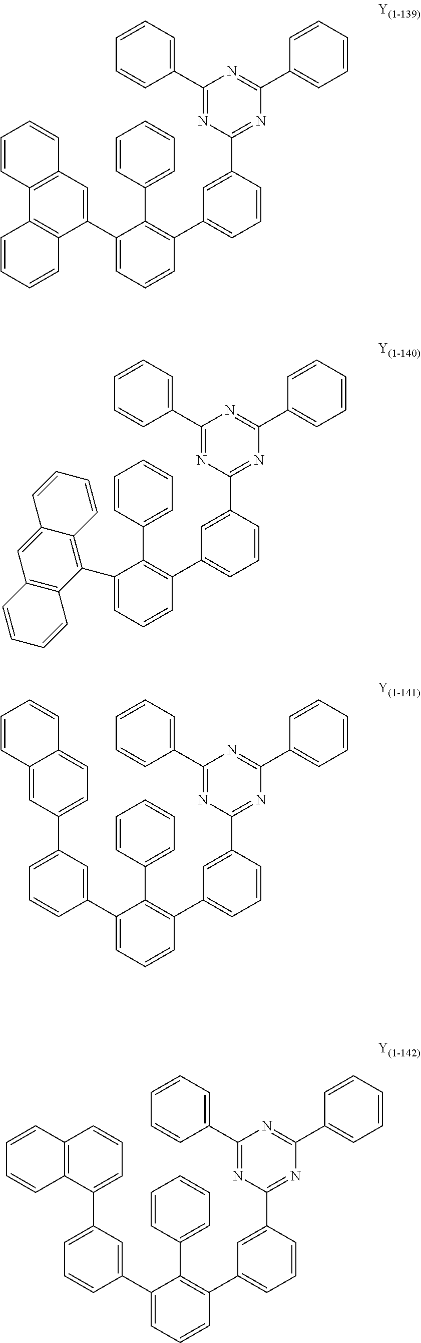 [SMILES: c1ccc(-c2nc(-c3ccccc3)nc(-c3cccc(-c4cccc(-c5cc6ccccc6c6ccccc56)c4-c4ccccc4)c3)n2)cc1 ; c1ccc(-c2nc(-c3ccccc3)nc(-c3cccc(-c4cccc(-c5c6ccccc6cc6ccccc56)c4-c4ccccc4)c3)n2)cc1 ; c1ccc(-c2nc(-c3ccccc3)nc(-c3cccc(-c4cccc(-c5cccc(-c6ccc7ccccc7c6)c5)c4-c4ccccc4)c3)n2)cc1 ; c1ccc(-c2nc(-c3ccccc3)nc(-c3cccc(-c4cccc(-c5cccc(-c6cccc7ccccc67)c5)c4-c4ccccc4)c3)n2)cc1]

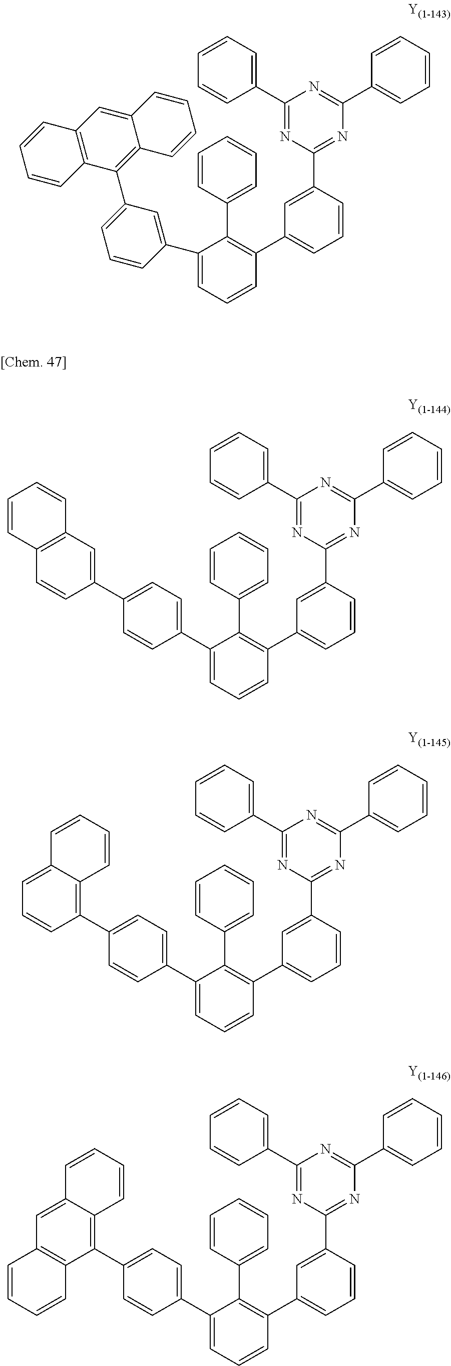 [SMILES: c1ccc(-c2nc(-c3ccccc3)nc(-c3cccc(-c4cccc(-c5cccc(-c6c7ccccc7cc7ccccc67)c5)c4-c4ccccc4)c3)n2)cc1 ; c1ccc(-c2nc(-c3ccccc3)nc(-c3cccc(-c4cccc(-c5ccc(-c6ccc7ccccc7c6)cc5)c4-c4ccccc4)c3)n2)cc1 ; c1ccc(-c2nc(-c3ccccc3)nc(-c3cccc(-c4cccc(-c5ccc(-c6cccc7ccccc67)cc5)c4-c4ccccc4)c3)n2)cc1 ; c1ccc(-c2nc(-c3ccccc3)nc(-c3cccc(-c4cccc(-c5ccc(-c6c7ccccc7cc7ccccc67)cc5)c4-c4ccccc4)c3)n2)cc1]

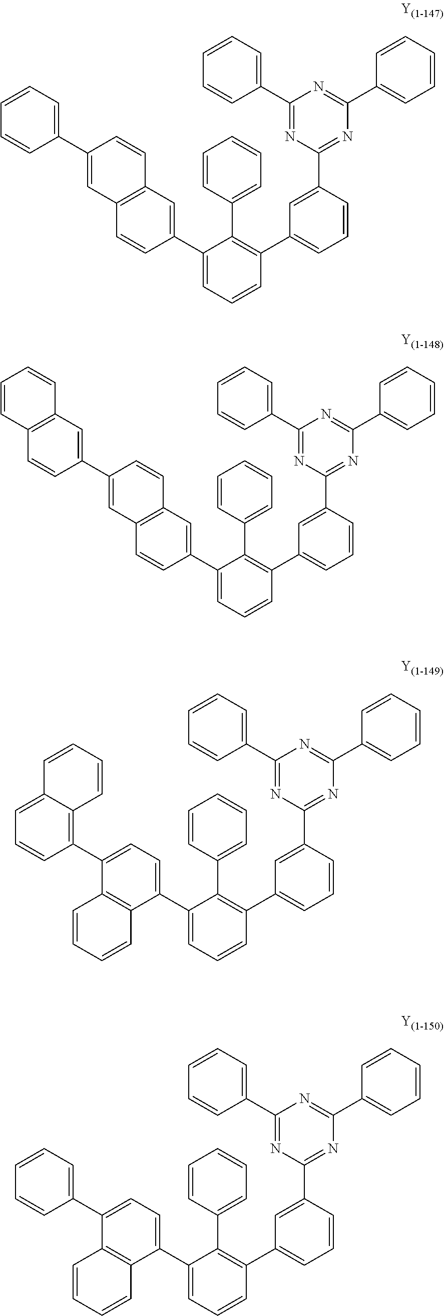 [SMILES: c1ccc(-c2ccc3cc(-c4cccc(-c5cccc(-c6nc(-c7ccccc7)nc(-c7ccccc7)n6)c5)c4-c4ccccc4)ccc3c2)cc1 ; c1ccc(-c2nc(-c3ccccc3)nc(-c3cccc(-c4cccc(-c5ccc6cc(-c7ccc8ccccc8c7)ccc6c5)c4-c4ccccc4)c3)n2)cc1 ; c1ccc(-c2nc(-c3ccccc3)nc(-c3cccc(-c4cccc(-c5ccc(-c6cccc7ccccc67)c6ccccc56)c4-c4ccccc4)c3)n2)cc1 ; c1ccc(-c2nc(-c3ccccc3)nc(-c3cccc(-c4cccc(-c5ccc(-c6ccccc6)c6ccccc56)c4-c4ccccc4)c3)n2)cc1]

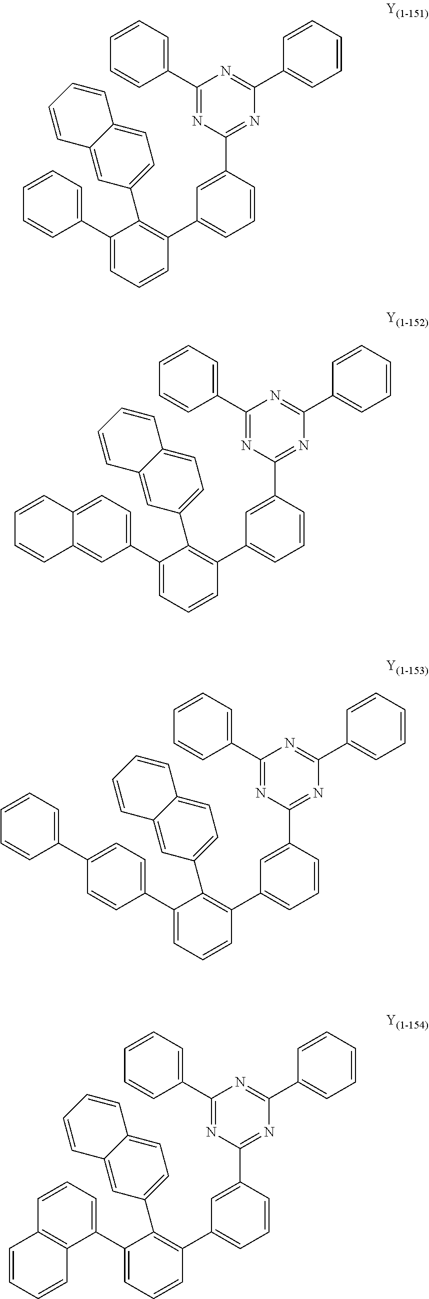 [SMILES: c1ccc(-c2nc(-c3ccccc3)nc(-c3cccc(-c4cccc(-c5ccccc5)c4-c4ccc5ccccc5c4)c3)n2)cc1 ; c1ccc(-c2nc(-c3ccccc3)nc(-c3cccc(-c4cccc(-c5ccc6ccccc6c5)c4-c4ccc5ccccc5c4)c3)n2)cc1 ; c1ccc(-c2ccc(-c3cccc(-c4cccc(-c5nc(-c6ccccc6)nc(-c6ccccc6)n5)c4)c3-c3ccc4ccccc4c3)cc2)cc1 ; c1ccc(-c2nc(-c3ccccc3)nc(-c3cccc(-c4cccc(-c5cccc6ccccc56)c4-c4ccc5ccccc5c4)c3)n2)cc1]

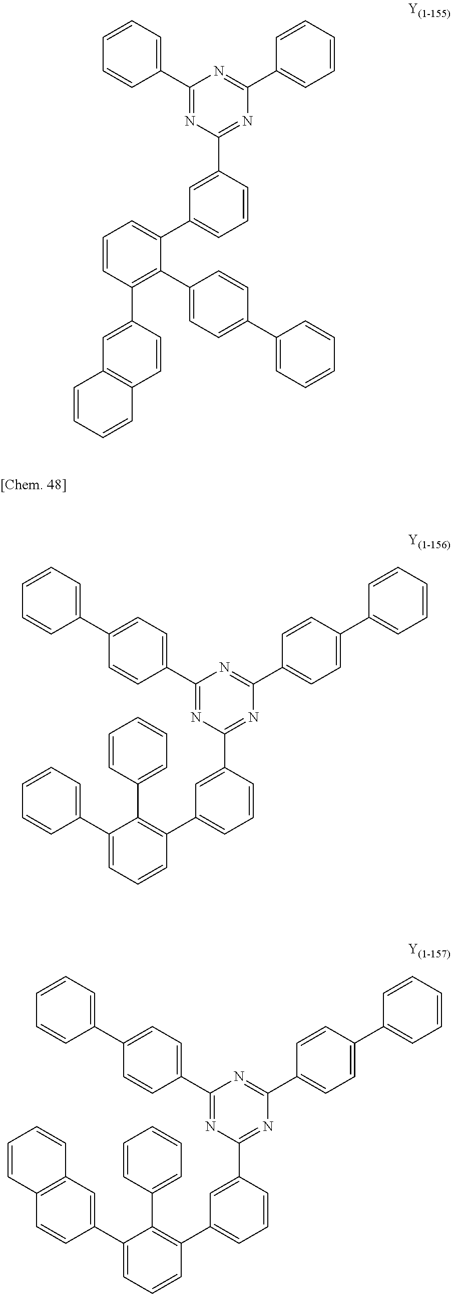 [SMILES: c1ccc(-c2ccc(-c3c(-c4cccc(-c5nc(-c6ccccc6)nc(-c6ccccc6)n5)c4)cccc3-c3ccc4ccccc4c3)cc2)cc1 ; c1ccc(-c2ccc(-c3nc(-c4ccc(-c5ccccc5)cc4)nc(-c4cccc(-c5cccc(-c6ccccc6)c5-c5ccccc5)c4)n3)cc2)cc1 ; c1ccc(-c2ccc(-c3nc(-c4ccc(-c5ccccc5)cc4)nc(-c4cccc(-c5cccc(-c6ccc7ccccc7c6)c5-c5ccccc5)c4)n3)cc2)cc1]

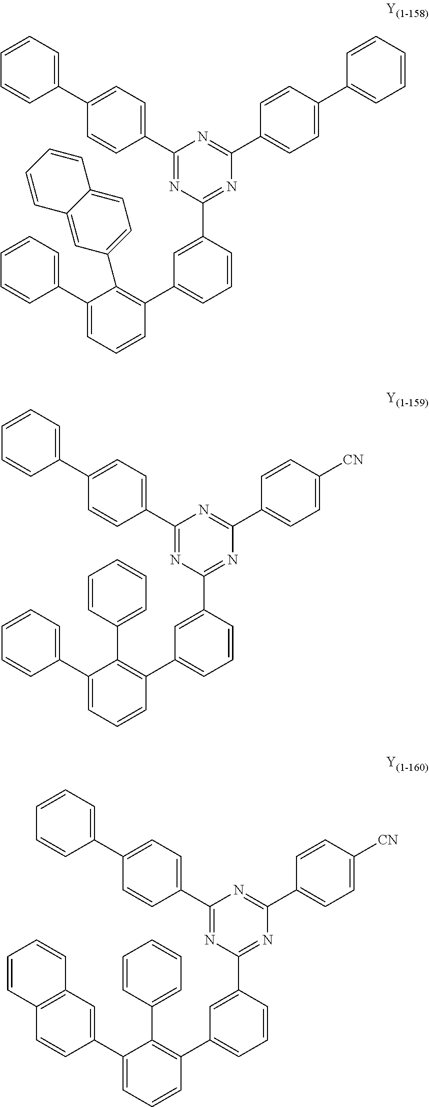 [SMILES: c1ccc(-c2ccc(-c3nc(-c4ccc(-c5ccccc5)cc4)nc(-c4cccc(-c5cccc(-c6ccccc6)c5-c5ccc6ccccc6c5)c4)n3)cc2)cc1 ; N#Cc1ccc(-c2nc(-c3ccc(-c4ccccc4)cc3)nc(-c3cccc(-c4cccc(-c5ccccc5)c4-c4ccccc4)c3)n2)cc1 ; N#Cc1ccc(-c2nc(-c3ccc(-c4ccccc4)cc3)nc(-c3cccc(-c4cccc(-c5ccc6ccccc6c5)c4-c4ccccc4)c3)n2)cc1]

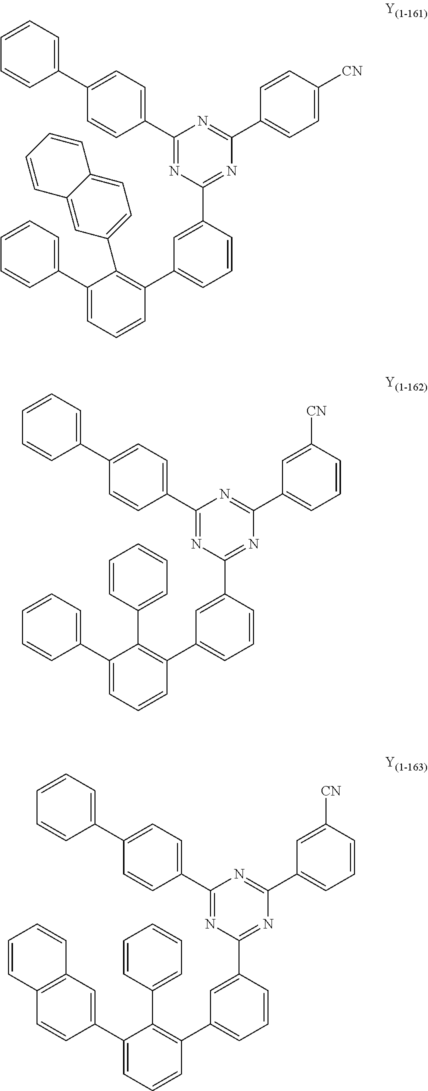 [SMILES: N#Cc1ccc(-c2nc(-c3ccc(-c4ccccc4)cc3)nc(-c3cccc(-c4cccc(-c5ccccc5)c4-c4ccc5ccccc5c4)c3)n2)cc1 ; N#Cc1cccc(-c2nc(-c3ccc(-c4ccccc4)cc3)nc(-c3cccc(-c4cccc(-c5ccccc5)c4-c4ccccc4)c3)n2)c1 ; N#Cc1cccc(-c2nc(-c3ccc(-c4ccccc4)cc3)nc(-c3cccc(-c4cccc(-c5ccc6ccccc6c5)c4-c4ccccc4)c3)n2)c1]

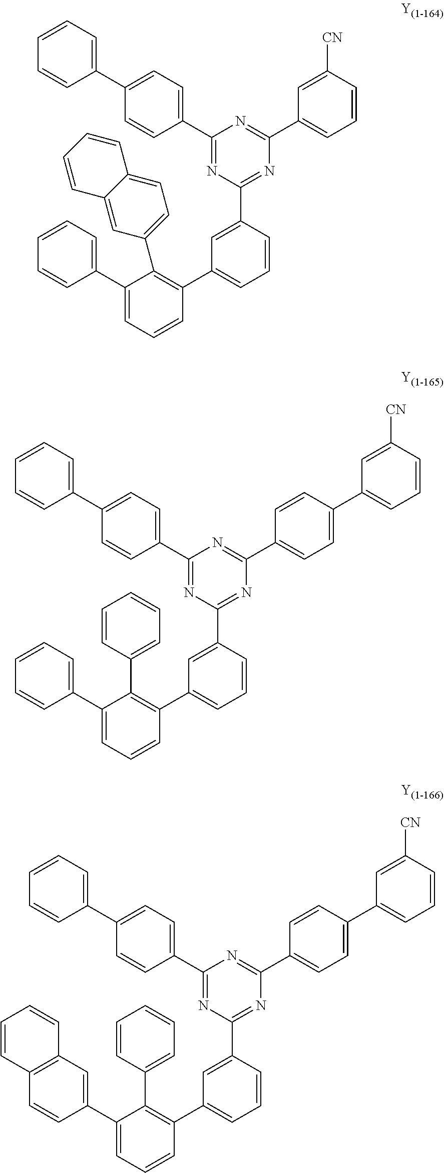 [SMILES: N#Cc1cccc(-c2nc(-c3ccc(-c4ccccc4)cc3)nc(-c3cccc(-c4cccc(-c5ccccc5)c4-c4ccc5ccccc5c4)c3)n2)c1 ; N#Cc1cccc(-c2ccc(-c3nc(-c4ccc(-c5ccccc5)cc4)nc(-c4cccc(-c5cccc(-c6ccccc6)c5-c5ccccc5)c4)n3)cc2)c1 ; N#Cc1cccc(-c2ccc(-c3nc(-c4ccc(-c5ccccc5)cc4)nc(-c4cccc(-c5cccc(-c6ccc7ccccc7c6)c5-c5ccccc5)c4)n3)cc2)c1]

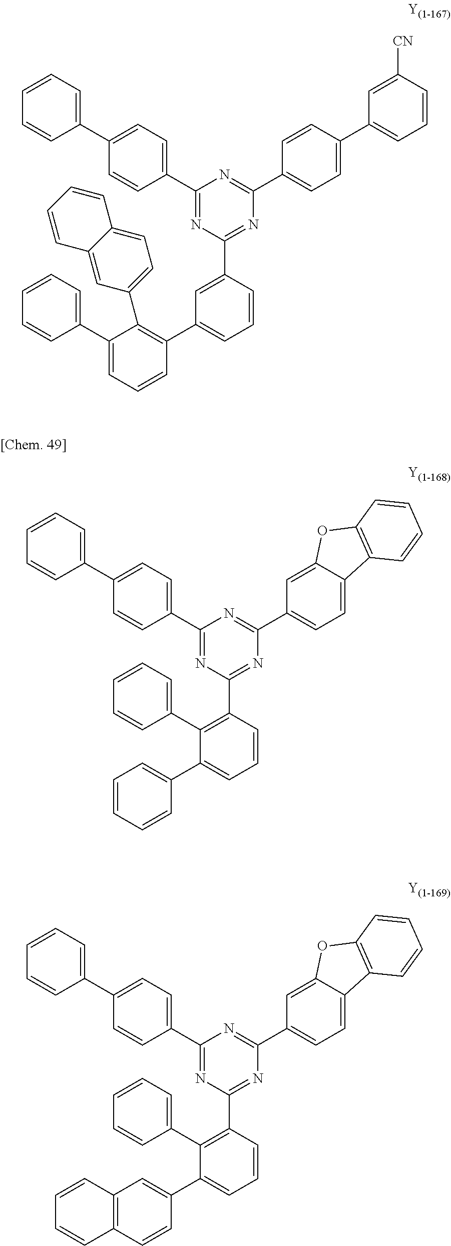 [SMILES: N#Cc1cccc(-c2ccc(-c3nc(-c4ccc(-c5ccccc5)cc4)nc(-c4cccc(-c5cccc(-c6ccccc6)c5-c5ccc6ccccc6c5)c4)n3)cc2)c1 ; c1ccc(-c2ccc(-c3nc(-c4ccc5c(c4)oc4ccccc45)nc(-c4cccc(-c5ccccc5)c4-c4ccccc4)n3)cc2)cc1 ; c1ccc(-c2ccc(-c3nc(-c4ccc5c(c4)oc4ccccc45)nc(-c4cccc(-c5ccc6ccccc6c5)c4-c4ccccc4)n3)cc2)cc1]

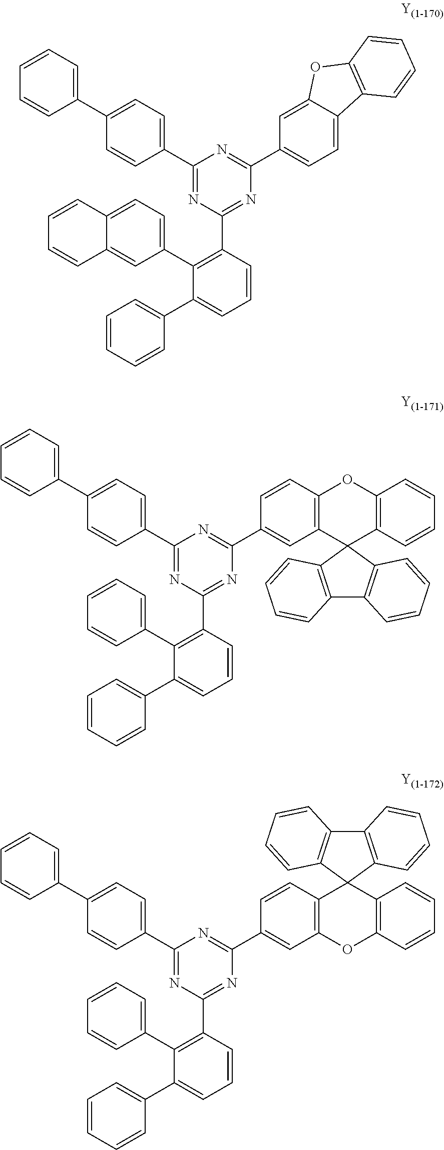 [SMILES: c1ccc(-c2ccc(-c3nc(-c4ccc5c(c4)oc4ccccc45)nc(-c4cccc(-c5ccccc5)c4-c4ccc5ccccc5c4)n3)cc2)cc1 ; c1ccc(-c2ccc(-c3nc(-c4ccc5c(c4)C4(c6ccccc6O5)c5ccccc5-c5ccccc54)nc(-c4cccc(-c5ccccc5)c4-c4ccccc4)n3)cc2)cc1 ; c1ccc(-c2ccc(-c3nc(-c4ccc5c(c4)Oc4ccccc4C54c5ccccc5-c5ccccc54)nc(-c4cccc(-c5ccccc5)c4-c4ccccc4)n3)cc2)cc1]

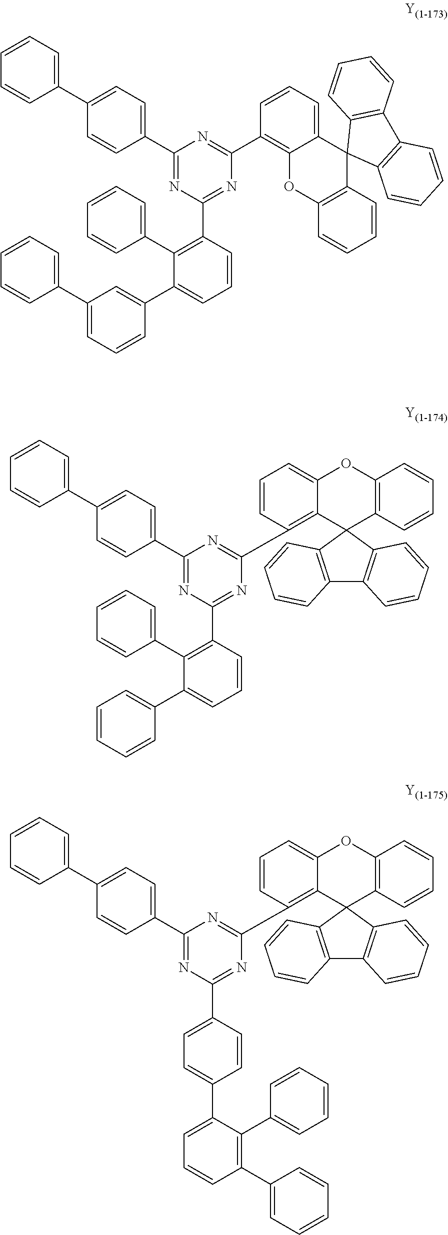 [SMILES: c1ccc(-c2ccc(-c3nc(-c4cccc5c4Oc4ccccc4C54c5ccccc5-c5ccccc54)nc(-c4cccc(-c5cccc(-c6ccccc6)c5)c4-c4ccccc4)n3)cc2)cc1 ; c1ccc(-c2ccc(-c3nc(-c4cccc(-c5ccccc5)c4-c4ccccc4)nc(-c4cccc5c4C4(c6ccccc6O5)c5ccccc5-c5ccccc54)n3)cc2)cc1 ; c1ccc(-c2ccc(-c3nc(-c4ccc(-c5cccc(-c6ccccc6)c5-c5ccccc5)cc4)nc(-c4cccc5c4C4(c6ccccc6O5)c5ccccc5-c5ccccc54)n3)cc2)cc1]

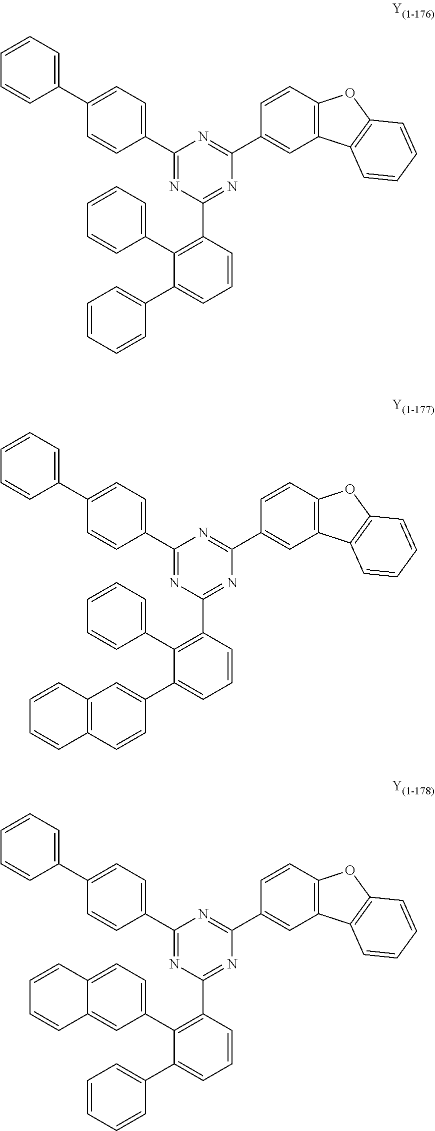 [SMILES: c1ccc(-c2ccc(-c3nc(-c4ccc5oc6ccccc6c5c4)nc(-c4cccc(-c5ccccc5)c4-c4ccccc4)n3)cc2)cc1 ; c1ccc(-c2ccc(-c3nc(-c4ccc5oc6ccccc6c5c4)nc(-c4cccc(-c5ccc6ccccc6c5)c4-c4ccccc4)n3)cc2)cc1 ; c1ccc(-c2ccc(-c3nc(-c4ccc5oc6ccccc6c5c4)nc(-c4cccc(-c5ccccc5)c4-c4ccc5ccccc5c4)n3)cc2)cc1]

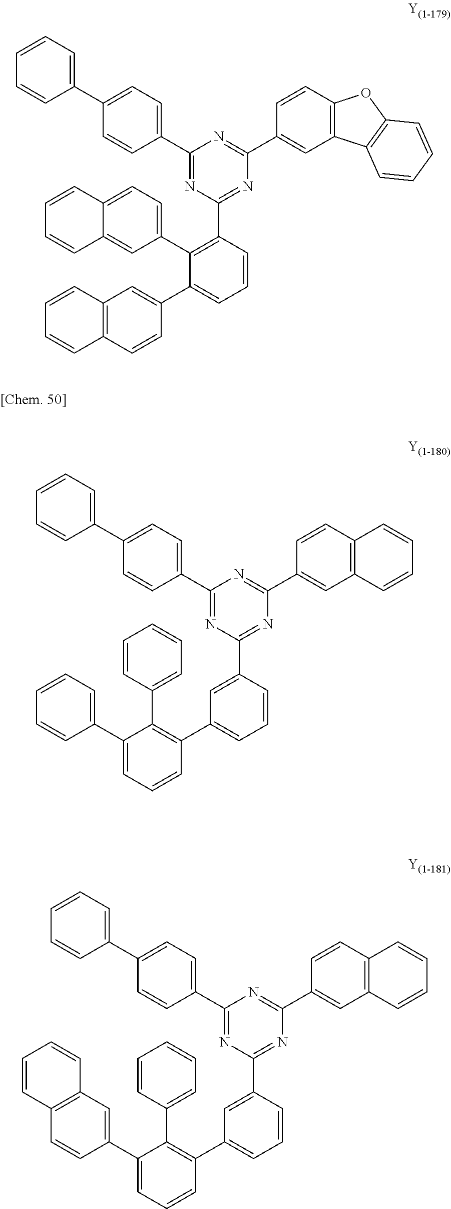 [SMILES: c1ccc(-c2ccc(-c3nc(-c4ccc5oc6ccccc6c5c4)nc(-c4cccc(-c5ccc6ccccc6c5)c4-c4ccc5ccccc5c4)n3)cc2)cc1 ; c1ccc(-c2ccc(-c3nc(-c4cccc(-c5cccc(-c6ccccc6)c5-c5ccccc5)c4)nc(-c4ccc5ccccc5c4)n3)cc2)cc1 ; c1ccc(-c2ccc(-c3nc(-c4cccc(-c5cccc(-c6ccc7ccccc7c6)c5-c5ccccc5)c4)nc(-c4ccc5ccccc5c4)n3)cc2)cc1]

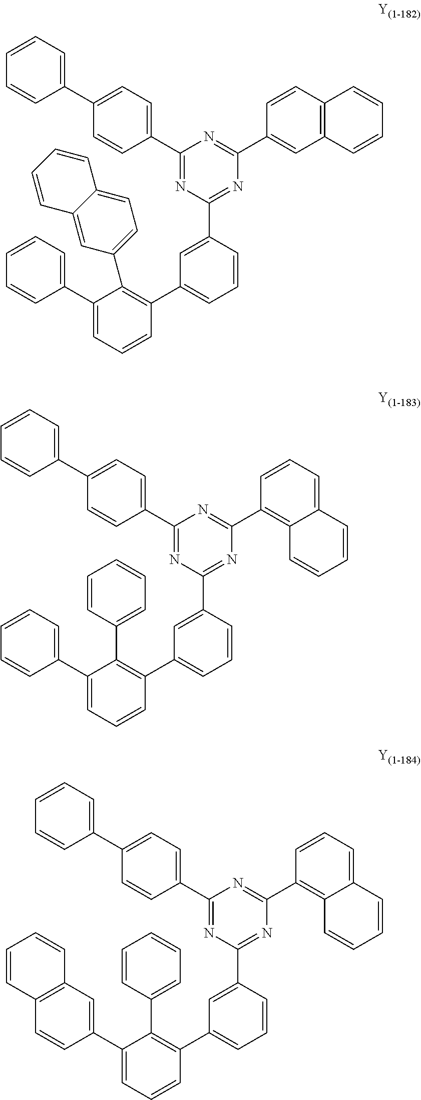 [SMILES: c1ccc(-c2ccc(-c3nc(-c4cccc(-c5cccc(-c6ccccc6)c5-c5ccc6ccccc6c5)c4)nc(-c4ccc5ccccc5c4)n3)cc2)cc1 ; c1ccc(-c2ccc(-c3nc(-c4cccc(-c5cccc(-c6ccccc6)c5-c5ccccc5)c4)nc(-c4cccc5ccccc45)n3)cc2)cc1 ; c1ccc(-c2ccc(-c3nc(-c4cccc(-c5cccc(-c6ccc7ccccc7c6)c5-c5ccccc5)c4)nc(-c4cccc5ccccc45)n3)cc2)cc1]

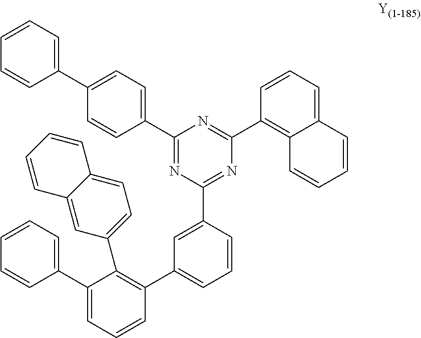 [SMILES: c1ccc(-c2ccc(-c3nc(-c4cccc(-c5cccc(-c6ccccc6)c5-c5ccc6ccccc6c5)c4)nc(-c4cccc5ccccc45)n3)cc2)cc1]

### <Triazine Compound Z(1)>

A triazine compound according to an aspect of the present invention is represented by formula Z(1):

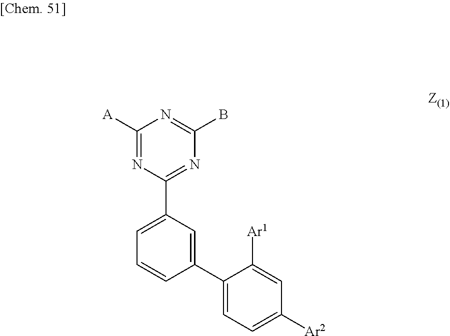

wherein in the case of the formula Z(1),
A represents a phenyl group, a biphenylyl group, or a naphthyl group, each optionally substituted with one or more substituent(s) selected from the group consisting of a fluorine atom, a methyl group and a cyano group;
B represents a phenyl group, a biphenylyl group, or a naphthyl group, each optionally substituted with one or more substituent(s) selected from the group consisting of a fluorine atom, a methyl group, and a cyano group;
Ar¹ represents a phenyl group or a naphthyl group, each optionally substituted with one or more substituent(s) selected from the group consisting of a fluorine atom, a methyl group, and a cyano group; and Are represents a phenyl group or a naphthyl group, each optionally substituted with one or more substituent(s) selected from the group consisting of a fluorine atom, a methyl group, and a cyano group.

### [A]

A represents a phenyl group, a biphenylyl group, or a naphthyl group, each optionally substituted with one or more group(s) selected from the group consisting of a fluorine atom, a methyl group, and a cyano group. From the viewpoint of the ease of the synthesis of the triazine compound represented by the formula Z(1) (hereinafter, also simply referred to as “triazine compound Z(1)”), A represents preferably an unsubstituted phenyl group, an unsubstituted biphenylyl group, or an unsubstituted naphthyl group, and more preferably an unsubstituted phenyl group or an unsubstituted biphenylyl group.

### [B]

B represents a phenyl group, a biphenylyl group, or a naphthyl group, each optionally substituted with one or more group(s) selected from the group consisting of a fluorine atom, a methyl group, and a cyano group. From the viewpoint of the ease of the synthesis of the triazine compound Z(1), B represents preferably an unsubstituted phenyl group, an unsubstituted biphenylyl group, or an unsubstituted naphthyl group, and more preferably an unsubstituted phenyl group or an unsubstituted biphenylyl group.

### [Ar¹]

Ar¹ represents a phenyl group or a naphthyl group, each optionally substituted with one or more substituent(s) selected from the group consisting of a fluorine atom, a methyl group, and a cyano group. From the viewpoint of the ease of the synthesis of the triazine compound Z(1), Ar¹ represents preferably an unsubstituted phenyl group or an unsubstituted naphthyl group, and more preferably an unsubstituted phenyl group.

### [Ar²]

Ar² represents a phenyl group or a naphthyl group, each optionally substituted with one or more substituent(s) selected from the group consisting of a fluorine atom, a methyl group, and a cyano group. From the viewpoint of the ease of the synthesis of the triazine compound Z(1), Ar² preferably represents an unsubstituted phenyl group or an unsubstituted naphthyl group.

### Specific Examples of Triazine Compound Z (1)

Among the triazine compounds according to an aspect of the present invention represented by the formula Z(1), specific examples of particularly preferable compounds include compounds represented by the following formulas Z(1-1) to Z(1-95), but the triazine compound according to an aspect of the present invention is not limited thereto.

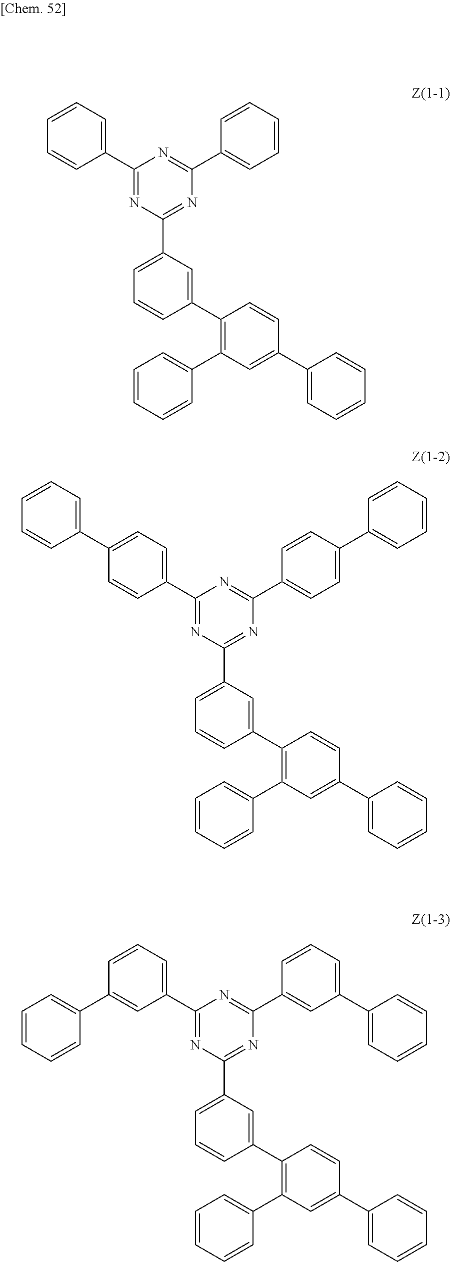 [SMILES: c1ccc(-c2ccc(-c3cccc(-c4nc(-c5ccccc5)nc(-c5ccccc5)n4)c3)c(-c3ccccc3)c2)cc1 ; c1ccc(-c2ccc(-c3nc(-c4ccc(-c5ccccc5)cc4)nc(-c4cccc(-c5ccc(-c6ccccc6)cc5-c5ccccc5)c4)n3)cc2)cc1 ; c1ccc(-c2cccc(-c3nc(-c4cccc(-c5ccccc5)c4)nc(-c4cccc(-c5ccc(-c6ccccc6)cc5-c5ccccc5)c4)n3)c2)cc1]

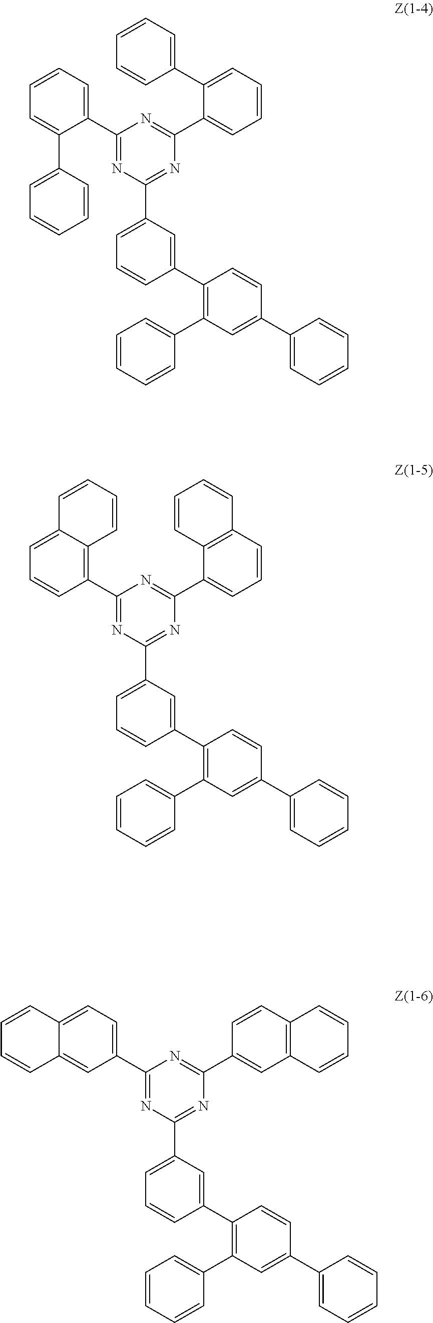 [SMILES: c1ccc(-c2ccc(-c3cccc(-c4nc(-c5ccccc5-c5ccccc5)nc(-c5ccccc5-c5ccccc5)n4)c3)c(-c3ccccc3)c2)cc1 ; c1ccc(-c2ccc(-c3cccc(-c4nc(-c5cccc6ccccc56)nc(-c5cccc6ccccc56)n4)c3)c(-c3ccccc3)c2)cc1 ; c1ccc(-c2ccc(-c3cccc(-c4nc(-c5ccc6ccccc6c5)nc(-c5ccc6ccccc6c5)n4)c3)c(-c3ccccc3)c2)cc1]

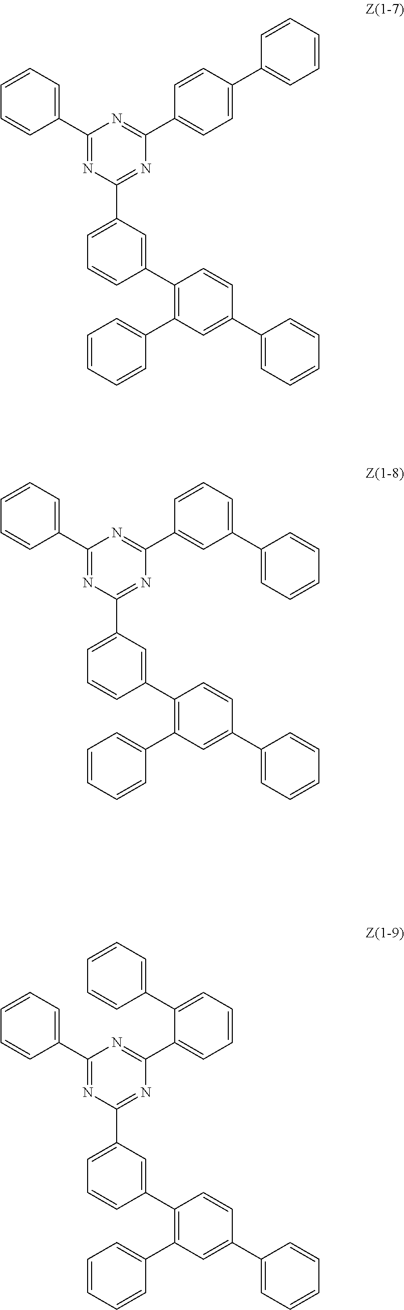 [SMILES: c1ccc(-c2ccc(-c3nc(-c4ccccc4)nc(-c4cccc(-c5ccc(-c6ccccc6)cc5-c5ccccc5)c4)n3)cc2)cc1 ; c1ccc(-c2cccc(-c3nc(-c4ccccc4)nc(-c4cccc(-c5ccc(-c6ccccc6)cc5-c5ccccc5)c4)n3)c2)cc1 ; c1ccc(-c2ccc(-c3cccc(-c4nc(-c5ccccc5)nc(-c5ccccc5-c5ccccc5)n4)c3)c(-c3ccccc3)c2)cc1]

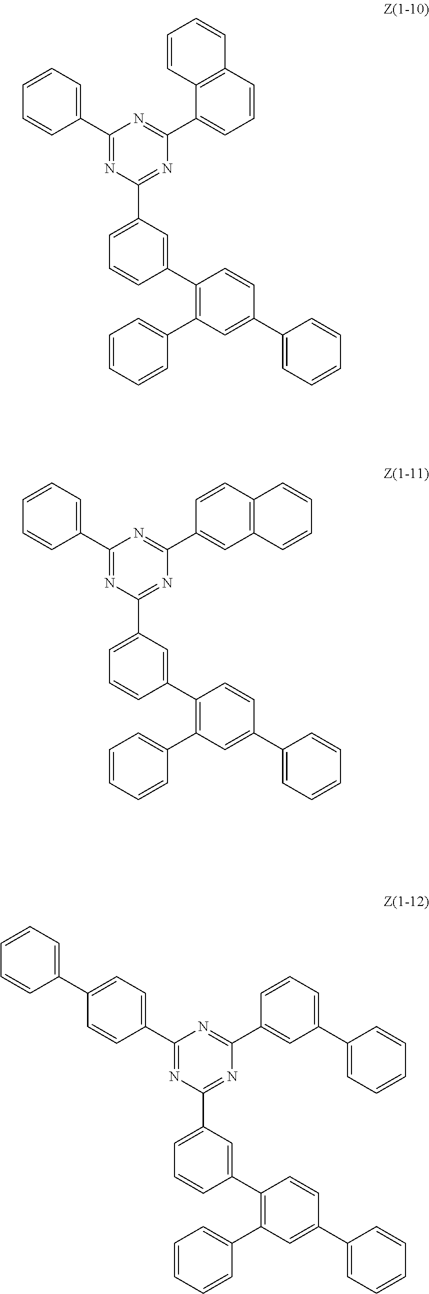 [SMILES: c1ccc(-c2ccc(-c3cccc(-c4nc(-c5ccccc5)nc(-c5cccc6ccccc56)n4)c3)c(-c3ccccc3)c2)cc1 ; c1ccc(-c2ccc(-c3cccc(-c4nc(-c5ccccc5)nc(-c5ccc6ccccc6c5)n4)c3)c(-c3ccccc3)c2)cc1 ; c1ccc(-c2ccc(-c3nc(-c4cccc(-c5ccccc5)c4)nc(-c4cccc(-c5ccc(-c6ccccc6)cc5-c5ccccc5)c4)n3)cc2)cc1]

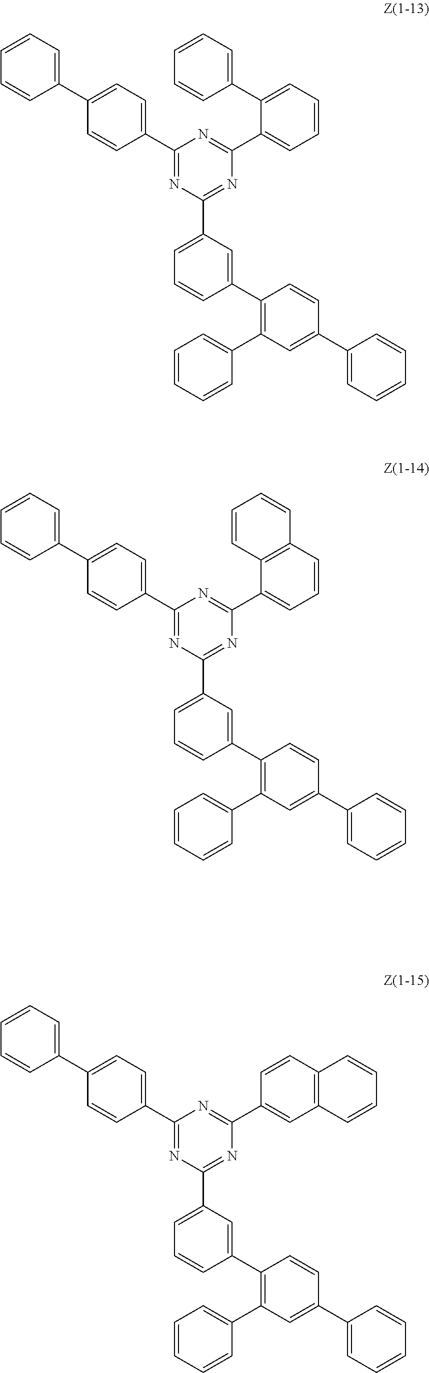 [SMILES: c1ccc(-c2ccc(-c3nc(-c4cccc(-c5ccc(-c6ccccc6)cc5-c5ccccc5)c4)nc(-c4ccccc4-c4ccccc4)n3)cc2)cc1 ; c1ccc(-c2ccc(-c3nc(-c4cccc(-c5ccc(-c6ccccc6)cc5-c5ccccc5)c4)nc(-c4cccc5ccccc45)n3)cc2)cc1 ; c1ccc(-c2ccc(-c3nc(-c4cccc(-c5ccc(-c6ccccc6)cc5-c5ccccc5)c4)nc(-c4ccc5ccccc5c4)n3)cc2)cc1]

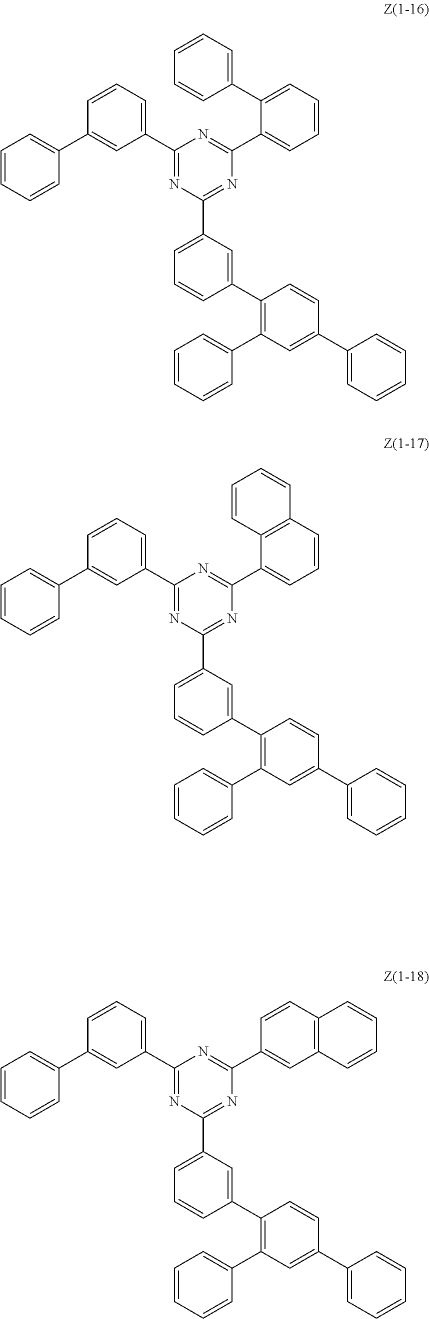 [SMILES: c1ccc(-c2cccc(-c3nc(-c4cccc(-c5ccc(-c6ccccc6)cc5-c5ccccc5)c4)nc(-c4ccccc4-c4ccccc4)n3)c2)cc1 ; c1ccc(-c2cccc(-c3nc(-c4cccc(-c5ccc(-c6ccccc6)cc5-c5ccccc5)c4)nc(-c4cccc5ccccc45)n3)c2)cc1 ; c1ccc(-c2cccc(-c3nc(-c4cccc(-c5ccc(-c6ccccc6)cc5-c5ccccc5)c4)nc(-c4ccc5ccccc5c4)n3)c2)cc1]

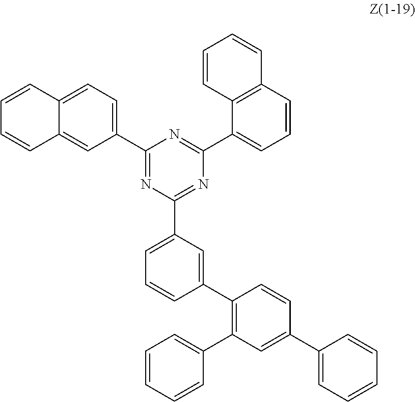 [SMILES: c1ccc(-c2ccc(-c3cccc(-c4nc(-c5ccc6ccccc6c5)nc(-c5cccc6ccccc56)n4)c3)c(-c3ccccc3)c2)cc1]

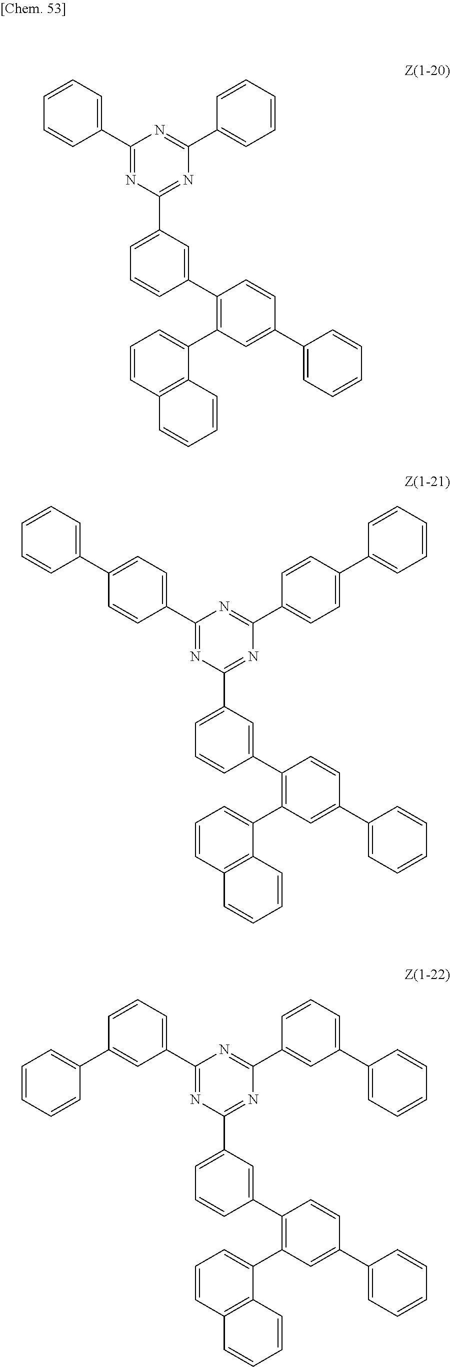 [SMILES: c1ccc(-c2ccc(-c3cccc(-c4nc(-c5ccccc5)nc(-c5ccccc5)n4)c3)c(-c3cccc4ccccc34)c2)cc1 ; c1ccc(-c2ccc(-c3nc(-c4ccc(-c5ccccc5)cc4)nc(-c4cccc(-c5ccc(-c6ccccc6)cc5-c5cccc6ccccc56)c4)n3)cc2)cc1 ; c1ccc(-c2cccc(-c3nc(-c4cccc(-c5ccccc5)c4)nc(-c4cccc(-c5ccc(-c6ccccc6)cc5-c5cccc6ccccc56)c4)n3)c2)cc1]

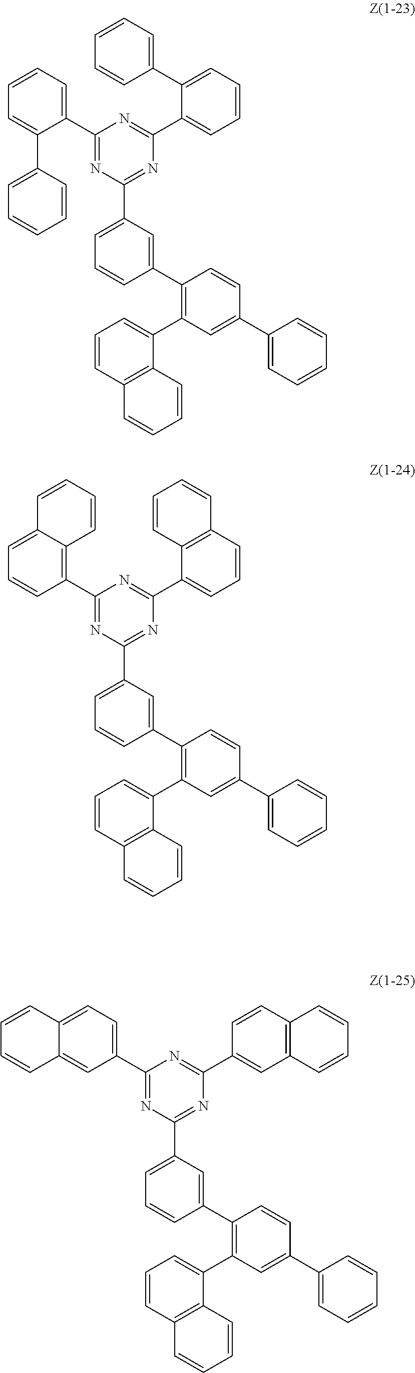 [SMILES: c1ccc(-c2ccc(-c3cccc(-c4nc(-c5ccccc5-c5ccccc5)nc(-c5ccccc5-c5ccccc5)n4)c3)c(-c3cccc4ccccc34)c2)cc1 ; c1ccc(-c2ccc(-c3cccc(-c4nc(-c5cccc6ccccc56)nc(-c5cccc6ccccc56)n4)c3)c(-c3cccc4ccccc34)c2)cc1 ; c1ccc(-c2ccc(-c3cccc(-c4nc(-c5ccc6ccccc6c5)nc(-c5ccc6ccccc6c5)n4)c3)c(-c3cccc4ccccc34)c2)cc1]

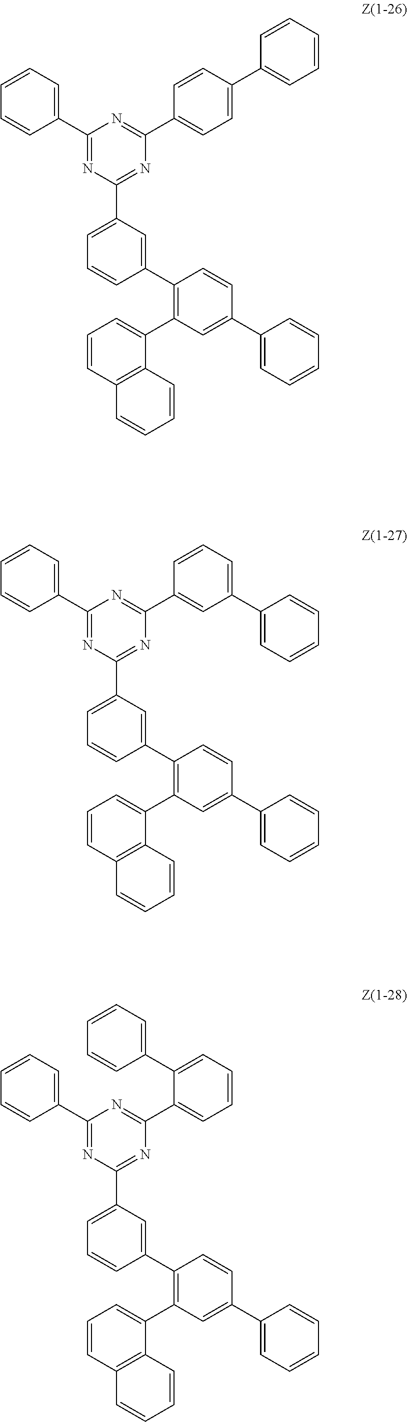 [SMILES: c1ccc(-c2ccc(-c3nc(-c4ccccc4)nc(-c4cccc(-c5ccc(-c6ccccc6)cc5-c5cccc6ccccc56)c4)n3)cc2)cc1 ; c1ccc(-c2cccc(-c3nc(-c4ccccc4)nc(-c4cccc(-c5ccc(-c6ccccc6)cc5-c5cccc6ccccc56)c4)n3)c2)cc1 ; c1ccc(-c2ccc(-c3cccc(-c4nc(-c5ccccc5)nc(-c5ccccc5-c5ccccc5)n4)c3)c(-c3cccc4ccccc34)c2)cc1]

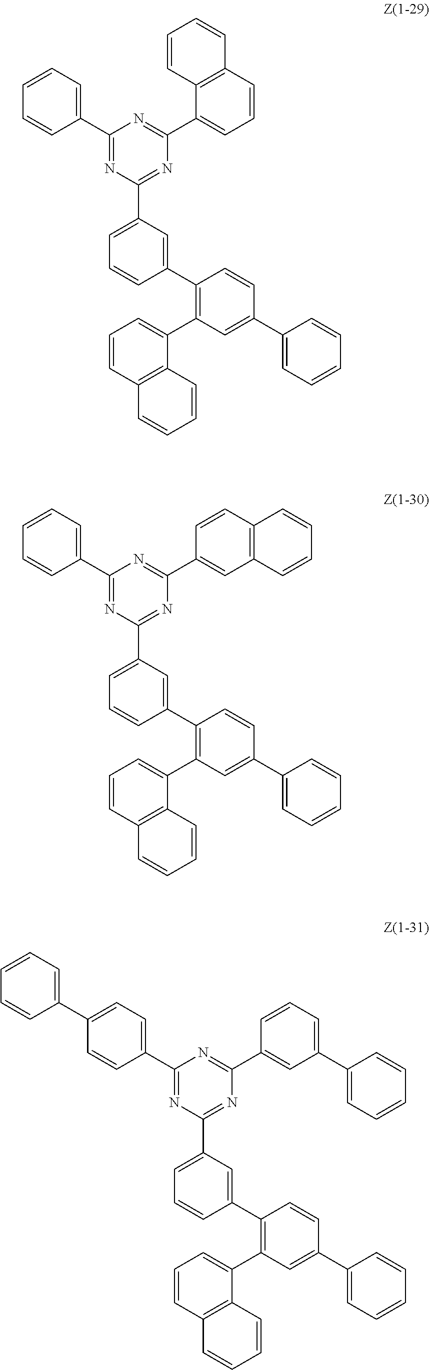 [SMILES: c1ccc(-c2ccc(-c3cccc(-c4nc(-c5ccccc5)nc(-c5cccc6ccccc56)n4)c3)c(-c3cccc4ccccc34)c2)cc1 ; c1ccc(-c2ccc(-c3cccc(-c4nc(-c5ccccc5)nc(-c5ccc6ccccc6c5)n4)c3)c(-c3cccc4ccccc34)c2)cc1 ; c1ccc(-c2ccc(-c3nc(-c4cccc(-c5ccccc5)c4)nc(-c4cccc(-c5ccc(-c6ccccc6)cc5-c5cccc6ccccc56)c4)n3)cc2)cc1]

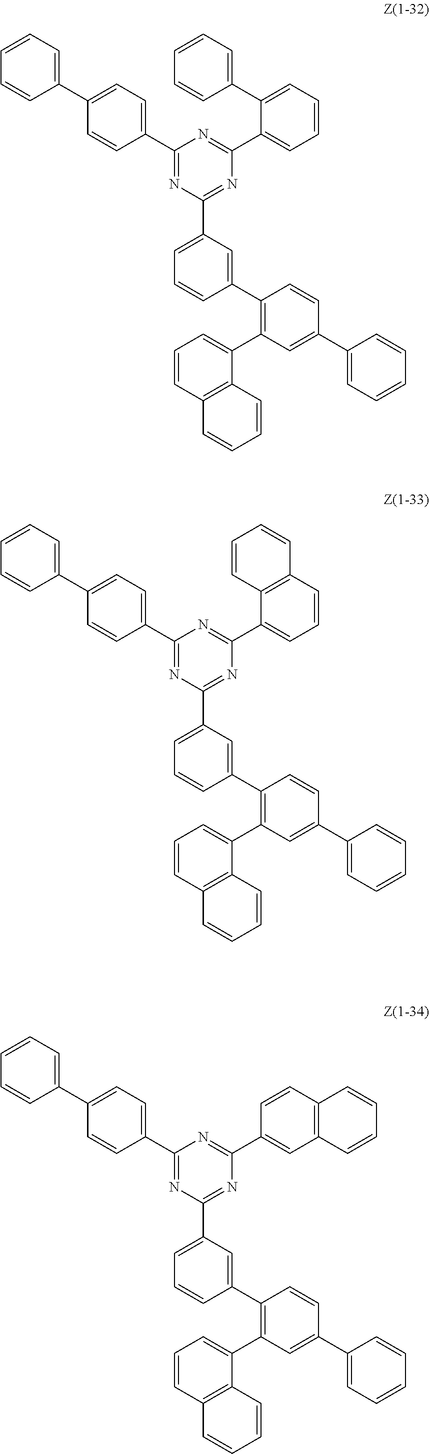 [SMILES: c1ccc(-c2ccc(-c3nc(-c4cccc(-c5ccc(-c6ccccc6)cc5-c5cccc6ccccc56)c4)nc(-c4ccccc4-c4ccccc4)n3)cc2)cc1 ; c1ccc(-c2ccc(-c3nc(-c4cccc(-c5ccc(-c6ccccc6)cc5-c5cccc6ccccc56)c4)nc(-c4cccc5ccccc45)n3)cc2)cc1 ; c1ccc(-c2ccc(-c3nc(-c4cccc(-c5ccc(-c6ccccc6)cc5-c5cccc6ccccc56)c4)nc(-c4ccc5ccccc5c4)n3)cc2)cc1]

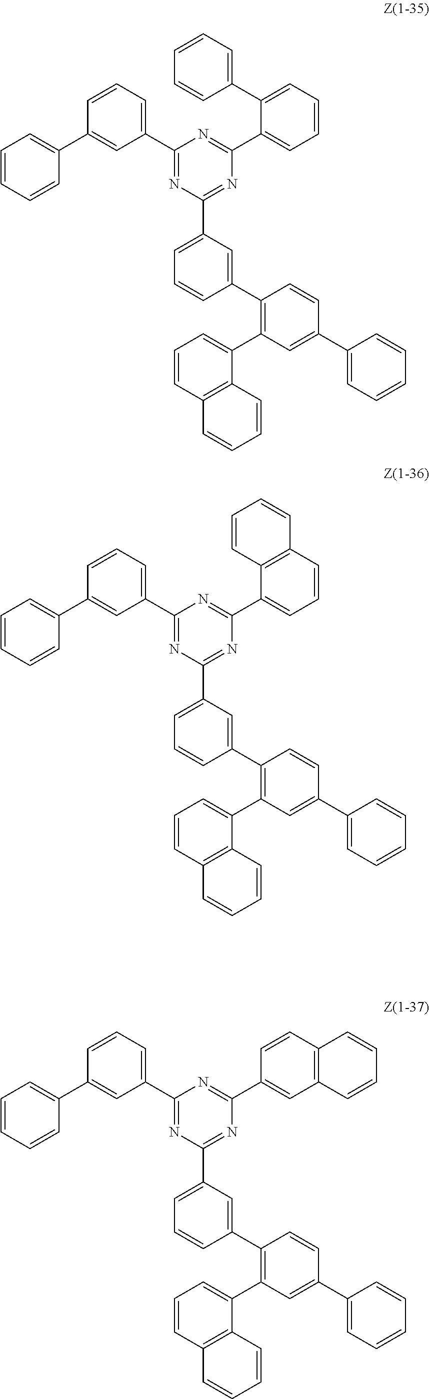 [SMILES: c1ccc(-c2cccc(-c3nc(-c4cccc(-c5ccc(-c6ccccc6)cc5-c5cccc6ccccc56)c4)nc(-c4ccccc4-c4ccccc4)n3)c2)cc1 ; c1ccc(-c2cccc(-c3nc(-c4cccc(-c5ccc(-c6ccccc6)cc5-c5cccc6ccccc56)c4)nc(-c4cccc5ccccc45)n3)c2)cc1 ; c1ccc(-c2cccc(-c3nc(-c4cccc(-c5ccc(-c6ccccc6)cc5-c5cccc6ccccc56)c4)nc(-c4ccc5ccccc5c4)n3)c2)cc1]

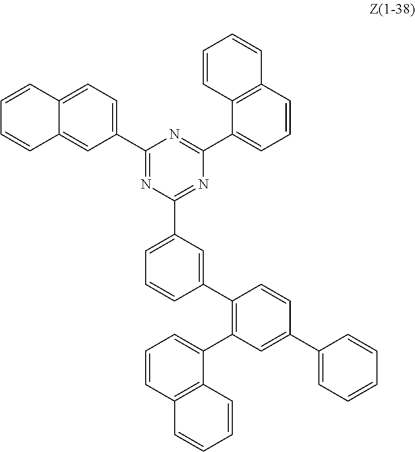 [SMILES: c1ccc(-c2ccc(-c3cccc(-c4nc(-c5ccc6ccccc6c5)nc(-c5cccc6ccccc56)n4)c3)c(-c3cccc4ccccc34)c2)cc1]

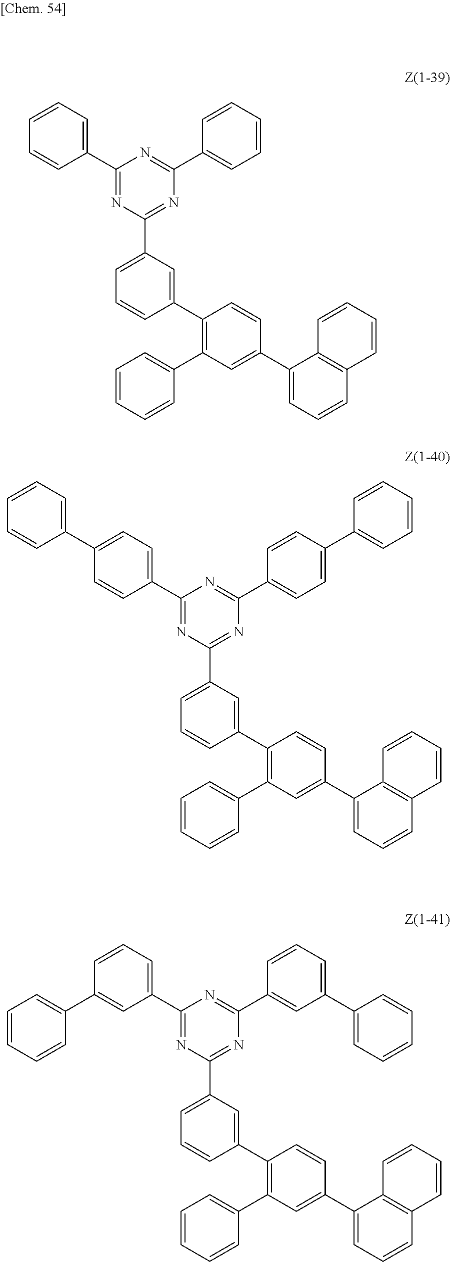 [SMILES: c1ccc(-c2nc(-c3ccccc3)nc(-c3cccc(-c4ccc(-c5cccc6ccccc56)cc4-c4ccccc4)c3)n2)cc1 ; c1ccc(-c2ccc(-c3nc(-c4ccc(-c5ccccc5)cc4)nc(-c4cccc(-c5ccc(-c6cccc7ccccc67)cc5-c5ccccc5)c4)n3)cc2)cc1 ; c1ccc(-c2cccc(-c3nc(-c4cccc(-c5ccccc5)c4)nc(-c4cccc(-c5ccc(-c6cccc7ccccc67)cc5-c5ccccc5)c4)n3)c2)cc1]

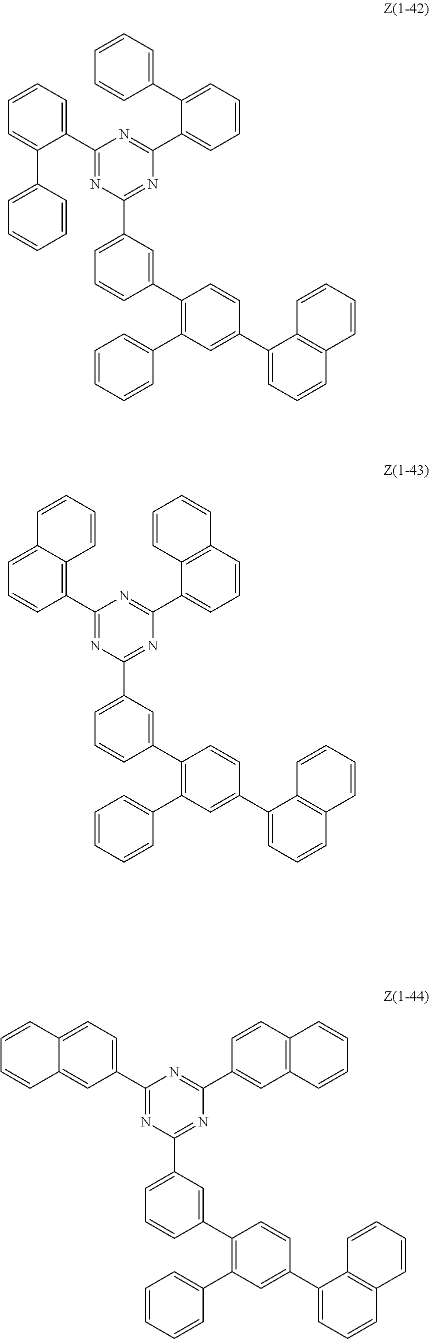 [SMILES: c1ccc(-c2cc(-c3cccc4ccccc34)ccc2-c2cccc(-c3nc(-c4ccccc4-c4ccccc4)nc(-c4ccccc4-c4ccccc4)n3)c2)cc1 ; c1ccc(-c2cc(-c3cccc4ccccc34)ccc2-c2cccc(-c3nc(-c4cccc5ccccc45)nc(-c4cccc5ccccc45)n3)c2)cc1 ; c1ccc(-c2cc(-c3cccc4ccccc34)ccc2-c2cccc(-c3nc(-c4ccc5ccccc5c4)nc(-c4ccc5ccccc5c4)n3)c2)cc1]

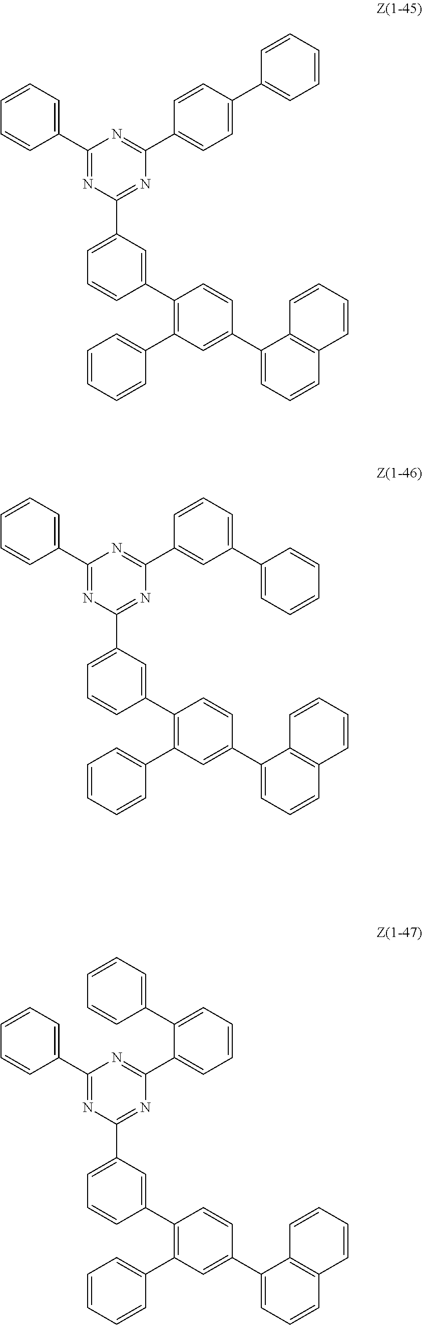 [SMILES: c1ccc(-c2ccc(-c3nc(-c4ccccc4)nc(-c4cccc(-c5ccc(-c6cccc7ccccc67)cc5-c5ccccc5)c4)n3)cc2)cc1 ; c1ccc(-c2cccc(-c3nc(-c4ccccc4)nc(-c4cccc(-c5ccc(-c6cccc7ccccc67)cc5-c5ccccc5)c4)n3)c2)cc1 ; c1ccc(-c2nc(-c3cccc(-c4ccc(-c5cccc6ccccc56)cc4-c4ccccc4)c3)nc(-c3ccccc3-c3ccccc3)n2)cc1]

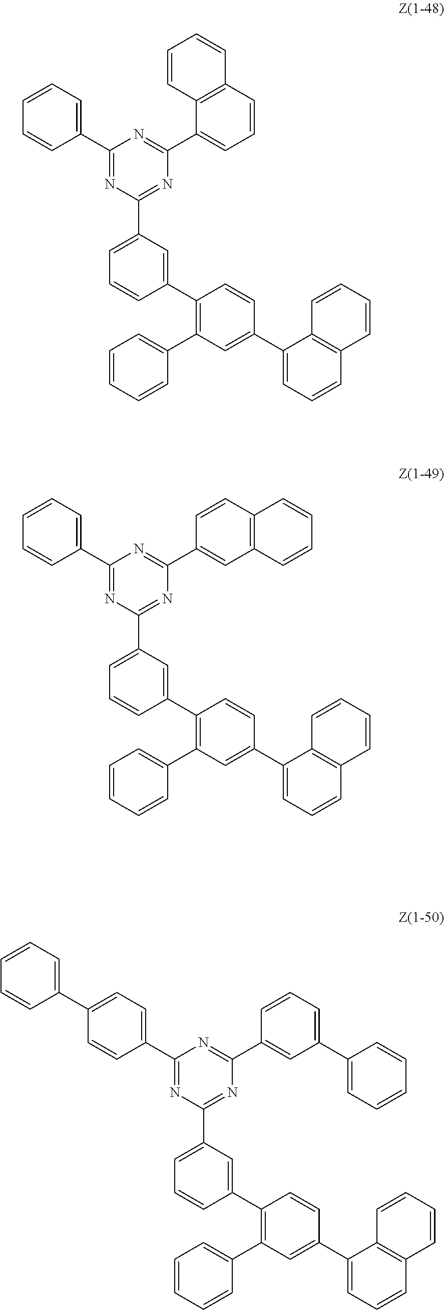 [SMILES: c1ccc(-c2nc(-c3cccc(-c4ccc(-c5cccc6ccccc56)cc4-c4ccccc4)c3)nc(-c3cccc4ccccc34)n2)cc1 ; c1ccc(-c2nc(-c3cccc(-c4ccc(-c5cccc6ccccc56)cc4-c4ccccc4)c3)nc(-c3ccc4ccccc4c3)n2)cc1 ; c1ccc(-c2ccc(-c3nc(-c4cccc(-c5ccccc5)c4)nc(-c4cccc(-c5ccc(-c6cccc7ccccc67)cc5-c5ccccc5)c4)n3)cc2)cc1]

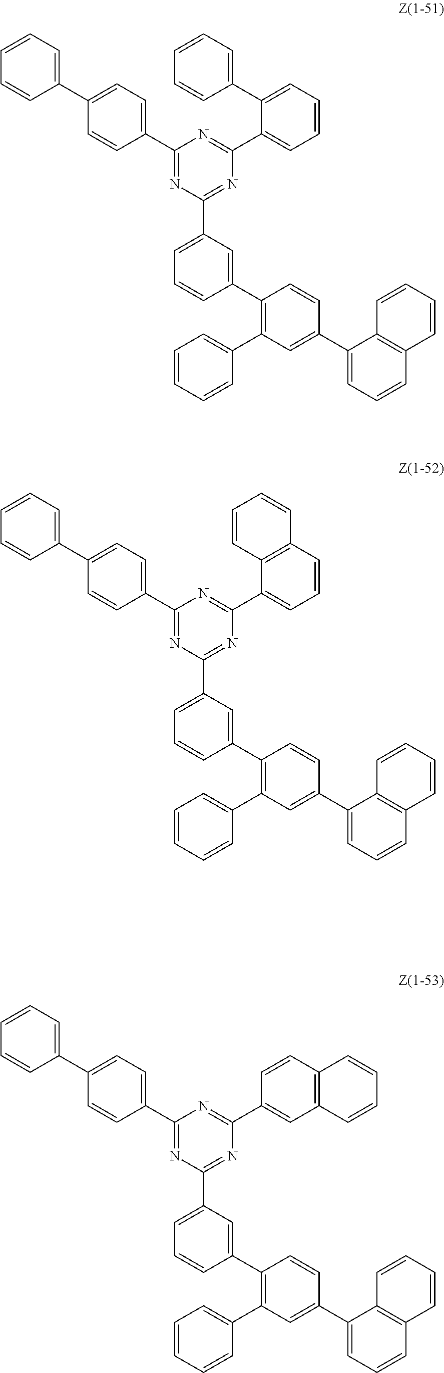 [SMILES: c1ccc(-c2ccc(-c3nc(-c4cccc(-c5ccc(-c6cccc7ccccc67)cc5-c5ccccc5)c4)nc(-c4ccccc4-c4ccccc4)n3)cc2)cc1 ; c1ccc(-c2ccc(-c3nc(-c4cccc(-c5ccc(-c6cccc7ccccc67)cc5-c5ccccc5)c4)nc(-c4cccc5ccccc45)n3)cc2)cc1 ; c1ccc(-c2ccc(-c3nc(-c4cccc(-c5ccc(-c6cccc7ccccc67)cc5-c5ccccc5)c4)nc(-c4ccc5ccccc5c4)n3)cc2)cc1]

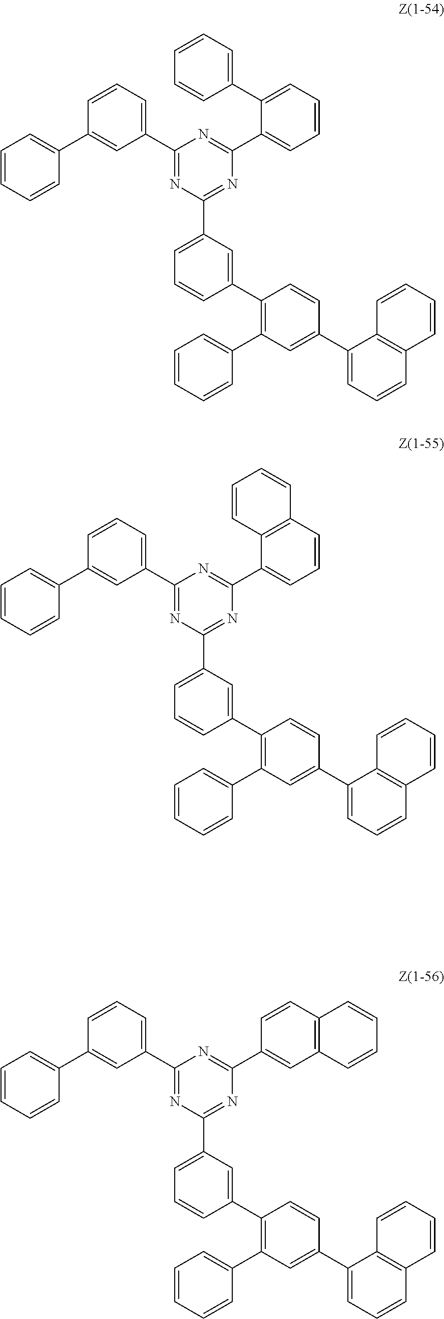 [SMILES: c1ccc(-c2cccc(-c3nc(-c4cccc(-c5ccc(-c6cccc7ccccc67)cc5-c5ccccc5)c4)nc(-c4ccccc4-c4ccccc4)n3)c2)cc1 ; c1ccc(-c2cccc(-c3nc(-c4cccc(-c5ccc(-c6cccc7ccccc67)cc5-c5ccccc5)c4)nc(-c4cccc5ccccc45)n3)c2)cc1 ; c1ccc(-c2cccc(-c3nc(-c4cccc(-c5ccc(-c6cccc7ccccc67)cc5-c5ccccc5)c4)nc(-c4ccc5ccccc5c4)n3)c2)cc1]

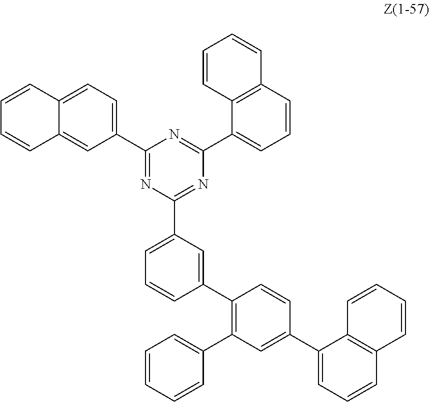 [SMILES: c1ccc(-c2cc(-c3cccc4ccccc34)ccc2-c2cccc(-c3nc(-c4ccc5ccccc5c4)nc(-c4cccc5ccccc45)n3)c2)cc1]

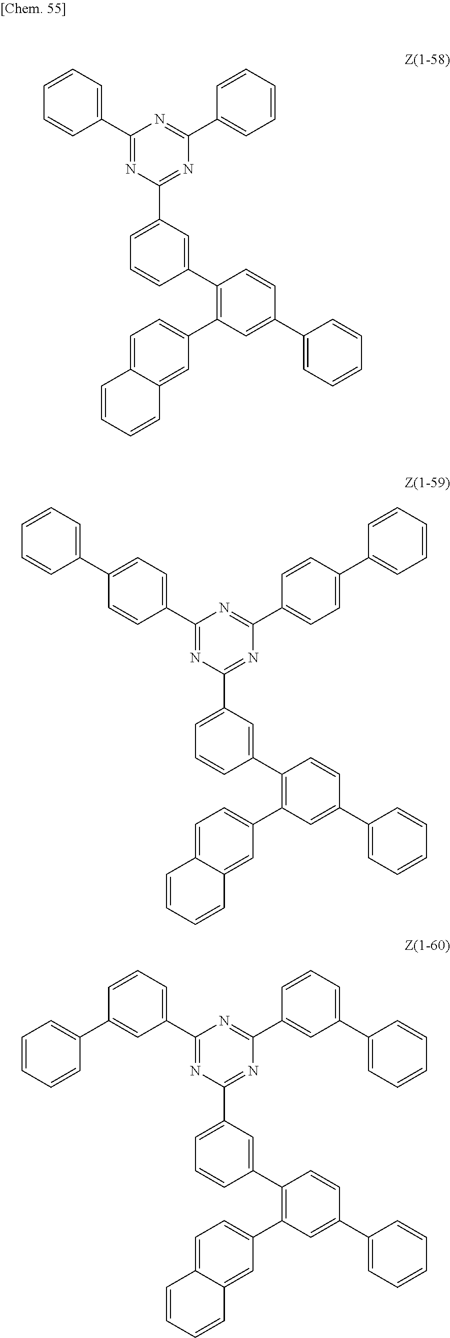 [SMILES: c1ccc(-c2ccc(-c3cccc(-c4nc(-c5ccccc5)nc(-c5ccccc5)n4)c3)c(-c3ccc4ccccc4c3)c2)cc1 ; c1ccc(-c2ccc(-c3nc(-c4ccc(-c5ccccc5)cc4)nc(-c4cccc(-c5ccc(-c6ccccc6)cc5-c5ccc6ccccc6c5)c4)n3)cc2)cc1 ; c1ccc(-c2cccc(-c3nc(-c4cccc(-c5ccccc5)c4)nc(-c4cccc(-c5ccc(-c6ccccc6)cc5-c5ccc6ccccc6c5)c4)n3)c2)cc1]

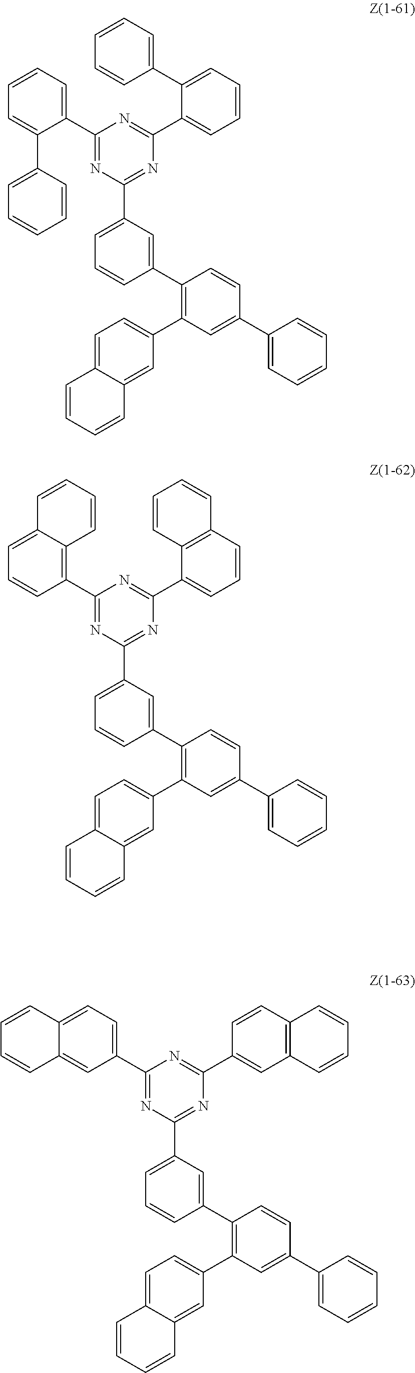 [SMILES: c1ccc(-c2ccc(-c3cccc(-c4nc(-c5ccccc5-c5ccccc5)nc(-c5ccccc5-c5ccccc5)n4)c3)c(-c3ccc4ccccc4c3)c2)cc1 ; c1ccc(-c2ccc(-c3cccc(-c4nc(-c5cccc6ccccc56)nc(-c5cccc6ccccc56)n4)c3)c(-c3ccc4ccccc4c3)c2)cc1 ; c1ccc(-c2ccc(-c3cccc(-c4nc(-c5ccc6ccccc6c5)nc(-c5ccc6ccccc6c5)n4)c3)c(-c3ccc4ccccc4c3)c2)cc1]

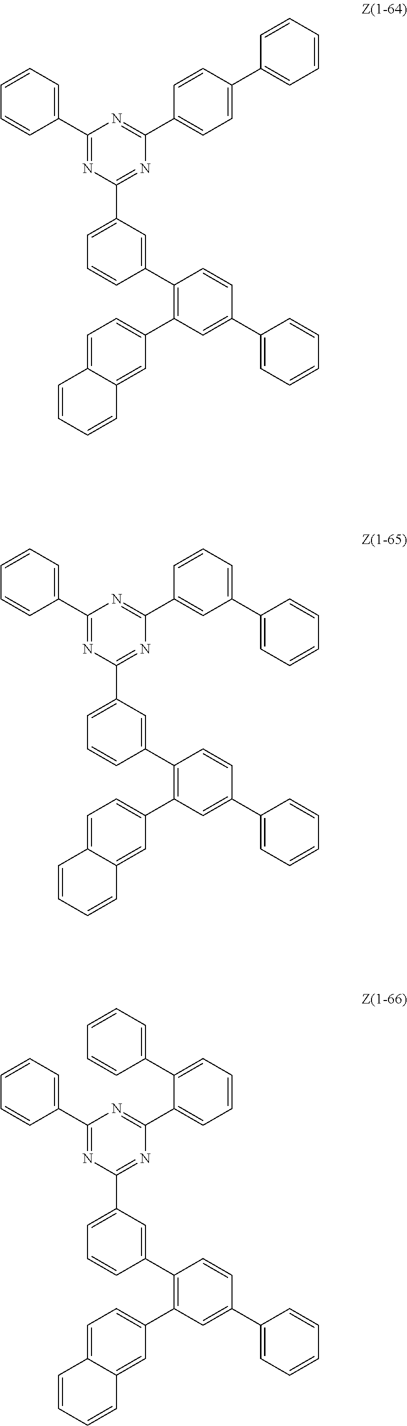 [SMILES: c1ccc(-c2ccc(-c3nc(-c4ccccc4)nc(-c4cccc(-c5ccc(-c6ccccc6)cc5-c5ccc6ccccc6c5)c4)n3)cc2)cc1 ; c1ccc(-c2cccc(-c3nc(-c4ccccc4)nc(-c4cccc(-c5ccc(-c6ccccc6)cc5-c5ccc6ccccc6c5)c4)n3)c2)cc1 ; c1ccc(-c2ccc(-c3cccc(-c4nc(-c5ccccc5)nc(-c5ccccc5-c5ccccc5)n4)c3)c(-c3ccc4ccccc4c3)c2)cc1]

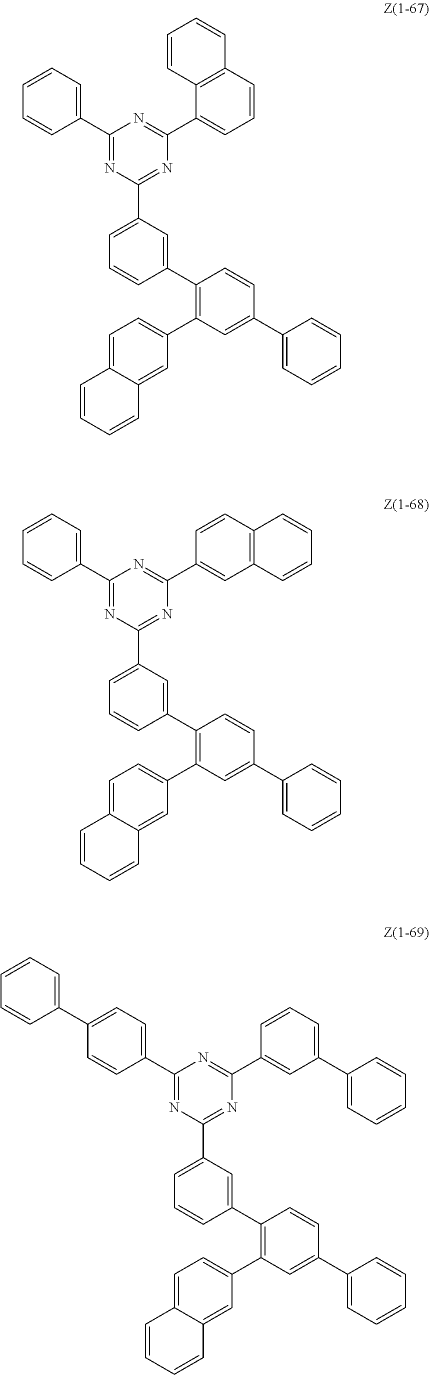 [SMILES: c1ccc(-c2ccc(-c3cccc(-c4nc(-c5ccccc5)nc(-c5cccc6ccccc56)n4)c3)c(-c3ccc4ccccc4c3)c2)cc1 ; c1ccc(-c2ccc(-c3cccc(-c4nc(-c5ccccc5)nc(-c5ccc6ccccc6c5)n4)c3)c(-c3ccc4ccccc4c3)c2)cc1 ; c1ccc(-c2ccc(-c3nc(-c4cccc(-c5ccccc5)c4)nc(-c4cccc(-c5ccc(-c6ccccc6)cc5-c5ccc6ccccc6c5)c4)n3)cc2)cc1]

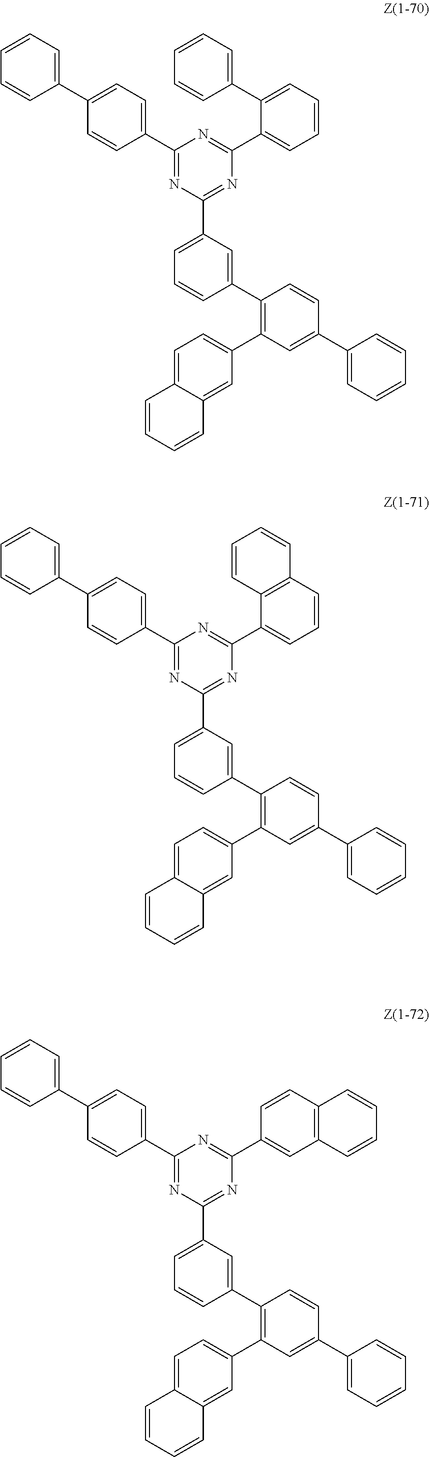 [SMILES: c1ccc(-c2ccc(-c3nc(-c4cccc(-c5ccc(-c6ccccc6)cc5-c5ccc6ccccc6c5)c4)nc(-c4ccccc4-c4ccccc4)n3)cc2)cc1 ; c1ccc(-c2ccc(-c3nc(-c4cccc(-c5ccc(-c6ccccc6)cc5-c5ccc6ccccc6c5)c4)nc(-c4cccc5ccccc45)n3)cc2)cc1 ; c1ccc(-c2ccc(-c3nc(-c4cccc(-c5ccc(-c6ccccc6)cc5-c5ccc6ccccc6c5)c4)nc(-c4ccc5ccccc5c4)n3)cc2)cc1]

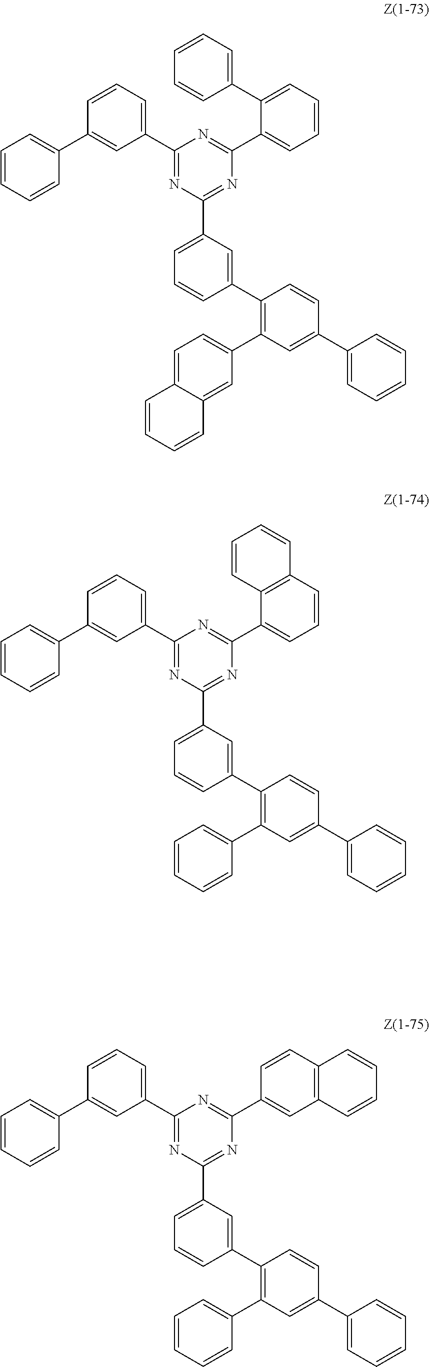 [SMILES: c1ccc(-c2cccc(-c3nc(-c4cccc(-c5ccc(-c6ccccc6)cc5-c5ccc6ccccc6c5)c4)nc(-c4ccccc4-c4ccccc4)n3)c2)cc1 ; c1ccc(-c2cccc(-c3nc(-c4cccc(-c5ccc(-c6ccccc6)cc5-c5ccccc5)c4)nc(-c4cccc5ccccc45)n3)c2)cc1 ; c1ccc(-c2cccc(-c3nc(-c4cccc(-c5ccc(-c6ccccc6)cc5-c5ccccc5)c4)nc(-c4ccc5ccccc5c4)n3)c2)cc1]

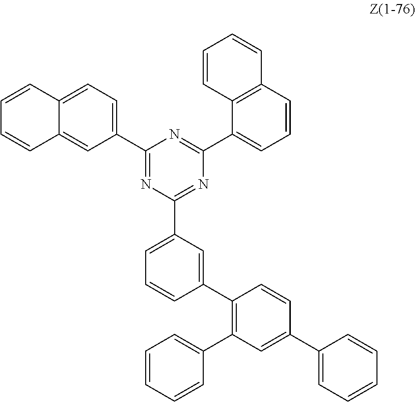 [SMILES: c1ccc(-c2ccc(-c3cccc(-c4nc(-c5ccc6ccccc6c5)nc(-c5cccc6ccccc56)n4)c3)c(-c3ccccc3)c2)cc1]

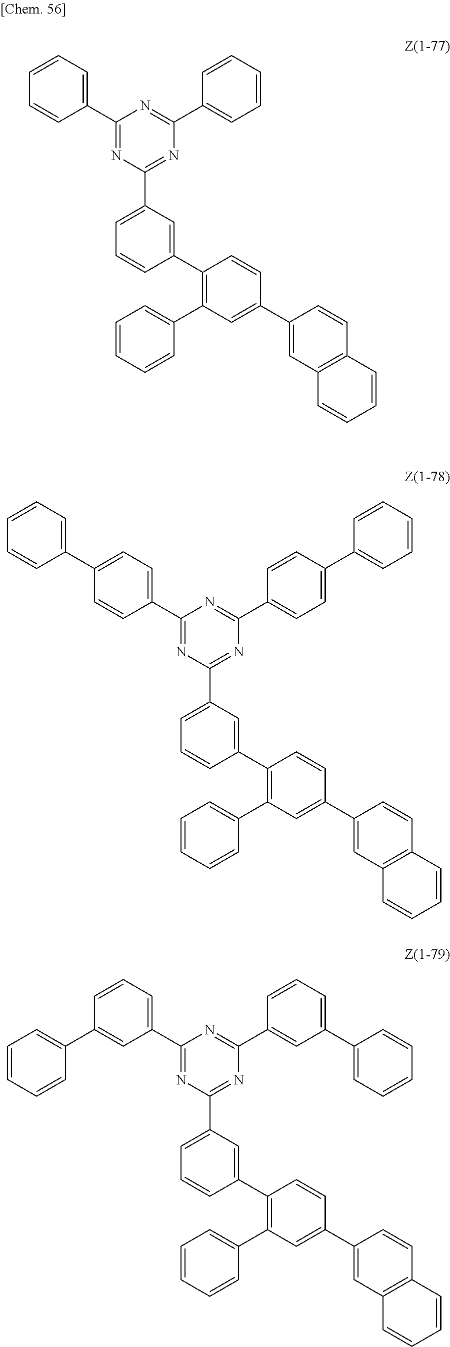 [SMILES: c1ccc(-c2nc(-c3ccccc3)nc(-c3cccc(-c4ccc(-c5ccc6ccccc6c5)cc4-c4ccccc4)c3)n2)cc1 ; c1ccc(-c2ccc(-c3nc(-c4ccc(-c5ccccc5)cc4)nc(-c4cccc(-c5ccc(-c6ccc7ccccc7c6)cc5-c5ccccc5)c4)n3)cc2)cc1 ; c1ccc(-c2cccc(-c3nc(-c4cccc(-c5ccccc5)c4)nc(-c4cccc(-c5ccc(-c6ccc7ccccc7c6)cc5-c5ccccc5)c4)n3)c2)cc1]

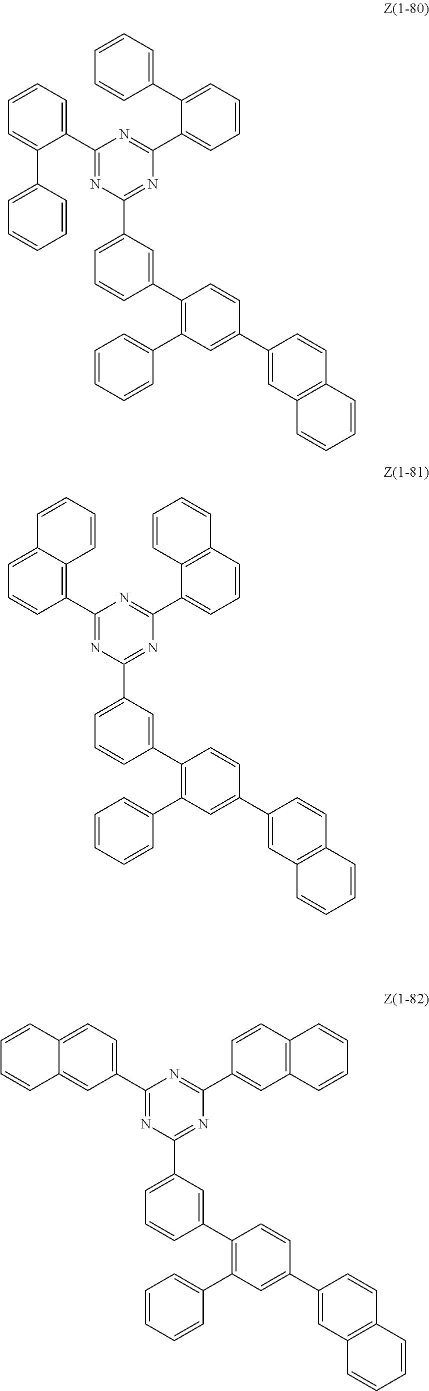 [SMILES: c1ccc(-c2cc(-c3ccc4ccccc4c3)ccc2-c2cccc(-c3nc(-c4ccccc4-c4ccccc4)nc(-c4ccccc4-c4ccccc4)n3)c2)cc1 ; c1ccc(-c2cc(-c3ccc4ccccc4c3)ccc2-c2cccc(-c3nc(-c4cccc5ccccc45)nc(-c4cccc5ccccc45)n3)c2)cc1 ; c1ccc(-c2cc(-c3ccc4ccccc4c3)ccc2-c2cccc(-c3nc(-c4ccc5ccccc5c4)nc(-c4ccc5ccccc5c4)n3)c2)cc1]

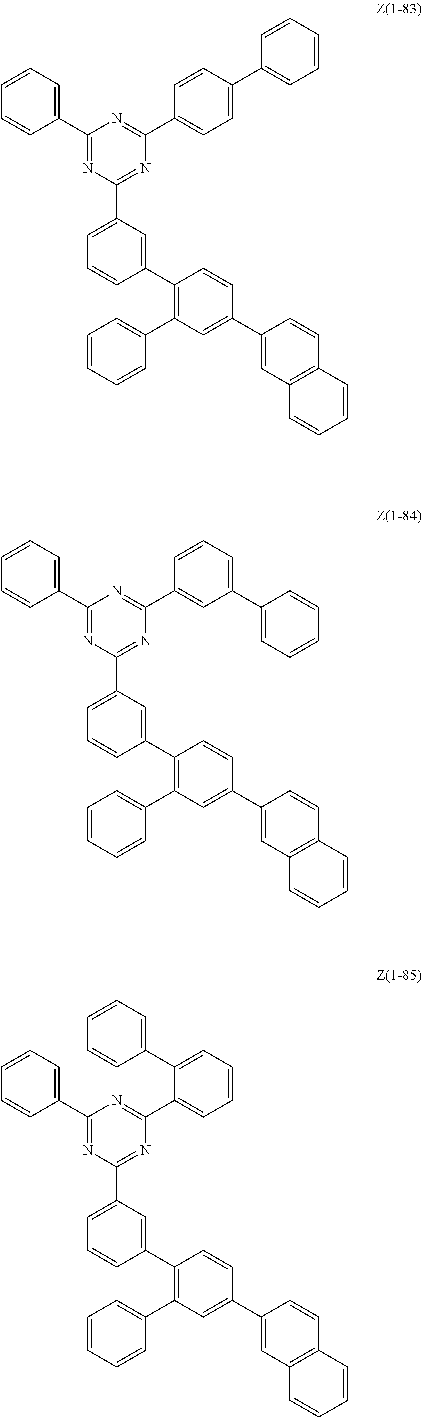 [SMILES: c1ccc(-c2ccc(-c3nc(-c4ccccc4)nc(-c4cccc(-c5ccc(-c6ccc7ccccc7c6)cc5-c5ccccc5)c4)n3)cc2)cc1 ; c1ccc(-c2cccc(-c3nc(-c4ccccc4)nc(-c4cccc(-c5ccc(-c6ccc7ccccc7c6)cc5-c5ccccc5)c4)n3)c2)cc1 ; c1ccc(-c2nc(-c3cccc(-c4ccc(-c5ccc6ccccc6c5)cc4-c4ccccc4)c3)nc(-c3ccccc3-c3ccccc3)n2)cc1]

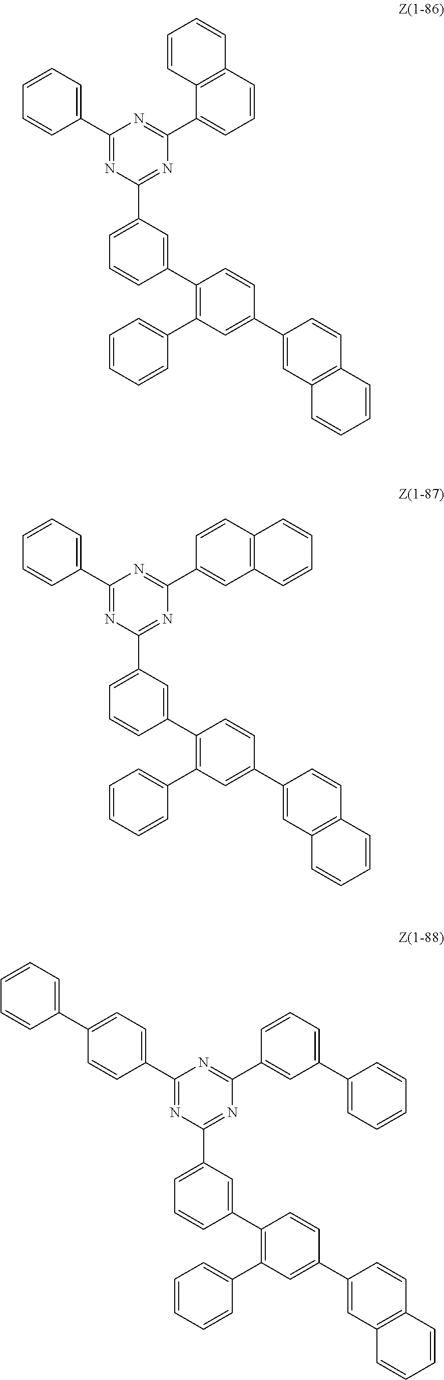 [SMILES: c1ccc(-c2nc(-c3cccc(-c4ccc(-c5ccc6ccccc6c5)cc4-c4ccccc4)c3)nc(-c3cccc4ccccc34)n2)cc1 ; c1ccc(-c2nc(-c3cccc(-c4ccc(-c5ccc6ccccc6c5)cc4-c4ccccc4)c3)nc(-c3ccc4ccccc4c3)n2)cc1 ; c1ccc(-c2ccc(-c3nc(-c4cccc(-c5ccccc5)c4)nc(-c4cccc(-c5ccc(-c6ccc7ccccc7c6)cc5-c5ccccc5)c4)n3)cc2)cc1]

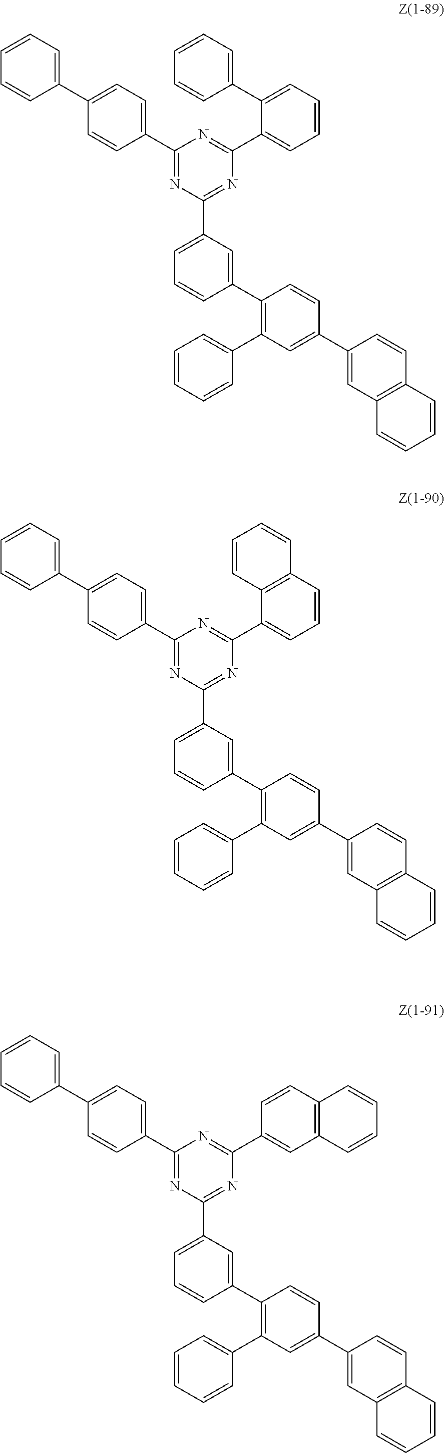 [SMILES: c1ccc(-c2ccc(-c3nc(-c4cccc(-c5ccc(-c6ccc7ccccc7c6)cc5-c5ccccc5)c4)nc(-c4ccccc4-c4ccccc4)n3)cc2)cc1 ; c1ccc(-c2ccc(-c3nc(-c4cccc(-c5ccc(-c6ccc7ccccc7c6)cc5-c5ccccc5)c4)nc(-c4cccc5ccccc45)n3)cc2)cc1 ; c1ccc(-c2ccc(-c3nc(-c4cccc(-c5ccc(-c6ccc7ccccc7c6)cc5-c5ccccc5)c4)nc(-c4ccc5ccccc5c4)n3)cc2)cc1]

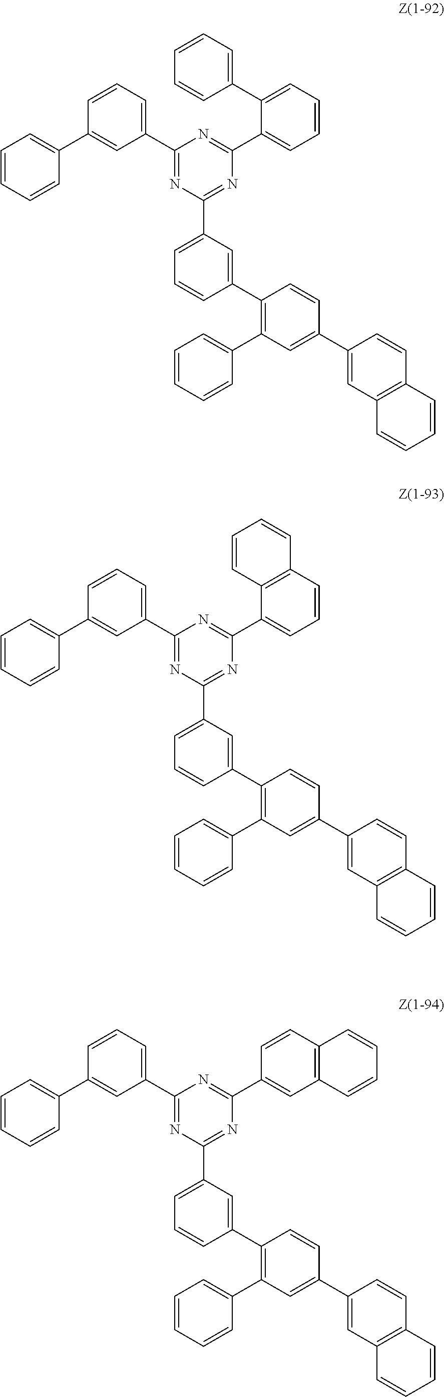 [SMILES: c1ccc(-c2cccc(-c3nc(-c4cccc(-c5ccc(-c6ccc7ccccc7c6)cc5-c5ccccc5)c4)nc(-c4ccccc4-c4ccccc4)n3)c2)cc1 ; c1ccc(-c2cccc(-c3nc(-c4cccc(-c5ccc(-c6ccc7ccccc7c6)cc5-c5ccccc5)c4)nc(-c4cccc5ccccc45)n3)c2)cc1 ; c1ccc(-c2cccc(-c3nc(-c4cccc(-c5ccc(-c6ccc7ccccc7c6)cc5-c5ccccc5)c4)nc(-c4ccc5ccccc5c4)n3)c2)cc1]

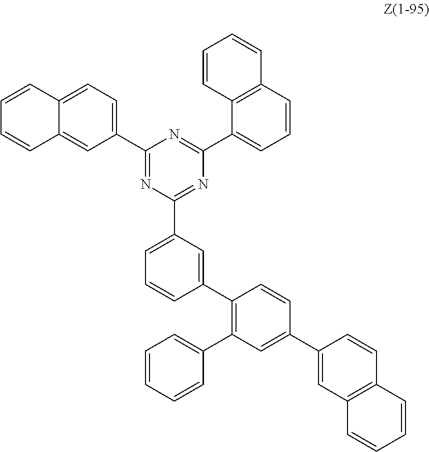 [SMILES: c1ccc(-c2cc(-c3ccc4ccccc4c3)ccc2-c2cccc(-c3nc(-c4ccc5ccccc5c4)nc(-c4cccc5ccccc45)n3)c2)cc1]

Hereinafter, applications of the triazine compound (1) will be described.

### <Material for Organic Light Emitting Diode, and Electron Transport Material for Organic Light Emitting Diode>

The triazine compound (1) may be used, for example, as a material for an organic light emitting diode, although the applications of the triazine compound (1) are not particularly limited thereto. In addition, the triazine compound (1) may be used, for example, as an electron transport material for an organic light emitting diode.

Specifically, a material for an organic light emitting diode according to an aspect of the present invention contains the triazine compound (1). Further, an electron transport material for an organic light emitting diode according to an aspect of the present invention contains the triazine compound (1). The material for an organic light emitting diode and the electron transport material for an organic light emitting diode, which contain the triazine compound (1), contribute to the manufacture of an organic light emitting diode exhibiting excellent driving voltage characteristics and excellent current efficiencies.

### <Organic Light Emitting Diode>

An organic light emitting diode according to an aspect of the present invention contains the triazine compound (1). Although the configuration of the organic light emitting diode is not particularly limited, examples thereof include configurations (i) to (vi) described below.
(i): anode/light emitting layer/cathode
(ii): anode/hole transport layer/light emitting layer/cathode (iii): anode/light emitting layer/electron transport layer/cathode
(iv): anode/hole transport layer/light emitting layer/electron transport layer/cathode (v): anode/hole injection layer/hole transport layer/light emitting layer/electron transport layer/electron injection layer/cathode (vi): anode/hole injection layer/charge generation layer/hole transport layer/light emitting layer/electron transport layer/cathode

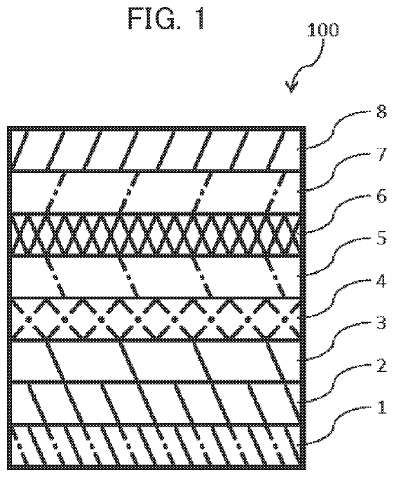

Hereinafter, the organic light emitting diode according to an aspect of the present invention will be described in more detail with reference to FIG. 1 using the configuration (vi) shown above as an example. FIG. 1 is a schematic cross-sectional view showing an example of a layered structure of the organic light emitting diode containing the triazine compound according to an aspect of the present invention.

It should be noted that, although the organic electroluminescence element shown in FIG. 1 has an element configuration of the so-called bottom-emission type, the organic electroluminescence element according to an aspect of the present invention is not limited to those having the element configuration of the bottom-emission type. In other words, the organic electroluminescence element according to an aspect of the present invention may have an element configuration of the top-emission type, or another known element configuration.

An organic light emitting diode 100 includes a substrate 1, an anode 2, a hole injection layer 3, a charge generation layer 4, a hole transport layer 5, a light emitting layer 6, an electron transport layer 7, and a cathode 8 in this order. However, some of these layers may be omitted, and, to the contrary, another layer may be added. For example, an electron injection layer may be provided between the electron transport layer 7 and the cathode 8; or the charge generation layer 4 may be omitted, and the hole transport layer 5 is provided directly on the hole injection layer 3.

Further, a configuration may be employed in which a single layer that combines functions exhibited by a plurality of layers, such as, for example, an electron injection and transport layer that combines the function of the electron injection layer and the function of the electron transport layer in a single layer, is provided in place of the plurality of layers. Furthermore, for example, the single layer of the hole transport layer 5, and the single layer of the electron transport layer 7 may be replaced by a plurality of hole transport layers and a plurality of electron transport layers, respectively.

### [Layer Containing Triazine Compound Represented by Formula (1)]

The organic light emitting diode contains the triazine compound represented by the formula (1) in one or more layers selected from the group consisting of the light emitting layer and the layers located between the light emitting layer and the cathode. Therefore, in the exemplary configuration shown in FIG. 1, the organic light emitting diode 100 contains the triazine compound (1) in at least one layer selected from the group consisting of the light emitting layer 6 and the electron transport layer 7.

In particular, it is preferable that the electron transport layer 7 contains the triazine compound (1). It should be noted that the triazine compound (1) may be contained in a plurality of layers included in the organic light emitting diode, and when the electron injection layer is provided between the electron transport layer and the cathode, the electron injection layer may contain the triazine compound (1). Incidentally, in the following, an organic light emitting diode 100 in which the electron transport layer 7 contains the triazine compound (1) will be described.

### [Substrate 1]

The substrate is not particularly limited, and examples thereof include a glass plate, a quartz plate, a plastic plate, and the like. In addition, in the case of a configuration in which the emitted light is extracted from the substrate 1 side, the substrate 1 is transparent with respect to the wavelength of the light.

Examples of an optically transparent plastic film include films made from polyethylene terephthalate (PET), polyethylene naphthalate (PEN), polyether sulfone (PES), polyether imide, polyether ether ketone, polyphenylene sulfide, polyarylate, polyimide, polycarbonate (PC), cellulose triacetate (TAC), cellulose acetate propionate (CAP), and the like.

### [Anode 2]

The anode 2 is provided on the substrate 1 (on the hole injection layer 3 side). In the case of an organic light emitting diode configured such that the emitted light is extracted through the anode, the anode is formed from a material that passes or substantially passes the emitted light.

A transparent material which may be used for the anode is not particularly limited, and examples thereof include indium tin oxide (ITO), indium zinc oxide (IZO), tin oxide, aluminum-doped tin oxide, magnesium indium oxide, nickel tungsten oxide, and other metal oxides, metal nitrides such as gallium nitride, metal selenides such as zinc selenide, metal sulfides such as zinc sulfide, and the like. It should be noted that in the case of an organic light emitting diode configured such that the light is extracted from the cathode side only, the light transmission property of the anode is not important. Therefore, examples of a material which may be used for the anode in this case include gold, iridium, molybdenum, palladium, platinum, and the like. A buffer layer (electrode interface layer) may be provided on the anode.

### [Hole Injection Layer 3 and Hole Transport Layer 5]

The hole injection layer 3, the charge generation layer 4 as described later, and the hole transport layer 5 are provided between the anode 2 and the light emitting layer 6 as described later, in the recited order from the anode 2 side. The hole injection layer and the hole transport layer function to transmit holes injected from the anode to the light emitting layer, and the presence of the hole injection layer and the hole transport layer between the anode and the light emitting layer enables more holes to be injected to the light emitting layer at a lower electric field.

In addition, the hole injection layer and the hole transport layer also function as an electron barrier layer. More specifically, electrons injected from the cathode and transported from the electron injection layer and/or the electron transport layer to the light emitting layer are inhibited from leaking into the hole injection layer and/or the hole transport layer by a barrier present in the interface between the light emitting layer and hole injection layer and/or the hole transport layer. Consequently, the electrons are accumulated in the interface on the light emitting layer, which exerts effects such as an improvement in current efficiency, leading to the formation of an organic light emitting diode with excellent light emitting performance.

A material for the hole injection layer and/or the hole transport layer has at least one of the following: hole injection properties, hole transporting properties, and electron barrier properties. The material for the hole injection layer and/or the hole transport layer may be either organic or inorganic.

Specific examples of the material for the hole injection layer and/or the hole transport layer include triazole derivatives, oxadiazole derivatives, imidazole derivatives, polyarylalkane derivatives, pyrazoline derivatives, pyrazolone derivatives, phenylenediamine derivatives, arylamine derivatives, amino-substituted chalcone derivatives, oxazole derivatives, styrylanthracene derivatives, fluorenone derivatives, hydrazone derivatives, stilbene derivatives, silazane derivatives, aniline-based copolymers, electroconductive high-molecular weight oligomers (thiophene oligomers, in particular), porphyrin compounds, aromatic tertiary amine compounds, styrylamine compounds, and the like. Of these, porphyrin compounds, aromatic tertiary amine compounds, and styrylamine compounds are preferable, and aromatic tertiary amine compounds are particularly preferable.

Specific examples of the aromatic tertiary amine compounds and the styrylamine compounds include N,N,N′,N′-tetraphenyl-4,4′-diaminophenyl, N,N′-diphenyl-N,N′-bis(3-methylphenyl)-[1,1′-biphenyl]-4,4′-diamine (TPD), 2,2-bis(4-di-p-tolylaminophenyl)propane, 1,1-bis(4-di-p-tolylaminophenyl) cyclohexane, N,N,N′,N′-tetra-p-tolyl-4,4′-diaminobiphenyl, 1,1-bis(4-di-p-tolylaminophenyl)-4-phenylcyclohexane, bis(4-dimethylamino-2-methylphenyl)phenylmethane, bis(4-di-p-tolylaminophenyl)phenylmethane, N,N′-diphenyl-N,N′-di(4-methoxyphenyl)-4,4′-diaminobiphenyl, N,N,N′,N′-tetraphenyl-4,4′-diaminodiphenyl ether, 4,4′-bis(diphenylamino)quadriphenyl, N,N,N-tri(p-tolyl)amine, 4-(di-p-tolylamino)-4′-[4-(di-p-tolylamino)styryl]stilbene, 4-N,N-diphenylamino-(2-diphenylvinyl)benzene, 3-methoxy-4′-N,N-diphenylaminostilbenezene, N-phenylcarbazole, 4,4′-bis[N-(1-naphthyl)-N-phenylamino]biphenyl (NPD), 4,4′,4″-tris[N-(3-methylphenyl)-N-phenylamino]triphenylamine (MTDATA), and the like. In addition, inorganic compounds such as p-type Si and p-type SiC may also be mentioned as an example of the material for the hole injection layer and the material for the hole transport layer.

The hole injection layer and the hole transport layer may each have a single-layer structure formed from one or two or more materials, or a layered structure formed of a plurality of layers having the same composition or different compositions.

### [Charge Generation Layer 4]

The charge generation layer 4 may be provided between the hole injection layer 3 and hole transport layer 5. A material of the charge generation layer is not particularly limited, and examples thereof include dipyrazino[2,3-f:2′,3′-h]quinoxaline-2,3,6,7,10,11-hexacarbonitrile (HAT-CN). The charge generation layer may have a single-layer structure formed from one or two or more materials, or a layered structure formed of a plurality of layers having the same composition or different compositions.

### [Light Emitting Layer 6]

The light emitting layer 6 is provided between the hole transport layer 5 and the electron transport layer 7 as described later. Examples of a material of the light emitting layer include phosphorescent light emitting materials, fluorescent light emitting materials, and thermally activated delayed fluorescent light emitting materials. In the light emitting layer, recombination of an electron-hole pair occurs, resulting in light emission.

The light emitting layer may be formed from a single low-molecular-weight material or a single polymer material, but more generally, the light emitting layer is formed from a host material doped with a guest compound. The light is primarily emitted from a dopant, and may exhibit any color.

Examples of the host material include compounds having a biphenyl group, a fluorenyl group, a triphenylsilyl group, a carbazole group, a pyrenyl group, or an anthryl group. More specifically, DPVBi (4,4′-bis(2,2-diphenylvinyl)-1,1′-biphenyl), BCzVBi (4,4′-bis(9-ethyl-3-carbazovinylene)-1,1′-biphenyl), TBADN (2-tert-butyl-9,10-di(2-naphthyl)anthracene), ADN (9,10-di(2-naphthyl)anthracene), CBP (4,4′-bis(carbazol-9-yl)biphenyl), CDBP (4,4′-bis(carbazol-9-yl)-2,2′-dimethylbiphenyl), 2-(9-phenylcarbazol-3-yl)-9-[4-(4-phenylphenylquinazolin-2-yl) carbazole, 9,10-bis(biphenyl)anthracene, and the like.

Examples of a fluorescent dopant include anthracene, pyrene, tetracene, xanthene, perylene, rubrene, coumarin, rhodamine, quinacridone, dicyanomethylenepyran compounds, thiopyran compounds, polymethine compounds, pyrylium, thiapyrylium compounds, fluorene derivatives, periflanthene derivatives, indenoperylene derivatives, bis(azinyl)amine boron compounds, bis(azinyl)methane compounds, carbostyryl compounds, and the like. The fluorescent dopant may be a combination of two or more selected from the above-listed materials.

Examples of a phosphorescent dopant include organic metal complexes of transition metals such as iridium, platinum, palladium, and osmium.

Specific examples of the fluorescent dopant and the phosphorescent dopant include Alq3 (tris(8-hydroxyquinoline)aluminum), DPAVBi (4,4′-bis[4-(di-p-tolylamino)styryl]biphenyl), perylene, bis[2-(4-n-hexylphenyl)quinoline](acetylacetonato)iridium(III), Ir(PPy)₃ (tris(2-phenylpyridine)iridium(III)), FIrPic (bis(3,5-difluoro-2-(2-pyridyl)phenyl-(2-carboxypyridyl)iridium(III))), and the like.

Further, the layer which may contain the light emitting material is not limited to the light emitting layer. For example, a layer adjacent to the light emitting layer (the hole transport layer 5, or the electron transport layer 7) may contain the light emitting material. This allows for further enhancement of the current efficiency of the organic light emitting diode.

The light emitting layer may have a single-layer structure formed from one or two or more materials, or a layered structure formed of a plurality of layers having the same composition or different compositions.

### [Electron Transport Layer 7]

The electron transport layer 7 is provided between the light emitting layer 6 and the cathode 8 as described later. The electron transport layer functions to transmit electrons injected from the cathode to the light emitting layer. The presence of the electron transport layer between the cathode and the light emitting layer enables the electrons to be injected into the light emitting layer at a lower electric field.

The electron transport layer preferably contains the triazine compound represented by the formula (1), as described above.

The electron transport layer may further contain a conventionally known electron transport material in addition to the triazine compound (1). Examples of the conventionally known electron transport material include 8-hydroxyquinolinatolithium (Liq), bis(8-hydroxyquinolinato)zinc, bis(8-hydroxyquinolinato)copper, bis(8-hydroxyquinolinato)manganese, tris(8-hydroxyquinolinato)aluminum, tris(2-methyl-8-hydroxyquinolinato)aluminum, tris(8-hydroxyquinolinato)gallium, bis(10-hydroxybenzo[h]quinolinato)beryllium, bis(10-hydroxybenzo[h]quinolinato)zinc, bis(2-methyl-8-quinolinato)chlorogallium, bis(2-methyl-8-quinolinato) (o-cresolato)gallium, bis(2-methyl-8-quinolinato)-1-naphtholatoaluminum, or bis(2-methyl-8-quinolinato)-2-naphtholatogallium, 2-[3-(9-phenanthrenyl)-5-(3-pyridinyl)phenyl]-4,6-diphenyl-1,3,5-triazine, 2-(4″-di-2-pyridinyl[1,1′:3′,1″-terphenyl]-5-yl)-4,6-diphenyl-1,3,5-triazine, BCP (2,9-dimethyl-4,7-diphenyl-1,10-phenanthroline), Bphen (4,7-diphenyl-1,10-phenanthroline), BAlq (bis(2-methyl-8-quinolinato)-4-(phenylphenolato)aluminum), bis(10-hydroxybenzo[h]quinolinato)beryllium), and the like.

The electron transport layer may have a single-layer structure formed from one or two or more materials, or a layered structure formed of a plurality of layers having the same composition or different compositions. In the case where the electron transport layer has a two-layer structure including a first electron transport layer on the light emitting layer side and a second electron transport layer on the cathode side, it is preferable that the second electron transport layer contains the triazine compound (1).

### [Cathode 8]

The cathode 8 is provided on the electron transport layer 7. In the case of an organic electroluminescence element configured such that only the emitted light having passed through the anode is extracted, the cathode may be formed from any electrically-conductive material. Examples of a material of the cathode include sodium, sodium-potassium alloys, magnesium, lithium, magnesium/copper mixtures, magnesium/silver mixtures, magnesium/aluminum mixtures, magnesium/indium mixtures, aluminum/aluminum oxide (Al₂O₃) mixtures, indium, lithium/aluminum mixtures, rare earth metals, and the like. A buffer layer (electrode interface layer) may be provided on the cathode (on the electron transport layer side thereof).

### [Forming Method of Each Layer]

The layers as described above except for the electrodes (the anode and the cathode) may be each formed by making a thin film of the material of each layer (together with a material such as a binder resin, and a solvent, as required) by a known method such as, for example, a vacuum deposition method, a spin coating method, a casting method, or an LB method (Langmuir-Blodgett method). The thickness of each layer formed thus is not particularly limited, and may be appropriately selected according to applications. The thickness of each layer is typically in the range of 5 nm to 5 μm.

The anode and the cathode may be formed by making a thin film of an electrode material by a technique such as vapor deposition or sputtering. A pattern may be formed using a mask having a desired shape during the vapor deposition or sputtering, and a pattern with a desired shape may be formed by photolithography after the formation of a thin film by the vapor deposition or sputtering, etc.

The anode and the cathode each have a thickness of preferably 1 μm or less, and more preferably 10 nm or more and 200 nm or less.

The organic light emitting diode according to an aspect of the present invention may be used as a type of lamp such as an illumination lamp and an exposure light source, or as a projection apparatus of the type to project an image or a display device (display) of the type for a viewer to directly view a static image and/or a video. In the case of the use as a display device to play a video, the drive system may be either a simple matrix (passive matrix) system or an active matrix system. Further, two or more organic light emitting diodes according to the present aspect which have different emission colors may be used to produce a full-color display device.

It should be noted that the triazine compound (1) according to an aspect of the present invention can be synthesized by an appropriate combination of known reactions (for example, Suzuki-Miyaura cross-coupling reaction, etc.).

### EXAMPLES

Hereinafter, the present invention is described in further detail by way of Examples, but the present invention should in no way be construed to be limited to these Examples.

¹H-NMR measurements were conducted on Gemini 200 (manufactured by Varian). FDMS measurements were conducted on M-80B manufactured by Hitachi, Ltd. Glass transition temperature measurements were conducted on DSC 7020 (manufactured by Hitachi High-Tech Corporation). The DSC measurements were conducted using aluminum oxide (Al₂O₃) as a reference, with 10 mg of a sample. As a pretreatment prior to the measurement, the temperature was increased from 30° C. to a temperature equal to or higher than the melting point of a sample at a rate of 10° C./min to melt the sample, and then the sample was brought into contact with dry ice to quench the sample. Subsequently, the temperature of the pretreated sample was increased from 30° C. at a rate of 10° C./min to measure its glass transition temperature. The light emission characteristics of the organic light emitting diodes were evaluated by applying direct current to the fabricated elements at room temperature (23° C., 50% RH), and by using a luminance meter (product name: BM-9, manufactured by Topcon Technohouse Corporation).

### <Triazine Compound X(1)>

### X Synthesis Example—1: Synthesis of Compound X(1-2)

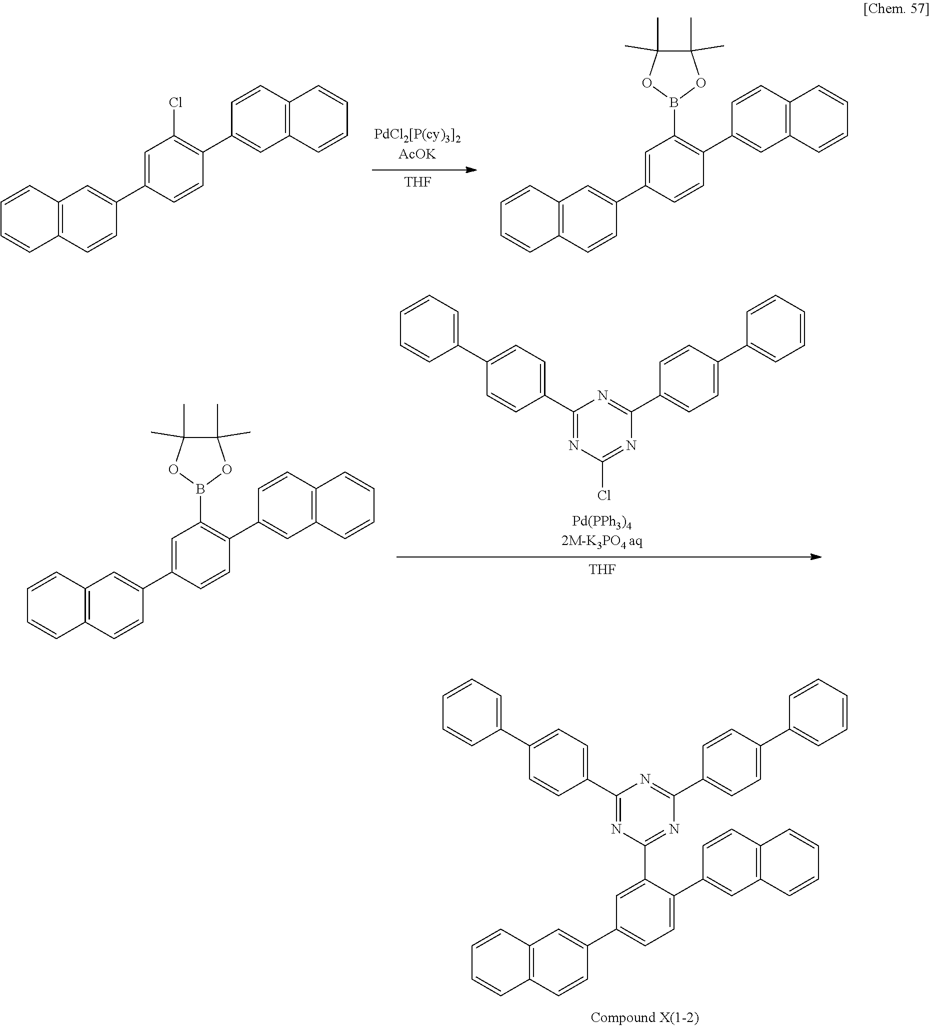

Under a nitrogen gas flow, 1,4-dioxane (69 ml) was added to a flask containing 1-chloro-2,5-di(naphthalen-2-yl)benzene (5.0 g, 13.7 mmol), bis(pinacolato)diboron (5.2 g, 20.6 mmol), PdCl₂[(Pcy₃)]₂ (202 mg, 0.27 mmol), and potassium acetate (4.0, 41.1 mmol), and the mixture was stirred at 100° C. for 21 hours. The mixture was allowed to cool to room temperature, solid was collected from the reaction solution via vacuum filtration, followed by washing with 1,4-dioxane. The obtained solid was recrystallized from a methanol (100 ml) solution, to yield 2-[2,5-di(naphthalen-2-yl)phenyl]-4,4,5,5-tetramethyl-1,3,2-dioxaborolan as a solid (amount: 5.1 g).

Under a nitrogen gas flow, tetrahydrofuran (191 ml) was added to a flask containing 2-chloro-4,6-di(biphenyl-4-yl)-1,3,5-triazine (4.0 g, 9.5 mmol), 2-[2,5-di(naphthalen-2-yl)phenyl]-4,4,5,5-tetramethyl-1,3,2-dioxaborolan (4.8 g, 10.5 mmol), and Pd(PPh₃)₄ (220 mg, 0.19 mmol). Further, a 2 M aqueous potassium phosphate solution (14 ml, 28.6 mmol) was added, and the mixture was stirred at 70° C. for 22 hours. The mixture was allowed to cool to room temperature, the precipitated solid was collected via vacuum filtration, and the obtained solid was washed with water and acetone. The obtained solid was dissolved in toluene (2000 ml), activated charcoal (1.2 g) was added thereto, and the mixture was heated with stirring at 100° C. for 1 hour. The activated charcoal was filtered off by vacuum filtration on a Kiriyama funnel with celite spread thereon, and recrystallization from the filtrate was conducted to yield compound X(1-2) as a white solid (amount: 4.5 g). The glass transition temperature was 116° C.

¹H-NMR (CDCl₃) δ (ppm): 8.85 (m, 1H), 8.26 (m, 5H), 7.90-8.10 (m, 7H), 7.72-7.86 (m, 3H), 7.44-7.65 (m, 16H), 7.39 (m, 3H).

### X Synthesis Example—2: Synthesis of Compound X(1-4)

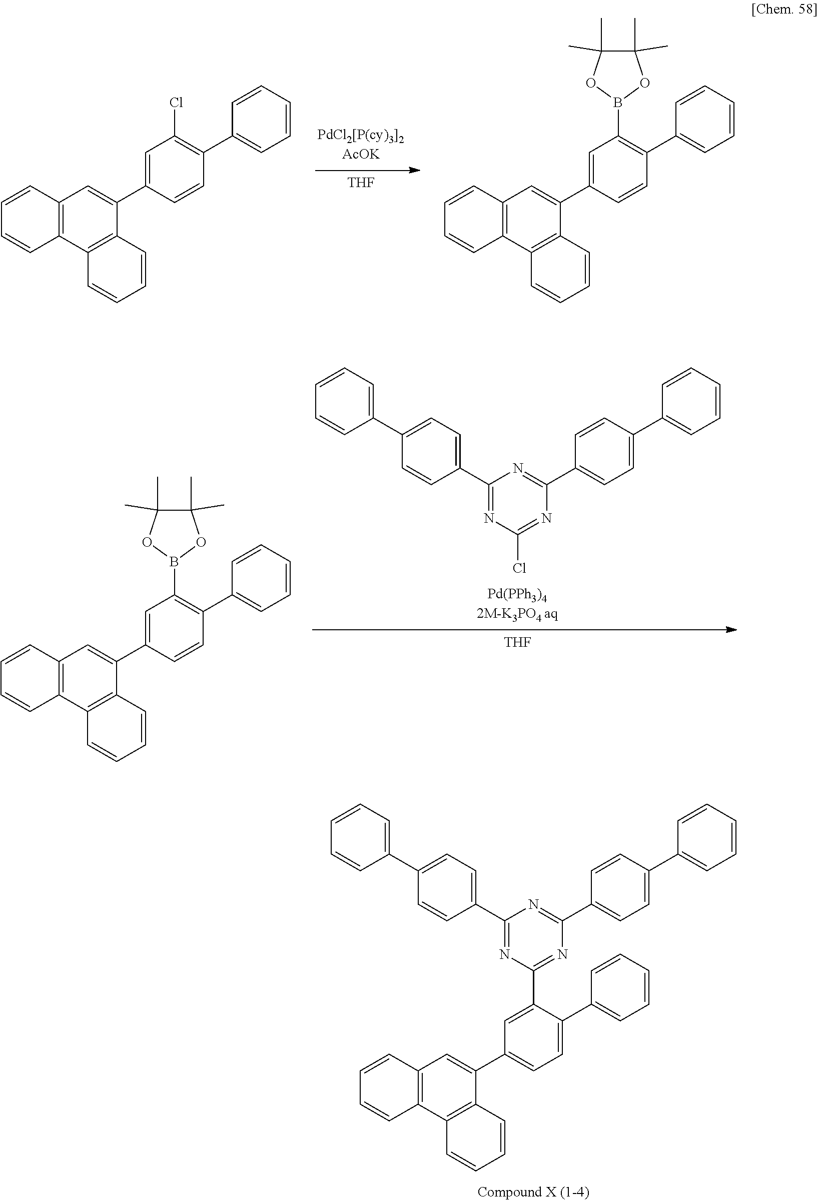

Under a nitrogen gas flow, tetrahydrofuran (110 ml) was added to a flask containing 2-chloro-4,6-di(biphenyl-4-yl)-1,3,5-triazine (2.3 g, 5.5 mmol), 2-[4-(9-phenanthrenyl) [1,1′-biphenyl]-2-yl]-4,4,5,5-tetramethyl-1,3,2-dioxaborolan (3.0 g, 6.57 mmol), and Pd(PPh₃)₄ (127 mg, 0.11 mmol). Further, a 2 M aqueous potassium phosphate solution (8 ml, 16.4 mmol) was added, and the mixture was stirred at 70° C. for 20 hours. The mixture was allowed to cool to room temperature, water and toluene were added, and liquid separation was carried out. The solvent was distilled off under reduced pressure, followed by the addition of water, the precipitated solid was collected via vacuum filtration, and the obtained solid was washed with water and acetone. The obtained solid was dissolved in toluene (250 ml), activated charcoal (0.4 g) was added thereto, and the mixture was heated with stirring at 100° C. for 1 hour. The activated charcoal was filtered off by vacuum filtration on a Kiriyama funnel with celite spread thereon, and the solvent was distilled off under reduced pressure. Recrystallization from a solution of a toluene/1-butanol mixture was conducted, to yield compound X(1-4) as a white solid (amount: 1.5 g). The glass transition temperature was 137° C.

¹H-NMR (CDCl₃) δ (ppm): 8.84 (d, 1H), 8.78 (d, 1H), 8.56 (m, 1H), 8.40 (m, 4H), 8.10 (d, 1H), 7.98 (d, 1H), 7.87 (s, 1H), 7.81 (m, 1H), 7.58-7.73 (m, 13H), 7.31-7.50 (m, 11H).

### X Synthesis Example—3: Synthesis of Compound X(1-5)

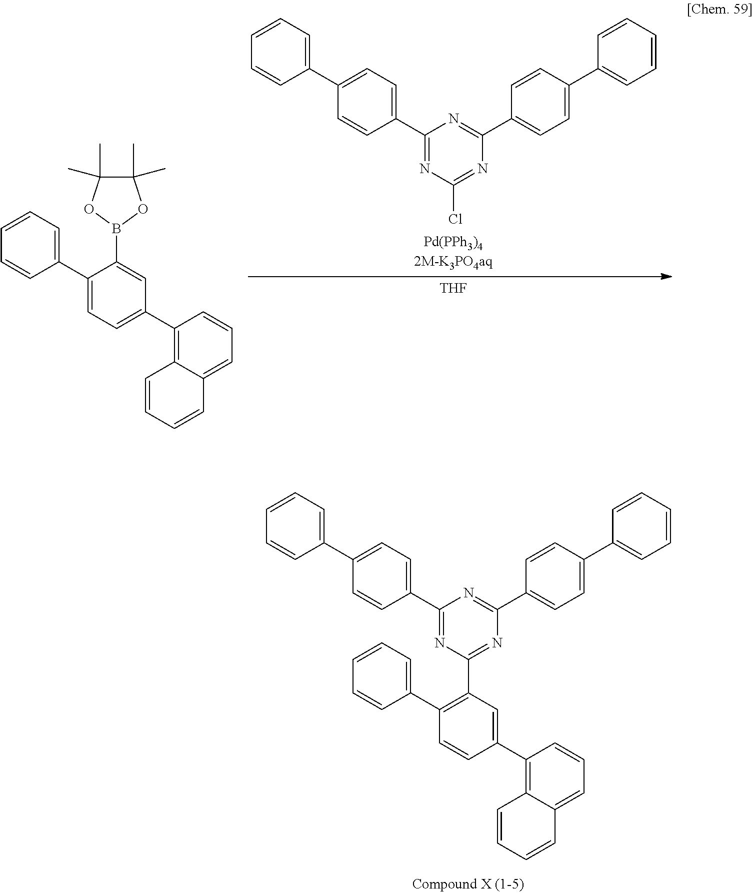

Experimental operations similar to X Synthesis Example—2 were conducted except that 2-[4-(9-phenanthrenyl) [1,1′-biphenyl]-2-yl]-4,4,5,5-tetramethyl-1,3,2-dioxaborolan was changed to 2-[4-(naphthalen-2-yl)[1,1′-biphenyl]-2-yl]-4,4,5,5-tetramethyl-1,3,2-dioxaborolan, to yield compound X(1-5) as a white solid (amount: 2.5 g).

FDMS: 663

### X Synthesis Example—4: Synthesis of Compound X(1-75)

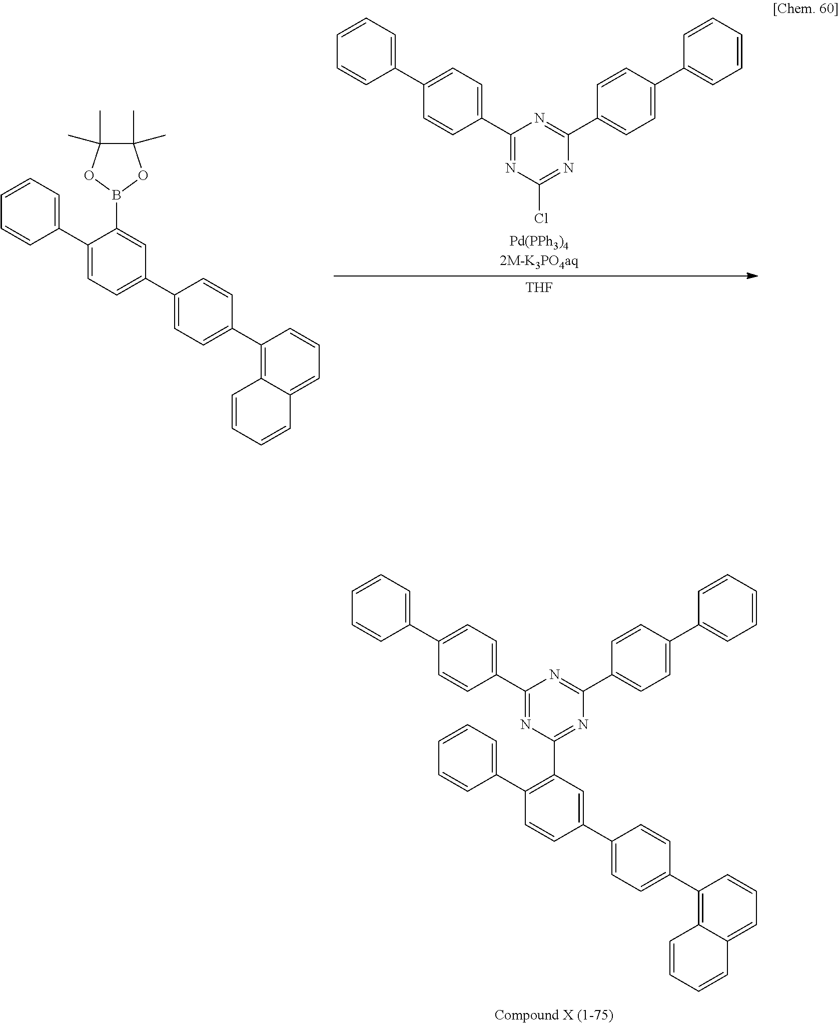 [SMILES: CC1(C)OB(c2cc(-c3ccc(-c4cccc5ccccc45)cc3)ccc2-c2ccccc2)OC1(C)C ; Clc1nc(-c2ccc(-c3ccccc3)cc2)nc(-c2ccc(-c3ccccc3)cc2)n1 ; c1ccc(-c2ccc(-c3nc(-c4ccc(-c5ccccc5)cc4)nc(-c4cc(-c5ccc(-c6cccc7ccccc67)cc5)ccc4-c4ccccc4)n3)cc2)cc1 ; "THF"]

Experimental operations similar to X Synthesis Example—2 were conducted except that 2-[4-(9-phenanthrenyl) [1,1′-biphenyl]-2-yl]-4,4,5,5-tetramethyl-1,3,2-dioxaborolan was changed to 2-[4-(naphthalen-2-yl)[1,1′: 4′,1″-terphenyl]-2′-yl]-4,4,5,5-tetramethyl-1,3,2-dioxaborolan, to yield compound X(1-75) as a white solid (amount: 1.4 g).

¹H-NMR (CDCl₃) δ (ppm): 8.69 (d, J=2.0 Hz, 1H), 8.47-8.41 (m, 5H), 8.03 (d, J=9.2 Hz, 1H), 7.96-7.88 (m, 5H), 7.72 (dd, J=3.6 Hz, 3.6 Hz, 2H), 7.69-7.61 (m, 8H), 7.58-7.54 (m, 2H), 7.52 (brd, J=1.6 Hz, 1H), 7.50-7.37 (m, 12H).

### X Synthesis Example—5: Synthesis of Compound X(1-80)

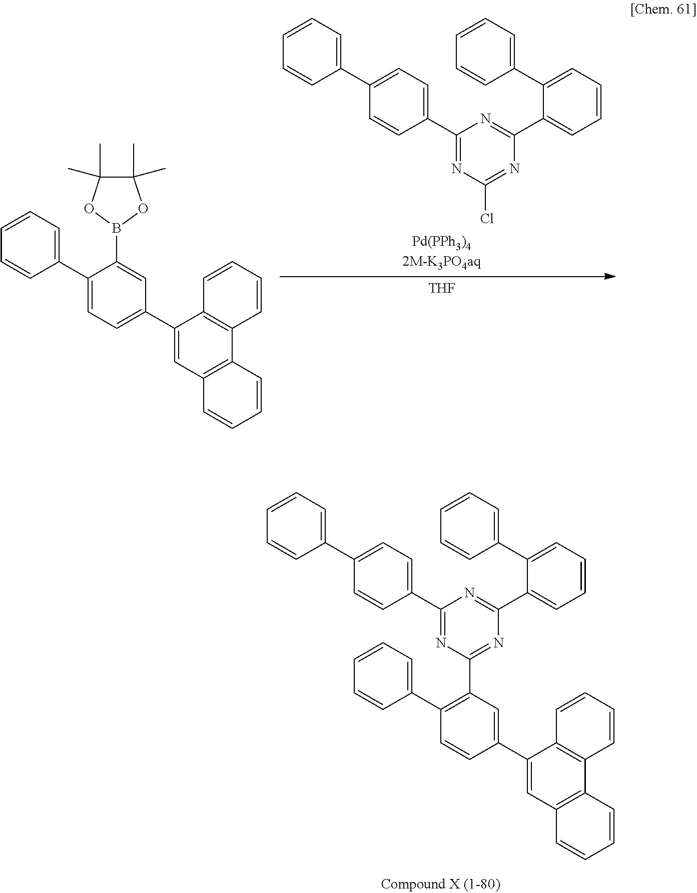

Experimental operations similar to X Synthesis Example—2 were conducted except that 2-chloro-4,6-di(biphenyl-4-yl)-1,3,5-triazine was changed to 6-chloro-2-[(1,1′-biphenyl)-2-yl]-4-[(1,1′-biphenyl)-4-yl]-1,3,5-triazine, to yield compound X(1-80) as a white solid (amount: 3.0 g). The glass transition temperature of the compound X(1-80) was 125° C.

¹H-NMR (CDCl₃) δ (ppm): 8.86 (d, J=9.2 Hz, 1H), 8.81 (d, J=8.0 Hz, 1H), 7.98 (brd, J=6.0 Hz, 1H), 7.88 (brd, J=8.0 Hz, 1H), 7.84-7.79 (m, 2H), 7.79 (d, J=1.6 Hz, 1H), 7.76-7.66 (m, 5H), 7.63-7.57 (m, 4H), 7.54-7.32 (m, 14H), 7.13 (dd, J=8.0 Hz, 1.2 Hz, 2H), 6.81 (brt, J=7.2 Hz, 2H), 6.42 (brt, J=7.6 Hz, 1H).

### <Triazine Compound Y(1)>

### Y Synthesis Example—1: Synthesis of Compound Y(1-96)

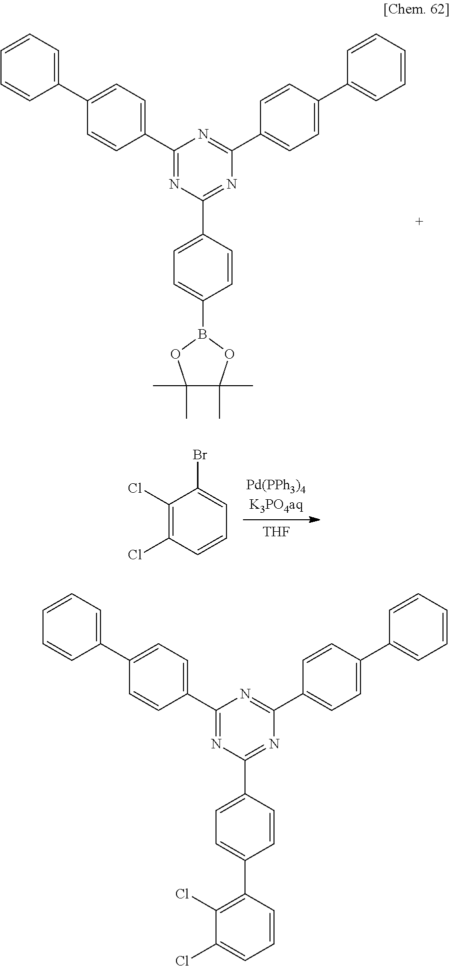

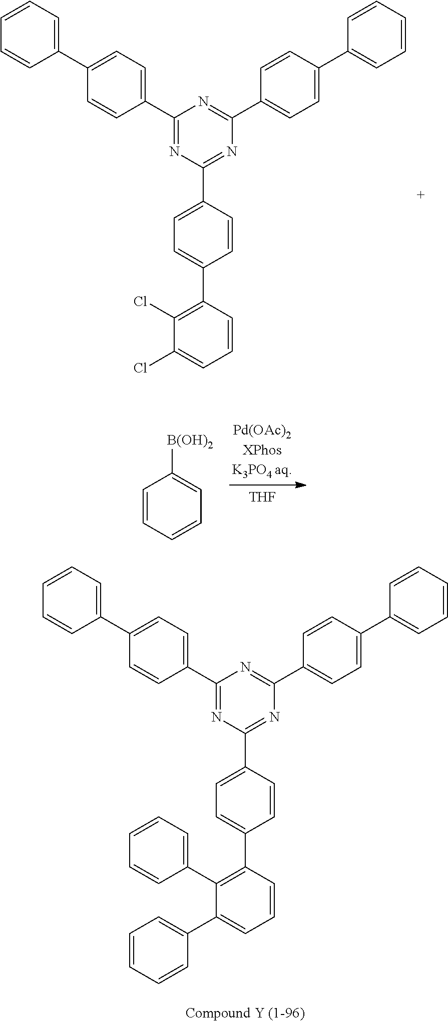 [SMILES: Clc1cccc(-c2ccc(-c3nc(-c4ccc(-c5ccccc5)cc4)nc(-c4ccc(-c5ccccc5)cc4)n3)cc2)c1Cl ; OB(O)c1ccccc1 ; c1ccc(-c2ccc(-c3nc(-c4ccc(-c5ccccc5)cc4)nc(-c4ccc(-c5cccc(-c6ccccc6)c5-c5ccccc5)cc4)n3)cc2)cc1 ; "THF"]

Under a nitrogen gas flow, tetrahydrofuran (170 mL) was added to a flask containing 2,4-bis([1,1′-biphenyl]-4-yl)-6-[4-(4,4,5,5-tetramethyl-1,3,2-dioxaborolan-2-yl)phenyl]-1,3,5-triazine (10.0 g, 17.02 mmol), 2,3-dichlorobromobenzene (4.2 g, 18.72 mmol), and Pd(PPh₃)₄ (393 mg, 0.34 mmol). Further, a 2 M aqueous potassium phosphate solution (25.5 mL, 51.06 mmol) was added, and the mixture was stirred at 70° C. for 24 hours. The mixture was allowed to cool to room temperature, the precipitated solid was collected via vacuum filtration, and washed with water, and then methanol. The obtained solid was dissolved in toluene, followed by recrystallization, to yield 2,4-bis([1,1′-biphenyl]-4-yl)-6-(2′,3′-dichloro[1,1′-biphenyl]-4-yl-1,3,5-triazine (amount: 9.1 g; yield: 88%).

Under a nitrogen gas flow, tetrahydrofuran (300 ml) was added to a flask containing 2,4-bis([1,1′-biphenyl]-4-yl)-6-(2′,3′-dichloro[1,1′-biphenyl]-4-yl-1,3,5-triazine (9.1 g, 15.05 mmol), phenylboronic acid (12.8 g, 105.4 mmol), palladium acetate (68 mg, 0.30 mmol), and 2-dicyclohexylphosphino-2′,4′,6′-triisopropylbiphenyl (X-Phos) (287 mg, 0.60 mmol). Further, a 2 M aqueous potassium phosphate solution (38 ml, 75.3 mmol) was added, and the mixture was stirred at 70° C. for 90 hours. The mixture was allowed to cool to room temperature, and the precipitated solid was collected via vacuum filtration, followed by washing with water and ethanol. The obtained solid was dissolved in toluene (1,000 mL), activated charcoal (1.2 g) was added thereto, and the mixture was heated with stirring at 100° C. for 2 hours. The activated charcoal was filtered off by vacuum filtration on a Kiriyama funnel with celite spread thereon, and the filtrate was distilled off under reduced pressure. Further, recrystallization from a toluene (800 ml) solution was conducted, to yield compound Y(1-96) as a white solid (amount: 6.9 g, yield 66%). The glass transition temperature was 158° C.

¹H-NMR (CDCl₃) δ (ppm): 8.83 (d, 4H), 8.62 (d, 2H), 7.80 (d, 4H), 7.71 (d, 4H), 7.47-7.57 (m, 7H), 7.42 (m, 2H), 7.32 (d, 2H), 7.17 (m, 3H), 7.08-7.13 (m, 2H), 6.98-7.05 (m, 3H), 6.89-6.94 (m, 2H).

### Y Synthesis Example—2: Synthesis of Compound Y(1-180)

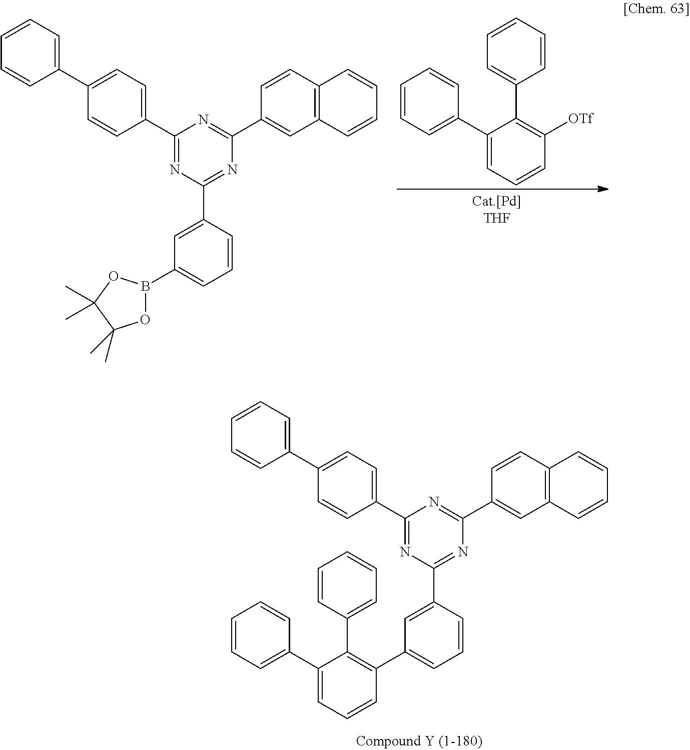

Under a nitrogen gas flow, tetrahydrofuran (270 ml) was added to a flask containing 2-[(1,1′-biphenyl)-4-yl]-4-(naphthalen-2-yl)-6-[3′-(4,4,5,5-tetramethyl-1,3,2-dioxaborolan-2-yl)phenyl]-1,3,5-triazine (15.0 g, 26.7 mmol), {(1,1′: 2′,1″-terphenyl)-3′-yl}trifluoromethanesulfonate (11.1 g, 29.4 mmol), and Pd(PPh₃)₄ (927 mg, 0.801 mmol). Further, a 2 M aqueous potassium phosphate solution (40.1 ml, 80.2 mmol) was added, and the mixture was stirred at 70° C. for 26 hours. The mixture was allowed to cool to room temperature, the precipitated solid was collected via vacuum filtration, and the obtained solid was washed with water and acetone. The obtained solid was dissolved in chlorobenzene (800 ml), activated charcoal (3.2 g) was added thereto, and the mixture was heated with stirring at 100° C. for 1 hour. The activated charcoal was filtered off by vacuum filtration on a Kiriyama funnel with celite spread thereon, and the filtrate was distilled off under reduced pressure. Further, recrystallization from a chlorobenzene (700 ml) solution was conducted, to yield compound Y(1-180) as a white solid (amount: 13.9 g). The glass transition temperature of the compound Y(1-180) was 11° C.

¹H-NMR (CDCl₃) δ (ppm): 6.95-7.04 (m, 5H), 7.14-7.23 (m, 5H), 7.31-7.33 (m, 1H), 7.38-7.45 (m, 2H), 7.50-7.65 (m, 7H), 7.74 (d, J=7.7 Hz, 2H), 7.83 (d, J=8.5 Hz, 2H), 7.94-7.96 (m, 1H), 8.03 (d, J=8.7 Hz, 1H), 8.11-8.13 (m, 1H), 8.64-8.67 (m, 2H), 8.8 (dd, J=8.5 Hz, 1.7 Hz, 1H), 8.86 (d, J=8.7 Hz, 2H), 9.32 (s, 1H).

### <Triazine Compound Z(1)>

### Z Synthesis Example—1: Synthesis of Compound Z(1-2)

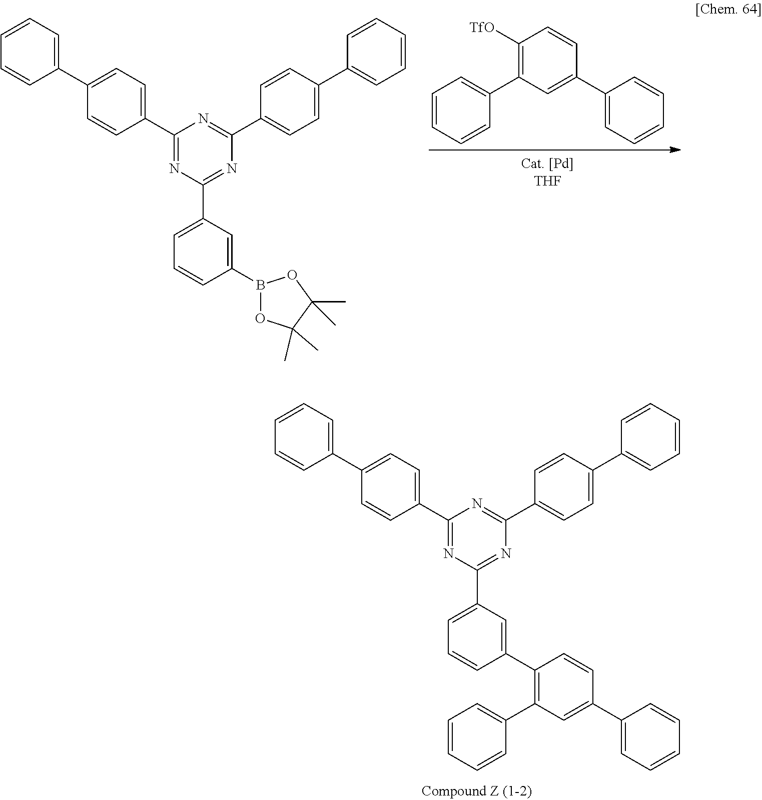

Under a nitrogen gas flow, tetrahydrofuran (51 ml) was added to a flask containing 2,4-bis[(1,1′-biphenyl)-4-yl]-6-[3′-(4,4,5,5-tetramethyl-1,3,2-dioxaborolan-2-yl)phenyl]-1,3,5-triazine (3.0 g, 5.1 mmol), {(1,1′: 3′,1″-terphenyl)-4′-yl}trifluoromethanesulfonate (2.1 g, 5.6 mmol), and Pd(PPh₃)₄ (133 mg, 0.12 mmol). Further, a 2 M aqueous potassium phosphate solution (7.7 ml, 16.0 mmol) was added, and the mixture was stirred at 70° C. for 20 hours. The mixture was allowed to cool to room temperature, the precipitated solid was collected via vacuum filtration, and the obtained solid was washed with water and acetone. The obtained solid was dissolved in toluene (500 ml), activated charcoal (0.5 g) was added thereto, and the mixture was heated with stirring at 100° C. for 1 hour. The activated charcoal was filtered off by vacuum filtration on a Kiriyama funnel with celite spread thereon, and recrystallization from the filtrate was conducted to yield compound Z(1-2) as a white solid (amount: 3.0 g). The glass transition temperature of the compound Z(1-2) was 119° C.

¹H-NMR (CDCl₃) δ (ppm): 8.84-8.79 (m, 4H), 8.71 (t, J=12.0 Hz, 1H), 8.64-8.69 (m, 1H), 7.79-7.84 (m, 4H), 7.68-7.77 (m, 9H), 7.27-7.67 (m, 15H), 7.18-7.24 (m, 1H).

### Z Synthesis Example—2: Synthesis of Compound Z(1-13)

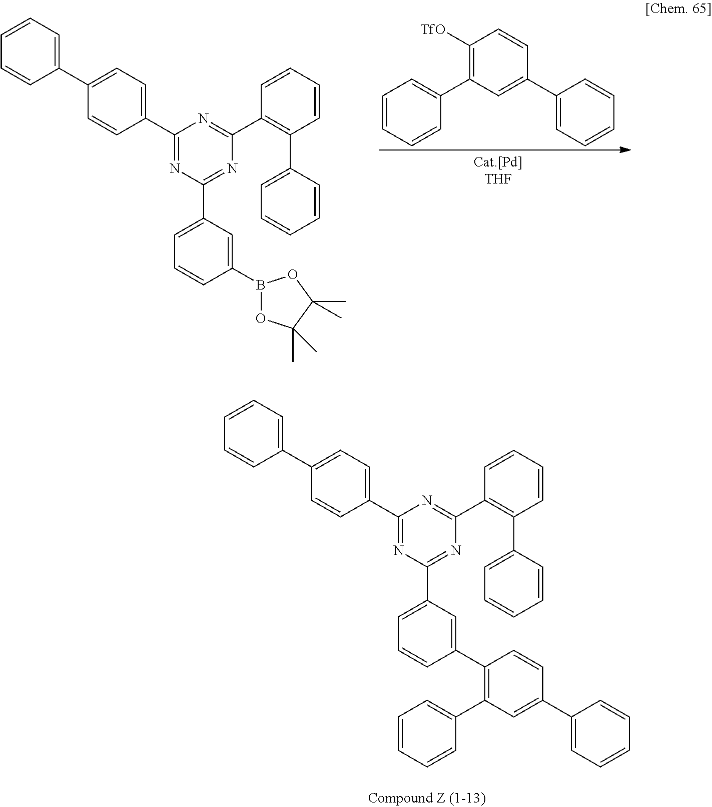 [SMILES: CC1(C)OB(c2cccc(-c3nc(-c4ccc(-c5ccccc5)cc4)nc(-c4ccccc4-c4ccccc4)n3)c2)OC1(C)C ; O=S(=O)(Oc1ccc(-c2ccccc2)cc1-c1ccccc1)C(F)(F)F ; c1ccc(-c2ccc(-c3nc(-c4cccc(-c5ccc(-c6ccccc6)cc5-c5ccccc5)c4)nc(-c4ccccc4-c4ccccc4)n3)cc2)cc1]

Experimental operations similar to Z Synthesis Example—1 were conducted except that 2,4-bis[(1,1′-biphenyl)-4-yl]-6-[3′-(4,4,5,5-tetramethyl-1,3,2-dioxaborolan-2-yl)phenyl]-1,3,5-triazine was changed to 2-[(1,1′-biphenyl)-2-yl]-4-[(1,1′-biphenyl)-4-yl]-6-[3′-(4,4,5,5-tetramethyl-1,3,2-dioxaborolan-2-yl)phenyl]-1,3,5-triazine, to yield compound Z(1-13) as a white solid (amount: 4.4 g). The glass transition temperature of the compound Z(1-13) was 107° C. FDMS: 689

### Z Synthesis Example—3: Synthesis of Compound Z(1-15)

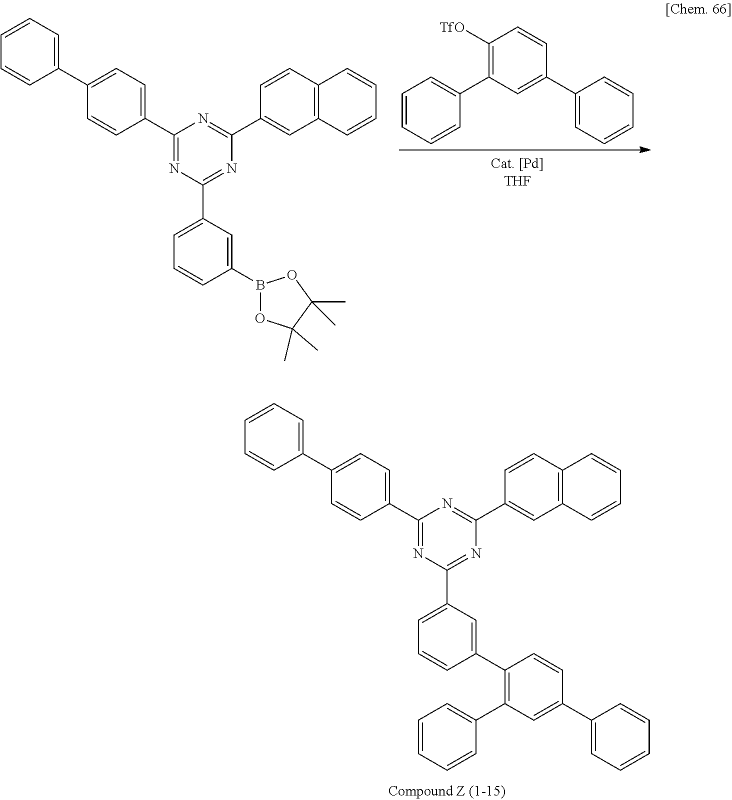

Experimental operations similar to Z Synthesis Example—1 were conducted except that 2,4-bis[(1,1′-biphenyl)-4-yl]-6-[3′-(4,4,5,5-tetramethyl-1,3,2-dioxaborolan-2-yl)phenyl]-1,3,5-triazine was changed to 2-[(1,1′-biphenyl)-2-yl]-6-(naphthalen-2-yl)-4-[3′-(4,4,5,5-tetramethyl-1,3,2-dioxaborolan-2-yl)phenyl]-1,3,5-triazine, to yield compound Z(1-15) as a white solid (amount: 9.7). The glass transition temperature of the compound Z(1-15) was 112° C.

¹H-NMR (CDCl₃) δ (ppm): 7.20 (t, J=7.4 Hz, 1H), 7.28 (t, J=7.3 Hz, 2H), 7.33-7.53 (m, 10H), 7.56-7.63 (m, 2H), 7.70-7.77 (m, 7H), 7.83 (d, J=8.6 Hz, 2H), 7.94-7.96 (m, 1H), 8.02 (d, J=8.7 Hz, 1H), 8.11-8.13 (m, 1H), 8.70 (dt, J=7.4 Hz, 1.7 Hz, 1H), 8.75 (t, J=1.5 Hz, 1H), 8.79 (dd, J=8.7 Hz, 1.5 Hz, 1H), 8.85 (d, J=8.5 Hz, 2H), 9.31 (s, 1H).

Next, element evaluations were performed using the obtained compounds.

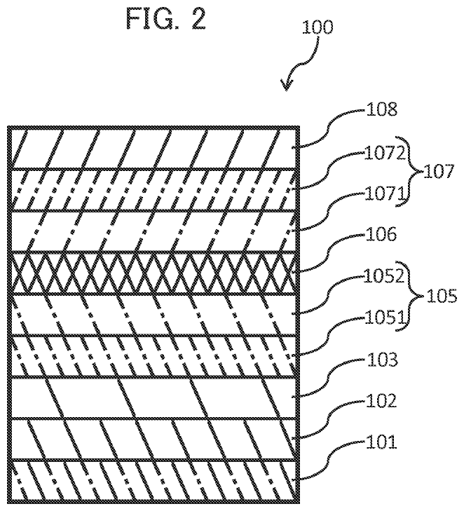

### X Element Example—1 (See FIG. 2)

### Preparation of Substrate 101 and Anode 102

As a substrate having an anode on the surface thereof, a glass substrate with an indium oxide-tin (ITO) transparent electrode formed of a 2 mm wide ITO film (thickness: 110 nm) and patterned in stripes was prepared. Then, this substrate was cleaned with isopropyl alcohol, and thereafter surface-treated by ozone ultraviolet ray cleaning.

### Preparation for Vacuum Deposition

Each layer was vacuum deposited on the cleaned and surface-treated substrate by vacuum deposition procedure to form and laminate the layers. First, the glass substrate was introduced to a vacuum deposition chamber, and the pressure within the vacuum deposition chamber was reduced to 1.0×10⁻⁴ Pa. Then, the layers were fabricated in the following order according to the respective film formation conditions.

### (Fabrication of Hole Injection Layer 103)

A hole injection layer 103 was fabricated by depositing sublimation-purified N-[1,1′-biphenyl]-4-yl-9,9-dimethyl-N-[4-(9-phenyl-9H-carbazol-3-yl)phenyl]-9H-fluorene-2-amine and 1,2,3-tris[(4-cyano-2,3,5,6-tetrafluorophenyl)methylene]cyclopropane at a rate of 0.15 nm/sec to achieve a film thickness of 10 nm.

### (Fabrication of First Hole Transport Layer 1051)

A first hole transport layer 1051 was fabricated by depositing sublimation-purified N-[1,1′-biphenyl]-4-yl-9,9-dimethyl-N-[4-(9-phenyl-9H-carbazol-3-yl)phenyl]-9H-fluorene-2-amine at a rate of 0.15 nm/sec to achieve a film thickness of 85 nm.

### (Fabrication of Second Hole Transport Layer 1052)

A second hole transport layer 1052 was fabricated by depositing sublimation-purified N-phenyl-N-(9,9-diphenylfluoren-2-yl)-N-(1,1′-biphenyl-4-yl)amine at a rate of 0.15 nm/sec to achieve a film thickness of 5 nm. According to the procedure described above, a hole transport layer 105 having a two-layer structure of the first hole transport layer 1051 and the second hole transport layer 1052 was fabricated.

### (Fabrication of Light Emitting Layer 106)

A light emitting layer 106 was fabricated by depositing sublimation-purified 3-(10-phenyl-9-anthryl)-dibenzofuran and 2,7-bis[N,N-di-(4-tert-butylphenyl)]amino-bisbenzofurano-9,9′-spirofluorene in a ratio of 95:5 (mass ratio) to achieve a film thickness of 20 nm. The film formation rate was 0.18 nm/sec.

### (Fabrication of First Electron Transport Layer 1071)

A first electron transport layer 1071 was fabricated by depositing sublimation-purified 2-[3′-(9,9-dimethyl-9H-fluoren-2-yl)[1,1′-biphenyl]-3-yl]-4,6-diphenyl-1,3,5-triazine at a rate of 0.05 nm/sec to achieve a film thickness of 6 nm.

### (Fabrication of Second Electron Transport Layer 1072)

A second electron transport layer 1072 was fabricated by depositing the compound X(1-2) synthesized in X Synthesis Example—1 and Liq in a ratio of 50:50 (mass ratio) to achieve a film thickness of 25 nm. The film formation rate was 0.15 nm/sec. According to the procedure described above, an electron transport layer 107 having a two-layer structure of the first electron transport layer 1071 and the second electron transport layer 1072 was fabricated.

### (Fabrication of Cathode 108)

Finally, a cathode 108 was formed using a metal mask placed so as to be perpendicular to the ITO stripes on the substrate. The cathode was formed by depositing silver/magnesium (mass ratio 1/10) and silver in this order to achieve a film thickness of 80 nm and 20 nm, respectively, and thus had a two-layer structure. The film formation rate for silver/magnesium was 0.5 nm/sec, and the film formation rate for silver was 0.2 nm/sec.

According to the procedure described above, an organic light emitting diode 100 having a light emission area of 4 mm², as shown in FIG. 2, was fabricated. It should be noted that the thickness of each layer was measured using a stylus profilometer (DEKTAK, manufactured by Bruker).

Furthermore, this element was encapsulated in a glove box under nitrogen atmosphere with an oxygen and moisture concentration of 1 ppm or less. The encapsulation was performed by encapsulating the deposition substrate (element) in a glass encapsulating cap using bisphenol F epoxy resin (manufactured by Nagase ChemteX Corporation).

Direct current was applied to the organic light emitting diode fabricated as described above, and light emission characteristics thereof were evaluated using a luminance meter (product name: BM-9, manufactured by Topcon Technohouse Corporation). Current efficiency (cd/A), driving voltage (V), and element lifetime in continuous lighting (h) under the application of a current density of 10 mA/cm² were measured as the light emission characteristics. For the element lifetime (h), when the fabricated element was driven in a continuous lighting mode at an initial luminance of 1,000 cd/m², a luminance decay time was measured, and the time required for a decrease in the luminance (cd/m²) by 5% was measured. It should be noted that the current efficiency, driving voltage, and element lifetime in continuous lighting (h) shown in Tables 1, 2 and 3 are expressed as relative values with respect to the results in X Element Reference Example 1, Y Element Reference Example 1 and Z Element Reference Example 1, respectively, as a reference value (100). The obtained measurement results are shown in Tables 1 to 3.

### X Element Example—2

An organic light emitting diode was fabricated according to the same method as X Element Example—1 except that compound X(1-4) was used in place of the compound X(1-2) in X Element Example—1, and evaluated. The obtained measurement results are shown in Table 1.

### X Element Example—3

An organic light emitting diode was fabricated according to the same method as X Element Example—1 except that compound X(1-5) synthesized in X Synthesis Example—3 was used in place of the compound X(1-2) in X Element Example—1, and evaluated. The obtained measurement results are shown in Table 1.

### X Element Example—4

An organic light emitting diode was fabricated according to the same method as X Element Example—1 except that the compound X(1-75) synthesized in X Synthesis Example—4 was used in place of the compound X(1-2) in X Element Example—1, and evaluated. The obtained measurement results are shown in Table 1.

### X Element Example—5

An organic light emitting diode was fabricated according to the same method as X Element Example—1 except that the compound X(1-80) synthesized in X Synthesis Example—5 was used in place of the compound X(1-2) in X Element Example—1, and evaluated. The obtained measurement results are shown in Table 1.

### X Element Reference Example—1

An organic light emitting diode was fabricated according to the same method as X Element Example—1 except that compound 24 described in Patent Document 1 was used in place of the compound X(1-2) in X Element Example—1, and evaluated. The obtained measurement results are shown in Table 1.

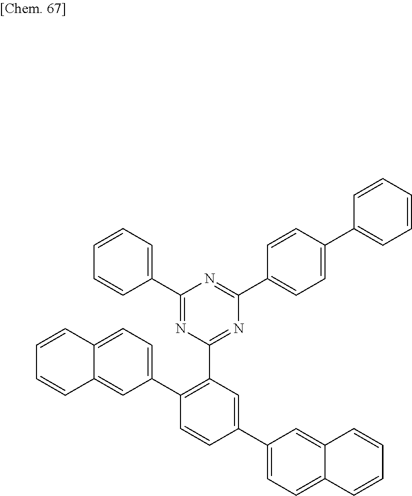 [SMILES: c1ccc(-c2ccc(-c3nc(-c4ccccc4)nc(-c4cc(-c5ccc6ccccc6c5)ccc4-c4ccc5ccccc5c4)n3)cc2)cc1]

TABLE 1

| | | Current | Driving | |
|---|---|---|---|---|
| | Compound | efficiency | voltage | Lifetime |
| X Element Example-1 | 1-2 | 100 | 98 | 130 |
| X Element Example-2 | 1-4 | 100 | 95 | 150 |
| X Element Example-3 | 1-5 | 101 | 95 | 160 |
| X Element Example-4 | 1-75 | 100 | 96 | 155 |
| X Element Example-5 | 1-80 | 106 | 99 | 115 |
| X Element Reference | Compound 24 | 100 | 100 | 100 |
| Example-1 | | | | |

### Y Element Example—1 (See FIG. 2)

An organic light emitting diode was fabricated according to the same method as X Element Example—1 except that the compound Y(1-96) synthesized in Y Synthesis Example—1 was used in place of the compound X(1-2) in X Element Example—1, and evaluated. The obtained measurement results are shown in Table 2.

### Y Element Example—2 (See FIG. 2)

An organic light emitting diode was fabricated according to the same method as X Element Example—1 except that the compound Y(1-180) synthesized in Y Synthesis Example—2 was used in place of the compound X(1-2) in X Element Example—1, and evaluated. The obtained measurement results are shown in Table 2.

### Y Element Reference Example—1

An organic light emitting diode was fabricated according to the same method as X Element Example—1 except that a compound disclosed in Example—9 of Patent Document 2 (Japanese Unexamined Patent Application, Publication No. 2018-95562) was used in place of the compound Y(1-96) in Y Element Example—1, and evaluated. The obtained measurement results are shown in Table 2.

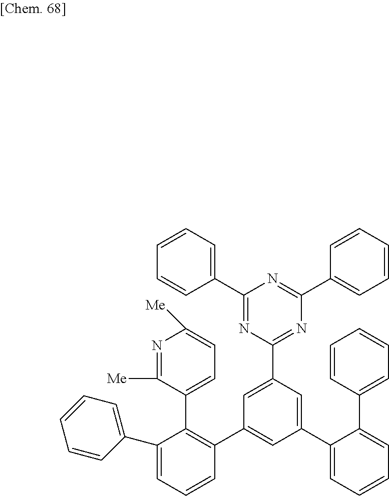 [SMILES: Cc1ccc(-c2c(-c3ccccc3)cccc2-c2cc(-c3nc(-c4ccccc4)nc(-c4ccccc4)n3)cc(-c3ccccc3-c3ccccc3)c2)c(C)n1]

TABLE 2

| | | | | Lifetime before |
|---|---|---|---|---|
| | | Current | Driving | decrease in |
| | Compound | efficiency | voltage | luminance by 5% |
| Y Element Example-1 | 1-96 | 100 | 95 | 250 |
| Y Element Example-2 | 1-180 | 104 | 97 | 185 |
| Y Element Reference | Example 9 | 100 | 100 | 100 |
| Example-1 | | | | |

### Z Element Example—1 (See FIG. 2)

An organic light emitting diode was fabricated according to the same method as X Element Example—1 except that the compound Z(1-2) synthesized in Z Synthesis Example—1 was used in place of the compound X(1-2) in X Element Example—1, and evaluated. The obtained measurement results are shown in Table 3.

### Z Element Example—2

An organic light emitting diode was fabricated according to the same method as X Element Example—1 except that the compound Z(1-13) synthesized in Z Synthesis Example—2 was used in place of the compound X(1-2) in X Element Example—1, and evaluated. The obtained measurement results are shown in Table 3.

### Z Element Example—3

An organic light emitting diode was fabricated according to the same method as X Element Example—1 except that the compound Z(1-15) synthesized in Z Synthesis Example—3 was used in place of the compound X(1-2) in X Element Example—1, and evaluated. The obtained measurement results are shown in Table 3.

### Z Element Reference Example—1

An organic light emitting diode was fabricated according to the same method as X Element Example—1 except that a compound (ETL-1) shown below and described in Patent Document 1 (PCT International Publication No. 2015/111848) was used in place of the compound Z(1-2) in Z Element Example—1, and evaluated. The obtained measurement results are shown in Table 3.

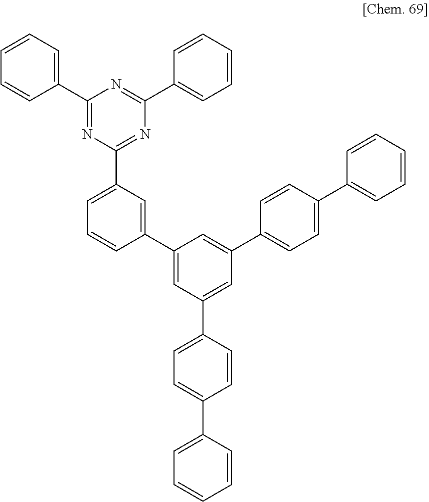 [SMILES: c1ccc(-c2ccc(-c3cc(-c4ccc(-c5ccccc5)cc4)cc(-c4cccc(-c5nc(-c6ccccc6)nc(-c6ccccc6)n5)c4)c3)cc2)cc1]

TABLE 3

| | | | Current | Element |
|---|---|---|---|---|
| | Compound | Voltage | efficiency | lifetime |
| Z Element Example-1 | 1-2 | 96 | 107 | 150 |
| Z Element Example-2 | 1-13 | 98 | 115 | 105 |
| Z Element Example-3 | 1-15 | 95 | 106 | 140 |
| Z Element Reference | ETL-1 | 100 | 100 | 100 |
| Example-1 | | | | |

The triazine compound (1) according to an aspect of the present invention has a wide band-gap, and a high triplet excitation level. Therefore, the triazine compound (1) can be suitably used for not only conventional fluorescence element applications, but also phosphorescence elements and organic light emitting diodes utilizing the thermally activated delayed fluorescence (TADF).

### EXPLANATION OF REFERENCE NUMERALS

1, 101 substrate
2, 102 anode
3, 103 hole injection layer
4, 104 charge generation layer
5, 105 hole transport layer
6, 106 light emitting layer
7, 107 electron transport layer
8, 108 cathode
51, 1051 first hole transport layer
52, 1052 second hole transport layer
71, 1071 first electron transport layer
72, 1072 second electron transport layer
100 organic light emitting diode

1. A triazine compound represented by formula (1): 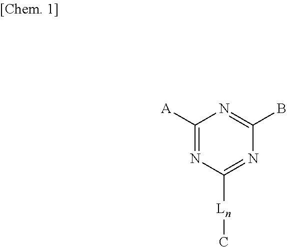
wherein in the formula (1),
A and B each independently represent an aryl group having 6 to 20 carbon atoms;
L represents a phenyl group or a naphthyl group;
n is 0 or 1; and
C represents any one of groups X, Y, and Z below, 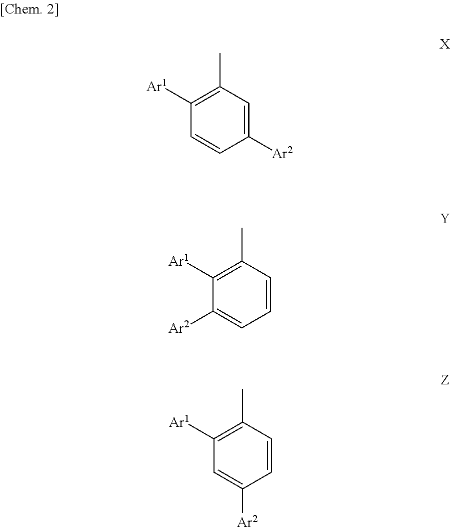
wherein Ar¹ and Ar² each independently represent an aromatic hydrocarbon group or a pyridyl group.

2. A triazine compound represented by formula X(1): 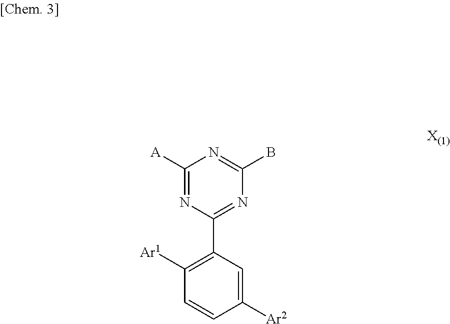
wherein in the formula X(1), A represents any one group selected from formulas X(A-1) to X(A-9): 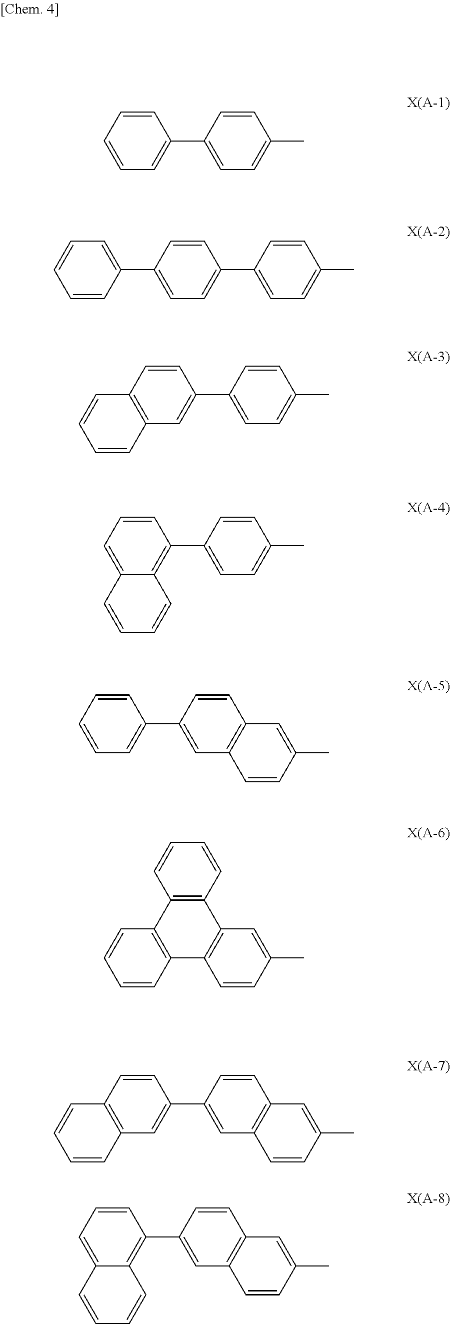 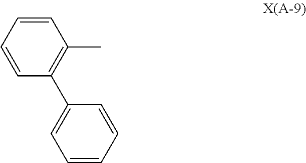
B represents any one group selected from formulas X(B-1) to X(B-15): 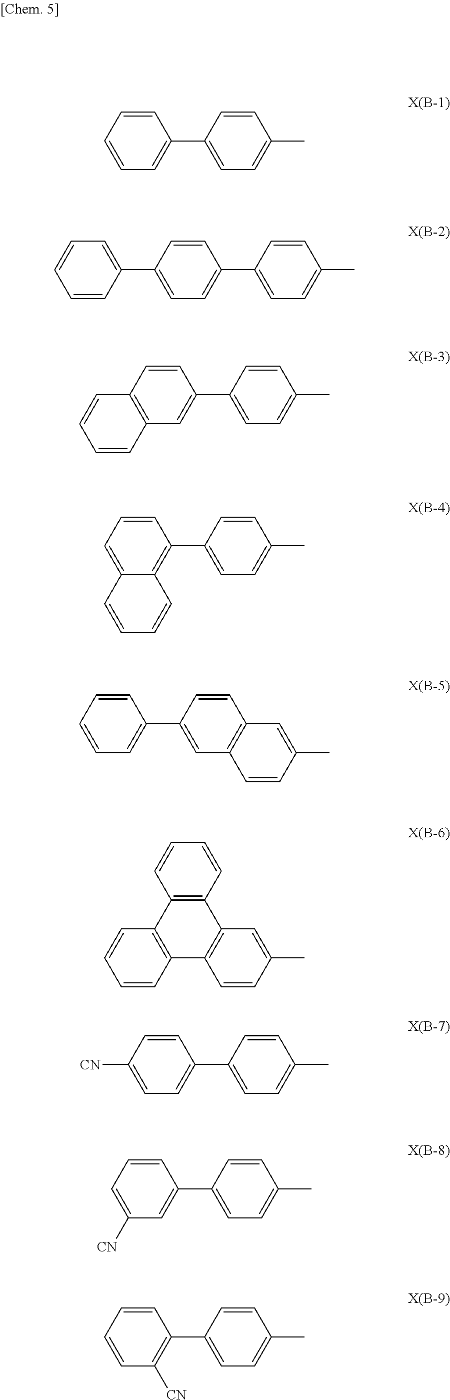 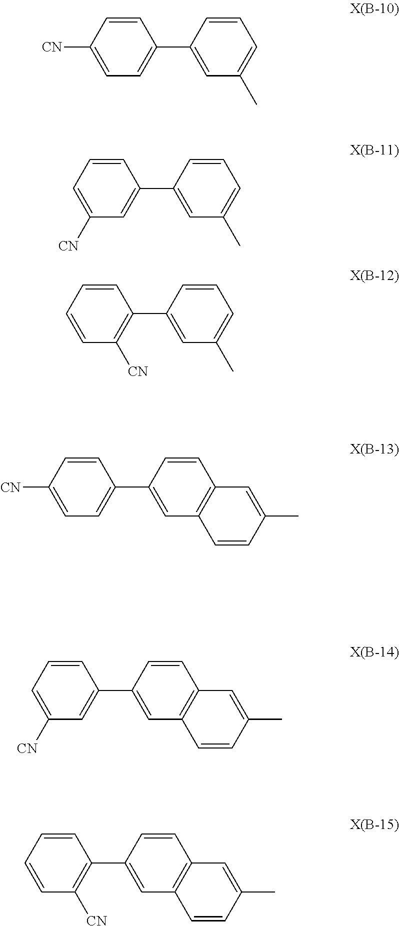
Ar¹ represents: an aryl group having 6 to 30 carbon atoms, optionally substituted with one or more selected from the group consisting of an alkyl group having 1 to 12 carbon atoms, a cycloalkyl group having 3 to 20 carbon atoms, a cyano group, a diarylboryl group, and a phosphine oxide group, or a pyridyl group optionally substituted with a methyl group or a phenyl group; and Ar² represents: an aryl group having 6 to 30 carbon atoms, optionally substituted with one or more selected from the group consisting of an alkyl group having 1 to 12 carbon atoms, a cycloalkyl group having 3 to 20 carbon atoms, a cyano group, a diarylboryl group, and a phosphine oxide group, or a pyridyl group optionally substituted with a methyl group or a phenyl group.

3. The triazine compound according to claim 2, wherein
A represents any one group selected from the formulas X(A-1) to X(A-9),
Ar¹ represents an aryl group having 6 to 30 carbon atoms, optionally substituted with one or more selected from the group consisting of an alkyl group having 1 to 12 carbon atoms, a cycloalkyl group having 3 to 20 carbon atoms, a cyano group, a diarylboryl group, and a phosphine oxide group, and
Ar² represents an aryl group having 6 to 30 carbon atoms, optionally substituted with one or more selected from the group consisting of an alkyl group having 1 to 12 carbon atoms, a cycloalkyl group having 3 to 20 carbon atoms, a cyano group, a diarylboryl group, and a phosphine oxide group.

4. The triazine compound according to claim 2, wherein
A represents any one group selected from the formulas X(A-1) to X(A-4), and B represents any one group selected from the formulas X(B-1) to X(B-4), X(B-7) to X(B-8), and X(B-10) to X(B-11).

5. The triazine compound according to claim 2, wherein
A represents any one group selected from the formulas X(A-1) to X(A-4), B represents a group selected from the formulas X(B-1) to X(B-4), X(B-7) to X(B-8), and X(B-10) to X(B-11),
Ar¹ represents an aryl group having 6 to 30 carbon atoms, optionally substituted with one or more selected from the group consisting of an alkyl group having 1 to 12 carbon atoms, a cycloalkyl group having 3 to 20 carbon atoms, a cyano group, a diarylboryl group, and a phosphine oxide group, and Ar² represents an aryl group having 6 to 30 carbon atoms, optionally substituted with one or more selected from the group consisting of an alkyl group having 1 to 12 carbon atoms, a cycloalkyl group having 3 to 20 carbon atoms, a cyano group, a diarylboryl group, and a phosphine oxide group.

6. The triazine compound according to claim 2, wherein
Ar¹ represents a phenyl group, a 1-naphthalenyl group, a 2-naphthalenyl group, a 2-biphenylyl group, a 3-biphenylyl group, a 4-biphenylyl group, a 2-(1-naphthalenyl)phenyl group, a 3-(1-naphthalenyl)phenyl group, a 4-(1-naphthalenyl)phenyl group, a 2-(2-naphthalenyl)phenyl group, a 3-(2-naphthalenyl)phenyl group, a 4-(2-naphthalenyl)phenyl group, a 4-phenylnaphthalen-1-yl group, a 5-phenylnaphthalen-1-yl group, a 6-phenylnaphthalen-2-yl group, a 7-phenylnaphthalen-2-yl group, a 2-phenanthrenyl group, a 3-phenanthrenyl group, a 9-phenanthrenyl group, a 9-anthracenyl group, a p-terphenyl group, or a 2-triphenylenyl group, each optionally substituted with one or more selected from the group consisting of an alkyl group having 1 to 12 carbon atoms, a cycloalkyl group having 3 to 20 carbon atoms, a cyano group, a diarylboryl group, and a phosphine oxide group, and
Ar² represents a phenyl group, a 1-naphthalenyl group, a 2-naphthalenyl group, a 2-biphenylyl group, a 3-biphenylyl group, a 4-biphenylyl group, a 2-(1-naphthalenyl)phenyl group, a 3-(1-naphthalenyl)phenyl group, a 4-(1-naphthalenyl)phenyl group, a 2-(2-naphthalenyl)phenyl group, a 3-(2-naphthalenyl)phenyl group, a 4-(2-naphthalenyl)phenyl group, a 4-phenylnaphthalen-1-yl group, a 5-phenylnaphthalen-1-yl group, a 6-phenylnaphthalen-2-yl group, a 7-phenylnaphthalen-2-yl group, a 2-phenanthrenyl group, a 3-phenanthrenyl group, a 9-phenanthrenyl group, a 9-anthracenyl group, a p-terphenyl group, or a 2-triphenylenyl group, each optionally substituted with one or more selected from the group consisting of an alkyl group having 1 to 12 carbon atoms, a cycloalkyl group having 3 to 20 carbon atoms, a cyano group, a diarylboryl group, and a phosphine oxide group.

7. The triazine compound according to claim 2, wherein
Ar¹ represents a phenyl group, a 1-naphthalenyl group, a 2-naphthalenyl group, a 2-biphenylyl group, a 3-biphenylyl group, or a 4-biphenylyl group, each optionally substituted with one or more selected from the group consisting of an alkyl group having 1 to 12 carbon atoms, a cycloalkyl group having 3 to 20 carbon atoms, a cyano group, a diarylboryl group, and a phosphine oxide group, and Ar² represents a phenyl group, a 1-naphthalenyl group, a 2-naphthalenyl group, a 2-biphenylyl group, a 3-biphenylyl group, or a 4-biphenylyl group, each optionally substituted with one or more selected from the group consisting of an alkyl group having 1 to 12 carbon atoms, a cycloalkyl group having 3 to 20 carbon atoms, a cyano group, a diarylboryl group, and a phosphine oxide group.

8. The triazine compound according to claim 2, wherein
A represents any one group selected from the formulas X(A-1) to X(A-4),
B represents any one group selected from the formulas X(B-1) to X(B-4),
Ar¹ represents a phenyl group, a 1-naphthalenyl group, a 2-naphthalenyl group, a 2-biphenylyl group, a 3-biphenylyl group, or a 4-biphenylyl group, and
Ar² represents a phenyl group, a 1-naphthalenyl group, a 2-naphthalenyl group, a 2-biphenylyl group, a 3-biphenylyl group, or a 4-biphenylyl group.

9. The triazine compound according to claim 2, wherein
A represents a group represented by the formula X(A-1), B represents a group represented by the formula X(B-1), Ar¹ represents a phenyl group, a 1-naphthalenyl group, a 2-naphthalenyl group, a 2-biphenylyl group, a 3-biphenylyl group, or a 4-biphenylyl group, and
Ar² represents a phenyl group, a 1-naphthalenyl group, a 2-naphthalenyl group, a 2-biphenylyl group, a 3-biphenylyl group, or a 4-biphenylyl group.

10. A triazine compound represented by the formula Y(1): 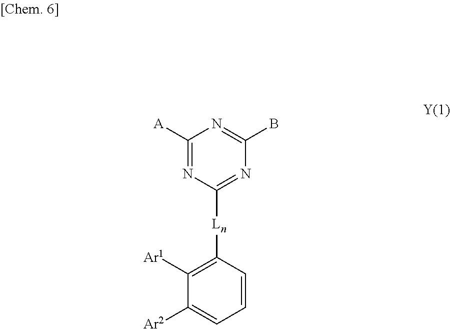
wherein in the formula Y(1), A represents an aryl group having 6 to 20 carbon atoms;
B represents:
an aryl group having 6 to 20 carbon atoms, optionally substituted with one or more selected from the group consisting of an alkyl group having 1 to 12 carbon atoms, a cycloalkyl group having 3 to 20 carbon atoms, a cyano group, a diarylboryl group, and a phosphine oxide group, or
a heteroaryl group having 4 to 30 carbon atoms and an oxygen or sulfur atom; Ar¹ and Ar² each independently represent: an aryl group having 6 to 26 carbon atoms, optionally substituted with one or more selected from the group consisting of an alkyl group having 1 to 12 carbon atoms, a cycloalkyl group having 3 to 20 carbon atoms, a cyano group, a diarylboryl group, and a phosphine oxide group, or a pyridinyl group optionally substituted with a methyl group or a phenyl group; n represents an integer of 0 to 1; and L represents any one group selected from formulas Y(2-1) to Y(2-5).

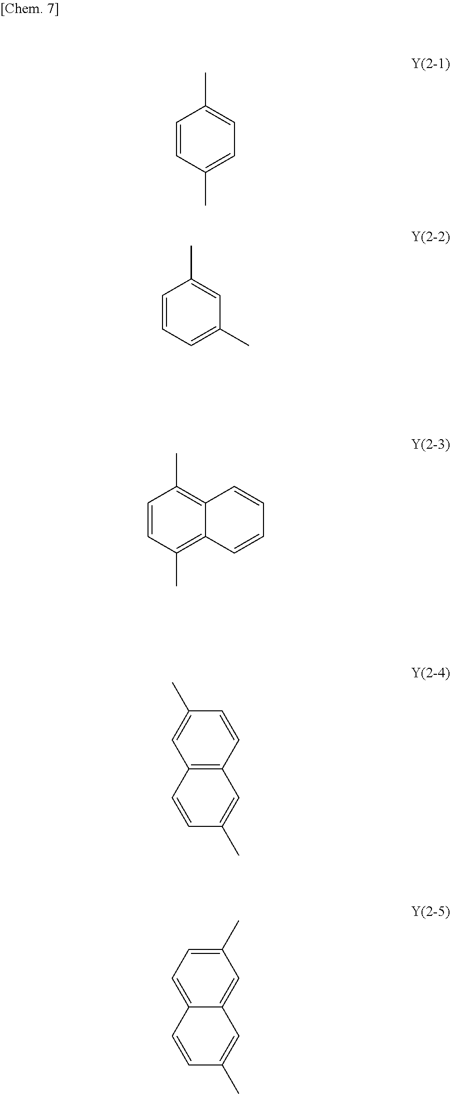

11. The triazine compound according to claim 10, wherein
A represents a phenyl group, a naphthalenyl group, a biphenylyl group, a naphthylphenyl group, a phenylnaphthyl group, a binaphthyl group, a phenanthrenyl group, an anthracenyl group, a terphenyl group, or a triphenylenyl group, and B represents a phenyl group, a naphthalenyl group, a biphenylyl group, a naphthylphenyl group, a phenylnaphthyl group, a binaphthyl group, a phenanthrenyl group, an anthracenyl group, a terphenyl group, a triphenylenyl group, a dibenzofuranyl group, a dibenzothienyl group, or a spiro[9H-fluorene-9,9′-[9H]xanthen]-yl group, each optionally substituted with one or more selected from the group consisting of an alkyl group having 1 to 12 carbon atoms, a cycloalkyl group having 3 to 20 carbon atoms, a cyano group, a diarylboryl group, and a phosphine oxide group.

12. The triazine compound according to claim 10, wherein
A represents a group selected from a phenyl group, a biphenylyl group, a naphthylphenyl group, a phenylnaphthyl group, a binaphthyl group, or a terphenyl group, and B represents a phenyl group, a biphenylyl group, a naphthylphenyl group, a phenylnaphthyl group, or a terphenyl group, each optionally substituted with a cyano group.

13. The triazine compound according to claim 10, wherein
A represents any one group selected from formulas Y(A-1) to Y(A-10), and B represents any one group selected from formulas Y(B-1) to Y(B-28).

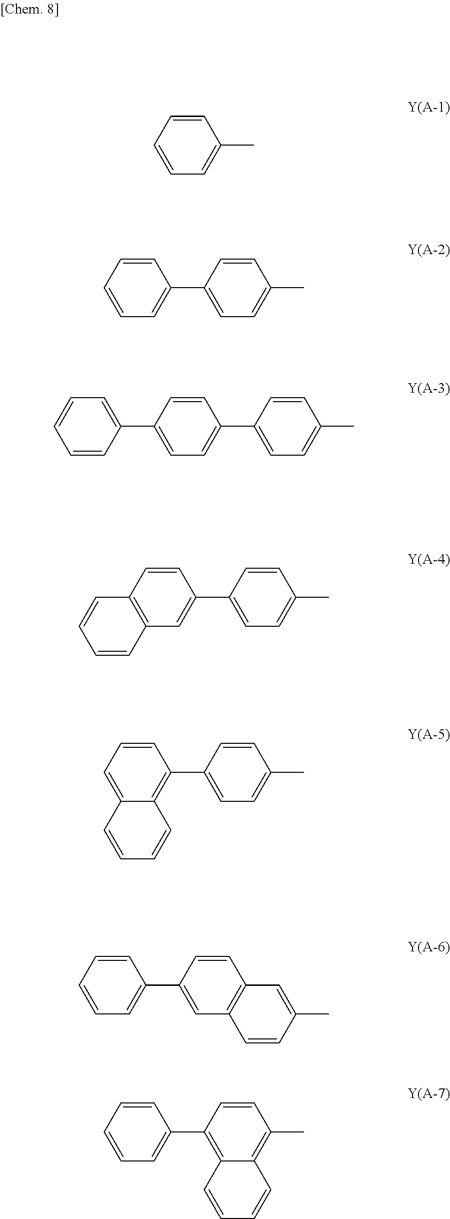

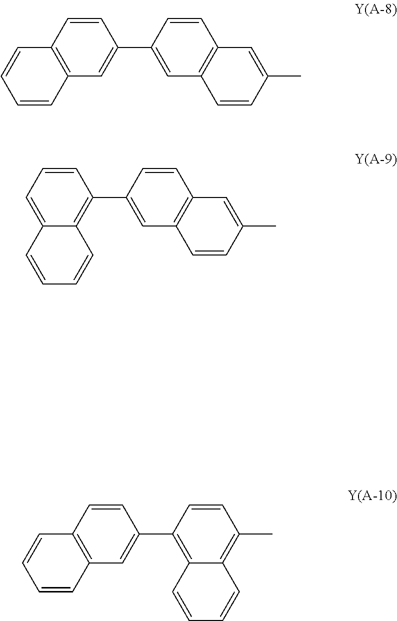

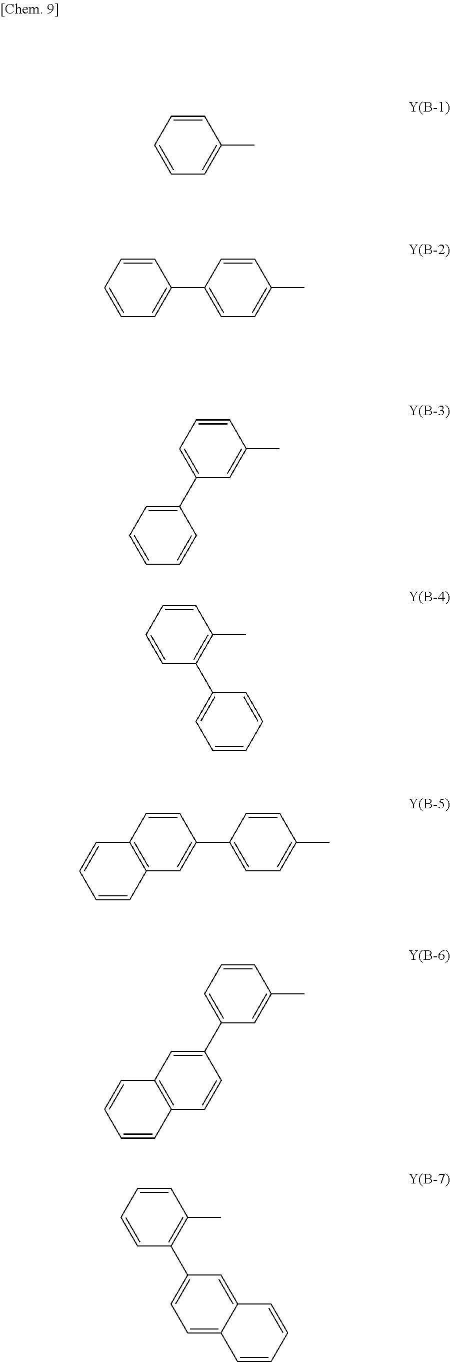

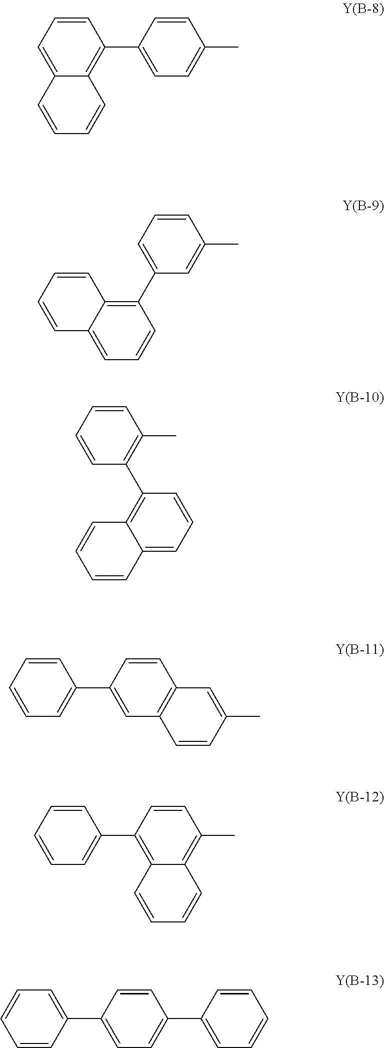

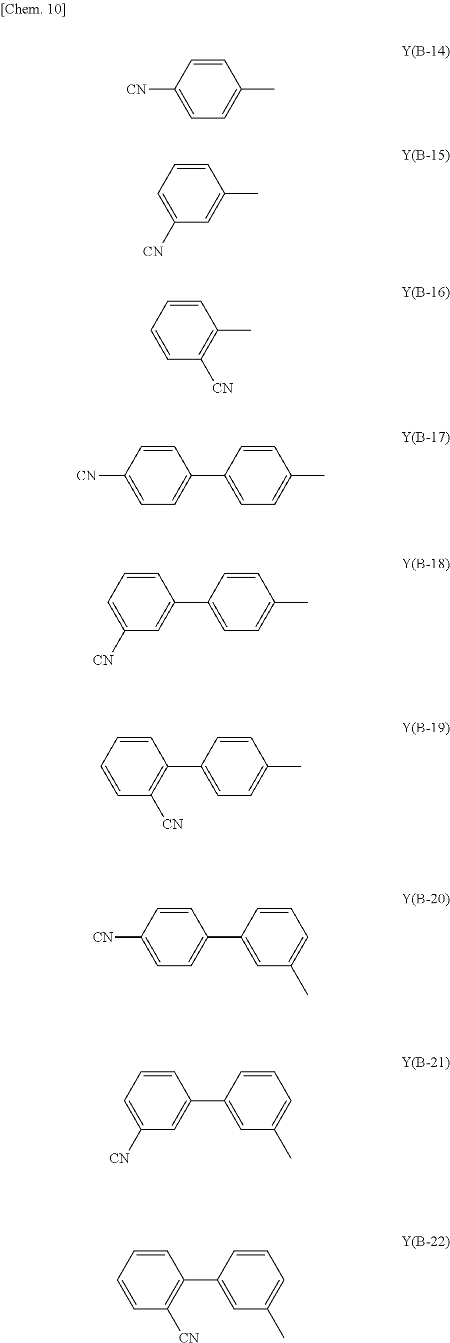

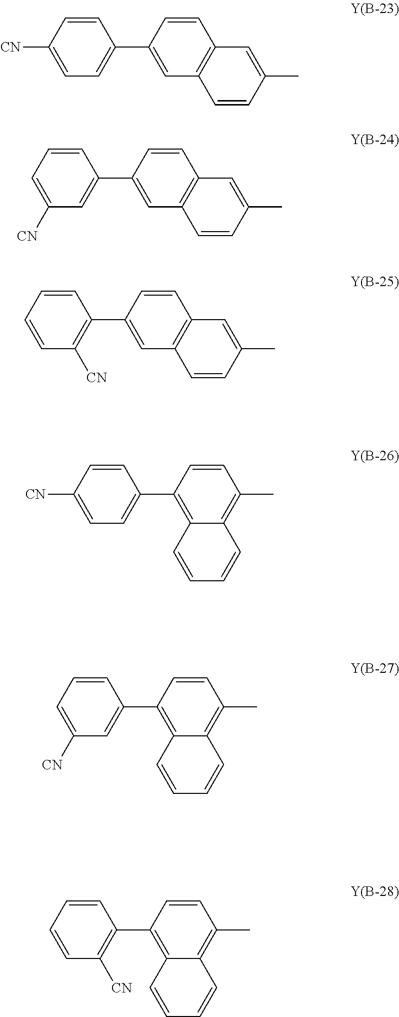

14. The triazine compound according to claim 10, wherein Ar¹ and Ar² each independently represent a phenyl group, a 1-naphthyl group, a 2-naphthyl group, a 2-pyridinyl group, a 3-pyridinyl group, a 4-pyridinyl group, a biphenylyl group, a naphthylphenyl group, a phenylnaphthyl group, a binaphthyl group, an anthracenylphenyl group, or a phenylanthracenylphenyl group, each optionally substituted with a cyano group.

15. The triazine compound according to claim 10, wherein Ar¹ and Ar² each independently represent an unsubstituted phenyl group, an unsubstituted 1-naphthyl group, an unsubstituted 2-naphthyl group, an unsubstituted 2-pyridinyl group, an unsubstituted 3-pyridinyl group, an unsubstituted 4-pyridinyl group, an unsubstituted biphenylyl group, an unsubstituted naphthylphenyl group, an unsubstituted phenylnaphthyl group, an unsubstituted binaphthyl group, an unsubstituted anthracenylphenyl group, or an unsubstituted phenylanthracenylphenyl group.

16. A triazine compound represented by formula Z(1): 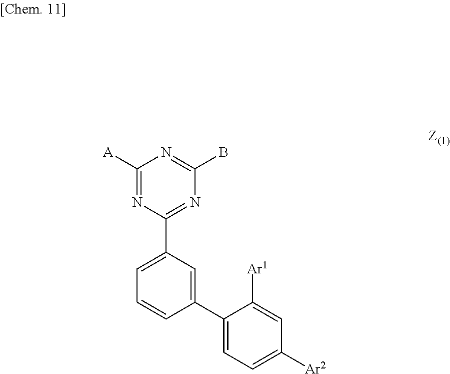
wherein in the formula Z(1),
A represents a phenyl group, a biphenylyl group, or a naphthyl group, each optionally substituted with one or more substituent(s) selected from the group consisting of a fluorine atom, a methyl group, and a cyano group;
B represents a phenyl group, a biphenylyl group, or a naphthyl group, each optionally substituted with one or more substituent(s) selected from the group consisting of a fluorine atom, a methyl group, and a cyano group; Ar¹ represents a phenyl group or a naphthyl group, each optionally substituted with one or more substituent(s) selected from the group consisting of a fluorine atom, a methyl group, and a cyano group; and Ar² represents a phenyl group or a naphthyl group, each optionally substituted with one or more substituent(s) selected from the group consisting of a fluorine atom, a methyl group, and a cyano group.

17. The triazine compound according to claim 16, wherein
Ar¹ represents an unsubstituted phenyl group or an unsubstituted naphthyl group, and
Ar² represents an unsubstituted phenyl group or an unsubstituted naphthyl group.

18. The triazine compound according to claim 16, wherein
A represents an unsubstituted phenyl group, an unsubstituted biphenylyl group, or an unsubstituted naphthyl group,
B represents an unsubstituted phenyl group, an unsubstituted biphenylyl group, or an unsubstituted naphthyl group.

19. The triazine compound according to claim 16, wherein
Ar¹ represents an unsubstituted phenyl group, and Ar² represents an unsubstituted phenyl group or an unsubstituted naphthyl group.

20. The triazine compound according to claim 16, represented by formula Z(1-2), Z(1-3), Z(1-4), Z(1-12), Z(1-13), or Z(1-16).

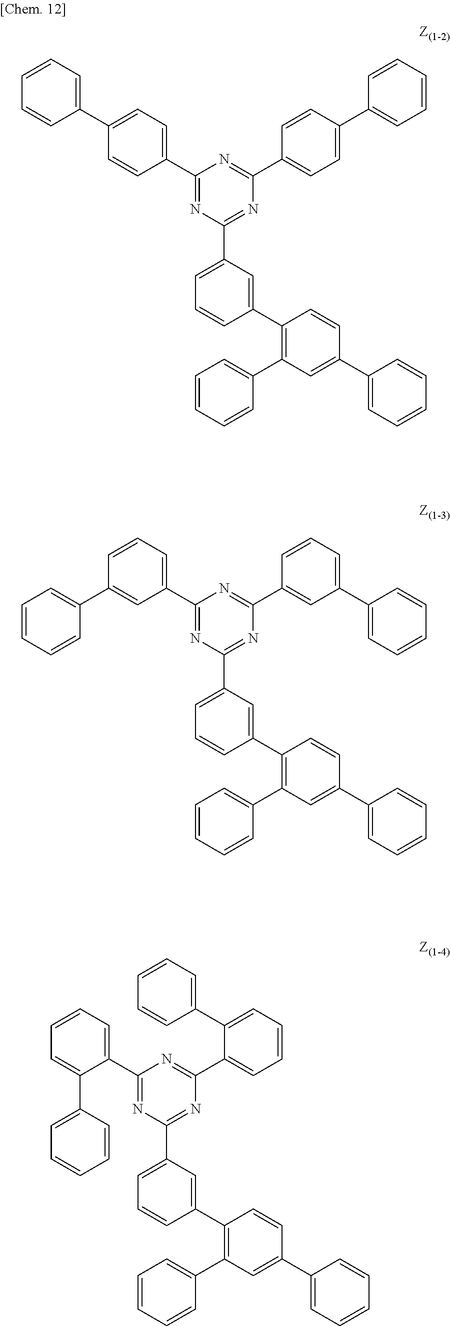 [SMILES: c1ccc(-c2ccc(-c3nc(-c4ccc(-c5ccccc5)cc4)nc(-c4cccc(-c5ccc(-c6ccccc6)cc5-c5ccccc5)c4)n3)cc2)cc1 ; c1ccc(-c2cccc(-c3nc(-c4cccc(-c5ccccc5)c4)nc(-c4cccc(-c5ccc(-c6ccccc6)cc5-c5ccccc5)c4)n3)c2)cc1 ; c1ccc(-c2ccc(-c3cccc(-c4nc(-c5ccccc5-c5ccccc5)nc(-c5ccccc5-c5ccccc5)n4)c3)c(-c3ccccc3)c2)cc1]

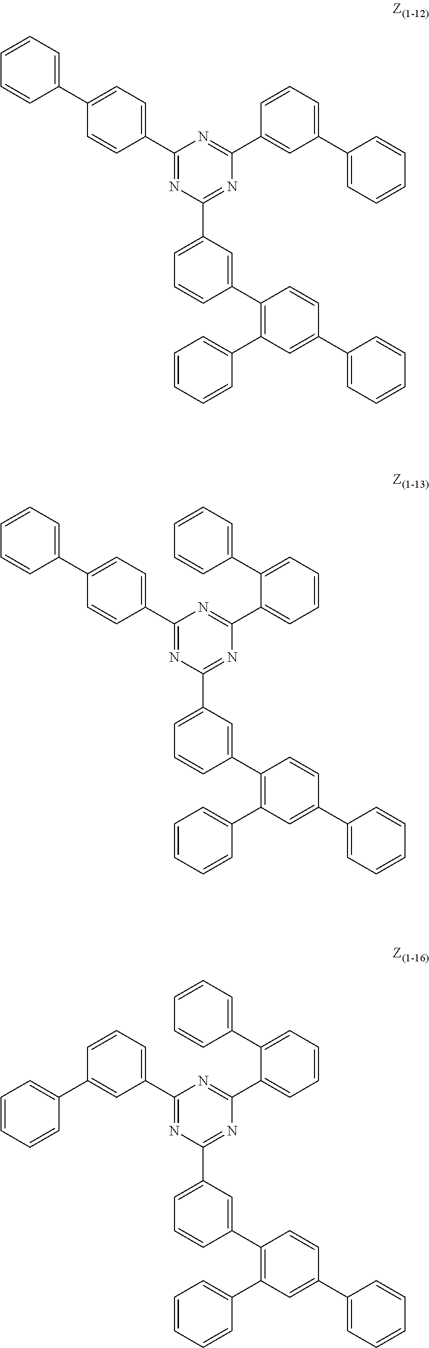 [SMILES: c1ccc(-c2ccc(-c3nc(-c4cccc(-c5ccccc5)c4)nc(-c4cccc(-c5ccc(-c6ccccc6)cc5-c5ccccc5)c4)n3)cc2)cc1 ; c1ccc(-c2ccc(-c3nc(-c4cccc(-c5ccc(-c6ccccc6)cc5-c5ccccc5)c4)nc(-c4ccccc4-c4ccccc4)n3)cc2)cc1 ; c1ccc(-c2cccc(-c3nc(-c4cccc(-c5ccc(-c6ccccc6)cc5-c5ccccc5)c4)nc(-c4ccccc4-c4ccccc4)n3)c2)cc1]

21. A material for an organic light emitting diode, comprising the triazine compound according to claim 1.

22. An electron transport material for an organic light emitting diode, comprising the triazine compound according to claim 1.

23. An organic light emitting diode comprising the triazine compound according to claim 1.
